SEQUENCE LISTING (1548 sequences)

SEQ ID NO: 1 - DNA, 20 bp, Artificial Sequence
  Description of Artificial Sequence Synthetic oligonucleotide
gggcacaagggcacagactt

SEQ ID NO: 2 - DNA, 20 bp, Artificial Sequence
  Description of Artificial Sequence Synthetic oligonucleotide
ggcacaagggcacagacttc

SEQ ID NO: 3 - DNA, 20 bp, Artificial Sequence
  Description of Artificial Sequence Synthetic oligonucleotide
gcacaagggcacagacttcc

SEQ ID NO: 4 - DNA, 20 bp, Artificial Sequence
  Description of Artificial Sequence Synthetic oligonucleotide
cacaagggcacagacttcca

SEQ ID NO: 5 - DNA, 20 bp, Artificial Sequence
  Description of Artificial Sequence Synthetic oligonucleotide
acaagggcacagacttccaa

SEQ ID NO: 6 - DNA, 20 bp, Artificial Sequence
  Description of Artificial Sequence Synthetic oligonucleotide
caagggcacagacttccaaa

SEQ ID NO: 7 - DNA, 20 bp, Artificial Sequence
  Description of Artificial Sequence Synthetic oligonucleotide
gggcacaagggcacagactt

SEQ ID NO: 8 - DNA, 20 bp, Artificial Sequence
  Description of Artificial Sequence Synthetic oligonucleotide
ggcacaagggcacagacttc

SEQ ID NO: 9 - DNA, 20 bp, Artificial Sequence
  Description of Artificial Sequence Synthetic oligonucleotide
gcauaagggcacagacttcc

SEQ ID NO: 10 - DNA, 20 bp, Artificial Sequence
  Description of Artificial Sequence Synthetic oligonucleotide
cacaagggcacagacttcca

SEQ ID NO: 11 - DNA, 20 bp, Artificial Sequence
  Description of Artificial Sequence Synthetic oligonucleotide
acaagggcacagacttccaa

SEQ ID NO: 12 - DNA, 20 bp, Artificial Sequence
  Description of Artificial Sequence Synthetic oligonucleotide
caagggcacagacttccaaa

SEQ ID NO: 13 - RNA, 20 nt, Artificial Sequence
  Description of Artificial Sequence Synthetic oligonucleotide
gggcacaagggcacagacuu

SEQ ID NO: 14 - RNA, 20 nt, Artificial Sequence
  Description of Artificial Sequence Synthetic oligonucleotide
ggcacaagggcacagacuuc

SEQ ID NO: 15 - RNA, 20 nt, Artificial Sequence
  Description of Artificial Sequence Synthetic oligonucleotide
gcacaagggcacagacuucc

SEQ ID NO: 16 - RNA, 20 nt, Artificial Sequence
  Description of Artificial Sequence Synthetic oligonucleotide
cauaagggcacagacuucca

SEQ ID NO: 17 - DNA, 20 bp, Artificial Sequence
  Description of Artificial Sequence Synthetic oligonucleotide
acaagggcacagactuccaa

SEQ ID NO: 18 - DNA, 20 bp, Artificial Sequence
  Description of Artificial Sequence Synthetic oligonucleotide
caagggcacagacttccaaa

SEQ ID NO: 19 - RNA, 20 nt, Artificial Sequence
  Description of Artificial Sequence Synthetic oligonucleotide
gcacaagggcacagacuucc

SEQ ID NO: 20 - RNA, 20 nt, Artificial Sequence
  Description of Artificial Sequence Synthetic oligonucleotide
cacaagggcacagacuucca

SEQ ID NO: 21 - RNA, 20 nt, Artificial Sequence
  Description of Artificial Sequence Synthetic oligonucleotide
acaagggcacagacuuccaa

SEQ ID NO: 22 - RNA, 20 nt, Artificial Sequence
  Description of Artificial Sequence Synthetic oligonucleotide
caagggcacagacuuccaaa

SEQ ID NO: 23 - RNA, 20 nt, Artificial Sequence
  Description of Artificial Sequence Synthetic oligonucleotide
gcacaagggcacagacuucc

SEQ ID NO: 24 - RNA, 20 nt, Artificial Sequence
  Description of Artificial Sequence Synthetic oligonucleotide
cacaagggcacagacuucca

SEQ ID NO: 25 - RNA, 20 nt, Artificial Sequence
  Description of Artificial Sequence Synthetic oligonucleotide
acaagggcacagacuuccaa

SEQ ID NO: 26 - RNA, 20 nt, Artificial Sequence
  Description of Artificial Sequence Synthetic oligonucleotide
caagggcacagacuuccaaa

SEQ ID NO: 27 - RNA, 20 nt, Artificial Sequence
  Description of Artificial Sequence Synthetic oligonucleotide
gggcacaagggcacagacuu

SEQ ID NO: 28 - RNA, 20 nt, Artificial Sequence
  Description of Artificial Sequence Synthetic oligonucleotide
ggcacaagggcacagacuuc

SEQ ID NO: 29 - RNA, 20 nt, Artificial Sequence
  Description of Artificial Sequence Synthetic oligonucleotide
gcacaagggcacagacuucc

SEQ ID NO: 30 - RNA, 20 nt, Artificial Sequence
  Description of Artificial Sequence Synthetic oligonucleotide
cacaagggcacagacuucca

SEQ ID NO: 31 - DNA, 20 bp, Artificial Sequence
  Description of Artificial Sequence Synthetic oligonucleotide
acaagggcacagactuccaa

SEQ ID NO: 32 - DNA, 20 bp, Artificial Sequence
  Description of Artificial Sequence Synthetic oligonucleotide
caagggcacagacttccaaa

SEQ ID NO: 33 - DNA, 20 bp, Artificial Sequence
  Description of Artificial Sequence Synthetic oligonucleotide
gggcacaagggcacagactt

SEQ ID NO: 34 - DNA, 20 bp, Artificial Sequence
  Description of Artificial Sequence Synthetic oligonucleotide
ggcacaagggcacagacttc

SEQ ID NO: 35 - DNA, 20 bp, Artificial Sequence
  Description of Artificial Sequence Synthetic oligonucleotide
gcacaagggcacagacttcc

SEQ ID NO: 36 - DNA, 20 bp, Artificial Sequence
  Description of Artificial Sequence Synthetic oligonucleotide
cacaagggcacagacttcca

SEQ ID NO: 37 - DNA, 20 bp, Artificial Sequence
  Description of Artificial Sequence Synthetic oligonucleotide
acaagggcacagacttccaa

SEQ ID NO: 38 - DNA, 20 bp, Artificial Sequence
  Description of Artificial Sequence Synthetic oligonucleotide
caagggcacagacttccaaa

SEQ ID NO: 39 - RNA, 20 nt, Artificial Sequence
  Description of Artificial Sequence Synthetic oligonucleotide
ggcacaagggcacagacuuc

SEQ ID NO: 40 - RNA, 20 nt, Artificial Sequence
  Description of Artificial Sequence Synthetic oligonucleotide
ggcacaagggcacagacuuc

SEQ ID NO: 41 - RNA, 20 nt, Artificial Sequence
  Description of Artificial Sequence Synthetic oligonucleotide
ggcacaagggcacagacuuc

SEQ ID NO: 42 - DNA, 20 bp, Artificial Sequence
  Description of Artificial Sequence Synthetic oligonucleotide
ggcacaagggcacagacttc

SEQ ID NO: 43 - DNA, 20 bp, Artificial Sequence
  Description of Artificial Sequence Synthetic oligonucleotide
ggcauaagggcacagacttc

SEQ ID NO: 44 - DNA, 20 bp, Artificial Sequence
  Description of Artificial Sequence Synthetic oligonucleotide
ggcacaagggcacagacttc

SEQ ID NO: 45 - RNA, 20 nt, Artificial Sequence
  Description of Artificial Sequence Synthetic oligonucleotide
ggcacaagggcacagacuuc

SEQ ID NO: 46 - RNA, 20 nt, Artificial Sequence
  Description of Artificial Sequence Synthetic oligonucleotide
ggcacaagggcacagacuuc

SEQ ID NO: 47 - RNA, 20 nt, Artificial Sequence
  Description of Artificial Sequence Synthetic oligonucleotide
gcagggcacaagggcacaga

SEQ ID NO: 48 - RNA, 20 nt, Artificial Sequence
  Description of Artificial Sequence Synthetic oligonucleotide
cagggcacaagggcacagac

SEQ ID NO: 49 - DNA, 20 bp, Artificial Sequence
  Description of Artificial Sequence Synthetic oligonucleotide
agggcacaagggcacagact

SEQ ID NO: 50 - DNA, 20 bp, Artificial Sequence
  Description of Artificial Sequence Synthetic oligonucleotide
aagggcacagacttccaaag

SEQ ID NO: 51 - DNA, 20 bp, Artificial Sequence
  Description of Artificial Sequence Synthetic oligonucleotide
agggcacagacttccaaagg

SEQ ID NO: 52 - DNA, 20 bp, Artificial Sequence
  Description of Artificial Sequence Synthetic oligonucleotide
gggcacagacttccaaaggc

SEQ ID NO: 53 - RNA, 20 nt, Artificial Sequence
  Description of Artificial Sequence Synthetic oligonucleotide
ggcacaagggcacagacuuc

SEQ ID NO: 54 - RNA, 20 nt, Artificial Sequence
  Description of Artificial Sequence Synthetic oligonucleotide
ggcacaagggcacagacuuc

SEQ ID NO: 55 - DNA, 20 bp, Artificial Sequence
  Description of Artificial Sequence Synthetic oligonucleotide
  modified_base (13)..(13): 2-amino purine
  misc_feature (13)..(13): n is a, c, g, or t
gggcacaagggcncagactt

SEQ ID NO: 56 - DNA, 20 bp, Artificial Sequence
  Description of Artificial Sequence Synthetic oligonucleotide
  modified_base (12)..(12): 2-amino purine
  misc_feature (12)..(12): n is a, c, g, or t
ggcacaagggcncagacttc

SEQ ID NO: 57 - DNA, 20 bp, Artificial Sequence
  Description of Artificial Sequence Synthetic oligonucleotide
  modified_base (11)..(11): 2-amino purine
  misc_feature (11)..(11): n is a, c, g, or t
gcacaagggcncagacttcc

SEQ ID NO: 58 - DNA, 20 bp, Artificial Sequence
  Description of Artificial Sequence Synthetic oligonucleotide
  modified_base (13)..(13): 2,6-diamino purine
  misc_feature (13)..(13): n is a, c, g, or t
gggcacaagggcncagactt

SEQ ID NO: 59 - DNA, 20 bp, Artificial Sequence
  Description of Artificial Sequence Synthetic oligonucleotide
  modified_base (12)..(12): 2,6-diamino purine
  misc_feature (12)..(12): n is a, c, g, or t
ggcacaagggcncagacttc

SEQ ID NO: 60 - DNA, 20 bp, Artificial Sequence
  Description of Artificial Sequence Synthetic oligonucleotide
  modified_base (11)..(11): 2,6-diamino purine
  misc_feature (11)..(11): n is a, c, g, or t
gcacaagggcncagacttcc

SEQ ID NO: 61 - RNA, 20 nt, Artificial Sequence
  Description of Artificial Sequence Synthetic oligonucleotide
ggcacaagggcacagacuuc

SEQ ID NO: 62 - RNA, 20 nt, Artificial Sequence
  Description of Artificial Sequence Synthetic oligonucleotide
ggcacaagggcacagacuuc

SEQ ID NO: 63 - RNA, 20 nt, Artificial Sequence
  Description of Artificial Sequence Synthetic oligonucleotide
ggcacaagggcacagacuuc

SEQ ID NO: 64 - DNA, 20 bp, Artificial Sequence
  Description of Artificial Sequence Synthetic oligonucleotide
ggcacaagggcacagacttc

SEQ ID NO: 65 - DNA, 20 bp, Artificial Sequence
  Description of Artificial Sequence Synthetic oligonucleotide
gagcagctgcaacctggcaa

SEQ ID NO: 66 - DNA, 20 bp, Artificial Sequence
  Description of Artificial Sequence Synthetic oligonucleotide
agcagctgcaacctggcaac

SEQ ID NO: 67 - DNA, 20 bp, Artificial Sequence
  Description of Artificial Sequence Synthetic oligonucleotide
gcagctgcaacctggcaaca

SEQ ID NO: 68 - DNA, 20 bp, Artificial Sequence
  Description of Artificial Sequence Synthetic oligonucleotide
cagctgcaacctggcaacaa

SEQ ID NO: 69 - DNA, 20 bp, Artificial Sequence
  Description of Artificial Sequence Synthetic oligonucleotide
agctgcaacctggcaacaac

SEQ ID NO: 70 - DNA, 20 bp, Artificial Sequence
  Description of Artificial Sequence Synthetic oligonucleotide
gctgcaacctggcaacaacc

SEQ ID NO: 71 - DNA, 20 bp, Artificial Sequence
  Description of Artificial Sequence Synthetic oligonucleotide
gagcagctgcaacctggcaa

SEQ ID NO: 72 - DNA, 20 bp, Artificial Sequence
  Description of Artificial Sequence Synthetic oligonucleotide
agcagctgcaacctggcaac

SEQ ID NO: 73 - DNA, 20 bp, Artificial Sequence
  Description of Artificial Sequence Synthetic oligonucleotide
gcagctgcaacctggcaaca

SEQ ID NO: 74 - DNA, 20 bp, Artificial Sequence
  Description of Artificial Sequence Synthetic oligonucleotide
cagcugcaacctggcaacaa

SEQ ID NO: 75 - DNA, 20 bp, Artificial Sequence
  Description of Artificial Sequence Synthetic oligonucleotide
agcugcaacctggcaacaac

SEQ ID NO: 76 - DNA, 20 bp, Artificial Sequence
  Description of Artificial Sequence Synthetic oligonucleotide
gcugcaacctggcaacaacc

SEQ ID NO: 77 - DNA, 20 bp, Artificial Sequence
  Description of Artificial Sequence Synthetic oligonucleotide
gagcagctgcaacctggcaa

SEQ ID NO: 78 - DNA, 20 bp, Artificial Sequence
  Description of Artificial Sequence Synthetic oligonucleotide
agcagctgcaacctggcaac

SEQ ID NO: 79 - DNA, 20 bp, Artificial Sequence
  Description of Artificial Sequence Synthetic oligonucleotide
gcagctgcaacctggcaaca

SEQ ID NO: 80 - DNA, 20 bp, Artificial Sequence
  Description of Artificial Sequence Synthetic oligonucleotide
cagcugcaacctggcaacaa

SEQ ID NO: 81 - DNA, 20 bp, Artificial Sequence
  Description of Artificial Sequence Synthetic oligonucleotide
agcugcaacctggcaacaac

SEQ ID NO: 82 - DNA, 20 bp, Artificial Sequence
  Description of Artificial Sequence Synthetic oligonucleotide
gcuggaacctggcaacaacc

SEQ ID NO: 83 - DNA, 20 bp, Artificial Sequence
  Description of Artificial Sequence Synthetic oligonucleotide
gagcagctgcaaccuggcaa

SEQ ID NO: 84 - DNA, 20 bp, Artificial Sequence
  Description of Artificial Sequence Synthetic oligonucleotide
gagcagctgcaaccuggcaa

SEQ ID NO: 85 - DNA, 20 bp, Artificial Sequence
  Description of Artificial Sequence Synthetic oligonucleotide
agcagctgcaacctggcaac

SEQ ID NO: 86 - DNA, 20 bp, Artificial Sequence
  Description of Artificial Sequence Synthetic oligonucleotide
agcagctgcaacctggcaac

SEQ ID NO: 87 - DNA, 20 bp, Artificial Sequence
  Description of Artificial Sequence Synthetic oligonucleotide
ggagctgcaaccgggcaaca

SEQ ID NO: 88 - DNA, 20 bp, Artificial Sequence
  Description of Artificial Sequence Synthetic oligonucleotide
gcagctgcaaccuggcaaca

SEQ ID NO: 89 - DNA, 20 bp, Artificial Sequence
  Description of Artificial Sequence Synthetic oligonucleotide
gagcagctgcaacctggcaa

SEQ ID NO: 90 - DNA, 20 bp, Artificial Sequence
  Description of Artificial Sequence Synthetic oligonucleotide
agcagctgcaacctggcaac

SEQ ID NO: 91 - DNA, 20 bp, Artificial Sequence
  Description of Artificial Sequence Synthetic oligonucleotide
gcagctgcaacctggcaaca

SEQ ID NO: 92 - DNA, 20 bp, Artificial Sequence
  Description of Artificial Sequence Synthetic oligonucleotide
cagcugcaacctggcaacaa

SEQ ID NO: 93 - DNA, 20 bp, Artificial Sequence
  Description of Artificial Sequence Synthetic oligonucleotide
agcugcaacctggcaacaac

SEQ ID NO: 94 - DNA, 20 bp, Artificial Sequence
  Description of Artificial Sequence Synthetic oligonucleotide
gcugcaacctggcaacaacc

SEQ ID NO: 95 - DNA, 20 bp, Artificial Sequence
  Description of Artificial Sequence Synthetic oligonucleotide
gagcagctgcaacctggcaa

SEQ ID NO: 96 - DNA, 19 bp, Artificial Sequence
  Description of Artificial Sequence Synthetic oligonucleotide
agcagcgcaacctggcaac

SEQ ID NO: 97 - DNA, 20 bp, Artificial Sequence
  Description of Artificial Sequence Synthetic oligonucleotide
gcagctgcaacctggcaaca

SEQ ID NO: 98 - DNA, 20 bp, Artificial Sequence
  Description of Artificial Sequence Synthetic oligonucleotide
cagctgcaacctggcaacaa

SEQ ID NO: 99 - DNA, 20 bp, Artificial Sequence
  Description of Artificial Sequence Synthetic oligonucleotide
agctgcaacctggcaacaac

SEQ ID NO: 100 - DNA, 20 bp, Artificial Sequence
  Description of Artificial Sequence Synthetic oligonucleotide
gctgcaacctggcaacaacc

SEQ ID NO: 101 - DNA, 20 bp, Artificial Sequence
  Description of Artificial Sequence Synthetic oligonucleotide
gggccaacagccagcctgca

SEQ ID NO: 102 - DNA, 20 bp, Artificial Sequence
  Description of Artificial Sequence Synthetic oligonucleotide
ggccaacagccagcctgcag

SEQ ID NO: 103 - DNA, 20 bp, Artificial Sequence
  Description of Artificial Sequence Synthetic oligonucleotide
gccaacagccagcctgtagg

SEQ ID NO: 104 - DNA, 20 bp, Artificial Sequence
  Description of Artificial Sequence Synthetic oligonucleotide
ccaacagccagcctgcagga

SEQ ID NO: 105 - DNA, 20 bp, Artificial Sequence
  Description of Artificial Sequence Synthetic oligonucleotide
caacagccagcctgcaggag

SEQ ID NO: 106 - DNA, 20 bp, Artificial Sequence
  Description of Artificial Sequence Synthetic oligonucleotide
aacagctagcctgcaggagg

SEQ ID NO: 107 - DNA, 20 bp, Artificial Sequence
  Description of Artificial Sequence Synthetic oligonucleotide
gggccaacagccagcctgca

SEQ ID NO: 108 - DNA, 20 bp, Artificial Sequence
  Description of Artificial Sequence Synthetic oligonucleotide
ggccaacagccagcctgcag

SEQ ID NO: 109 - DNA, 20 bp, Artificial Sequence
  Description of Artificial Sequence Synthetic oligonucleotide
gccaacagccagcctgcagg

SEQ ID NO: 110 - DNA, 20 bp, Artificial Sequence
  Description of Artificial Sequence Synthetic oligonucleotide
ccaacagccagcctgcagga

SEQ ID NO: 111 - DNA, 20 bp, Artificial Sequence
  Description of Artificial Sequence Synthetic oligonucleotide
caacagccagcctgcaggag

SEQ ID NO: 112 - DNA, 20 bp, Artificial Sequence
  Description of Artificial Sequence Synthetic oligonucleotide
aacagtcagcctgcaggagg

SEQ ID NO: 113 - RNA, 20 nt, Artificial Sequence
  Description of Artificial Sequence Synthetic oligonucleotide
gggccaacagccagccugca

SEQ ID NO: 114 - RNA, 20 nt, Artificial Sequence
  Description of Artificial Sequence Synthetic oligonucleotide
ggccaacagccagccugcag

SEQ ID NO: 115 - DNA, 20 bp, Artificial Sequence
  Description of Artificial Sequence Synthetic oligonucleotide
gccaacagccagcctguagg

SEQ ID NO: 116 - DNA, 20 bp, Artificial Sequence
  Description of Artificial Sequence Synthetic oligonucleotide
ccaacagccagcctgcagga

SEQ ID NO: 117 - DNA, 20 bp, Artificial Sequence
  Description of Artificial Sequence Synthetic oligonucleotide
caauagccagcctgcaggag

SEQ ID NO: 118 - DNA, 20 bp, Artificial Sequence
  Description of Artificial Sequence Synthetic oligonucleotide
aacagccagcctgcaggagg

SEQ ID NO: 119 - RNA, 20 nt, Artificial Sequence
  Description of Artificial Sequence Synthetic oligonucleotide
gggccaacagccagccugca

SEQ ID NO: 120 - RNA, 20 nt, Artificial Sequence
  Description of Artificial Sequence Synthetic oligonucleotide
gggccaacagccagccugca

SEQ ID NO: 121 - RNA, 20 nt, Artificial Sequence
  Description of Artificial Sequence Synthetic oligonucleotide
ggccaacagccagccugcag

SEQ ID NO: 122 - RNA, 20 nt, Artificial Sequence
  Description of Artificial Sequence Synthetic oligonucleotide
ggccaacagccagccugcag

SEQ ID NO: 123 - RNA, 20 nt, Artificial Sequence
  Description of Artificial Sequence Synthetic oligonucleotide
gccaacagccagccugcagg

SEQ ID NO: 124 - RNA, 20 nt, Artificial Sequence
  Description of Artificial Sequence Synthetic oligonucleotide
gccaacagccagccugcagg

SEQ ID NO: 125 - RNA, 20 nt, Artificial Sequence
  Description of Artificial Sequence Synthetic oligonucleotide
gggccaacagccagccugca

SEQ ID NO: 126 - RNA, 20 nt, Artificial Sequence
  Description of Artificial Sequence Synthetic oligonucleotide
ggccaacagccagccugcag

SEQ ID NO: 127 - DNA, 20 bp, Artificial Sequence
  Description of Artificial Sequence Synthetic oligonucleotide
gccaacagccagcctgcagg

SEQ ID NO: 128 - DNA, 20 bp, Artificial Sequence
  Description of Artificial Sequence Synthetic oligonucleotide
ccaacagccagcctgcagga

SEQ ID NO: 129 - DNA, 20 bp, Artificial Sequence
  Description of Artificial Sequence Synthetic oligonucleotide
caacagccagcctgcaggag

SEQ ID NO: 130 - DNA, 20 bp, Artificial Sequence
  Description of Artificial Sequence Synthetic oligonucleotide
aacagccagcctgcaggagg

SEQ ID NO: 131 - DNA, 20 bp, Artificial Sequence
  Description of Artificial Sequence Synthetic oligonucleotide
gggccaacagccagcctgca

SEQ ID NO: 132 - DNA, 20 bp, Artificial Sequence
  Description of Artificial Sequence Synthetic oligonucleotide
ggccaacagccagcctgcag

SEQ ID NO: 133 - DNA, 20 bp, Artificial Sequence
  Description of Artificial Sequence Synthetic oligonucleotide
gccaacagccagcctgcagg

SEQ ID NO: 134 - DNA, 20 bp, Artificial Sequence
  Description of Artificial Sequence Synthetic oligonucleotide
ccaacagccagcctgcagga

SEQ ID NO: 135 - DNA, 20 bp, Artificial Sequence
  Description of Artificial Sequence Synthetic oligonucleotide
caacagccagcctgcaggag

SEQ ID NO: 136 - DNA, 20 bp, Artificial Sequence
  Description of Artificial Sequence Synthetic oligonucleotide
aacagccagcctgcaggagg

SEQ ID NO: 137 - DNA, 20 bp, Artificial Sequence
  Description of Artificial Sequence Synthetic oligonucleotide
attaataaattgtcatcacc

SEQ ID NO: 138 - DNA, 20 bp, Artificial Sequence
  Description of Artificial Sequence Synthetic oligonucleotide
attaataaattgtcatcacc

SEQ ID NO: 139 - DNA, 20 bp, Artificial Sequence
  Description of Artificial Sequence Synthetic oligonucleotide
attaataaattgtcatcacc

SEQ ID NO: 140 - DNA, 20 bp, Artificial Sequence
  Description of Artificial Sequence Synthetic oligonucleotide
auuaauaaattgtcatcacc

SEQ ID NO: 141 - DNA, 20 bp, Artificial Sequence
  Description of Artificial Sequence Synthetic oligonucleotide
auuaauaaattgtcatcacc

SEQ ID NO: 142 - DNA, 20 bp, Artificial Sequence
  Description of Artificial Sequence Synthetic oligonucleotide
auuaauaaattgtcatcacc

SEQ ID NO: 143 - DNA, 20 bp, Artificial Sequence
  Description of Artificial Sequence Synthetic oligonucleotide
auuaauaaattgtcatcacc

SEQ ID NO: 144 - DNA, 20 bp, Artificial Sequence
  Description of Artificial Sequence Synthetic oligonucleotide
auuaauaaattgtcatcacc

SEQ ID NO: 145 - DNA, 20 bp, Artificial Sequence
  Description of Artificial Sequence Synthetic oligonucleotide
auuaauaaattgtcatcacc

SEQ ID NO: 146 - DNA, 20 bp, Artificial Sequence
  Description of Artificial Sequence Synthetic oligonucleotide
auuaauaaattgtcatcacc

SEQ ID NO: 147 - DNA, 20 bp, Artificial Sequence
  Description of Artificial Sequence Synthetic oligonucleotide
auuaauaaattgtcatcacc

SEQ ID NO: 148 - DNA, 20 bp, Artificial Sequence
  Description of Artificial Sequence Synthetic oligonucleotide
auuaauaaattgtcatcacc

SEQ ID NO: 149 - DNA, 20 bp, Artificial Sequence
  Description of Artificial Sequence Synthetic oligonucleotide
auuaauaaattgtcatcacc

SEQ ID NO: 150 - DNA, 20 bp, Artificial Sequence
  Description of Artificial Sequence Synthetic oligonucleotide
tgtcatcaccagaaaaaguc

SEQ ID NO: 151 - DNA, 20 bp, Artificial Sequence
  Description of Artificial Sequence Synthetic oligonucleotide
utgtcatcaccagaaaaagu

SEQ ID NO: 152 - DNA, 20 bp, Artificial Sequence
  Description of Artificial Sequence Synthetic oligonucleotide
ttgtcatcaccagaaaaagu

SEQ ID NO: 153 - DNA, 20 bp, Artificial Sequence
  Description of Artificial Sequence Synthetic oligonucleotide
autgtcatcaccagaaaaag

SEQ ID NO: 154 - DNA, 20 bp, Artificial Sequence
  Description of Artificial Sequence Synthetic oligonucleotide
attgtcatcaccagaaaaag

SEQ ID NO: 155 - DNA, 20 bp, Artificial Sequence
  Description of Artificial Sequence Synthetic oligonucleotide
aautgtcatcaccagaaaaa

SEQ ID NO: 156 - DNA, 20 bp, Artificial Sequence
  Description of Artificial Sequence Synthetic oligonucleotide
aattgtcatcaccagaaaaa

SEQ ID NO: 157 - DNA, 20 bp, Artificial Sequence
  Description of Artificial Sequence Synthetic oligonucleotide
aaattgtcatcaccagaaaa

SEQ ID NO: 158 - DNA, 20 bp, Artificial Sequence
  Description of Artificial Sequence Synthetic oligonucleotide
aaautgtcatcaccagaaaa

SEQ ID NO: 159 - DNA, 19 bp, Artificial Sequence
  Description of Artificial Sequence Synthetic oligonucleotide
aaautgtcatcaccagaaa

SEQ ID NO: 160 - DNA, 19 bp, Artificial Sequence
  Description of Artificial Sequence Synthetic oligonucleotide
aaautgtcatcaccagaaa

SEQ ID NO: 161 - DNA, 20 bp, Artificial Sequence
  Description of Artificial Sequence Synthetic oligonucleotide
auaaattgtcatcaccagaa

SEQ ID NO: 162 - DNA, 20 bp, Artificial Sequence
  Description of Artificial Sequence Synthetic oligonucleotide
auaaattgtcatcaccagaa

SEQ ID NO: 163 - DNA, 20 bp, Artificial Sequence
  Description of Artificial Sequence Synthetic oligonucleotide
aauaaattgtcatcaccaga

SEQ ID NO: 164 - DNA, 20 bp, Artificial Sequence
  Description of Artificial Sequence Synthetic oligonucleotide
aauaaattgtcatcaccaga

SEQ ID NO: 165 - DNA, 20 bp, Artificial Sequence
  Description of Artificial Sequence Synthetic oligonucleotide
aauaaattgtcatcaccaga

SEQ ID NO: 166 - DNA, 20 bp, Artificial Sequence
  Description of Artificial Sequence Synthetic oligonucleotide
uaauaaattgtcatcaccag

SEQ ID NO: 167 - DNA, 20 bp, Artificial Sequence
  Description of Artificial Sequence Synthetic oligonucleotide
uaauaaattgtcatcaccag

SEQ ID NO: 168 - DNA, 20 bp, Artificial Sequence
  Description of Artificial Sequence Synthetic oligonucleotide
uaauaaattgtcatcaccag

SEQ ID NO: 169 - DNA, 20 bp, Artificial Sequence
  Description of Artificial Sequence Synthetic oligonucleotide
uaauaaattgtcatcaccag

SEQ ID NO: 170 - DNA, 20 bp, Artificial Sequence
  Description of Artificial Sequence Synthetic oligonucleotide
uuaauaaattgtcatcacca

SEQ ID NO: 171 - DNA, 20 bp, Artificial Sequence
  Description of Artificial Sequence Synthetic oligonucleotide
uuaauaaattgtcatcacca

SEQ ID NO: 172 - DNA, 20 bp, Artificial Sequence
  Description of Artificial Sequence Synthetic oligonucleotide
uuaauaaattgtcatcacca

SEQ ID NO: 173 - DNA, 20 bp, Artificial Sequence
  Description of Artificial Sequence Synthetic oligonucleotide
uuaauaaattgtcatcacca

SEQ ID NO: 174 - DNA, 20 bp, Artificial Sequence
  Description of Artificial Sequence Synthetic oligonucleotide
auuaataaattgtcatcacc

SEQ ID NO: 175 - DNA, 20 bp, Artificial Sequence
  Description of Artificial Sequence Synthetic oligonucleotide
auuaataaattgtcatcacc

SEQ ID NO: 176 - DNA, 20 bp, Artificial Sequence
  Description of Artificial Sequence Synthetic oligonucleotide
auuaataaattgtcatcacc

SEQ ID NO: 177 - DNA, 20 bp, Artificial Sequence
  Description of Artificial Sequence Synthetic oligonucleotide
auuaataaattgtcatcacc

SEQ ID NO: 178 - DNA, 20 bp, Artificial Sequence
  Description of Artificial Sequence Synthetic oligonucleotide
uauuaataaattgtcatcac

SEQ ID NO: 179 - DNA, 20 bp, Artificial Sequence
  Description of Artificial Sequence Synthetic oligonucleotide
uauuaataaattgtcatcac

SEQ ID NO: 180 - DNA, 20 bp, Artificial Sequence
  Description of Artificial Sequence Synthetic oligonucleotide
uauuaataaattgtcatcac

SEQ ID NO: 181 - DNA, 20 bp, Artificial Sequence
  Description of Artificial Sequence Synthetic oligonucleotide
cuauuaataaattgtcatca

SEQ ID NO: 182 - DNA, 20 bp, Artificial Sequence
  Description of Artificial Sequence Synthetic oligonucleotide
cuauuaataaattgtcatca

SEQ ID NO: 183 - DNA, 20 bp, Artificial Sequence
  Description of Artificial Sequence Synthetic oligonucleotide
acuautaataaattgtcatc

SEQ ID NO: 184 - DNA, 20 bp, Artificial Sequence
  Description of Artificial Sequence Synthetic oligonucleotide
tgtcatcaccagaaaaaguc

SEQ ID NO: 185 - DNA, 20 bp, Artificial Sequence
  Description of Artificial Sequence Synthetic oligonucleotide
utgtcatcaccagaaaaagu

SEQ ID NO: 186 - DNA, 20 bp, Artificial Sequence
  Description of Artificial Sequence Synthetic oligonucleotide
ttgtcatcaccagaaaaagu

SEQ ID NO: 187 - DNA, 20 bp, Artificial Sequence
  Description of Artificial Sequence Synthetic oligonucleotide
autgtcatcaccagaaaaag

SEQ ID NO: 188 - DNA, 20 bp, Artificial Sequence
  Description of Artificial Sequence Synthetic oligonucleotide
attgtcatcaccagaaaaag

SEQ ID NO: 189 - DNA, 20 bp, Artificial Sequence
  Description of Artificial Sequence Synthetic oligonucleotide
aautgtcatcaccagaaaaa

SEQ ID NO: 190 - DNA, 20 bp, Artificial Sequence
  Description of Artificial Sequence Synthetic oligonucleotide
aattgtcatcaccagaaaaa

SEQ ID NO: 191 - DNA, 20 bp, Artificial Sequence
  Description of Artificial Sequence Synthetic oligonucleotide
aaattgtcatcaccagaaaa

SEQ ID NO: 192 - DNA, 20 bp, Artificial Sequence
  Description of Artificial Sequence Synthetic oligonucleotide
aaautgtcatcaccagaaaa

SEQ ID NO: 193 - DNA, 20 bp, Artificial Sequence
  Description of Artificial Sequence Synthetic oligonucleotide
uaaautgtcatcaccagaaa

SEQ ID NO: 194 - DNA, 20 bp, Artificial Sequence
  Description of Artificial Sequence Synthetic oligonucleotide
uaaautgtcatcaccagaaa

SEQ ID NO: 195 - DNA, 20 bp, Artificial Sequence
  Description of Artificial Sequence Synthetic oligonucleotide
auaaattgtcatcaccagaa

SEQ ID NO: 196 - DNA, 20 bp, Artificial Sequence
  Description of Artificial Sequence Synthetic oligonucleotide
auaaattgtcatcaccagaa

SEQ ID NO: 197 - DNA, 20 bp, Artificial Sequence
  Description of Artificial Sequence Synthetic oligonucleotide
aauaaattgtcatcaccaga

SEQ ID NO: 198 - DNA, 20 bp, Artificial Sequence
  Description of Artificial Sequence Synthetic oligonucleotide
aauaaattgtcatcaccaga

SEQ ID NO: 199 - DNA, 20 bp, Artificial Sequence
  Description of Artificial Sequence Synthetic oligonucleotide
aauaaattgtcatcaccaga

SEQ ID NO: 200 - DNA, 20 bp, Artificial Sequence
  Description of Artificial Sequence Synthetic oligonucleotide
uaauaaattgtcatcaccag

SEQ ID NO: 201 - DNA, 20 bp, Artificial Sequence
  Description of Artificial Sequence Synthetic oligonucleotide
uaauaaattgtcatcaccag

SEQ ID NO: 202 - DNA, 20 bp, Artificial Sequence
  Description of Artificial Sequence Synthetic oligonucleotide
uaauaaattgtcatcaccag

SEQ ID NO: 203 - DNA, 20 bp, Artificial Sequence
  Description of Artificial Sequence Synthetic oligonucleotide
uaauaaattgtcatcaccag

SEQ ID NO: 204 - DNA, 20 bp, Artificial Sequence
  Description of Artificial Sequence Synthetic oligonucleotide
uuaauaaattgtcatcacca

SEQ ID NO: 205 - DNA, 20 bp, Artificial Sequence
  Description of Artificial Sequence Synthetic oligonucleotide
uuaauaaattgtcatcacca

SEQ ID NO: 206 - DNA, 20 bp, Artificial Sequence
  Description of Artificial Sequence Synthetic oligonucleotide
uuaauaaattgtcatcacca

SEQ ID NO: 207 - DNA, 20 bp, Artificial Sequence
  Description of Artificial Sequence Synthetic oligonucleotide
uuaauaaattgtcatcacca

SEQ ID NO: 208 - DNA, 20 bp, Artificial Sequence
  Description of Artificial Sequence Synthetic oligonucleotide
auuaataaattgtcatcacc

SEQ ID NO: 209 - DNA, 20 bp, Artificial Sequence
  Description of Artificial Sequence Synthetic oligonucleotide
auuaataaattgtcatcacc

SEQ ID NO: 210 - DNA, 20 bp, Artificial Sequence
  Description of Artificial Sequence Synthetic oligonucleotide
auuaataaattgtcatcacc

SEQ ID NO: 211 - DNA, 20 bp, Artificial Sequence
  Description of Artificial Sequence Synthetic oligonucleotide
auuaataaattgtcatcacc

SEQ ID NO: 212 - DNA, 20 bp, Artificial Sequence
  Description of Artificial Sequence Synthetic oligonucleotide
uauuaataaattgtcatcac

SEQ ID NO: 213 - DNA, 20 bp, Artificial Sequence
  Description of Artificial Sequence Synthetic oligonucleotide
uauuaataaattgtcatcac

SEQ ID NO: 214 - DNA, 20 bp, Artificial Sequence
  Description of Artificial Sequence Synthetic oligonucleotide
uauuaataaattgtcatcac

SEQ ID NO: 215 - DNA, 20 bp, Artificial Sequence
  Description of Artificial Sequence Synthetic oligonucleotide
cuauuaataaattgtcatca

SEQ ID NO: 216 - DNA, 20 bp, Artificial Sequence
  Description of Artificial Sequence Synthetic oligonucleotide
cuauuaataaattgtcatca

SEQ ID NO: 217 - DNA, 20 bp, Artificial Sequence
  Description of Artificial Sequence Synthetic oligonucleotide
acuautaataaattgtcatc

SEQ ID NO: 218 - DNA, 20 bp, Artificial Sequence
  Description of Artificial Sequence Synthetic oligonucleotide
gggcacaagggcacagactt

SEQ ID NO: 219 - DNA, 20 bp, Artificial Sequence
  Description of Artificial Sequence Synthetic oligonucleotide
ggcacaagggcacagacttc

SEQ ID NO: 220 - DNA, 20 bp, Artificial Sequence
  Description of Artificial Sequence Synthetic oligonucleotide
gcacaagggcacagacttcc

SEQ ID NO: 221 - DNA, 20 bp, Artificial Sequence
  Description of Artificial Sequence Synthetic oligonucleotide
cacaagggcacagacttcca

SEQ ID NO: 222 - DNA, 20 bp, Artificial Sequence
  Description of Artificial Sequence Synthetic oligonucleotide
acaagggcacagacttccaa

SEQ ID NO: 223 - DNA, 20 bp, Artificial Sequence
  Description of Artificial Sequence Synthetic oligonucleotide
caaggccacagacttccaaa

SEQ ID NO: 224 - RNA, 20 nt, Artificial Sequence
  Description of Artificial Sequence Synthetic oligonucleotide
ggcacaagggcacagacuuc

SEQ ID NO: 225 - RNA, 20 nt, Artificial Sequence
  Description of Artificial Sequence Synthetic oligonucleotide
ggcacaagggcacagacuuc

SEQ ID NO: 226 - RNA, 20 nt, Artificial Sequence
  Description of Artificial Sequence Synthetic oligonucleotide
ggcacaagggcacagacuuc

SEQ ID NO: 227 - DNA, 20 bp, Artificial Sequence
  Description of Artificial Sequence Synthetic oligonucleotide
ggcacaagggcacagacttc

SEQ ID NO: 228 - DNA, 20 bp, Artificial Sequence
  Description of Artificial Sequence Synthetic oligonucleotide
ggcacaagggcacagacttc

SEQ ID NO: 229 - DNA, 20 bp, Artificial Sequence
  Description of Artificial Sequence Synthetic oligonucleotide
ggcacaagggcacagacttc

SEQ ID NO: 230 - RNA, 20 nt, Artificial Sequence
  Description of Artificial Sequence Synthetic oligonucleotide
ggcacaagggcacagacuuc

SEQ ID NO: 231 - RNA, 20 nt, Artificial Sequence
  Description of Artificial Sequence Synthetic oligonucleotide
ggcacaagggcacagacuuc

SEQ ID NO: 232 - RNA, 20 nt, Artificial Sequence
  Description of Artificial Sequence Synthetic oligonucleotide
gcagggcacaagggcacaga

SEQ ID NO: 233 - RNA, 20 nt, Artificial Sequence
  Description of Artificial Sequence Synthetic oligonucleotide
cagggcacaagggcacagac

SEQ ID NO: 234 - DNA, 20 bp, Artificial Sequence
  Description of Artificial Sequence Synthetic oligonucleotide
agggcacaagggcacagact

SEQ ID NO: 235 - DNA, 20 bp, Artificial Sequence
  Description of Artificial Sequence Synthetic oligonucleotide
aagggcacagacttccaaag

SEQ ID NO: 236 - DNA, 20 bp, Artificial Sequence
  Description of Artificial Sequence Synthetic oligonucleotide
agggcacagacttccaaagg

SEQ ID NO: 237 - DNA, 20 bp, Artificial Sequence
  Description of Artificial Sequence Synthetic oligonucleotide
gggcacagacttccaaaggc

SEQ ID NO: 238 - RNA, 20 nt, Artificial Sequence
  Description of Artificial Sequence Synthetic oligonucleotide
ggcacaagggcacagacuuc

SEQ ID NO: 239 - DNA, 20 bp, Artificial Sequence
  Description of Artificial Sequence Synthetic oligonucleotide
gggccaacagccagcctgca

SEQ ID NO: 240 - DNA, 20 bp, Artificial Sequence
  Description of Artificial Sequence Synthetic oligonucleotide
ggccaacagccagcctgcag

SEQ ID NO: 241 - DNA, 20 bp, Artificial Sequence
  Description of Artificial Sequence Synthetic oligonucleotide
gccaacagccagcctgcagg

SEQ ID NO: 242 - DNA, 20 bp, Artificial Sequence
  Description of Artificial Sequence Synthetic oligonucleotide
ccaacagccagcctgcagga

SEQ ID NO: 243 - DNA, 20 bp, Artificial Sequence
  Description of Artificial Sequence Synthetic oligonucleotide
caacagccagcctgcaggag

SEQ ID NO: 244 - DNA, 20 bp, Artificial Sequence
  Description of Artificial Sequence Synthetic oligonucleotide
aacagccagcctgcaggagg

SEQ ID NO: 245 - DNA, 20 bp, Artificial Sequence
  Description of Artificial Sequence Synthetic oligonucleotide
gagcagctgcaacctggcaa

SEQ ID NO: 246 - DNA, 20 bp, Artificial Sequence
  Description of Artificial Sequence Synthetic oligonucleotide
agcagctgcaacctggcaac

SEQ ID NO: 247 - DNA, 19 bp, Artificial Sequence
  Description of Artificial Sequence Synthetic oligonucleotide
gcagctgcacctggcaaca

SEQ ID NO: 248 - DNA, 20 bp, Artificial Sequence
  Description of Artificial Sequence Synthetic oligonucleotide
cagctgcaacctggcaacaa

SEQ ID NO: 249 - DNA, 20 bp, Artificial Sequence
  Description of Artificial Sequence Synthetic oligonucleotide
agctgcaacctggcaacaac

SEQ ID NO: 250 - DNA, 20 bp, Artificial Sequence
  Description of Artificial Sequence Synthetic oligonucleotide
gctgcaacctggcaacaacc

SEQ ID NO: 251 - DNA, 20 bp, Artificial Sequence
  Description of Artificial Sequence Synthetic oligonucleotide
attaataaattgtcatcacc

SEQ ID NO: 252 - DNA, 20 bp, Artificial Sequence
  Description of Artificial Sequence Synthetic oligonucleotide
attaataaattgtcatcacc

SEQ ID NO: 253 - DNA, 20 bp, Artificial Sequence
  Description of Artificial Sequence Synthetic oligonucleotide
attaataaattgtcatcacc

SEQ ID NO: 254 - DNA, 20 bp, Artificial Sequence
  Description of Artificial Sequence Synthetic oligonucleotide
auuaauaaattgtcatcacc

SEQ ID NO: 255 - DNA, 20 bp, Artificial Sequence
  Description of Artificial Sequence Synthetic oligonucleotide
auuaauaaattgtcatcacc

SEQ ID NO: 256 - DNA, 20 bp, Artificial Sequence
  Description of Artificial Sequence Synthetic oligonucleotide
auuaauaaattgtcatcacc

SEQ ID NO: 257 - DNA, 20 bp, Artificial Sequence
  Description of Artificial Sequence Synthetic oligonucleotide
auuaauaaattgtcatcacc

SEQ ID NO: 258 - DNA, 20 bp, Artificial Sequence
  Description of Artificial Sequence Synthetic oligonucleotide
auuaauaaattgtcatcacc

SEQ ID NO: 259 - DNA, 20 bp, Artificial Sequence
  Description of Artificial Sequence Synthetic oligonucleotide
auuaauaaattgtcatcacc

SEQ ID NO: 260 - DNA, 20 bp, Artificial Sequence
  Description of Artificial Sequence Synthetic oligonucleotide
auuaauaaattgtcatcacc

SEQ ID NO: 261 - DNA, 20 bp, Artificial Sequence
  Description of Artificial Sequence Synthetic oligonucleotide
auuaauaaattgtcatcacc

SEQ ID NO: 262 - DNA, 20 bp, Artificial Sequence
  Description of Artificial Sequence Synthetic oligonucleotide
auuaauaaattgtcatcacc

SEQ ID NO: 263 - DNA, 20 bp, Artificial Sequence
  Description of Artificial Sequence Synthetic oligonucleotide
ccgtcgcccttcagcacgca

SEQ ID NO: 264 - DNA, 20 bp, Artificial Sequence
  Description of Artificial Sequence Synthetic oligonucleotide
ccgtcgcccttcagcacgca

SEQ ID NO: 265 - DNA, 20 bp, Artificial Sequence
  Description of Artificial Sequence Synthetic oligonucleotide
ccgtcgcccttcagcacgca

SEQ ID NO: 266 - DNA, 20 bp, Artificial Sequence
  Description of Artificial Sequence Synthetic oligonucleotide
ccgtcgcccttcagcacgca

SEQ ID NO: 267 - DNA, 20 bp, Artificial Sequence
  Description of Artificial Sequence Synthetic oligonucleotide
ccgtcgcccttcagcacgca

SEQ ID NO: 268 - DNA, 20 bp, Artificial Sequence
  Description of Artificial Sequence Synthetic oligonucleotide
ccgtcgcccttcagcacgca

SEQ ID NO: 269 - DNA, 20 bp, Artificial Sequence
  Description of Artificial Sequence Synthetic oligonucleotide
gtccctgaagatgtcaatgc

SEQ ID NO: 270 - DNA, 20 bp, Artificial Sequence
  Description of Artificial Sequence Synthetic oligonucleotide
gtccctgaagatgtcaatgc

SEQ ID NO: 271 - DNA, 20 bp, Artificial Sequence
  Description of Artificial Sequence Synthetic oligonucleotide
gtccctgaagatgtcaatgc

SEQ ID NO: 272 - DNA, 20 bp, Artificial Sequence
  Description of Artificial Sequence Synthetic oligonucleotide
gtccctgaagatgtcaatgc

SEQ ID NO: 273 - DNA, 20 bp, Artificial Sequence
  Description of Artificial Sequence Synthetic oligonucleotide
gtccctgaagatgtcaatgc

SEQ ID NO: 274 - RNA, 20 nt, Artificial Sequence
  Description of Artificial Sequence Synthetic oligonucleotide
ggcacaagggcacagacuuc

SEQ ID NO: 275 - RNA, 20 nt, Artificial Sequence
  Description of Artificial Sequence Synthetic oligonucleotide
ggcacaagggcacagacuuc

SEQ ID NO: 276 - RNA, 20 nt, Artificial Sequence
  Description of Artificial Sequence Synthetic oligonucleotide
ggcacaagggcacagacuuc

SEQ ID NO: 277 - RNA, 20 nt, Artificial Sequence
  Description of Artificial Sequence Synthetic oligonucleotide
ggcacaagggcacagacuuc

SEQ ID NO: 278 - RNA, 20 nt, Artificial Sequence
  Description of Artificial Sequence Synthetic oligonucleotide
ggcacaagggcacagacuuc

SEQ ID NO: 279 - RNA, 20 nt, Artificial Sequence
  Description of Artificial Sequence Synthetic oligonucleotide
ggcacaagggcacagacuuc

SEQ ID NO: 280 - RNA, 20 nt, Artificial Sequence
  Description of Artificial Sequence Synthetic oligonucleotide
ggcacaagggcacagacuuc

SEQ ID NO: 281 - RNA, 20 nt, Artificial Sequence
  Description of Artificial Sequence Synthetic oligonucleotide
ggcacaagggcacagacuuc

SEQ ID NO: 282 - RNA, 20 nt, Artificial Sequence
  Description of Artificial Sequence Synthetic oligonucleotide
ggcacaagggcacagacuuc

SEQ ID NO: 283 - DNA, 20 bp, Artificial Sequence
  Description of Artificial Sequence Synthetic oligonucleotide
ggcacaagggcacagacttc

SEQ ID NO: 284 - RNA, 20 nt, Artificial Sequence
  Description of Artificial Sequence Synthetic oligonucleotide
ucaaggaagauggcauuucu

SEQ ID NO: 285 - RNA, 20 nt, Artificial Sequence
  Description of Artificial Sequence Synthetic oligonucleotide
ucaaggaagauggcauuucu

SEQ ID NO: 286 - RNA, 20 nt, Artificial Sequence
  Description of Artificial Sequence Synthetic oligonucleotide
ucaaggaagauggcauuucu

SEQ ID NO: 287 - RNA, 20 nt, Artificial Sequence
  Description of Artificial Sequence Synthetic oligonucleotide
ucaaggaagauggcauuucu

SEQ ID NO: 288 - RNA, 20 nt, Artificial Sequence
  Description of Artificial Sequence Synthetic oligonucleotide
ucaaggaagauggcauuucu

SEQ ID NO: 289 - RNA, 20 nt, Artificial Sequence
  Description of Artificial Sequence Synthetic oligonucleotide
ucaaggaagauggcauuucu

SEQ ID NO: 290 - RNA, 20 nt, Artificial Sequence
  Description of Artificial Sequence Synthetic oligonucleotide
ucaaggaagauggcauuucu

SEQ ID NO: 291 - RNA, 20 nt, Artificial Sequence
  Description of Artificial Sequence Synthetic oligonucleotide
ucaaggaagauggcauuucu

SEQ ID NO: 292 - RNA, 20 nt, Artificial Sequence
  Description of Artificial Sequence Synthetic oligonucleotide
ucaaggaagauggcauuucu

SEQ ID NO: 293 - RNA, 20 nt, Artificial Sequence
  Description of Artificial Sequence Synthetic oligonucleotide
ucaaggaagauggcauuucu

SEQ ID NO: 294 - RNA, 20 nt, Artificial Sequence
  Description of Artificial Sequence Synthetic oligonucleotide
ucaaggaagauggcauuucu

SEQ ID NO: 295 - RNA, 20 nt, Artificial Sequence
  Description of Artificial Sequence Synthetic oligonucleotide
ucaaggaagauggcauuucu

SEQ ID NO: 296 - RNA, 20 nt, Artificial Sequence
  Description of Artificial Sequence Synthetic oligonucleotide
ucaaggaagauggcauuucu

SEQ ID NO: 297 - RNA, 20 nt, Artificial Sequence
  Description of Artificial Sequence Synthetic oligonucleotide
ucaaggaagauggcauuucu

SEQ ID NO: 298 - RNA, 20 nt, Artificial Sequence
  Description of Artificial Sequence Synthetic oligonucleotide
ucaaggaagauggcauuucu

SEQ ID NO: 299 - RNA, 20 nt, Artificial Sequence
  Description of Artificial Sequence Synthetic oligonucleotide
ucaaggaagauggcauuucu

SEQ ID NO: 300 - DNA, 20 bp, Artificial Sequence
  Description of Artificial Sequence Synthetic oligonucleotide
tcaaggaagatggcatttct

SEQ ID NO: 301 - RNA, 20 nt, Artificial Sequence
  Description of Artificial Sequence Synthetic oligonucleotide
ucaaggaagauggcauuucu

SEQ ID NO: 302 - DNA, 20 bp, Artificial Sequence
  Description of Artificial Sequence Synthetic oligonucleotide
tcaaggaagatggcatttct

SEQ ID NO: 303 - DNA, 20 bp, Artificial Sequence
  Description of Artificial Sequence Synthetic oligonucleotide
tcaaggaagatggcatttct

SEQ ID NO: 304 - DNA, 20 bp, Artificial Sequence
  Description of Artificial Sequence Synthetic oligonucleotide
tcaaggaagauggcatttct

SEQ ID NO: 305 - DNA, 20 bp, Artificial Sequence
  Description of Artificial Sequence Synthetic oligonucleotide
ucaaggaagatggcatuucu

SEQ ID NO: 306 - DNA, 20 bp, Artificial Sequence
  Description of Artificial Sequence Synthetic oligonucleotide
tcaaggaagatggcautucu

SEQ ID NO: 307 - DNA, 20 bp, Artificial Sequence
  Description of Artificial Sequence Synthetic oligonucleotide
ucaaggaagauggcatutcu

SEQ ID NO: 308 - DNA, 20 bp, Artificial Sequence
  Description of Artificial Sequence Synthetic oligonucleotide
tcaaggaagatggcauutcu

SEQ ID NO: 309 - DNA, 20 bp, Artificial Sequence
  Description of Artificial Sequence Synthetic oligonucleotide
tcaaggaagauggcauttcu

SEQ ID NO: 310 - DNA, 20 bp, Artificial Sequence
  Description of Artificial Sequence Synthetic oligonucleotide
tcaaggaagatggcatttcu

SEQ ID NO: 311 - DNA, 20 bp, Artificial Sequence
  Description of Artificial Sequence Synthetic oligonucleotide
tcaaggaagatggcauttcu

SEQ ID NO: 312 - DNA, 20 bp, Artificial Sequence
  Description of Artificial Sequence Synthetic oligonucleotide
ucaaggaagauggcatttcu

SEQ ID NO: 313 - DNA, 20 bp, Artificial Sequence
  Description of Artificial Sequence Synthetic oligonucleotide
tcaaggaagatggcatttcu

SEQ ID NO: 314 - DNA, 20 bp, Artificial Sequence
  Description of Artificial Sequence Synthetic oligonucleotide
tcaaggaagauggcatttct

SEQ ID NO: 315 - DNA, 20 bp, Artificial Sequence
  Description of Artificial Sequence Synthetic oligonucleotide
ucaaggaagatggcatuucu

SEQ ID NO: 316 - DNA, 20 bp, Artificial Sequence
  Description of Artificial Sequence Synthetic oligonucleotide
tcaaggaagatggcautucu

SEQ ID NO: 317 - DNA, 20 bp, Artificial Sequence
  Description of Artificial Sequence Synthetic oligonucleotide
ucaaggaagauggcatutcu

SEQ ID NO: 318 - DNA, 20 bp, Artificial Sequence
  Description of Artificial Sequence Synthetic oligonucleotide
tcaaggaagatggcauutcu

SEQ ID NO: 319 - DNA, 20 bp, Artificial Sequence
  Description of Artificial Sequence Synthetic oligonucleotide
tcaaggaagauggcauttcu

SEQ ID NO: 320 - DNA, 20 bp, Artificial Sequence
  Description of Artificial Sequence Synthetic oligonucleotide
tcaaggaagatggcatttcu

SEQ ID NO: 321 - DNA, 20 bp, Artificial Sequence
  Description of Artificial Sequence Synthetic oligonucleotide
tcaaggaagatggcauttcu

SEQ ID NO: 322 - DNA, 20 bp, Artificial Sequence
  Description of Artificial Sequence Synthetic oligonucleotide
ucaaggaagauggcatttcu

SEQ ID NO: 323 - DNA, 20 bp, Artificial Sequence
  Description of Artificial Sequence Synthetic oligonucleotide
tcaaggaagatggcatttcu

SEQ ID NO: 324 - RNA, 20 nt, Artificial Sequence
  Description of Artificial Sequence Synthetic oligonucleotide
ucaaggaagauggcauuucu

SEQ ID NO: 325 - RNA, 20 nt, Artificial Sequence
  Description of Artificial Sequence Synthetic oligonucleotide
ucaaggaagauggcauuucu

SEQ ID NO: 326 - RNA, 20 nt, Artificial Sequence
  Description of Artificial Sequence Synthetic oligonucleotide
ucaaggaagauggcauuucu

SEQ ID NO: 327 - RNA, 20 nt, Artificial Sequence
  Description of Artificial Sequence Synthetic oligonucleotide
ucaaggaagauggcauuucu

SEQ ID NO: 328 - RNA, 20 nt, Artificial Sequence
  Description of Artificial Sequence Synthetic oligonucleotide
ucaaggaagauggcauuucu

SEQ ID NO: 329 - RNA, 20 nt, Artificial Sequence
  Description of Artificial Sequence Synthetic oligonucleotide
ucaaggaagauggcauuucu

SEQ ID NO: 330 - RNA, 20 nt, Artificial Sequence
  Description of Artificial Sequence Synthetic oligonucleotide
ucaaggaagauggcauuucu

SEQ ID NO: 331 - RNA, 20 nt, Artificial Sequence
  Description of Artificial Sequence Synthetic oligonucleotide
ucaaggaagauggcauuucu

SEQ ID NO: 332 - RNA, 20 nt, Artificial Sequence
  Description of Artificial Sequence Synthetic oligonucleotide
ucaaggaagauggcauuucu

SEQ ID NO: 333 - RNA, 20 nt, Artificial Sequence
  Description of Artificial Sequence Synthetic oligonucleotide
ucaaggaagauggcauuucu

SEQ ID NO: 334 - RNA, 20 nt, Artificial Sequence
  Description of Artificial Sequence Synthetic oligonucleotide
ucaaggaagauggcauuucu

SEQ ID NO: 335 - RNA, 20 nt, Artificial Sequence
  Description of Artificial Sequence Synthetic oligonucleotide
ucaaggaagauggcauuucu

SEQ ID NO: 336 - RNA, 20 nt, Artificial Sequence
  Description of Artificial Sequence Synthetic oligonucleotide
ucaaggaagauggcauuucu

SEQ ID NO: 337 - RNA, 20 nt, Artificial Sequence
  Description of Artificial Sequence Synthetic oligonucleotide
ucaaggaagauggcauuucu

SEQ ID NO: 338 - RNA, 20 nt, Artificial Sequence
  Description of Artificial Sequence Synthetic oligonucleotide
ucaaggaagauggcauuucu

SEQ ID NO: 339 - RNA, 20 nt, Artificial Sequence
  Description of Artificial Sequence Synthetic oligonucleotide
ucaaggaagauggcauuucu

SEQ ID NO: 340 - RNA, 20 nt, Artificial Sequence
  Description of Artificial Sequence Synthetic oligonucleotide
ucaaggaagauggcauuucu

SEQ ID NO: 341 - RNA, 20 nt, Artificial Sequence
  Description of Artificial Sequence Synthetic oligonucleotide
ucaaggaagauggcauuucu

SEQ ID NO: 342 - RNA, 20 nt, Artificial Sequence
  Description of Artificial Sequence Synthetic oligonucleotide
ucaaggaagauggcauuucu

SEQ ID NO: 343 - RNA, 20 nt, Artificial Sequence
  Description of Artificial Sequence Synthetic oligonucleotide
ucaaggaagauggcauuucu

SEQ ID NO: 344 - RNA, 20 nt, Artificial Sequence
  Description of Artificial Sequence Synthetic oligonucleotide
ucaaggaagauggcauuucu

SEQ ID NO: 345 - RNA, 20 nt, Artificial Sequence
  Description of Artificial Sequence Synthetic oligonucleotide
ucaaggaagauggcauuucu

SEQ ID NO: 346 - RNA, 20 nt, Artificial Sequence
  Description of Artificial Sequence Synthetic oligonucleotide
ucaaggaagauggcauuucu

SEQ ID NO: 347 - RNA, 20 nt, Artificial Sequence
  Description of Artificial Sequence Synthetic oligonucleotide
ucaaggaagauggcauuucu

SEQ ID NO: 348 - RNA, 20 nt, Artificial Sequence
  Description of Artificial Sequence Synthetic oligonucleotide
ucaaggaagauggcauuucu

SEQ ID NO: 349 - RNA, 20 nt, Artificial Sequence
  Description of Artificial Sequence Synthetic oligonucleotide
ucaaggaagauggcauuucu

SEQ ID NO: 350 - RNA, 20 nt, Artificial Sequence
  Description of Artificial Sequence Synthetic oligonucleotide
ucaaggaagauggcauuucu

SEQ ID NO: 351 - RNA, 20 nt, Artificial Sequence
  Description of Artificial Sequence Synthetic oligonucleotide
ucaaggaagauggcauuucu

SEQ ID NO: 352 - RNA, 20 nt, Artificial Sequence
  Description of Artificial Sequence Synthetic oligonucleotide
ucaaggaagauggcauuucu

SEQ ID NO: 353 - RNA, 20 nt, Artificial Sequence
  Description of Artificial Sequence Synthetic oligonucleotide
ucaaggaagauggcauuucu

SEQ ID NO: 354 - RNA, 20 nt, Artificial Sequence
  Description of Artificial Sequence Synthetic oligonucleotide
ucaaggaagauggcauuucu

SEQ ID NO: 355 - RNA, 20 nt, Artificial Sequence
  Description of Artificial Sequence Synthetic oligonucleotide
ucaaggaagauggcauuucu

SEQ ID NO: 356 - RNA, 20 nt, Artificial Sequence
  Description of Artificial Sequence Synthetic oligonucleotide
ucaaggaagauggcauuucu

SEQ ID NO: 357 - RNA, 20 nt, Artificial Sequence
  Description of Artificial Sequence Synthetic oligonucleotide
ucaaggaagauggcauuucu

SEQ ID NO: 358 - RNA, 20 nt, Artificial Sequence
  Description of Artificial Sequence Synthetic oligonucleotide
ucaaggaagauggcauuucu

SEQ ID NO: 359 - RNA, 20 nt, Artificial Sequence
  Description of Artificial Sequence Synthetic oligonucleotide
ucaaggaagauggcauuucu

SEQ ID NO: 360 - RNA, 20 nt, Artificial Sequence
  Description of Artificial Sequence Synthetic oligonucleotide
ucaaggaagauggcauuucu

SEQ ID NO: 361 - RNA, 20 nt, Artificial Sequence
  Description of Artificial Sequence Synthetic oligonucleotide
ucaaggaagauggcauuucu

SEQ ID NO: 362 - RNA, 20 nt, Artificial Sequence
  Description of Artificial Sequence Synthetic oligonucleotide
ucaaggaagauggcauuucu

SEQ ID NO: 363 - RNA, 20 nt, Artificial Sequence
  Description of Artificial Sequence Synthetic oligonucleotide
ucaaggaagauggcauuucu

SEQ ID NO: 364 - RNA, 20 nt, Artificial Sequence
  Description of Artificial Sequence Synthetic oligonucleotide
ucaaggaagauggcauuucu

SEQ ID NO: 365 - RNA, 20 nt, Artificial Sequence
  Description of Artificial Sequence Synthetic oligonucleotide
ucaaggaagauggcauuucu

SEQ ID NO: 366 - RNA, 20 nt, Artificial Sequence
  Description of Artificial Sequence Synthetic oligonucleotide
ucaaggaagauggcauuucu

SEQ ID NO: 367 - RNA, 20 nt, Artificial Sequence
  Description of Artificial Sequence Synthetic oligonucleotide
ucaaggaagauggcauuucu

SEQ ID NO: 368 - RNA, 20 nt, Artificial Sequence
  Description of Artificial Sequence Synthetic oligonucleotide
ucaaggaagauggcauuucu

SEQ ID NO: 369 - RNA, 20 nt, Artificial Sequence
  Description of Artificial Sequence Synthetic oligonucleotide
ucaaggaagauggcauuucu

SEQ ID NO: 370 - RNA, 20 nt, Artificial Sequence
  Description of Artificial Sequence Synthetic oligonucleotide
ucaaggaagauggcauuucu

SEQ ID NO: 371 - RNA, 20 nt, Artificial Sequence
  Description of Artificial Sequence Synthetic oligonucleotide
ucaaggaagauggcauuucu

SEQ ID NO: 372 - RNA, 20 nt, Artificial Sequence
  Description of Artificial Sequence Synthetic oligonucleotide
ucaaggaagauggcauuucu

SEQ ID NO: 373 - RNA, 20 nt, Artificial Sequence
  Description of Artificial Sequence Synthetic oligonucleotide
ucaaggaagauggcauuucu

SEQ ID NO: 374 - RNA, 20 nt, Artificial Sequence
  Description of Artificial Sequence Synthetic oligonucleotide
ucaaggaagauggcauuucu

SEQ ID NO: 375 - RNA, 20 nt, Artificial Sequence
  Description of Artificial Sequence Synthetic oligonucleotide
ucaaggaagauggcauuucu

SEQ ID NO: 376 - RNA, 20 nt, Artificial Sequence
  Description of Artificial Sequence Synthetic oligonucleotide
ucaaggaagauggcauuucu

SEQ ID NO: 377 - RNA, 20 nt, Artificial Sequence
  Description of Artificial Sequence Synthetic oligonucleotide
ucaaggaagauggcauuucu

SEQ ID NO: 378 - RNA, 20 nt, Artificial Sequence
  Description of Artificial Sequence Synthetic oligonucleotide
ucaaggaagauggcauuucu

SEQ ID NO: 379 - RNA, 20 nt, Artificial Sequence
  Description of Artificial Sequence Synthetic oligonucleotide
ucaaggaagauggcauuucu

SEQ ID NO: 380 - RNA, 20 nt, Artificial Sequence
  Description of Artificial Sequence Synthetic oligonucleotide
ucaaggaagauggcauuucu

SEQ ID NO: 381 - RNA, 20 nt, Artificial Sequence
  Description of Artificial Sequence Synthetic oligonucleotide
ucaaggaagauggcauuucu

SEQ ID NO: 382 - RNA, 20 nt, Artificial Sequence
  Description of Artificial Sequence Synthetic oligonucleotide
ucaaggaagauggcauuucu

SEQ ID NO: 383 - RNA, 20 nt, Artificial Sequence
  Description of Artificial Sequence Synthetic oligonucleotide
ucaaggaagauggcauuucu

SEQ ID NO: 384 - RNA, 20 nt, Artificial Sequence
  Description of Artificial Sequence Synthetic oligonucleotide
ucaaggaagauggcauuucu

SEQ ID NO: 385 - RNA, 20 nt, Artificial Sequence
  Description of Artificial Sequence Synthetic oligonucleotide
ucaaggaagauggcauuucu

SEQ ID NO: 386 - RNA, 20 nt, Artificial Sequence
  Description of Artificial Sequence Synthetic oligonucleotide
ucaaggaagauggcauuucu

SEQ ID NO: 387 - RNA, 20 nt, Artificial Sequence
  Description of Artificial Sequence Synthetic oligonucleotide
ucaaggaagauggcauuucu

SEQ ID NO: 388 - RNA, 20 nt, Artificial Sequence
  Description of Artificial Sequence Synthetic oligonucleotide
ucaaggaagauggcauuucu

SEQ ID NO: 389 - RNA, 20 nt, Artificial Sequence
  Description of Artificial Sequence Synthetic oligonucleotide
ucaaggaagauggcauuucu

SEQ ID NO: 390 - RNA, 20 nt, Artificial Sequence
  Description of Artificial Sequence Synthetic oligonucleotide
ucaaggaagauggcauuucu

SEQ ID NO: 391 - RNA, 20 nt, Artificial Sequence
  Description of Artificial Sequence Synthetic oligonucleotide
ucaaggaagauggcauuucu

SEQ ID NO: 392 - RNA, 20 nt, Artificial Sequence
  Description of Artificial Sequence Synthetic oligonucleotide
ucaaggaagauggcauuucu

SEQ ID NO: 393 - RNA, 20 nt, Artificial Sequence
  Description of Artificial Sequence Synthetic oligonucleotide
ucaaggaagauggcauuucu

SEQ ID NO: 394 - RNA, 20 nt, Artificial Sequence
  Description of Artificial Sequence Synthetic oligonucleotide
ucaaggaagauggcauuucu

SEQ ID NO: 395 - RNA, 20 nt, Artificial Sequence
  Description of Artificial Sequence Synthetic oligonucleotide
ggccaaaccucggcuuaccu

SEQ ID NO: 396 - RNA, 30 nt, Artificial Sequence
  Description of Artificial Sequence Synthetic oligonucleotide
cuccaacaucaaggaagauggcauuucuag

SEQ ID NO: 397 - RNA, 25 nt, Artificial Sequence
  Description of Artificial Sequence Synthetic oligonucleotide
accagaguaacagucugaguaggag

SEQ ID NO: 398 - RNA, 25 nt, Artificial Sequence
  Description of Artificial Sequence Synthetic oligonucleotide
caccagaguaacagucugaguagga

SEQ ID NO: 399 - RNA, 25 nt, Artificial Sequence
  Description of Artificial Sequence Synthetic oligonucleotide
ucaccagaguaacagucugaguagg

SEQ ID NO: 400 - RNA, 25 nt, Artificial Sequence
  Description of Artificial Sequence Synthetic oligonucleotide
gucaccagaguaacagucugaguag

SEQ ID NO: 401 - RNA, 25 nt, Artificial Sequence
  Description of Artificial Sequence Synthetic oligonucleotide
guugugucaccagaguaacagucug

SEQ ID NO: 402 - RNA, 25 nt, Artificial Sequence
  Description of Artificial Sequence Synthetic oligonucleotide
gguugugucaccagaguaacagucu

SEQ ID NO: 403 - RNA, 25 nt, Artificial Sequence
  Description of Artificial Sequence Synthetic oligonucleotide
agguugugucaccagaguaacaguc

SEQ ID NO: 404 - RNA, 25 nt, Artificial Sequence
  Description of Artificial Sequence Synthetic oligonucleotide
cagguugugucaccagaguaacagu

SEQ ID NO: 405 - RNA, 25 nt, Artificial Sequence
  Description of Artificial Sequence Synthetic oligonucleotide
acagguugugucaccagaguaacag

SEQ ID NO: 406 - RNA, 25 nt, Artificial Sequence
  Description of Artificial Sequence Synthetic oligonucleotide
ccacagguugugucaccagaguaac

SEQ ID NO: 407 - RNA, 25 nt, Artificial Sequence
  Description of Artificial Sequence Synthetic oligonucleotide
accacagguugugucaccagaguaa

SEQ ID NO: 408 - RNA, 25 nt, Artificial Sequence
  Description of Artificial Sequence Synthetic oligonucleotide
aaccacagguugugucaccagagua

SEQ ID NO: 409 - RNA, 25 nt, Artificial Sequence
  Description of Artificial Sequence Synthetic oligonucleotide
uaaccacagguugugucaccagagu

SEQ ID NO: 410 - RNA, 25 nt, Artificial Sequence
  Description of Artificial Sequence Synthetic oligonucleotide
guaaccacagguugugucaccagag

SEQ ID NO: 411 - RNA, 25 nt, Artificial Sequence
  Description of Artificial Sequence Synthetic oligonucleotide
aguaaccacagguugugucaccaga

SEQ ID NO: 412 - RNA, 25 nt, Artificial Sequence
  Description of Artificial Sequence Synthetic oligonucleotide
uaguaaccacagguugugucaccag

SEQ ID NO: 413 - RNA, 25 nt, Artificial Sequence
  Description of Artificial Sequence Synthetic oligonucleotide
uuaguaaccacagguugugucacca

SEQ ID NO: 414 - RNA, 25 nt, Artificial Sequence
  Description of Artificial Sequence Synthetic oligonucleotide
cuuaguaaccacagguugugucacc

SEQ ID NO: 415 - RNA, 25 nt, Artificial Sequence
  Description of Artificial Sequence Synthetic oligonucleotide
ccuuaguaaccacagguugugucac

SEQ ID NO: 416 - RNA, 25 nt, Artificial Sequence
  Description of Artificial Sequence Synthetic oligonucleotide
uccuuaguaaccacagguuguguca

SEQ ID NO: 417 - RNA, 25 nt, Artificial Sequence
  Description of Artificial Sequence Synthetic oligonucleotide
guuuccuuaguaaccacagguugug

SEQ ID NO: 418 - RNA, 25 nt, Artificial Sequence
  Description of Artificial Sequence Synthetic oligonucleotide
aguuuccuuaguaaccacagguugu

SEQ ID NO: 419 - RNA, 25 nt, Artificial Sequence
  Description of Artificial Sequence Synthetic oligonucleotide
caguuuccuuaguaaccacagguug

SEQ ID NO: 420 - RNA, 25 nt, Artificial Sequence
  Description of Artificial Sequence Synthetic oligonucleotide
gcaguuuccuuaguaaccacagguu

SEQ ID NO: 421 - RNA, 25 nt, Artificial Sequence
  Description of Artificial Sequence Synthetic oligonucleotide
ggcaguuuccuuaguaaccacaggu

SEQ ID NO: 422 - RNA, 25 nt, Artificial Sequence
  Description of Artificial Sequence Synthetic oligonucleotide
uggcaguuuccuuaguaaccacagg

SEQ ID NO: 423 - RNA, 25 nt, Artificial Sequence
  Description of Artificial Sequence Synthetic oligonucleotide
auggcaguuuccuuaguaaccacag

SEQ ID NO: 424 - RNA, 25 nt, Artificial Sequence
  Description of Artificial Sequence Synthetic oligonucleotide
agauggcaguuuccuuaguaaccac

SEQ ID NO: 425 - RNA, 25 nt, Artificial Sequence
  Description of Artificial Sequence Synthetic oligonucleotide
gagauggcaguuuccuuaguaacca

SEQ ID NO: 426 - RNA, 25 nt, Artificial Sequence
  Description of Artificial Sequence Synthetic oligonucleotide
ggagauggcaguuuccuuaguaacc

SEQ ID NO: 427 - RNA, 25 nt, Artificial Sequence
  Description of Artificial Sequence Synthetic oligonucleotide
uggagauggcaguuuccuuaguaac

SEQ ID NO: 428 - RNA, 25 nt, Artificial Sequence
  Description of Artificial Sequence Synthetic oligonucleotide
uuggagauggcaguuuccuuaguaa

SEQ ID NO: 429 - RNA, 25 nt, Artificial Sequence
  Description of Artificial Sequence Synthetic oligonucleotide
uuuggagauggcaguuuccuuagua

SEQ ID NO: 430 - RNA, 25 nt, Artificial Sequence
  Description of Artificial Sequence Synthetic oligonucleotide
aguuuggagauggcaguuuccuuag

SEQ ID NO: 431 - RNA, 25 nt, Artificial Sequence
  Description of Artificial Sequence Synthetic oligonucleotide
uaguuuggagauggcaguuuccuua

SEQ ID NO: 432 - RNA, 25 nt, Artificial Sequence
  Description of Artificial Sequence Synthetic oligonucleotide
cuaguuuggagauggcaguuuccuu

SEQ ID NO: 433 - RNA, 25 nt, Artificial Sequence
  Description of Artificial Sequence Synthetic oligonucleotide
ucuaguuuggagauggcaguuuccu

SEQ ID NO: 434 - RNA, 25 nt, Artificial Sequence
  Description of Artificial Sequence Synthetic oligonucleotide
uucuaguuuggagauggcaguuucc

SEQ ID NO: 435 - RNA, 25 nt, Artificial Sequence
  Description of Artificial Sequence Synthetic oligonucleotide
cauuucuaguuuggagauggcaguu

SEQ ID NO: 436 - RNA, 25 nt, Artificial Sequence
  Description of Artificial Sequence Synthetic oligonucleotide
gcauuucuaguuuggagauggcagu

SEQ ID NO: 437 - RNA, 25 nt, Artificial Sequence
  Description of Artificial Sequence Synthetic oligonucleotide
auggcauuucuaguuuggagauggc

SEQ ID NO: 438 - RNA, 25 nt, Artificial Sequence
  Description of Artificial Sequence Synthetic oligonucleotide
gaagauggcauuucuaguuuggaga

SEQ ID NO: 439 - RNA, 25 nt, Artificial Sequence
  Description of Artificial Sequence Synthetic oligonucleotide
aggaagauggcauuucuaguuugga

SEQ ID NO: 440 - RNA, 25 nt, Artificial Sequence
  Description of Artificial Sequence Synthetic oligonucleotide
aaggaagauggcauuucuaguuugg

SEQ ID NO: 441 - RNA, 25 nt, Artificial Sequence
  Description of Artificial Sequence Synthetic oligonucleotide
caaggaagauggcauuucuaguuug

SEQ ID NO: 442 - RNA, 25 nt, Artificial Sequence
  Description of Artificial Sequence Synthetic oligonucleotide
caucaaggaagauggcauuucuagu

SEQ ID NO: 443 - RNA, 25 nt, Artificial Sequence
  Description of Artificial Sequence Synthetic oligonucleotide
acaucaaggaagauggcauuucuag

SEQ ID NO: 444 - RNA, 25 nt, Artificial Sequence
  Description of Artificial Sequence Synthetic oligonucleotide
aacaucaaggaagauggcauuucua

SEQ ID NO: 445 - RNA, 25 nt, Artificial Sequence
  Description of Artificial Sequence Synthetic oligonucleotide
caacaucaaggaagauggcauuucu

SEQ ID NO: 446 - RNA, 25 nt, Artificial Sequence
  Description of Artificial Sequence Synthetic oligonucleotide
cuccaacaucaaggaagauggcauu

SEQ ID NO: 447 - RNA, 25 nt, Artificial Sequence
  Description of Artificial Sequence Synthetic oligonucleotide
accuccaacaucaaggaagauggca

SEQ ID NO: 448 - RNA, 25 nt, Artificial Sequence
  Description of Artificial Sequence Synthetic oligonucleotide
guaccuccaacaucaaggaagaugg

SEQ ID NO: 449 - RNA, 25 nt, Artificial Sequence
  Description of Artificial Sequence Synthetic oligonucleotide
agguaccuccaacaucaaggaagau

SEQ ID NO: 450 - RNA, 25 nt, Artificial Sequence
  Description of Artificial Sequence Synthetic oligonucleotide
agagcagguaccuccaacaucaagg

SEQ ID NO: 451 - RNA, 25 nt, Artificial Sequence
  Description of Artificial Sequence Synthetic oligonucleotide
cagagcagguaccuccaacaucaag

SEQ ID NO: 452 - RNA, 25 nt, Artificial Sequence
  Description of Artificial Sequence Synthetic oligonucleotide
cugccagagcagguaccuccaacau

SEQ ID NO: 453 - RNA, 25 nt, Artificial Sequence
  Description of Artificial Sequence Synthetic oligonucleotide
ucugccagagcagguaccuccaaca

SEQ ID NO: 454 - RNA, 25 nt, Artificial Sequence
  Description of Artificial Sequence Synthetic oligonucleotide
aucugccagagcagguaccuccaac

SEQ ID NO: 455 - RNA, 25 nt, Artificial Sequence
  Description of Artificial Sequence Synthetic oligonucleotide
aaucugccagagcagguaccuccaa

SEQ ID NO: 456 - RNA, 25 nt, Artificial Sequence
  Description of Artificial Sequence Synthetic oligonucleotide
aaaucugccagagcagguaccucca

SEQ ID NO: 457 - RNA, 25 nt, Artificial Sequence
  Description of Artificial Sequence Synthetic oligonucleotide
gaaaucugccagagcagguaccucc

SEQ ID NO: 458 - RNA, 25 nt, Artificial Sequence
  Description of Artificial Sequence Synthetic oligonucleotide
ugaaaucugccagagcagguaccuc

SEQ ID NO: 459 - RNA, 25 nt, Artificial Sequence
  Description of Artificial Sequence Synthetic oligonucleotide
uugaaaucugccagagcagguaccu

SEQ ID NO: 460 - RNA, 25 nt, Artificial Sequence
  Description of Artificial Sequence Synthetic oligonucleotide
cccgguugaaaucugccagagcagg

SEQ ID NO: 461 - RNA, 25 nt, Artificial Sequence
  Description of Artificial Sequence Synthetic oligonucleotide
ccaagcccgguugaaaucugccaga

SEQ ID NO: 462 - RNA, 25 nt, Artificial Sequence
  Description of Artificial Sequence Synthetic oligonucleotide
uccaagcccgguugaaaucugccag

SEQ ID NO: 463 - RNA, 25 nt, Artificial Sequence
  Description of Artificial Sequence Synthetic oligonucleotide
guccaagcccgguugaaaucugcca

SEQ ID NO: 464 - RNA, 25 nt, Artificial Sequence
  Description of Artificial Sequence Synthetic oligonucleotide
ucuguccaagcccgguugaaaucug

SEQ ID NO: 465 - RNA, 25 nt, Artificial Sequence
  Description of Artificial Sequence Synthetic oligonucleotide
uucuguccaagcccgguugaaaucu

SEQ ID NO: 466 - RNA, 25 nt, Artificial Sequence
  Description of Artificial Sequence Synthetic oligonucleotide
guucuguccaagcccgguugaaauc

SEQ ID NO: 467 - RNA, 25 nt, Artificial Sequence
  Description of Artificial Sequence Synthetic oligonucleotide
aguucuguccaagcccgguugaaau

SEQ ID NO: 468 - RNA, 25 nt, Artificial Sequence
  Description of Artificial Sequence Synthetic oligonucleotide
aaguucuguccaagcccgguugaaa

SEQ ID NO: 469 - RNA, 25 nt, Artificial Sequence
  Description of Artificial Sequence Synthetic oligonucleotide
uaaguucuguccaagcccgguugaa

SEQ ID NO: 470 - RNA, 25 nt, Artificial Sequence
  Description of Artificial Sequence Synthetic oligonucleotide
guaaguucuguccaagcccgguuga

SEQ ID NO: 471 - RNA, 25 nt, Artificial Sequence
  Description of Artificial Sequence Synthetic oligonucleotide
gguaaguucuguccaagcccgguug

SEQ ID NO: 472 - RNA, 25 nt, Artificial Sequence
  Description of Artificial Sequence Synthetic oligonucleotide
cgguaaguucuguccaagcccgguu

SEQ ID NO: 473 - RNA, 25 nt, Artificial Sequence
  Description of Artificial Sequence Synthetic oligonucleotide
ucgguaaguucuguccaagcccggu

SEQ ID NO: 474 - RNA, 25 nt, Artificial Sequence
  Description of Artificial Sequence Synthetic oligonucleotide
gucgguaaguucuguccaagcccgg

SEQ ID NO: 475 - RNA, 25 nt, Artificial Sequence
  Description of Artificial Sequence Synthetic oligonucleotide
agucgguaaguucuguccaagcccg

SEQ ID NO: 476 - RNA, 25 nt, Artificial Sequence
  Description of Artificial Sequence Synthetic oligonucleotide
cagucgguaaguucuguccaagccc

SEQ ID NO: 477 - RNA, 25 nt, Artificial Sequence
  Description of Artificial Sequence Synthetic oligonucleotide
aaagccagucgguaaguucugucca

SEQ ID NO: 478 - RNA, 25 nt, Artificial Sequence
  Description of Artificial Sequence Synthetic oligonucleotide
gaaagccagucgguaaguucugucc

SEQ ID NO: 479 - RNA, 25 nt, Artificial Sequence
  Description of Artificial Sequence Synthetic oligonucleotide
gucacccaccaucacccucugugau

SEQ ID NO: 480 - RNA, 25 nt, Artificial Sequence
  Description of Artificial Sequence Synthetic oligonucleotide
ggucacccaccaucacccucuguga

SEQ ID NO: 481 - RNA, 25 nt, Artificial Sequence
  Description of Artificial Sequence Synthetic oligonucleotide
aaggucacccaccaucacccucugu

SEQ ID NO: 482 - RNA, 25 nt, Artificial Sequence
  Description of Artificial Sequence Synthetic oligonucleotide
caaggucacccaccaucacccucug

SEQ ID NO: 483 - RNA, 25 nt, Artificial Sequence
  Description of Artificial Sequence Synthetic oligonucleotide
ucaaggucacccaccaucacccucu

SEQ ID NO: 484 - RNA, 25 nt, Artificial Sequence
  Description of Artificial Sequence Synthetic oligonucleotide
cucaaggucacccaccaucacccuc

SEQ ID NO: 485 - RNA, 25 nt, Artificial Sequence
  Description of Artificial Sequence Synthetic oligonucleotide
cuugaucaagcagagaaagccaguc

SEQ ID NO: 486 - RNA, 25 nt, Artificial Sequence
  Description of Artificial Sequence Synthetic oligonucleotide
auaacuugaucaagcagagaaagcc

SEQ ID NO: 487 - RNA, 20 nt, Artificial Sequence
  Description of Artificial Sequence Synthetic oligonucleotide
aguaacagucugaguaggag

SEQ ID NO: 488 - RNA, 20 nt, Artificial Sequence
  Description of Artificial Sequence Synthetic oligonucleotide
gaguaacagucugaguagga

SEQ ID NO: 489 - RNA, 20 nt, Artificial Sequence
  Description of Artificial Sequence Synthetic oligonucleotide
agaguaacagucugaguagg

SEQ ID NO: 490 - RNA, 20 nt, Artificial Sequence
  Description of Artificial Sequence Synthetic oligonucleotide
cagaguaacagucugaguag

SEQ ID NO: 491 - RNA, 20 nt, Artificial Sequence
  Description of Artificial Sequence Synthetic oligonucleotide
gucaccagaguaacagucug

SEQ ID NO: 492 - RNA, 20 nt, Artificial Sequence
  Description of Artificial Sequence Synthetic oligonucleotide
ugucaccagaguaacagucu

SEQ ID NO: 493 - RNA, 20 nt, Artificial Sequence
  Description of Artificial Sequence Synthetic oligonucleotide
gugucaccagaguaacaguc

SEQ ID NO: 494 - RNA, 20 nt, Artificial Sequence
  Description of Artificial Sequence Synthetic oligonucleotide
ugugucaccagaguaacagu

SEQ ID NO: 495 - RNA, 20 nt, Artificial Sequence
  Description of Artificial Sequence Synthetic oligonucleotide
uugugucaccagaguaacag

SEQ ID NO: 496 - RNA, 20 nt, Artificial Sequence
  Description of Artificial Sequence Synthetic oligonucleotide
gguugugucaccagaguaac

SEQ ID NO: 497 - RNA, 20 nt, Artificial Sequence
  Description of Artificial Sequence Synthetic oligonucleotide
agguugugucaccagaguaa

SEQ ID NO: 498 - RNA, 20 nt, Artificial Sequence
  Description of Artificial Sequence Synthetic oligonucleotide
cagguugugucaccagagua

SEQ ID NO: 499 - RNA, 20 nt, Artificial Sequence
  Description of Artificial Sequence Synthetic oligonucleotide
acagguugugucaccagagu

SEQ ID NO: 500 - RNA, 20 nt, Artificial Sequence
  Description of Artificial Sequence Synthetic oligonucleotide
cacagguugugucaccagag

SEQ ID NO: 501 - RNA, 20 nt, Artificial Sequence
  Description of Artificial Sequence Synthetic oligonucleotide
ccacagguugugucaccaga

SEQ ID NO: 502 - RNA, 20 nt, Artificial Sequence
  Description of Artificial Sequence Synthetic oligonucleotide
accacagguugugucaccag

SEQ ID NO: 503 - RNA, 20 nt, Artificial Sequence
  Description of Artificial Sequence Synthetic oligonucleotide
aaccacagguugugucacca

SEQ ID NO: 504 - RNA, 20 nt, Artificial Sequence
  Description of Artificial Sequence Synthetic oligonucleotide
uaaccacagguugugucacc

SEQ ID NO: 505 - RNA, 20 nt, Artificial Sequence
  Description of Artificial Sequence Synthetic oligonucleotide
guaaccacagguugugucac

SEQ ID NO: 506 - RNA, 20 nt, Artificial Sequence
  Description of Artificial Sequence Synthetic oligonucleotide
aguaaccacagguuguguca

SEQ ID NO: 507 - RNA, 20 nt, Artificial Sequence
  Description of Artificial Sequence Synthetic oligonucleotide
cuuaguaaccacagguugug

SEQ ID NO: 508 - RNA, 20 nt, Artificial Sequence
  Description of Artificial Sequence Synthetic oligonucleotide
ccuuaguaaccacagguugu

SEQ ID NO: 509 - RNA, 20 nt, Artificial Sequence
  Description of Artificial Sequence Synthetic oligonucleotide
uccuuaguaaccacagguug

SEQ ID NO: 510 - RNA, 20 nt, Artificial Sequence
  Description of Artificial Sequence Synthetic oligonucleotide
uuccuuaguaaccacagguu

SEQ ID NO: 511 - RNA, 20 nt, Artificial Sequence
  Description of Artificial Sequence Synthetic oligonucleotide
uuuccuuaguaaccacaggu

SEQ ID NO: 512 - RNA, 20 nt, Artificial Sequence
  Description of Artificial Sequence Synthetic oligonucleotide
guuuccuuaguaaccacagg

SEQ ID NO: 513 - RNA, 20 nt, Artificial Sequence
  Description of Artificial Sequence Synthetic oligonucleotide
aguuuccuuaguaaccacag

SEQ ID NO: 514 - RNA, 20 nt, Artificial Sequence
  Description of Artificial Sequence Synthetic oligonucleotide
gcaguuuccuuaguaaccac

SEQ ID NO: 515 - RNA, 20 nt, Artificial Sequence
  Description of Artificial Sequence Synthetic oligonucleotide
ggcaguuuccuuaguaacca

SEQ ID NO: 516 - RNA, 20 nt, Artificial Sequence
  Description of Artificial Sequence Synthetic oligonucleotide
uggcaguuuccuuaguaacc

SEQ ID NO: 517 - RNA, 20 nt, Artificial Sequence
  Description of Artificial Sequence Synthetic oligonucleotide
auggcaguuuccuuaguaac

SEQ ID NO: 518 - RNA, 20 nt, Artificial Sequence
  Description of Artificial Sequence Synthetic oligonucleotide
gauggcaguuuccuuaguaa

SEQ ID NO: 519 - RNA, 20 nt, Artificial Sequence
  Description of Artificial Sequence Synthetic oligonucleotide
agauggcaguuuccuuagua

SEQ ID NO: 520 - RNA, 20 nt, Artificial Sequence
  Description of Artificial Sequence Synthetic oligonucleotide
ggagauggcaguuuccuuag

SEQ ID NO: 521 - RNA, 20 nt, Artificial Sequence
  Description of Artificial Sequence Synthetic oligonucleotide
uggagauggcaguuuccuua

SEQ ID NO: 522 - RNA, 20 nt, Artificial Sequence
  Description of Artificial Sequence Synthetic oligonucleotide
uuggagauggcaguuuccuu

SEQ ID NO: 523 - RNA, 20 nt, Artificial Sequence
  Description of Artificial Sequence Synthetic oligonucleotide
uuuggagauggcaguuuccu

SEQ ID NO: 524 - RNA, 20 nt, Artificial Sequence
  Description of Artificial Sequence Synthetic oligonucleotide
guuuggagauggcaguuucc

SEQ ID NO: 525 - RNA, 20 nt, Artificial Sequence
  Description of Artificial Sequence Synthetic oligonucleotide
cuaguuuggagauggcaguu

SEQ ID NO: 526 - RNA, 20 nt, Artificial Sequence
  Description of Artificial Sequence Synthetic oligonucleotide
ucuaguuuggagauggcagu

SEQ ID NO: 527 - RNA, 20 nt, Artificial Sequence
  Description of Artificial Sequence Synthetic oligonucleotide
auuucuaguuuggagauggc

SEQ ID NO: 528 - RNA, 20 nt, Artificial Sequence
  Description of Artificial Sequence Synthetic oligonucleotide
uggcauuucuaguuuggaga

SEQ ID NO: 529 - RNA, 20 nt, Artificial Sequence
  Description of Artificial Sequence Synthetic oligonucleotide
gauggcauuucuaguuugga

SEQ ID NO: 530 - RNA, 20 nt, Artificial Sequence
  Description of Artificial Sequence Synthetic oligonucleotide
agauggcauuucuaguuugg

SEQ ID NO: 531 - RNA, 20 nt, Artificial Sequence
  Description of Artificial Sequence Synthetic oligonucleotide
aagauggcauuucuaguuug

SEQ ID NO: 532 - RNA, 20 nt, Artificial Sequence
  Description of Artificial Sequence Synthetic oligonucleotide
aggaagauggcauuucuagu

SEQ ID NO: 533 - RNA, 20 nt, Artificial Sequence
  Description of Artificial Sequence Synthetic oligonucleotide
aaggaagauggcauuucuag

SEQ ID NO: 534 - RNA, 20 nt, Artificial Sequence
  Description of Artificial Sequence Synthetic oligonucleotide
caaggaagauggcauuucua

SEQ ID NO: 535 - RNA, 20 nt, Artificial Sequence
  Description of Artificial Sequence Synthetic oligonucleotide
ucaaggaagauggcauuucu

SEQ ID NO: 536 - RNA, 20 nt, Artificial Sequence
  Description of Artificial Sequence Synthetic oligonucleotide
acaucaaggaagauggcauu

SEQ ID NO: 537 - RNA, 20 nt, Artificial Sequence
  Description of Artificial Sequence Synthetic oligonucleotide
caacaucaaggaagauggca

SEQ ID NO: 538 - RNA, 20 nt, Artificial Sequence
  Description of Artificial Sequence Synthetic oligonucleotide
uccaacaucaaggaagaugg

SEQ ID NO: 539 - RNA, 20 nt, Artificial Sequence
  Description of Artificial Sequence Synthetic oligonucleotide
ccuccaacaucaaggaagau

SEQ ID NO: 540 - RNA, 20 nt, Artificial Sequence
  Description of Artificial Sequence Synthetic oligonucleotide
agguaccuccaacaucaagg

SEQ ID NO: 541 - RNA, 20 nt, Artificial Sequence
  Description of Artificial Sequence Synthetic oligonucleotide
cagguaccuccaacaucaag

SEQ ID NO: 542 - RNA, 20 nt, Artificial Sequence
  Description of Artificial Sequence Synthetic oligonucleotide
agagcagguaccuccaacau

SEQ ID NO: 543 - RNA, 20 nt, Artificial Sequence
  Description of Artificial Sequence Synthetic oligonucleotide
cagagcagguaccuccaaca

SEQ ID NO: 544 - RNA, 20 nt, Artificial Sequence
  Description of Artificial Sequence Synthetic oligonucleotide
ccagagcagguaccuccaac

SEQ ID NO: 545 - RNA, 20 nt, Artificial Sequence
  Description of Artificial Sequence Synthetic oligonucleotide
gccagagcagguaccuccaa

SEQ ID NO: 546 - RNA, 20 nt, Artificial Sequence
  Description of Artificial Sequence Synthetic oligonucleotide
ugccagagcagguaccucca

SEQ ID NO: 547 - RNA, 20 nt, Artificial Sequence
  Description of Artificial Sequence Synthetic oligonucleotide
cugccagagcagguaccucc

SEQ ID NO: 548 - RNA, 20 nt, Artificial Sequence
  Description of Artificial Sequence Synthetic oligonucleotide
ucugccagagcagguaccuc

SEQ ID NO: 549 - RNA, 20 nt, Artificial Sequence
  Description of Artificial Sequence Synthetic oligonucleotide
aucugccagagcagguaccu

SEQ ID NO: 550 - RNA, 20 nt, Artificial Sequence
  Description of Artificial Sequence Synthetic oligonucleotide
uugaaaucugccagagcagg

SEQ ID NO: 551 - RNA, 20 nt, Artificial Sequence
  Description of Artificial Sequence Synthetic oligonucleotide
cccgguugaaaucugccaga

SEQ ID NO: 552 - RNA, 20 nt, Artificial Sequence
  Description of Artificial Sequence Synthetic oligonucleotide
gcccgguugaaaucugccag

SEQ ID NO: 553 - RNA, 20 nt, Artificial Sequence
  Description of Artificial Sequence Synthetic oligonucleotide
agcccgguugaaaucugcca

SEQ ID NO: 554 - RNA, 20 nt, Artificial Sequence
  Description of Artificial Sequence Synthetic oligonucleotide
ccaagcccgguugaaaucug

SEQ ID NO: 555 - RNA, 20 nt, Artificial Sequence
  Description of Artificial Sequence Synthetic oligonucleotide
uccaagcccgguugaaaucu

SEQ ID NO: 556 - RNA, 20 nt, Artificial Sequence
  Description of Artificial Sequence Synthetic oligonucleotide
guccaagcccgguugaaauc

SEQ ID NO: 557 - RNA, 20 nt, Artificial Sequence
  Description of Artificial Sequence Synthetic oligonucleotide
uguccaagcccgguugaaau

SEQ ID NO: 558 - RNA, 20 nt, Artificial Sequence
  Description of Artificial Sequence Synthetic oligonucleotide
cuguccaagcccgguugaaa

SEQ ID NO: 559 - RNA, 20 nt, Artificial Sequence
  Description of Artificial Sequence Synthetic oligonucleotide
ucuguccaagcccgguugaa

SEQ ID NO: 560 - RNA, 20 nt, Artificial Sequence
  Description of Artificial Sequence Synthetic oligonucleotide
uucuguccaagcccgguuga

SEQ ID NO: 561 - RNA, 20 nt, Artificial Sequence
  Description of Artificial Sequence Synthetic oligonucleotide
guucuguccaagcccgguug

SEQ ID NO: 562 - RNA, 20 nt, Artificial Sequence
  Description of Artificial Sequence Synthetic oligonucleotide
aguucuguccaagcccgguu

SEQ ID NO: 563 - RNA, 20 nt, Artificial Sequence
  Description of Artificial Sequence Synthetic oligonucleotide
aaguucuguccaagcccggu

SEQ ID NO: 564 - RNA, 20 nt, Artificial Sequence
  Description of Artificial Sequence Synthetic oligonucleotide
uaaguucuguccaagcccgg

SEQ ID NO: 565 - RNA, 20 nt, Artificial Sequence
  Description of Artificial Sequence Synthetic oligonucleotide
guaaguucuguccaagcccg

SEQ ID NO: 566 - RNA, 20 nt, Artificial Sequence
  Description of Artificial Sequence Synthetic oligonucleotide
gguaaguucuguccaagccc

SEQ ID NO: 567 - RNA, 20 nt, Artificial Sequence
  Description of Artificial Sequence Synthetic oligonucleotide
cagucgguaaguucugucca

SEQ ID NO: 568 - RNA, 20 nt, Artificial Sequence
  Description of Artificial Sequence Synthetic oligonucleotide
ccagucgguaaguucugucc

SEQ ID NO: 569 - RNA, 20 nt, Artificial Sequence
  Description of Artificial Sequence Synthetic oligonucleotide
ccaccaucacccucugugau

SEQ ID NO: 570 - RNA, 20 nt, Artificial Sequence
  Description of Artificial Sequence Synthetic oligonucleotide
cccaccaucacccucuguga

SEQ ID NO: 571 - RNA, 20 nt, Artificial Sequence
  Description of Artificial Sequence Synthetic oligonucleotide
cacccaccaucacccucugu

SEQ ID NO: 572 - RNA, 20 nt, Artificial Sequence
  Description of Artificial Sequence Synthetic oligonucleotide
ucacccaccaucacccucug

SEQ ID NO: 573 - RNA, 20 nt, Artificial Sequence
  Description of Artificial Sequence Synthetic oligonucleotide
gucacccaccaucacccucu

SEQ ID NO: 574 - RNA, 20 nt, Artificial Sequence
  Description of Artificial Sequence Synthetic oligonucleotide
ggucacccaccaucacccuc

SEQ ID NO: 575 - RNA, 20 nt, Artificial Sequence
  Description of Artificial Sequence Synthetic oligonucleotide
ucaagcagagaaagccaguc

SEQ ID NO: 576 - RNA, 20 nt, Artificial Sequence
  Description of Artificial Sequence Synthetic oligonucleotide
uugaucaagcagagaaagcc

SEQ ID NO: 577 - RNA, 25 nt, Artificial Sequence
  Description of Artificial Sequence Synthetic oligonucleotide
caaagaagauggcauuucuaguuug

SEQ ID NO: 578 - RNA, 20 nt, Artificial Sequence
  Description of Artificial Sequence Synthetic oligonucleotide
gcaaagaagauggcauuucu

SEQ ID NO: 579 - RNA, 20 nt, Artificial Sequence
  Description of Artificial Sequence Synthetic oligonucleotide
gcaaagaagauggcauuucu

SEQ ID NO: 580 - RNA, 20 nt, Artificial Sequence
  Description of Artificial Sequence Synthetic oligonucleotide
ucaaggaagauggcauuucu

SEQ ID NO: 581 - RNA, 20 nt, Artificial Sequence
  Description of Artificial Sequence Synthetic oligonucleotide
ucaaggaagauggcauuucu

SEQ ID NO: 582 - RNA, 20 nt, Artificial Sequence
  Description of Artificial Sequence Synthetic oligonucleotide
ucaaggaagauggcauuucu

SEQ ID NO: 583 - RNA, 20 nt, Artificial Sequence
  Description of Artificial Sequence Synthetic oligonucleotide
ucaaggaagauggcauuucu

SEQ ID NO: 584 - RNA, 20 nt, Artificial Sequence
  Description of Artificial Sequence Synthetic oligonucleotide
ucaaggaagauggcauuucu

SEQ ID NO: 585 - RNA, 20 nt, Artificial Sequence
  Description of Artificial Sequence Synthetic oligonucleotide
ucaaggaagauggcauuucu

SEQ ID NO: 586 - RNA, 20 nt, Artificial Sequence
  Description of Artificial Sequence Synthetic oligonucleotide
ucaaggaagauggcauuucu

SEQ ID NO: 587 - RNA, 20 nt, Artificial Sequence
  Description of Artificial Sequence Synthetic oligonucleotide
ucaaggaagauggcauuucu

SEQ ID NO: 588 - RNA, 20 nt, Artificial Sequence
  Description of Artificial Sequence Synthetic oligonucleotide
ucaaggaagauggcauuucu

SEQ ID NO: 589 - RNA, 20 nt, Artificial Sequence
  Description of Artificial Sequence Synthetic oligonucleotide
ucaaggaagauggcauuucu

SEQ ID NO: 590 - RNA, 20 nt, Artificial Sequence
  Description of Artificial Sequence Synthetic oligonucleotide
ucaaggaagauggcauuucu

SEQ ID NO: 591 - RNA, 20 nt, Artificial Sequence
  Description of Artificial Sequence Synthetic oligonucleotide
ucaaggaagauggcauuucu

SEQ ID NO: 592 - RNA, 20 nt, Artificial Sequence
  Description of Artificial Sequence Synthetic oligonucleotide
ucaaggaagauggcauuucu

SEQ ID NO: 593 - RNA, 20 nt, Artificial Sequence
  Description of Artificial Sequence Synthetic oligonucleotide
ucaaggaagauggcauuucu

SEQ ID NO: 594 - RNA, 20 nt, Artificial Sequence
  Description of Artificial Sequence Synthetic oligonucleotide
ucaaggaagauggcauuucu

SEQ ID NO: 595 - RNA, 20 nt, Artificial Sequence
  Description of Artificial Sequence Synthetic oligonucleotide
ucaaggaagauggcauuucu

SEQ ID NO: 596 - RNA, 20 nt, Artificial Sequence
  Description of Artificial Sequence Synthetic oligonucleotide
ucaaggaagauggcauuucu

SEQ ID NO: 597 - RNA, 20 nt, Artificial Sequence
  Description of Artificial Sequence Synthetic oligonucleotide
ucaaggaagauggcauuucu

SEQ ID NO: 598 - RNA, 20 nt, Artificial Sequence
  Description of Artificial Sequence Synthetic oligonucleotide
ucaaggaagauggcauuucu

SEQ ID NO: 599 - RNA, 20 nt, Artificial Sequence
  Description of Artificial Sequence Synthetic oligonucleotide
ucaaggaagauggcauuucu

SEQ ID NO: 600 - RNA, 20 nt, Artificial Sequence
  Description of Artificial Sequence Synthetic oligonucleotide
ucaaggaagauggcauuucu

SEQ ID NO: 601 - RNA, 20 nt, Artificial Sequence
  Description of Artificial Sequence Synthetic oligonucleotide
ucaaggaagauggcauuucu

SEQ ID NO: 602 - RNA, 20 nt, Artificial Sequence
  Description of Artificial Sequence Synthetic oligonucleotide
ucaaggaagauggcauuucu

SEQ ID NO: 603 - RNA, 20 nt, Artificial Sequence
  Description of Artificial Sequence Synthetic oligonucleotide
ucaaggaagauggcauuucu

SEQ ID NO: 604 - RNA, 20 nt, Artificial Sequence
  Description of Artificial Sequence Synthetic oligonucleotide
ucaaggaagauggcauuucu

SEQ ID NO: 605 - RNA, 20 nt, Artificial Sequence
  Description of Artificial Sequence Synthetic oligonucleotide
ucaaggaagauggcauuucu

SEQ ID NO: 606 - RNA, 20 nt, Artificial Sequence
  Description of Artificial Sequence Synthetic oligonucleotide
ucaaggaagauggcauuucu

SEQ ID NO: 607 - RNA, 20 nt, Artificial Sequence
  Description of Artificial Sequence Synthetic oligonucleotide
ucaaggaagauggcauuucu

SEQ ID NO: 608 - RNA, 20 nt, Artificial Sequence
  Description of Artificial Sequence Synthetic oligonucleotide
ucaaggaagauggcauuucu

SEQ ID NO: 609 - RNA, 20 nt, Artificial Sequence
  Description of Artificial Sequence Synthetic oligonucleotide
ggccaaaccucggcuuaccu

SEQ ID NO: 610 - RNA, 20 nt, Artificial Sequence
  Description of Artificial Sequence Synthetic oligonucleotide
ggccaaaccucggcuuaccu

SEQ ID NO: 611 - RNA, 20 nt, Artificial Sequence
  Description of Artificial Sequence Synthetic oligonucleotide
ggccaaaccucggcuuaccu

SEQ ID NO: 612 - RNA, 20 nt, Artificial Sequence
  Description of Artificial Sequence Synthetic oligonucleotide
ggccaaaccucggcuuaccu

SEQ ID NO: 613 - RNA, 20 nt, Artificial Sequence
  Description of Artificial Sequence Synthetic oligonucleotide
ggccaaaccucggcuuaccu

SEQ ID NO: 614 - RNA, 20 nt, Artificial Sequence
  Description of Artificial Sequence Synthetic oligonucleotide
ggccaaaccucggcuuaccu

SEQ ID NO: 615 - RNA, 20 nt, Artificial Sequence
  Description of Artificial Sequence Synthetic oligonucleotide
ggccaaaccucggcuuaccu

SEQ ID NO: 616 - RNA, 20 nt, Artificial Sequence
  Description of Artificial Sequence Synthetic oligonucleotide
ggccaaaccucggcuuaccu

SEQ ID NO: 617 - RNA, 20 nt, Artificial Sequence
  Description of Artificial Sequence Synthetic oligonucleotide
ucaaggaagauggcauuucu

SEQ ID NO: 618 - RNA, 20 nt, Artificial Sequence
  Description of Artificial Sequence Synthetic oligonucleotide
ucaaggaagauggcauuucu

SEQ ID NO: 619 - RNA, 20 nt, Artificial Sequence
  Description of Artificial Sequence Synthetic oligonucleotide
ucaaggaagauggcauuucu

SEQ ID NO: 620 - RNA, 20 nt, Artificial Sequence
  Description of Artificial Sequence Synthetic oligonucleotide
ucaaggaagauggcauuucu

SEQ ID NO: 621 - RNA, 20 nt, Artificial Sequence
  Description of Artificial Sequence Synthetic oligonucleotide
ucaaggaagauggcauuucu

SEQ ID NO: 622 - RNA, 20 nt, Artificial Sequence
  Description of Artificial Sequence Synthetic oligonucleotide
ucaaggaagauggcauuucu

SEQ ID NO: 623 - RNA, 20 nt, Artificial Sequence
  Description of Artificial Sequence Synthetic oligonucleotide
ucaaggaagauggcauuucu

SEQ ID NO: 624 - RNA, 20 nt, Artificial Sequence
  Description of Artificial Sequence Synthetic oligonucleotide
ucaaggaagauggcauuucu

SEQ ID NO: 625 - DNA, 20 bp, Artificial Sequence
  Description of Artificial Sequence Synthetic oligonucleotide
ggccaaacctcggcttacct

SEQ ID NO: 626 - RNA, 20 nt, Artificial Sequence
  Description of Artificial Sequence Synthetic oligonucleotide
ggccaaaccucggcuuaccu

SEQ ID NO: 627 - DNA, 20 bp, Artificial Sequence
  Description of Artificial Sequence Synthetic oligonucleotide
ggccaaacctcggcttacct

SEQ ID NO: 628 - DNA, 20 bp, Artificial Sequence
  Description of Artificial Sequence Synthetic oligonucleotide
ggccaaacctcggcttacct

SEQ ID NO: 629 - DNA, 20 bp, Artificial Sequence
  Description of Artificial Sequence Synthetic oligonucleotide
ggccaaaccucggcttacct

SEQ ID NO: 630 - DNA, 20 bp, Artificial Sequence
  Description of Artificial Sequence Synthetic oligonucleotide
ggccaaacctcggcttaccu

SEQ ID NO: 631 - DNA, 20 bp, Artificial Sequence
  Description of Artificial Sequence Synthetic oligonucleotide
ggccaaaccucggctuaccu

SEQ ID NO: 632 - DNA, 20 bp, Artificial Sequence
  Description of Artificial Sequence Synthetic oligonucleotide
ggccaaacctcggcutaccu

SEQ ID NO: 633 - DNA, 20 bp, Artificial Sequence
  Description of Artificial Sequence Synthetic oligonucleotide
ggccaaacctcggctuaccu

SEQ ID NO: 634 - RNA, 20 nt, Artificial Sequence
  Description of Artificial Sequence Synthetic oligonucleotide
ggccaaaccucggcuuaccu

SEQ ID NO: 635 - DNA, 20 bp, Artificial Sequence
  Description of Artificial Sequence Synthetic oligonucleotide
ggccaaacctcggcutaccu

SEQ ID NO: 636 - DNA, 20 bp, Artificial Sequence
  Description of Artificial Sequence Synthetic oligonucleotide
ggccaaacctcggctuaccu

SEQ ID NO: 637 - DNA, 20 bp, Artificial Sequence
  Description of Artificial Sequence Synthetic oligonucleotide
ggccaaaccucggcttaccu

SEQ ID NO: 638 - DNA, 20 bp, Artificial Sequence
  Description of Artificial Sequence Synthetic oligonucleotide
ggccaaaccucggcttacct

SEQ ID NO: 639 - DNA, 20 bp, Artificial Sequence
  Description of Artificial Sequence Synthetic oligonucleotide
ggccaaacctcggcttaccu

SEQ ID NO: 640 - DNA, 20 bp, Artificial Sequence
  Description of Artificial Sequence Synthetic oligonucleotide
ggccaaaccucggctuaccu

SEQ ID NO: 641 - DNA, 20 bp, Artificial Sequence
  Description of Artificial Sequence Synthetic oligonucleotide
ggccaaacctcggcutaccu

SEQ ID NO: 642 - DNA, 20 bp, Artificial Sequence
  Description of Artificial Sequence Synthetic oligonucleotide
ggccaaacctcggctuaccu

SEQ ID NO: 643 - RNA, 20 nt, Artificial Sequence
  Description of Artificial Sequence Synthetic oligonucleotide
ggccaaaccucggcuuaccu

SEQ ID NO: 644 - DNA, 20 bp, Artificial Sequence
  Description of Artificial Sequence Synthetic oligonucleotide
ggccaaacctcggcutaccu

SEQ ID NO: 645 - DNA, 20 bp, Artificial Sequence
  Description of Artificial Sequence Synthetic oligonucleotide
ggccaaacctcggctuaccu

SEQ ID NO: 646 - DNA, 20 bp, Artificial Sequence
  Description of Artificial Sequence Synthetic oligonucleotide
ggccaaaccucggcttaccu

SEQ ID NO: 647 - DNA, 20 bp, Artificial Sequence
  Description of Artificial Sequence Synthetic oligonucleotide
ggccaaaccucggcutaccu

SEQ ID NO: 648 - RNA, 20 nt, Artificial Sequence
  Description of Artificial Sequence Synthetic oligonucleotide
ggccaaaccucggcuuaccu

SEQ ID NO: 649 - RNA, 20 nt, Artificial Sequence
  Description of Artificial Sequence Synthetic oligonucleotide
ggccaaaccucggcuuaccu

SEQ ID NO: 650 - RNA, 20 nt, Artificial Sequence
  Description of Artificial Sequence Synthetic oligonucleotide
ggccaaaccucggcuuaccu

SEQ ID NO: 651 - RNA, 20 nt, Artificial Sequence
  Description of Artificial Sequence Synthetic oligonucleotide
ggccaaaccucggcuuaccu

SEQ ID NO: 652 - RNA, 20 nt, Artificial Sequence
  Description of Artificial Sequence Synthetic oligonucleotide
ggccaaaccucggcuuaccu

SEQ ID NO: 653 - RNA, 20 nt, Artificial Sequence
  Description of Artificial Sequence Synthetic oligonucleotide
ggccaaaccucggcuuaccu

SEQ ID NO: 654 - RNA, 20 nt, Artificial Sequence
  Description of Artificial Sequence Synthetic oligonucleotide
ggccaaaccucggcuuaccu

SEQ ID NO: 655 - RNA, 20 nt, Artificial Sequence
  Description of Artificial Sequence Synthetic oligonucleotide
ggccaaaccucggcuuaccu

SEQ ID NO: 656 - RNA, 20 nt, Artificial Sequence
  Description of Artificial Sequence Synthetic oligonucleotide
ggccaaaccucggcuuaccu

SEQ ID NO: 657 - RNA, 20 nt, Artificial Sequence
  Description of Artificial Sequence Synthetic oligonucleotide
ggccaaaccucggcuuaccu

SEQ ID NO: 658 - RNA, 20 nt, Artificial Sequence
  Description of Artificial Sequence Synthetic oligonucleotide
ggccaaaccucggcuuaccu

SEQ ID NO: 659 - RNA, 35 nt, Artificial Sequence
  Description of Artificial Sequence Synthetic oligonucleotide
ggccaaaccucggcuuaccugaaaggccaaaccuc

SEQ ID NO: 660 - DNA, 20 bp, Artificial Sequence
  Description of Artificial Sequence Synthetic oligonucleotide
ggccaaaccucggcutaccu

SEQ ID NO: 661 - DNA, 20 bp, Artificial Sequence
  Description of Artificial Sequence Synthetic oligonucleotide
tcaaggaagatggcatttct

SEQ ID NO: 662 - RNA, 20 nt, Artificial Sequence
  Description of Artificial Sequence Synthetic oligonucleotide
ucaaggaagauggcauuucu

SEQ ID NO: 663 - DNA, 20 bp, Artificial Sequence
  Description of Artificial Sequence Synthetic oligonucleotide
tcaaggaagatggcatttct

SEQ ID NO: 664 - DNA, 20 bp, Artificial Sequence
  Description of Artificial Sequence Synthetic oligonucleotide
tcaaggaagatggcatttct

SEQ ID NO: 665 - DNA, 20 bp, Artificial Sequence
  Description of Artificial Sequence Synthetic oligonucleotide
tcaaggaagauggcatttct

SEQ ID NO: 666 - DNA, 20 bp, Artificial Sequence
  Description of Artificial Sequence Synthetic oligonucleotide
ucaaggaagatggcatuucu

SEQ ID NO: 667 - DNA, 20 bp, Artificial Sequence
  Description of Artificial Sequence Synthetic oligonucleotide
tcaaggaagatggcautucu

SEQ ID NO: 668 - DNA, 20 bp, Artificial Sequence
  Description of Artificial Sequence Synthetic oligonucleotide
ucaaggaagauggcatutcu

SEQ ID NO: 669 - DNA, 20 bp, Artificial Sequence
  Description of Artificial Sequence Synthetic oligonucleotide
tcaaggaagatggcauutcu

SEQ ID NO: 670 - DNA, 20 bp, Artificial Sequence
  Description of Artificial Sequence Synthetic oligonucleotide
tcaaggaagauggcauttcu

SEQ ID NO: 671 - DNA, 20 bp, Artificial Sequence
  Description of Artificial Sequence Synthetic oligonucleotide
tcaaggaagatggcatttcu

SEQ ID NO: 672 - DNA, 20 bp, Artificial Sequence
  Description of Artificial Sequence Synthetic oligonucleotide
tcaaggaagatggcauttcu

SEQ ID NO: 673 - DNA, 20 bp, Artificial Sequence
  Description of Artificial Sequence Synthetic oligonucleotide
ucaaggaagauggcatttcu

SEQ ID NO: 674 - DNA, 20 bp, Artificial Sequence
  Description of Artificial Sequence Synthetic oligonucleotide
tcaaggaagatggcatttcu

SEQ ID NO: 675 - DNA, 20 bp, Artificial Sequence
  Description of Artificial Sequence Synthetic oligonucleotide
tcaaggaagauggcatttct

SEQ ID NO: 676 - DNA, 20 bp, Artificial Sequence
  Description of Artificial Sequence Synthetic oligonucleotide
ucaaggaagatggcatuucu

SEQ ID NO: 677 - DNA, 20 bp, Artificial Sequence
  Description of Artificial Sequence Synthetic oligonucleotide
tcaaggaagatggcautucu

SEQ ID NO: 678 - DNA, 20 bp, Artificial Sequence
  Description of Artificial Sequence Synthetic oligonucleotide
ucaaggaagauggcatutcu

SEQ ID NO: 679 - DNA, 20 bp, Artificial Sequence
  Description of Artificial Sequence Synthetic oligonucleotide
tcaaggaagatggcauutcu

SEQ ID NO: 680 - DNA, 20 bp, Artificial Sequence
  Description of Artificial Sequence Synthetic oligonucleotide
tcaaggaagauggcauttcu

SEQ ID NO: 681 - DNA, 20 bp, Artificial Sequence
  Description of Artificial Sequence Synthetic oligonucleotide
tcaaggaagatggcatttcu

SEQ ID NO: 682 - DNA, 20 bp, Artificial Sequence
  Description of Artificial Sequence Synthetic oligonucleotide
tcaaggaagatggcauttcu

SEQ ID NO: 683 - DNA, 20 bp, Artificial Sequence
  Description of Artificial Sequence Synthetic oligonucleotide
ucaaggaagauggcatttcu

SEQ ID NO: 684 - DNA, 20 bp, Artificial Sequence
  Description of Artificial Sequence Synthetic oligonucleotide
tcaaggaagatggcatttcu

SEQ ID NO: 685 - RNA, 20 nt, Artificial Sequence
  Description of Artificial Sequence Synthetic oligonucleotide
ucaaggaagauggcauuucu

SEQ ID NO: 686 - RNA, 20 nt, Artificial Sequence
  Description of Artificial Sequence Synthetic oligonucleotide
ucaaggaagauggcauuucu

SEQ ID NO: 687 - RNA, 20 nt, Artificial Sequence
  Description of Artificial Sequence Synthetic oligonucleotide
ucaaggaagauggcauuucu

SEQ ID NO: 688 - RNA, 20 nt, Artificial Sequence
  Description of Artificial Sequence Synthetic oligonucleotide
ucaaggaagauggcauuucu

SEQ ID NO: 689 - RNA, 20 nt, Artificial Sequence
  Description of Artificial Sequence Synthetic oligonucleotide
ucaaggaagauggcauuucu

SEQ ID NO: 690 - RNA, 20 nt, Artificial Sequence
  Description of Artificial Sequence Synthetic oligonucleotide
ucaaggaagauggcauuucu

SEQ ID NO: 691 - RNA, 20 nt, Artificial Sequence
  Description of Artificial Sequence Synthetic oligonucleotide
ucaaggaagauggcauuucu

SEQ ID NO: 692 - RNA, 20 nt, Artificial Sequence
  Description of Artificial Sequence Synthetic oligonucleotide
ucaaggaagauggcauuucu

SEQ ID NO: 693 - RNA, 20 nt, Artificial Sequence
  Description of Artificial Sequence Synthetic oligonucleotide
ucaaggaagauggcauuucu

SEQ ID NO: 694 - RNA, 20 nt, Artificial Sequence
  Description of Artificial Sequence Synthetic oligonucleotide
ucaaggaagauggcauuucu

SEQ ID NO: 695 - RNA, 20 nt, Artificial Sequence
  Description of Artificial Sequence Synthetic oligonucleotide
agaaaugccaucuuccuuga

SEQ ID NO: 696 - RNA, 20 nt, Artificial Sequence
  Description of Artificial Sequence Synthetic oligonucleotide
ucaaggaagauggcauuucu

SEQ ID NO: 697 - RNA, 20 nt, Artificial Sequence
  Description of Artificial Sequence Synthetic oligonucleotide
ucaaggaagauggcauuucu

SEQ ID NO: 698 - RNA, 20 nt, Artificial Sequence
  Description of Artificial Sequence Synthetic oligonucleotide
ucaaggaagauggcauuucu

SEQ ID NO: 699 - RNA, 20 nt, Artificial Sequence
  Description of Artificial Sequence Synthetic oligonucleotide
ucaaggaagauggcauuucu

SEQ ID NO: 700 - RNA, 20 nt, Artificial Sequence
  Description of Artificial Sequence Synthetic oligonucleotide
ucaaggaagauggcauuucu

SEQ ID NO: 701 - RNA, 20 nt, Artificial Sequence
  Description of Artificial Sequence Synthetic oligonucleotide
ucaaggaagauggcauuucu

SEQ ID NO: 702 - RNA, 20 nt, Artificial Sequence
  Description of Artificial Sequence Synthetic oligonucleotide
ucaaggaagauggcauuucu

SEQ ID NO: 703 - RNA, 20 nt, Artificial Sequence
  Description of Artificial Sequence Synthetic oligonucleotide
ucaaggaagauggcauuucu

SEQ ID NO: 704 - RNA, 20 nt, Artificial Sequence
  Description of Artificial Sequence Synthetic oligonucleotide
ucaaggaagauggcauuucu

SEQ ID NO: 705 - RNA, 20 nt, Artificial Sequence
  Description of Artificial Sequence Synthetic oligonucleotide
ucaaggaagauggcauuucu

SEQ ID NO: 706 - RNA, 20 nt, Artificial Sequence
  Description of Artificial Sequence Synthetic oligonucleotide
ucaaggaagauggcauuucu

SEQ ID NO: 707 - RNA, 20 nt, Artificial Sequence
  Description of Artificial Sequence Synthetic oligonucleotide
ucaaggaagauggcauuucu

SEQ ID NO: 708 - RNA, 20 nt, Artificial Sequence
  Description of Artificial Sequence Synthetic oligonucleotide
ucaaggaagauggcauuucu

SEQ ID NO: 709 - RNA, 20 nt, Artificial Sequence
  Description of Artificial Sequence Synthetic oligonucleotide
ucaaggaagauggcauuucu

SEQ ID NO: 710 - RNA, 20 nt, Artificial Sequence
  Description of Artificial Sequence Synthetic oligonucleotide
ucaaggaagauggcauuucu

SEQ ID NO: 711 - RNA, 20 nt, Artificial Sequence
  Description of Artificial Sequence Synthetic oligonucleotide
ucaaggaagauggcauuucu

SEQ ID NO: 712 - RNA, 20 nt, Artificial Sequence
  Description of Artificial Sequence Synthetic oligonucleotide
ucaaggaagauggcauuucu

SEQ ID NO: 713 - RNA, 20 nt, Artificial Sequence
  Description of Artificial Sequence Synthetic oligonucleotide
ucaaggaagauggcauuucu

SEQ ID NO: 714 - RNA, 20 nt, Artificial Sequence
  Description of Artificial Sequence Synthetic oligonucleotide
ucaaggaagauggcauuucu

SEQ ID NO: 715 - RNA, 20 nt, Artificial Sequence
  Description of Artificial Sequence Synthetic oligonucleotide
ucaaggaagauggcauuucu

SEQ ID NO: 716 - RNA, 20 nt, Artificial Sequence
  Description of Artificial Sequence Synthetic oligonucleotide
ucaaggaagauggcauuucu

SEQ ID NO: 717 - RNA, 20 nt, Artificial Sequence
  Description of Artificial Sequence Synthetic oligonucleotide
ucaaggaagauggcauuucu

SEQ ID NO: 718 - RNA, 20 nt, Artificial Sequence
  Description of Artificial Sequence Synthetic oligonucleotide
ucaaggaagauggcauuucu

SEQ ID NO: 719 - RNA, 20 nt, Artificial Sequence
  Description of Artificial Sequence Synthetic oligonucleotide
ucaaggaagauggcauuucu

SEQ ID NO: 720 - RNA, 20 nt, Artificial Sequence
  Description of Artificial Sequence Synthetic oligonucleotide
ucaaggaagauggcauuucu

SEQ ID NO: 721 - RNA, 20 nt, Artificial Sequence
  Description of Artificial Sequence Synthetic oligonucleotide
ucaaggaagauggcauuucu

SEQ ID NO: 722 - RNA, 20 nt, Artificial Sequence
  Description of Artificial Sequence Synthetic oligonucleotide
ucaaggaagauggcauuucu

SEQ ID NO: 723 - RNA, 20 nt, Artificial Sequence
  Description of Artificial Sequence Synthetic oligonucleotide
ucaaggaagauggcauuucu

SEQ ID NO: 724 - RNA, 20 nt, Artificial Sequence
  Description of Artificial Sequence Synthetic oligonucleotide
ucaaggaagauggcauuucu

SEQ ID NO: 725 - RNA, 20 nt, Artificial Sequence
  Description of Artificial Sequence Synthetic oligonucleotide
ucaaggaagauggcauuucu

SEQ ID NO: 726 - RNA, 20 nt, Artificial Sequence
  Description of Artificial Sequence Synthetic oligonucleotide
ucaaggaagauggcauuucu

SEQ ID NO: 727 - RNA, 20 nt, Artificial Sequence
  Description of Artificial Sequence Synthetic oligonucleotide
ucaaggaagauggcauuucu

SEQ ID NO: 728 - RNA, 20 nt, Artificial Sequence
  Description of Artificial Sequence Synthetic oligonucleotide
ucaaggaagauggcauuucu

SEQ ID NO: 729 - RNA, 20 nt, Artificial Sequence
  Description of Artificial Sequence Synthetic oligonucleotide
ucaaggaagauggcauuucu

SEQ ID NO: 730 - RNA, 20 nt, Artificial Sequence
  Description of Artificial Sequence Synthetic oligonucleotide
ucaaggaagauggcauuucu

SEQ ID NO: 731 - RNA, 20 nt, Artificial Sequence
  Description of Artificial Sequence Synthetic oligonucleotide
ucaaggaagauggcauuucu

SEQ ID NO: 732 - RNA, 20 nt, Artificial Sequence
  Description of Artificial Sequence Synthetic oligonucleotide
ucaaggaagauggcauuucu

SEQ ID NO: 733 - RNA, 20 nt, Artificial Sequence
  Description of Artificial Sequence Synthetic oligonucleotide
ucaaggaagauggcauuucu

SEQ ID NO: 734 - RNA, 20 nt, Artificial Sequence
  Description of Artificial Sequence Synthetic oligonucleotide
ucaaggaagauggcauuucu

SEQ ID NO: 735 - RNA, 20 nt, Artificial Sequence
  Description of Artificial Sequence Synthetic oligonucleotide
ucaaggaagauggcauuucu

SEQ ID NO: 736 - RNA, 20 nt, Artificial Sequence
  Description of Artificial Sequence Synthetic oligonucleotide
ucaaggaagauggcauuucu

SEQ ID NO: 737 - RNA, 20 nt, Artificial Sequence
  Description of Artificial Sequence Synthetic oligonucleotide
ucaaggaagauggcauuucu

SEQ ID NO: 738 - RNA, 20 nt, Artificial Sequence
  Description of Artificial Sequence Synthetic oligonucleotide
ucaaggaagauggcauuucu

SEQ ID NO: 739 - RNA, 20 nt, Artificial Sequence
  Description of Artificial Sequence Synthetic oligonucleotide
ucaaggaagauggcauuucu

SEQ ID NO: 740 - RNA, 20 nt, Artificial Sequence
  Description of Artificial Sequence Synthetic oligonucleotide
ucaaggaagauggcauuucu

SEQ ID NO: 741 - RNA, 20 nt, Artificial Sequence
  Description of Artificial Sequence Synthetic oligonucleotide
ucaaggaagauggcauuucu

SEQ ID NO: 742 - RNA, 20 nt, Artificial Sequence
  Description of Artificial Sequence Synthetic oligonucleotide
ucaaggaagauggcauuucu

SEQ ID NO: 743 - RNA, 20 nt, Artificial Sequence
  Description of Artificial Sequence Synthetic oligonucleotide
ucaaggaagauggcauuucu

SEQ ID NO: 744 - RNA, 20 nt, Artificial Sequence
  Description of Artificial Sequence Synthetic oligonucleotide
ucaaggaagauggcauuucu

SEQ ID NO: 745 - RNA, 20 nt, Artificial Sequence
  Description of Artificial Sequence Synthetic oligonucleotide
ucaaggaagauggcauuucu

SEQ ID NO: 746 - RNA, 20 nt, Artificial Sequence
  Description of Artificial Sequence Synthetic oligonucleotide
ucaaggaagauggcauuucu

SEQ ID NO: 747 - RNA, 20 nt, Artificial Sequence
  Description of Artificial Sequence Synthetic oligonucleotide
ucaaggaagauggcauuucu

SEQ ID NO: 748 - RNA, 20 nt, Artificial Sequence
  Description of Artificial Sequence Synthetic oligonucleotide
ucaaggaagauggcauuucu

SEQ ID NO: 749 - RNA, 20 nt, Artificial Sequence
  Description of Artificial Sequence Synthetic oligonucleotide
ucaaggaagauggcauuucu

SEQ ID NO: 750 - RNA, 20 nt, Artificial Sequence
  Description of Artificial Sequence Synthetic oligonucleotide
ucaaggaagauggcauuucu

SEQ ID NO: 751 - RNA, 20 nt, Artificial Sequence
  Description of Artificial Sequence Synthetic oligonucleotide
ucaaggaagauggcauuucu

SEQ ID NO: 752 - RNA, 20 nt, Artificial Sequence
  Description of Artificial Sequence Synthetic oligonucleotide
ucaaggaagauggcauuucu

SEQ ID NO: 753 - RNA, 20 nt, Artificial Sequence
  Description of Artificial Sequence Synthetic oligonucleotide
ucaaggaagauggcauuucu

SEQ ID NO: 754 - RNA, 20 nt, Artificial Sequence
  Description of Artificial Sequence Synthetic oligonucleotide
ucaaggaagauggcauuucu

SEQ ID NO: 755 - RNA, 20 nt, Artificial Sequence
  Description of Artificial Sequence Synthetic oligonucleotide
ucaaggaagauggcauuucu

SEQ ID NO: 756 - RNA, 20 nt, Artificial Sequence
  Description of Artificial Sequence Synthetic oligonucleotide
ucaaggaagauggcauuucu

SEQ ID NO: 757 - RNA, 20 nt, Artificial Sequence
  Description of Artificial Sequence Synthetic oligonucleotide
ucaaggaagauggcauuucu

SEQ ID NO: 758 - RNA, 20 nt, Artificial Sequence
  Description of Artificial Sequence Synthetic oligonucleotide
ucaaggaagauggcauuucu

SEQ ID NO: 759 - RNA, 20 nt, Artificial Sequence
  Description of Artificial Sequence Synthetic oligonucleotide
ucaaggaagauggcauuuuu

SEQ ID NO: 760 - RNA, 20 nt, Artificial Sequence
  Description of Artificial Sequence Synthetic oligonucleotide
ucaaggaagauggcauuucu

SEQ ID NO: 761 - RNA, 20 nt, Artificial Sequence
  Description of Artificial Sequence Synthetic oligonucleotide
ucaaggaagauggcauuucu

SEQ ID NO: 762 - RNA, 20 nt, Artificial Sequence
  Description of Artificial Sequence Synthetic oligonucleotide
ucaaggaagauggcauuucu

SEQ ID NO: 763 - RNA, 20 nt, Artificial Sequence
  Description of Artificial Sequence Synthetic oligonucleotide
ucaaggaagauggcauuucu

SEQ ID NO: 764 - RNA, 20 nt, Artificial Sequence
  Description of Artificial Sequence Synthetic oligonucleotide
ucaaggaagauggcauuucu

SEQ ID NO: 765 - RNA, 20 nt, Artificial Sequence
  Description of Artificial Sequence Synthetic oligonucleotide
ucaaggaagauggcauuucu

SEQ ID NO: 766 - RNA, 20 nt, Artificial Sequence
  Description of Artificial Sequence Synthetic oligonucleotide
ucaaggaagauggcauuucu

SEQ ID NO: 767 - RNA, 20 nt, Artificial Sequence
  Description of Artificial Sequence Synthetic oligonucleotide
ucaaggaagauggcauuucu

SEQ ID NO: 768 - RNA, 20 nt, Artificial Sequence
  Description of Artificial Sequence Synthetic oligonucleotide
ucaaggaagauggcauuucu

SEQ ID NO: 769 - RNA, 20 nt, Artificial Sequence
  Description of Artificial Sequence Synthetic oligonucleotide
ucaaggaagauggcauuucu

SEQ ID NO: 770 - RNA, 20 nt, Artificial Sequence
  Description of Artificial Sequence Synthetic oligonucleotide
ucaaggaagauggcauuucu

SEQ ID NO: 771 - RNA, 20 nt, Artificial Sequence
  Description of Artificial Sequence Synthetic oligonucleotide
ucaaggaagauggcauuucu

SEQ ID NO: 772 - RNA, 20 nt, Artificial Sequence
  Description of Artificial Sequence Synthetic oligonucleotide
ucaaggaagauggcauuucu

SEQ ID NO: 773 - RNA, 20 nt, Artificial Sequence
  Description of Artificial Sequence Synthetic oligonucleotide
ucaaggaagauggcauuucu

SEQ ID NO: 774 - RNA, 20 nt, Artificial Sequence
  Description of Artificial Sequence Synthetic oligonucleotide
ucaaggaagauggcauuucu

SEQ ID NO: 775 - RNA, 20 nt, Artificial Sequence
  Description of Artificial Sequence Synthetic oligonucleotide
ucaaggaagauggcauuucu

SEQ ID NO: 776 - RNA, 20 nt, Artificial Sequence
  Description of Artificial Sequence Synthetic oligonucleotide
ucaaggaagauggcauuucu

SEQ ID NO: 777 - RNA, 20 nt, Artificial Sequence
  Description of Artificial Sequence Synthetic oligonucleotide
ucaaggaagauggcauuucu

SEQ ID NO: 778 - RNA, 20 nt, Artificial Sequence
  Description of Artificial Sequence Synthetic oligonucleotide
ucaaggaagauggcauuucu

SEQ ID NO: 779 - RNA, 20 nt, Artificial Sequence
  Description of Artificial Sequence Synthetic oligonucleotide
ucaaggaagauggcauuucu

SEQ ID NO: 780 - RNA, 20 nt, Artificial Sequence
  Description of Artificial Sequence Synthetic oligonucleotide
ucaaggaagauggcauuucu

SEQ ID NO: 781 - RNA, 20 nt, Artificial Sequence
  Description of Artificial Sequence Synthetic oligonucleotide
ucaaggaagauggcauuucu

SEQ ID NO: 782 - RNA, 20 nt, Artificial Sequence
  Description of Artificial Sequence Synthetic oligonucleotide
ucaaggaagauggcauuucu

SEQ ID NO: 783 - RNA, 20 nt, Artificial Sequence
  Description of Artificial Sequence Synthetic oligonucleotide
ucaagcaagauggcauuucu

SEQ ID NO: 784 - RNA, 19 nt, Artificial Sequence
  Description of Artificial Sequence Synthetic oligonucleotide
ucaaggaagaugcauuucu

SEQ ID NO: 785 - RNA, 20 nt, Artificial Sequence
  Description of Artificial Sequence Synthetic oligonucleotide
ucaaggaagauggcauuucu

SEQ ID NO: 786 - RNA, 20 nt, Artificial Sequence
  Description of Artificial Sequence Synthetic oligonucleotide
ucaaggaagauggcauuucu

SEQ ID NO: 787 - RNA, 20 nt, Artificial Sequence
  Description of Artificial Sequence Synthetic oligonucleotide
ucaaggaagauggcauuucu

SEQ ID NO: 788 - RNA, 20 nt, Artificial Sequence
  Description of Artificial Sequence Synthetic oligonucleotide
ucaaggaagauggcauuucu

SEQ ID NO: 789 - RNA, 20 nt, Artificial Sequence
  Description of Artificial Sequence Synthetic oligonucleotide
ucaaggaacauggcauuucu

SEQ ID NO: 790 - RNA, 20 nt, Artificial Sequence
  Description of Artificial Sequence Synthetic oligonucleotide
ucaaggaagauggcauuucu

SEQ ID NO: 791 - RNA, 20 nt, Artificial Sequence
  Description of Artificial Sequence Synthetic oligonucleotide
ucaaggaagauggcauuucu

SEQ ID NO: 792 - RNA, 20 nt, Artificial Sequence
  Description of Artificial Sequence Synthetic oligonucleotide
ucaaggaagauggcauuucu

SEQ ID NO: 793 - RNA, 20 nt, Artificial Sequence
  Description of Artificial Sequence Synthetic oligonucleotide
ucaaggaagauggcauuucu

SEQ ID NO: 794 - RNA, 20 nt, Artificial Sequence
  Description of Artificial Sequence Synthetic oligonucleotide
ucaaggaagauggcauuucu

SEQ ID NO: 795 - RNA, 20 nt, Artificial Sequence
  Description of Artificial Sequence Synthetic oligonucleotide
ucaaggaagaugccauuucu

SEQ ID NO: 796 - RNA, 20 nt, Artificial Sequence
  Description of Artificial Sequence Synthetic oligonucleotide
ucaaggaagauggcauuucu

SEQ ID NO: 797 - RNA, 20 nt, Artificial Sequence
  Description of Artificial Sequence Synthetic oligonucleotide
ucaaggaagauggcauuucu

SEQ ID NO: 798 - RNA, 20 nt, Artificial Sequence
  Description of Artificial Sequence Synthetic oligonucleotide
ucaaggaagauggcacuucu

SEQ ID NO: 799 - RNA, 20 nt, Artificial Sequence
  Description of Artificial Sequence Synthetic oligonucleotide
ucaaggaagauggcauuucu

SEQ ID NO: 800 - RNA, 20 nt, Artificial Sequence
  Description of Artificial Sequence Synthetic oligonucleotide
ucaaggaagauggcauuucu

SEQ ID NO: 801 - RNA, 20 nt, Artificial Sequence
  Description of Artificial Sequence Synthetic oligonucleotide
ucaaggaagauggcauuucu

SEQ ID NO: 802 - RNA, 20 nt, Artificial Sequence
  Description of Artificial Sequence Synthetic oligonucleotide
ucaaggaagauggcauuucu

SEQ ID NO: 803 - RNA, 20 nt, Artificial Sequence
  Description of Artificial Sequence Synthetic oligonucleotide
ucaaggaagauggcauuucu

SEQ ID NO: 804 - RNA, 20 nt, Artificial Sequence
  Description of Artificial Sequence Synthetic oligonucleotide
ucaaggaagauggcauuucu

SEQ ID NO: 805 - RNA, 20 nt, Artificial Sequence
  Description of Artificial Sequence Synthetic oligonucleotide
ucaaggaagauggcauuucu

SEQ ID NO: 806 - RNA, 20 nt, Artificial Sequence
  Description of Artificial Sequence Synthetic oligonucleotide
ucaaggaagauggcauuucu

SEQ ID NO: 807 - RNA, 20 nt, Artificial Sequence
  Description of Artificial Sequence Synthetic oligonucleotide
ucaaggaagauggcauuucu

SEQ ID NO: 808 - RNA, 20 nt, Artificial Sequence
  Description of Artificial Sequence Synthetic oligonucleotide
ucaaggaagauggcauuucu

SEQ ID NO: 809 - RNA, 20 nt, Artificial Sequence
  Description of Artificial Sequence Synthetic oligonucleotide
ucaaggaagauggcauuucu

SEQ ID NO: 810 - RNA, 20 nt, Artificial Sequence
  Description of Artificial Sequence Synthetic oligonucleotide
ucaaggaagauggcauuucu

SEQ ID NO: 811 - RNA, 20 nt, Artificial Sequence
  Description of Artificial Sequence Synthetic oligonucleotide
ucaaggaagauggcauuucu

SEQ ID NO: 812 - RNA, 20 nt, Artificial Sequence
  Description of Artificial Sequence Synthetic oligonucleotide
ucaaggaagauggcauuucu

SEQ ID NO: 813 - RNA, 20 nt, Artificial Sequence
  Description of Artificial Sequence Synthetic oligonucleotide
ucaaggaagauggcauuucu

SEQ ID NO: 814 - RNA, 20 nt, Artificial Sequence
  Description of Artificial Sequence Synthetic oligonucleotide
ucaaggaagauggcauuucu

SEQ ID NO: 815 - RNA, 20 nt, Artificial Sequence
  Description of Artificial Sequence Synthetic oligonucleotide
ucaaggaagauggcauuucu

SEQ ID NO: 816 - RNA, 20 nt, Artificial Sequence
  Description of Artificial Sequence Synthetic oligonucleotide
ucaaggaagauggcauuucu

SEQ ID NO: 817 - RNA, 20 nt, Artificial Sequence
  Description of Artificial Sequence Synthetic oligonucleotide
ucaaggaagauggcauuucu

SEQ ID NO: 818 - RNA, 20 nt, Artificial Sequence
  Description of Artificial Sequence Synthetic oligonucleotide
ucaaggaagauggcauuucu

SEQ ID NO: 819 - RNA, 20 nt, Artificial Sequence
  Description of Artificial Sequence Synthetic oligonucleotide
ucaaggaagauggcauuucu

SEQ ID NO: 820 - RNA, 20 nt, Artificial Sequence
  Description of Artificial Sequence Synthetic oligonucleotide
ucaaggaagauggcauuucu

SEQ ID NO: 821 - RNA, 20 nt, Artificial Sequence
  Description of Artificial Sequence Synthetic oligonucleotide
ucaaggaagauggcauuucu

SEQ ID NO: 822 - RNA, 20 nt, Artificial Sequence
  Description of Artificial Sequence Synthetic oligonucleotide
ucaaggaagauggcauuucu

SEQ ID NO: 823 - RNA, 20 nt, Artificial Sequence
  Description of Artificial Sequence Synthetic oligonucleotide
ucaaggaagauggcauuucu

SEQ ID NO: 824 - RNA, 20 nt, Artificial Sequence
  Description of Artificial Sequence Synthetic oligonucleotide
ucaaggaagauggcauuucu

SEQ ID NO: 825 - RNA, 20 nt, Artificial Sequence
  Description of Artificial Sequence Synthetic oligonucleotide
ucaaggaagauggcauuucu

SEQ ID NO: 826 - RNA, 20 nt, Artificial Sequence
  Description of Artificial Sequence Synthetic oligonucleotide
ucaaggaagauggcauuucu

SEQ ID NO: 827 - RNA, 20 nt, Artificial Sequence
  Description of Artificial Sequence Synthetic oligonucleotide
ucaaggaagauggcauuucu

SEQ ID NO: 828 - RNA, 20 nt, Artificial Sequence
  Description of Artificial Sequence Synthetic oligonucleotide
ucaaggaagauggcauuucu

SEQ ID NO: 829 - RNA, 20 nt, Artificial Sequence
  Description of Artificial Sequence Synthetic oligonucleotide
ucaaggaagauggcauuucu

SEQ ID NO: 830 - RNA, 20 nt, Artificial Sequence
  Description of Artificial Sequence Synthetic oligonucleotide
ucaaggaagauggcauuucu

SEQ ID NO: 831 - RNA, 20 nt, Artificial Sequence
  Description of Artificial Sequence Synthetic oligonucleotide
ucaaggaagauggcauuucu

SEQ ID NO: 832 - RNA, 20 nt, Artificial Sequence
  Description of Artificial Sequence Synthetic oligonucleotide
ucaaggaagauggcauuucu

SEQ ID NO: 833 - RNA, 20 nt, Artificial Sequence
  Description of Artificial Sequence Synthetic oligonucleotide
ucaaggaagauggcauuucu

SEQ ID NO: 834 - RNA, 20 nt, Artificial Sequence
  Description of Artificial Sequence Synthetic oligonucleotide
ucaaggaagauggcauuucu

SEQ ID NO: 835 - RNA, 20 nt, Artificial Sequence
  Description of Artificial Sequence Synthetic oligonucleotide
ucaaggaagauggcauuucu

SEQ ID NO: 836 - RNA, 20 nt, Artificial Sequence
  Description of Artificial Sequence Synthetic oligonucleotide
ucaaggaagauggcauuucu

SEQ ID NO: 837 - RNA, 20 nt, Artificial Sequence
  Description of Artificial Sequence Synthetic oligonucleotide
ucaaggaagauggcauuucu

SEQ ID NO: 838 - RNA, 20 nt, Artificial Sequence
  Description of Artificial Sequence Synthetic oligonucleotide
ucaaggaagauggcauuucu

SEQ ID NO: 839 - RNA, 20 nt, Artificial Sequence
  Description of Artificial Sequence Synthetic oligonucleotide
ucaaggaagauggcauuucu

SEQ ID NO: 840 - RNA, 20 nt, Artificial Sequence
  Description of Artificial Sequence Synthetic oligonucleotide
ucaaggaagauggcauuucu

SEQ ID NO: 841 - RNA, 20 nt, Artificial Sequence
  Description of Artificial Sequence Synthetic oligonucleotide
ucaaggaagauggcauuucu

SEQ ID NO: 842 - RNA, 20 nt, Artificial Sequence
  Description of Artificial Sequence Synthetic oligonucleotide
ucaaggaagauggcauuucu

SEQ ID NO: 843 - RNA, 20 nt, Artificial Sequence
  Description of Artificial Sequence Synthetic oligonucleotide
ucaaggaagauggcauuucu

SEQ ID NO: 844 - RNA, 20 nt, Artificial Sequence
  Description of Artificial Sequence Synthetic oligonucleotide
ucaaggaagauggcauuucu

SEQ ID NO: 845 - RNA, 20 nt, Artificial Sequence
  Description of Artificial Sequence Synthetic oligonucleotide
ucaaggaagauggcauuucu

SEQ ID NO: 846 - RNA, 20 nt, Artificial Sequence
  Description of Artificial Sequence Synthetic oligonucleotide
ucaaggaagauggcauuucu

SEQ ID NO: 847 - RNA, 20 nt, Artificial Sequence
  Description of Artificial Sequence Synthetic oligonucleotide
ucaaggaagauggcauuucu

SEQ ID NO: 848 - RNA, 20 nt, Artificial Sequence
  Description of Artificial Sequence Synthetic oligonucleotide
ucaaggaagauggcauuucu

SEQ ID NO: 849 - RNA, 20 nt, Artificial Sequence
  Description of Artificial Sequence Synthetic oligonucleotide
ucaaggaagauggcauuucu

SEQ ID NO: 850 - RNA, 20 nt, Artificial Sequence
  Description of Artificial Sequence Synthetic oligonucleotide
ucaaggaagauggcauuucu

SEQ ID NO: 851 - RNA, 20 nt, Artificial Sequence
  Description of Artificial Sequence Synthetic oligonucleotide
ucaaggaagauggcauuucu

SEQ ID NO: 852 - RNA, 20 nt, Artificial Sequence
  Description of Artificial Sequence Synthetic oligonucleotide
ucaaggaagauggcauuucu

SEQ ID NO: 853 - RNA, 20 nt, Artificial Sequence
  Description of Artificial Sequence Synthetic oligonucleotide
ucaaggaagauggcauuucu

SEQ ID NO: 854 - RNA, 20 nt, Artificial Sequence
  Description of Artificial Sequence Synthetic oligonucleotide
ucaaggaagauggcauuucu

SEQ ID NO: 855 - RNA, 20 nt, Artificial Sequence
  Description of Artificial Sequence Synthetic oligonucleotide
ucaaggaagauggcauuucu

SEQ ID NO: 856 - RNA, 20 nt, Artificial Sequence
  Description of Artificial Sequence Synthetic oligonucleotide
ucaaggaagauggcauuucu

SEQ ID NO: 857 - RNA, 20 nt, Artificial Sequence
  Description of Artificial Sequence Synthetic oligonucleotide
ucaaggaagauggcauuucu

SEQ ID NO: 858 - RNA, 20 nt, Artificial Sequence
  Description of Artificial Sequence Synthetic oligonucleotide
ucaaggaagauggcauuucu

SEQ ID NO: 859 - RNA, 20 nt, Artificial Sequence
  Description of Artificial Sequence Synthetic oligonucleotide
ucaaggaagauggcauuucu

SEQ ID NO: 860 - RNA, 20 nt, Artificial Sequence
  Description of Artificial Sequence Synthetic oligonucleotide
ucaaggaagauggcauuucu

SEQ ID NO: 861 - RNA, 20 nt, Artificial Sequence
  Description of Artificial Sequence Synthetic oligonucleotide
ucaaggaagauggcauuucu

SEQ ID NO: 862 - RNA, 20 nt, Artificial Sequence
  Description of Artificial Sequence Synthetic oligonucleotide
ucaaggaagauggcauuucu

SEQ ID NO: 863 - RNA, 20 nt, Artificial Sequence
  Description of Artificial Sequence Synthetic oligonucleotide
ucaaggaagauggcauuucu

SEQ ID NO: 864 - RNA, 20 nt, Artificial Sequence
  Description of Artificial Sequence Synthetic oligonucleotide
ucaaggaagauggcauuucu

SEQ ID NO: 865 - RNA, 20 nt, Artificial Sequence
  Description of Artificial Sequence Synthetic oligonucleotide
ucaaggaagauggcauuucu

SEQ ID NO: 866 - RNA, 20 nt, Artificial Sequence
  Description of Artificial Sequence Synthetic oligonucleotide
ucaaggaagauggcauuucu

SEQ ID NO: 867 - RNA, 20 nt, Artificial Sequence
  Description of Artificial Sequence Synthetic oligonucleotide
ucaaggaagauggcauuucu

SEQ ID NO: 868 - RNA, 20 nt, Artificial Sequence
  Description of Artificial Sequence Synthetic oligonucleotide
ucaaggaagauggcauuucu

SEQ ID NO: 869 - RNA, 20 nt, Artificial Sequence
  Description of Artificial Sequence Synthetic oligonucleotide
ucaaggaagauggcauuucu

SEQ ID NO: 870 - RNA, 20 nt, Artificial Sequence
  Description of Artificial Sequence Synthetic oligonucleotide
ucaaggaagauggcauuucu

SEQ ID NO: 871 - RNA, 20 nt, Artificial Sequence
  Description of Artificial Sequence Synthetic oligonucleotide
ucaaggaagauggcauuucu

SEQ ID NO: 872 - RNA, 20 nt, Artificial Sequence
  Description of Artificial Sequence Synthetic oligonucleotide
ucaaggaagauggcauuucu

SEQ ID NO: 873 - RNA, 20 nt, Artificial Sequence
  Description of Artificial Sequence Synthetic oligonucleotide
ucaaggaagauggcauuucu

SEQ ID NO: 874 - RNA, 20 nt, Artificial Sequence
  Description of Artificial Sequence Synthetic oligonucleotide
ucaaggaagauggcauuucu

SEQ ID NO: 875 - RNA, 20 nt, Artificial Sequence
  Description of Artificial Sequence Synthetic oligonucleotide
ucaaggaagauggcauuucu

SEQ ID NO: 876 - RNA, 20 nt, Artificial Sequence
  Description of Artificial Sequence Synthetic oligonucleotide
ucaaggaagauggcauuucu

SEQ ID NO: 877 - RNA, 20 nt, Artificial Sequence
  Description of Artificial Sequence Synthetic oligonucleotide
ucaaggaagauggcauuucu

SEQ ID NO: 878 - RNA, 20 nt, Artificial Sequence
  Description of Artificial Sequence Synthetic oligonucleotide
ucaaggaagauggcauuucu

SEQ ID NO: 879 - RNA, 20 nt, Artificial Sequence
  Description of Artificial Sequence Synthetic oligonucleotide
ucaaggaagauggcauuucu

SEQ ID NO: 880 - RNA, 20 nt, Artificial Sequence
  Description of Artificial Sequence Synthetic oligonucleotide
ucaaggaagauggcauuucu

SEQ ID NO: 881 - RNA, 20 nt, Artificial Sequence
  Description of Artificial Sequence Synthetic oligonucleotide
ucaaggaagauggcauuucu

SEQ ID NO: 882 - RNA, 20 nt, Artificial Sequence
  Description of Artificial Sequence Synthetic oligonucleotide
ucaaggaagauggcauuucu

SEQ ID NO: 883 - RNA, 20 nt, Artificial Sequence
  Description of Artificial Sequence Synthetic oligonucleotide
ucaaggaagauggcauuucu

SEQ ID NO: 884 - RNA, 20 nt, Artificial Sequence
  Description of Artificial Sequence Synthetic oligonucleotide
ucaaggaagauggcauuucu

SEQ ID NO: 885 - RNA, 17 nt, Artificial Sequence
  Description of Artificial Sequence Synthetic oligonucleotide
ucaaggaagauggcucu

SEQ ID NO: 886 - RNA, 19 nt, Artificial Sequence
  Description of Artificial Sequence Synthetic oligonucleotide
ucaagaagauggcauuucu

SEQ ID NO: 887 - RNA, 20 nt, Artificial Sequence
  Description of Artificial Sequence Synthetic oligonucleotide
ucaaggaagauggcauuucu

SEQ ID NO: 888 - RNA, 20 nt, Artificial Sequence
  Description of Artificial Sequence Synthetic oligonucleotide
ucaaggaagauggcauuucu

SEQ ID NO: 889 - RNA, 20 nt, Artificial Sequence
  Description of Artificial Sequence Synthetic oligonucleotide
ucaaggaagauggcauuucu

SEQ ID NO: 890 - RNA, 20 nt, Artificial Sequence
  Description of Artificial Sequence Synthetic oligonucleotide
ucaaggaagauggcauuucu

SEQ ID NO: 891 - RNA, 20 nt, Artificial Sequence
  Description of Artificial Sequence Synthetic oligonucleotide
ucaaggaagauggcauuucu

SEQ ID NO: 892 - RNA, 20 nt, Artificial Sequence
  Description of Artificial Sequence Synthetic oligonucleotide
ucaaggaagauggcauuucu

SEQ ID NO: 893 - RNA, 20 nt, Artificial Sequence
  Description of Artificial Sequence Synthetic oligonucleotide
ucaaggaagauggcauuucu

SEQ ID NO: 894 - RNA, 20 nt, Artificial Sequence
  Description of Artificial Sequence Synthetic oligonucleotide
ucaaggaagauggcauuucu

SEQ ID NO: 895 - RNA, 20 nt, Artificial Sequence
  Description of Artificial Sequence Synthetic oligonucleotide
ucaaggaagauggcauuucu

SEQ ID NO: 896 - RNA, 20 nt, Artificial Sequence
  Description of Artificial Sequence Synthetic oligonucleotide
ucaaggaagauggcauuucu

SEQ ID NO: 897 - RNA, 20 nt, Artificial Sequence
  Description of Artificial Sequence Synthetic oligonucleotide
ucaaggaagauggcauuucu

SEQ ID NO: 898 - RNA, 20 nt, Artificial Sequence
  Description of Artificial Sequence Synthetic oligonucleotide
ucaaggaagauggcauuucu

SEQ ID NO: 899 - RNA, 20 nt, Artificial Sequence
  Description of Artificial Sequence Synthetic oligonucleotide
ucaaggaagauggcauuucu

SEQ ID NO: 900 - RNA, 20 nt, Artificial Sequence
  Description of Artificial Sequence Synthetic oligonucleotide
ucaaggaagauggcauuucu

SEQ ID NO: 901 - RNA, 20 nt, Artificial Sequence
  Description of Artificial Sequence Synthetic oligonucleotide
ucaaggaagauggcauuucu

SEQ ID NO: 902 - RNA, 20 nt, Artificial Sequence
  Description of Artificial Sequence Synthetic oligonucleotide
ucaaggaagauggcauuucu

SEQ ID NO: 903 - RNA, 20 nt, Artificial Sequence
  Description of Artificial Sequence Synthetic oligonucleotide
ucaaggaagauggcauuucu

SEQ ID NO: 904 - RNA, 20 nt, Artificial Sequence
  Description of Artificial Sequence Synthetic oligonucleotide
ucaaggaagauggcauuucu

SEQ ID NO: 905 - RNA, 20 nt, Artificial Sequence
  Description of Artificial Sequence Synthetic oligonucleotide
ucaaggaagauggcauuucu

SEQ ID NO: 906 - RNA, 20 nt, Artificial Sequence
  Description of Artificial Sequence Synthetic oligonucleotide
ucaaggaagauggcauuucu

SEQ ID NO: 907 - RNA, 20 nt, Artificial Sequence
  Description of Artificial Sequence Synthetic oligonucleotide
ucaaggaagauggcauuucu

SEQ ID NO: 908 - RNA, 20 nt, Artificial Sequence
  Description of Artificial Sequence Synthetic oligonucleotide
ucaaggaagauggcauuucu

SEQ ID NO: 909 - RNA, 20 nt, Artificial Sequence
  Description of Artificial Sequence Synthetic oligonucleotide
ucaaggaagauggcauuucu

SEQ ID NO: 910 - RNA, 20 nt, Artificial Sequence
  Description of Artificial Sequence Synthetic oligonucleotide
ucaaggaagauggcauuucu

SEQ ID NO: 911 - RNA, 20 nt, Artificial Sequence
  Description of Artificial Sequence Synthetic oligonucleotide
ucaaggaagauggcauuucu

SEQ ID NO: 912 - RNA, 20 nt, Artificial Sequence
  Description of Artificial Sequence Synthetic oligonucleotide
ucaaggaagauggcauuucu

SEQ ID NO: 913 - RNA, 20 nt, Artificial Sequence
  Description of Artificial Sequence Synthetic oligonucleotide
ucaaggaagauggcauuucu

SEQ ID NO: 914 - RNA, 20 nt, Artificial Sequence
  Description of Artificial Sequence Synthetic oligonucleotide
ucaaggaagauggcauuucu

SEQ ID NO: 915 - RNA, 20 nt, Artificial Sequence
  Description of Artificial Sequence Synthetic oligonucleotide
ucaaggaagauggcauuucu

SEQ ID NO: 916 - RNA, 19 nt, Artificial Sequence
  Description of Artificial Sequence Synthetic oligonucleotide
ucaaggaagauggcauuuc

SEQ ID NO: 917 - RNA, 20 nt, Artificial Sequence
  Description of Artificial Sequence Synthetic oligonucleotide
ucaaggaagauggcauuucu

SEQ ID NO: 918 - RNA, 20 nt, Artificial Sequence
  Description of Artificial Sequence Synthetic oligonucleotide
ucaaggaagauggcauuucu

SEQ ID NO: 919 - RNA, 20 nt, Artificial Sequence
  Description of Artificial Sequence Synthetic oligonucleotide
ucaaggaagauggcauuucu

SEQ ID NO: 920 - RNA, 20 nt, Artificial Sequence
  Description of Artificial Sequence Synthetic oligonucleotide
ucaaggaagauggcauuucu

SEQ ID NO: 921 - RNA, 20 nt, Artificial Sequence
  Description of Artificial Sequence Synthetic oligonucleotide
ucaaggaagauggcauuucu

SEQ ID NO: 922 - RNA, 20 nt, Artificial Sequence
  Description of Artificial Sequence Synthetic oligonucleotide
ucaaggaagauggcauuucu

SEQ ID NO: 923 - RNA, 20 nt, Artificial Sequence
  Description of Artificial Sequence Synthetic oligonucleotide
ucaaggaagauggcauuucu

SEQ ID NO: 924 - RNA, 20 nt, Artificial Sequence
  Description of Artificial Sequence Synthetic oligonucleotide
ucaaggaagauggcauuucu

SEQ ID NO: 925 - RNA, 20 nt, Artificial Sequence
  Description of Artificial Sequence Synthetic oligonucleotide
ucaaggaagauggcauuucu

SEQ ID NO: 926 - RNA, 20 nt, Artificial Sequence
  Description of Artificial Sequence Synthetic oligonucleotide
ucaaggaagauggcauuucu

SEQ ID NO: 927 - RNA, 30 nt, Artificial Sequence
  Description of Artificial Sequence Synthetic oligonucleotide
gccaacugggagcuggagcgcaccaaccag

SEQ ID NO: 928 - RNA, 20 nt, Artificial Sequence
  Description of Artificial Sequence Synthetic oligonucleotide
ucaaggaagauggcauuucu

SEQ ID NO: 929 - RNA, 20 nt, Artificial Sequence
  Description of Artificial Sequence Synthetic oligonucleotide
ucaaggaagauggcauuucu

SEQ ID NO: 930 - RNA, 20 nt, Artificial Sequence
  Description of Artificial Sequence Synthetic oligonucleotide
ucaaggaagauggcauuucu

SEQ ID NO: 931 - RNA, 20 nt, Artificial Sequence
  Description of Artificial Sequence Synthetic oligonucleotide
ucaaggaagauggcauuucu

SEQ ID NO: 932 - RNA, 20 nt, Artificial Sequence
  Description of Artificial Sequence Synthetic oligonucleotide
ccuucccugaagguuccucc

SEQ ID NO: 933 - RNA, 20 nt, Artificial Sequence
  Description of Artificial Sequence Synthetic oligonucleotide
ccuucccugaagguuccucc

SEQ ID NO: 934 - RNA, 20 nt, Artificial Sequence
  Description of Artificial Sequence Synthetic oligonucleotide
ccuucccugaagguuccucc

SEQ ID NO: 935 - RNA, 20 nt, Artificial Sequence
  Description of Artificial Sequence Synthetic oligonucleotide
ccuucccugaagguuccucc

SEQ ID NO: 936 - RNA, 20 nt, Artificial Sequence
  Description of Artificial Sequence Synthetic oligonucleotide
ccuucccugaagguuccucc

SEQ ID NO: 937 - RNA, 20 nt, Artificial Sequence
  Description of Artificial Sequence Synthetic oligonucleotide
ccuucccugaagguuccucc

SEQ ID NO: 938 - RNA, 20 nt, Artificial Sequence
  Description of Artificial Sequence Synthetic oligonucleotide
ucaaggaagauggcauuucu

SEQ ID NO: 939 - RNA, 20 nt, Artificial Sequence
  Description of Artificial Sequence Synthetic oligonucleotide
ucaaggaagauggcauuucu

SEQ ID NO: 940 - RNA, 19 nt, Artificial Sequence
  Description of Artificial Sequence Synthetic oligonucleotide
ucaaggaagauggcauuuc

SEQ ID NO: 941 - RNA, 19 nt, Artificial Sequence
  Description of Artificial Sequence Synthetic oligonucleotide
ucaaggaagauggcauuuc

SEQ ID NO: 942 - RNA, 18 nt, Artificial Sequence
  Description of Artificial Sequence Synthetic oligonucleotide
ucaaggaagauggcauuu

SEQ ID NO: 943 - RNA, 19 nt, Artificial Sequence
  Description of Artificial Sequence Synthetic oligonucleotide
caaggaagauggcauuucu

SEQ ID NO: 944 - RNA, 20 nt, Artificial Sequence
  Description of Artificial Sequence Synthetic oligonucleotide
ggccaaaccucggcuuaccu

SEQ ID NO: 945 - RNA, 20 nt, Artificial Sequence
  Description of Artificial Sequence Synthetic oligonucleotide
ggccaaaccucggcuuaccu

SEQ ID NO: 946 - RNA, 20 nt, Artificial Sequence
  Description of Artificial Sequence Synthetic oligonucleotide
uucuguaagguuuuuaugug

SEQ ID NO: 947 - RNA, 20 nt, Artificial Sequence
  Description of Artificial Sequence Synthetic oligonucleotide
auuucuguaagguuuuuaug

SEQ ID NO: 948 - RNA, 20 nt, Artificial Sequence
  Description of Artificial Sequence Synthetic oligonucleotide
ccauuucuguaagguuuuua

SEQ ID NO: 949 - RNA, 20 nt, Artificial Sequence
  Description of Artificial Sequence Synthetic oligonucleotide
auccauuucuguaagguuuu

SEQ ID NO: 950 - RNA, 20 nt, Artificial Sequence
  Description of Artificial Sequence Synthetic oligonucleotide
cauccauuucuguaagguuu

SEQ ID NO: 951 - RNA, 20 nt, Artificial Sequence
  Description of Artificial Sequence Synthetic oligonucleotide
ccauccauuucuguaagguu

SEQ ID NO: 952 - RNA, 20 nt, Artificial Sequence
  Description of Artificial Sequence Synthetic oligonucleotide
gccauccauuucuguaaggu

SEQ ID NO: 953 - RNA, 20 nt, Artificial Sequence
  Description of Artificial Sequence Synthetic oligonucleotide
agccauccauuucuguaagg

SEQ ID NO: 954 - RNA, 20 nt, Artificial Sequence
  Description of Artificial Sequence Synthetic oligonucleotide
cagccauccauuucuguaag

SEQ ID NO: 955 - RNA, 20 nt, Artificial Sequence
  Description of Artificial Sequence Synthetic oligonucleotide
ucagccauccauuucuguaa

SEQ ID NO: 956 - RNA, 20 nt, Artificial Sequence
  Description of Artificial Sequence Synthetic oligonucleotide
uucagccauccauuucugua

SEQ ID NO: 957 - RNA, 20 nt, Artificial Sequence
  Description of Artificial Sequence Synthetic oligonucleotide
cuucacccauccauuucugu

SEQ ID NO: 958 - RNA, 20 nt, Artificial Sequence
  Description of Artificial Sequence Synthetic oligonucleotide
acuucagccauccauuucug

SEQ ID NO: 959 - RNA, 20 nt, Artificial Sequence
  Description of Artificial Sequence Synthetic oligonucleotide
aacuucagccauccauuucu

SEQ ID NO: 960 - RNA, 20 nt, Artificial Sequence
  Description of Artificial Sequence Synthetic oligonucleotide
caacuucagccauccauuuc

SEQ ID NO: 961 - RNA, 20 nt, Artificial Sequence
  Description of Artificial Sequence Synthetic oligonucleotide
ucaacuucagccauccauuu

SEQ ID NO: 962 - RNA, 20 nt, Artificial Sequence
  Description of Artificial Sequence Synthetic oligonucleotide
ccagggcaggccauuccucu

SEQ ID NO: 963 - RNA, 20 nt, Artificial Sequence
  Description of Artificial Sequence Synthetic oligonucleotide
cccagggcaggccauuccuc

SEQ ID NO: 964 - RNA, 20 nt, Artificial Sequence
  Description of Artificial Sequence Synthetic oligonucleotide
ccccagggcaggccauuccu

SEQ ID NO: 965 - RNA, 20 nt, Artificial Sequence
  Description of Artificial Sequence Synthetic oligonucleotide
cccccagggcaggccauucc

SEQ ID NO: 966 - RNA, 20 nt, Artificial Sequence
  Description of Artificial Sequence Synthetic oligonucleotide
ucccccagggcaggccauuc

SEQ ID NO: 967 - RNA, 20 nt, Artificial Sequence
  Description of Artificial Sequence Synthetic oligonucleotide
aucccccagggcaggccauu

SEQ ID NO: 968 - RNA, 20 nt, Artificial Sequence
  Description of Artificial Sequence Synthetic oligonucleotide
caucccccagggcaggccau

SEQ ID NO: 969 - RNA, 20 nt, Artificial Sequence
  Description of Artificial Sequence Synthetic oligonucleotide
gcaucccccagggcaggcca

SEQ ID NO: 970 - RNA, 20 nt, Artificial Sequence
  Description of Artificial Sequence Synthetic oligonucleotide
agcaucccccagggcaggcc

SEQ ID NO: 971 - RNA, 20 nt, Artificial Sequence
  Description of Artificial Sequence Synthetic oligonucleotide
cagcaucccccagggcaggc

SEQ ID NO: 972 - RNA, 20 nt, Artificial Sequence
  Description of Artificial Sequence Synthetic oligonucleotide
ucagcaucccccagggcagg

SEQ ID NO: 973 - RNA, 20 nt, Artificial Sequence
  Description of Artificial Sequence Synthetic oligonucleotide
uucagcaucccccagggcag

SEQ ID NO: 974 - RNA, 20 nt, Artificial Sequence
  Description of Artificial Sequence Synthetic oligonucleotide
uuucagcaucccccagggca

SEQ ID NO: 975 - RNA, 20 nt, Artificial Sequence
  Description of Artificial Sequence Synthetic oligonucleotide
auuucagcaucccccagggc

SEQ ID NO: 976 - RNA, 20 nt, Artificial Sequence
  Description of Artificial Sequence Synthetic oligonucleotide
gauuucagcaucccccaggg

SEQ ID NO: 977 - RNA, 20 nt, Artificial Sequence
  Description of Artificial Sequence Synthetic oligonucleotide
ggauuucagcaucccccagg

SEQ ID NO: 978 - RNA, 20 nt, Artificial Sequence
  Description of Artificial Sequence Synthetic oligonucleotide
aggauuucagcaucccccag

SEQ ID NO: 979 - RNA, 20 nt, Artificial Sequence
  Description of Artificial Sequence Synthetic oligonucleotide
caggauuucagcauccccca

SEQ ID NO: 980 - RNA, 20 nt, Artificial Sequence
  Description of Artificial Sequence Synthetic oligonucleotide
ucaggauuucagcauccccc

SEQ ID NO: 981 - RNA, 20 nt, Artificial Sequence
  Description of Artificial Sequence Synthetic oligonucleotide
uucaggauuucagcaucccc

SEQ ID NO: 982 - RNA, 20 nt, Artificial Sequence
  Description of Artificial Sequence Synthetic oligonucleotide
uuucaggauuucagcauccc

SEQ ID NO: 983 - RNA, 20 nt, Artificial Sequence
  Description of Artificial Sequence Synthetic oligonucleotide
uuuucaggauuucagcaucc

SEQ ID NO: 984 - RNA, 20 nt, Artificial Sequence
  Description of Artificial Sequence Synthetic oligonucleotide
uuuuucaggauuucagcauc

SEQ ID NO: 985 - RNA, 20 nt, Artificial Sequence
  Description of Artificial Sequence Synthetic oligonucleotide
uuuuuucaggauuucagcau

SEQ ID NO: 986 - RNA, 20 nt, Artificial Sequence
  Description of Artificial Sequence Synthetic oligonucleotide
guuuuuucaggauuucagca

SEQ ID NO: 987 - RNA, 20 nt, Artificial Sequence
  Description of Artificial Sequence Synthetic oligonucleotide
uguuuuuucaggauuucagc

SEQ ID NO: 988 - RNA, 20 nt, Artificial Sequence
  Description of Artificial Sequence Synthetic oligonucleotide
cuguuuuuucaggauuucag

SEQ ID NO: 989 - RNA, 20 nt, Artificial Sequence
  Description of Artificial Sequence Synthetic oligonucleotide
gcuguuuuuucaggauuuca

SEQ ID NO: 990 - RNA, 20 nt, Artificial Sequence
  Description of Artificial Sequence Synthetic oligonucleotide
agcuguuuuuucaggauuuc

SEQ ID NO: 991 - RNA, 20 nt, Artificial Sequence
  Description of Artificial Sequence Synthetic oligonucleotide
gagcuguuuuuucaggauuu

SEQ ID NO: 992 - RNA, 20 nt, Artificial Sequence
  Description of Artificial Sequence Synthetic oligonucleotide
ugagcuguuuuuucaggauu

SEQ ID NO: 993 - RNA, 20 nt, Artificial Sequence
  Description of Artificial Sequence Synthetic oligonucleotide
uugagcuguuuuuucaggau

SEQ ID NO: 994 - RNA, 20 nt, Artificial Sequence
  Description of Artificial Sequence Synthetic oligonucleotide
uuugagcuguuuuuucagga

SEQ ID NO: 995 - RNA, 20 nt, Artificial Sequence
  Description of Artificial Sequence Synthetic oligonucleotide
guuugagcuguuuuuucagg

SEQ ID NO: 996 - RNA, 20 nt, Artificial Sequence
  Description of Artificial Sequence Synthetic oligonucleotide
uuguuugagcuguuuuuuca

SEQ ID NO: 997 - RNA, 20 nt, Artificial Sequence
  Description of Artificial Sequence Synthetic oligonucleotide
cauuguuugagcuguuuuuu

SEQ ID NO: 998 - RNA, 20 nt, Artificial Sequence
  Description of Artificial Sequence Synthetic oligonucleotide
gcauuguuugagcuguuuuu

SEQ ID NO: 999 - RNA, 20 nt, Artificial Sequence
  Description of Artificial Sequence Synthetic oligonucleotide
ugcauuguuugagcuguuuu

SEQ ID NO: 1000 - RNA, 20 nt, Artificial Sequence
  Description of Artificial Sequence Synthetic oligonucleotide
cugcauuguuugagcuguuu

SEQ ID NO: 1001 - RNA, 20 nt, Artificial Sequence
  Description of Artificial Sequence Synthetic oligonucleotide
ucugcauuguuugagcuguu

SEQ ID NO: 1002 - RNA, 20 nt, Artificial Sequence
  Description of Artificial Sequence Synthetic oligonucleotide
cucugcauuguuugagcugu

SEQ ID NO: 1003 - RNA, 20 nt, Artificial Sequence
  Description of Artificial Sequence Synthetic oligonucleotide
acucugcauuguuugagcug

SEQ ID NO: 1004 - RNA, 20 nt, Artificial Sequence
  Description of Artificial Sequence Synthetic oligonucleotide
uacucugcauuguuugagcu

SEQ ID NO: 1005 - RNA, 20 nt, Artificial Sequence
  Description of Artificial Sequence Synthetic oligonucleotide
uuacucugcauuguuugagc

SEQ ID NO: 1006 - RNA, 20 nt, Artificial Sequence
  Description of Artificial Sequence Synthetic oligonucleotide
cuuacucugcauuguuugag

SEQ ID NO: 1007 - RNA, 20 nt, Artificial Sequence
  Description of Artificial Sequence Synthetic oligonucleotide
ucuuacucugcauuguuuga

SEQ ID NO: 1008 - RNA, 20 nt, Artificial Sequence
  Description of Artificial Sequence Synthetic oligonucleotide
aucuuacucugcauuguuug

SEQ ID NO: 1009 - RNA, 20 nt, Artificial Sequence
  Description of Artificial Sequence Synthetic oligonucleotide
aaucuuacucugcauuguuu

SEQ ID NO: 1010 - RNA, 20 nt, Artificial Sequence
  Description of Artificial Sequence Synthetic oligonucleotide
caaaucuuacucugcauugu

SEQ ID NO: 1011 - RNA, 20 nt, Artificial Sequence
  Description of Artificial Sequence Synthetic oligonucleotide
gauacaaaucuuacucugca

SEQ ID NO: 1012 - DNA, 20 bp, Artificial Sequence
  Description of Artificial Sequence Synthetic oligonucleotide
gggtcagctgccaatgctag

SEQ ID NO: 1013 - DNA, 20 bp, Artificial Sequence
  Description of Artificial Sequence Synthetic oligonucleotide
gggtcagctgccaatgctag

SEQ ID NO: 1014 - DNA, 20 bp, Artificial Sequence
  Description of Artificial Sequence Synthetic oligonucleotide
gggtcagctgccaatgctag

SEQ ID NO: 1015 - DNA, 20 bp, Artificial Sequence
  Description of Artificial Sequence Synthetic oligonucleotide
gggtcagctgccaatgctag

SEQ ID NO: 1016 - DNA, 20 bp, Artificial Sequence
  Description of Artificial Sequence Synthetic oligonucleotide
gggtcagctgccaatgctag

SEQ ID NO: 1017 - RNA, 70 nt, Artificial Sequence
  Description of Artificial Sequence Synthetic oligonucleotide
guggaauaguauaacaauaugcuaaauguuguuauaguaucccacacugagcaaugccguagucagcaau

SEQ ID NO: 1018 - RNA, 75 nt, Artificial Sequence
  Description of Artificial Sequence Synthetic oligonucleotide
guggaauaguauaacaauaugcuaaauguuguuauaguaucccacguacugagcaaugccguagucagcaaucuu

SEQ ID NO: 1019 - RNA, 66 nt, Artificial Sequence
  Description of Artificial Sequence Synthetic oligonucleotide
ggaauaguauaacaauaugcuaaauguuguuauaguaucccacugagcaaugccguagucagcaau

SEQ ID NO: 1020 - RNA, 71 nt, Artificial Sequence
  Description of Artificial Sequence Synthetic oligonucleotide
ggaauaguauaacaauaugcuaaauguuguuauaguaucccguacugagcaaugccguagucagcaaucuu

SEQ ID NO: 1021 - DNA, 31 bp, Artificial Sequence
  Description of Artificial Sequence Synthetic oligonucleotide
cgcgcgttttcgcgcggcugaaccacugcac

SEQ ID NO: 1022 - DNA, 31 bp, Artificial Sequence
  Description of Artificial Sequence Synthetic oligonucleotide
cgcgcgttttcgcgcggagauacucacaauu

SEQ ID NO: 1023 - DNA, 31 bp, Artificial Sequence
  Description of Artificial Sequence Synthetic oligonucleotide
cgcgcgttttcgcgcgcguugaccuccacuc

SEQ ID NO: 1024 - RNA, 66 nt, Artificial Sequence
  Description of Artificial Sequence Synthetic oligonucleotide
ggaauaguauaacaauaugcuaaauguuguuauaguauccccagucccuuucucgucgauggucag

SEQ ID NO: 1025 - RNA, 66 nt, Artificial Sequence; identical to SEQ ID NO: 1024
  Description of Artificial Sequence Synthetic oligonucleotide

SEQ ID NO: 1026 - RNA, 44 nt, Artificial Sequence
  Description of Artificial Sequence Synthetic oligonucleotide
guguuggccauggaacauauaacaauaugcuaaauguuguuaua

SEQ ID NO: 1027 - RNA, 44 nt, Artificial Sequence
  Description of Artificial Sequence Synthetic oligonucleotide
uauaacaauaugcuaaauguuguuauaguguuggccauggaaca

SEQ ID NO: 1028 - RNA, 44 nt, Artificial Sequence; identical to SEQ ID NO: 1026
  Description of Artificial Sequence Synthetic oligonucleotide

SEQ ID NO: 1029 - RNA, 52 nt, Artificial Sequence
  Description of Artificial Sequence Synthetic oligonucleotide
guguuggccauggaacaauaguauaacaauaugcuaaauguuguuauaguau

SEQ ID NO: 1030 - RNA, 58 nt, Artificial Sequence
  Description of Artificial Sequence Synthetic oligonucleotide
ggaauaguauaacaauaugcuaaauguuguuauaguaucccguguuggccauggaaca

SEQ ID NO: 1031 - RNA, 58 nt, Artificial Sequence
  Description of Artificial Sequence Synthetic oligonucleotide
guguuggccauggaacaggaauaguauaacaauaugcuaaauguuguuauaguauccc

SEQ ID NO: 1032 - RNA, 58 nt, Artificial Sequence; identical to SEQ ID NO: 1030
  Description of Artificial Sequence Synthetic oligonucleotide

SEQ ID NO: 1033 - RNA, 31 nt, Artificial Sequence
  Description of Artificial Sequence Synthetic oligonucleotide
ucagucccuuucucgucgauggucagcacag

SEQ ID NO: 1034 - RNA, 31 nt, Artificial Sequence; identical to SEQ ID NO: 1033
  Description of Artificial Sequence Synthetic oligonucleotide

SEQ ID NO: 1035 - RNA, 31 nt, Artificial Sequence; identical to SEQ ID NO: 1033
  Description of Artificial Sequence Synthetic oligonucleotide

SEQ ID NO: 1036 - RNA, 31 nt, Artificial Sequence; identical to SEQ ID NO: 1033
  Description of Artificial Sequence Synthetic oligonucleotide

SEQ ID NO: 1037 - RNA, 31 nt, Artificial Sequence; identical to SEQ ID NO: 1033
  Description of Artificial Sequence Synthetic oligonucleotide

SEQ ID NO: 1038 - DNA, 31 bp, Artificial Sequence
  Description of Artificial Sequence Synthetic oligonucleotide
ucagucccuuuctcgucgauggucagcacag

SEQ ID NO: 1039 - RNA, 31 nt, Artificial Sequence; identical to SEQ ID NO: 1033
  Description of Artificial Sequence Synthetic oligonucleotide

SEQ ID NO: 1040 - RNA, 31 nt, Artificial Sequence; identical to SEQ ID NO: 1033
  Description of Artificial Sequence Synthetic oligonucleotide

SEQ ID NO: 1041 - RNA, 31 nt, Artificial Sequence; identical to SEQ ID NO: 1033
  Description of Artificial Sequence Synthetic oligonucleotide

SEQ ID NO: 1042 - RNA, 31 nt, Artificial Sequence
  Description of Artificial Sequence Synthetic oligonucleotide
  modified_base (15)..(15): Inosine
  misc_feature (15)..(15): n is a, c, g, or u
ucagucccuuucucnucgauggucagcacag

SEQ ID NO: 1043 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1044 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1045 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1046 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1047 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1048 - RNA, 31 nt, Artificial Sequence
  Description of Artificial Sequence Synthetic oligonucleotide
  modified_base (13)..(13): 2,6-diamino purine
  misc_feature (13)..(13): n is a, c, g, or u
  modified_base (15)..(15): Inosine
  misc_feature (15)..(15): n is a, c, g, or u
ucagucccuuucncnucgauggucagcacag

SEQ ID NO: 1049 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1050 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1051 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1052 - RNA, 31 nt, Artificial Sequence; identical to SEQ ID NO: 1033
  Description of Artificial Sequence Synthetic oligonucleotide

SEQ ID NO: 1053 - RNA, 31 nt, Artificial Sequence; identical to SEQ ID NO: 1033
  Description of Artificial Sequence Synthetic oligonucleotide

SEQ ID NO: 1054 - RNA, 32 nt, Artificial Sequence
  Description of Artificial Sequence Synthetic oligonucleotide
ucagucccuuucucgvucgauggucagcacag

SEQ ID NO: 1055 - RNA, 31 nt, Artificial Sequence; identical to SEQ ID NO: 1033
  Description of Artificial Sequence Synthetic oligonucleotide

SEQ ID NO: 1056 - RNA, 34 nt, Artificial Sequence
  Description of Artificial Sequence Synthetic oligonucleotide
ucagucccuuucuacagaucgauggucagcacag

SEQ ID NO: 1057 - RNA, 31 nt, Artificial Sequence; identical to SEQ ID NO: 1033
  Description of Artificial Sequence Synthetic oligonucleotide

SEQ ID NO: 1058 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1059 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1060 - RNA, 31 nt, Artificial Sequence; identical to SEQ ID NO: 1042
  Description of Artificial Sequence Synthetic oligonucleotide
  modified_base (15)..(15): Inosine
  misc_feature (15)..(15): n is a, c, g, or u

SEQ ID NO: 1061 - RNA, 31 nt, Artificial Sequence; identical to SEQ ID NO: 1033
  Description of Artificial Sequence Synthetic oligonucleotide

SEQ ID NO: 1062 - RNA, 31 nt, Artificial Sequence; identical to SEQ ID NO: 1033
  Description of Artificial Sequence Synthetic oligonucleotide

SEQ ID NO: 1063 - RNA, 31 nt, Artificial Sequence; identical to SEQ ID NO: 1033
  Description of Artificial Sequence Synthetic oligonucleotide

SEQ ID NO: 1064 - RNA, 31 nt, Artificial Sequence; identical to SEQ ID NO: 1033
  Description of Artificial Sequence Synthetic oligonucleotide

SEQ ID NO: 1065 - RNA, 33 nt, Artificial Sequence
  Description of Artificial Sequence Synthetic oligonucleotide
ccugcgacacuucggcccagagcugcuccucau

SEQ ID NO: 1066 - RNA, 33 nt, Artificial Sequence; identical to SEQ ID NO: 1065
  Description of Artificial Sequence Synthetic oligonucleotide

SEQ ID NO: 1067 - RNA, 33 nt, Artificial Sequence; identical to SEQ ID NO: 1065
  Description of Artificial Sequence Synthetic oligonucleotide

SEQ ID NO: 1068 - RNA, 33 nt, Artificial Sequence; identical to SEQ ID NO: 1065
  Description of Artificial Sequence Synthetic oligonucleotide

SEQ ID NO: 1069 - RNA, 33 nt, Artificial Sequence; identical to SEQ ID NO: 1065
  Description of Artificial Sequence Synthetic oligonucleotide

SEQ ID NO: 1070 - RNA, 33 nt, Artificial Sequence; identical to SEQ ID NO: 1065
  Description of Artificial Sequence Synthetic oligonucleotide

SEQ ID NO: 1071 - RNA, 33 nt, Artificial Sequence
  Description of Artificial Sequence Synthetic oligonucleotide
  modified_base (19)..(19): Inosine
  misc_feature (19)..(19): n is a, c, g, or u
ccugcgacacuucggcccngagcugcuccucau

SEQ ID NO: 1072 - RNA, 53 nt, Artificial Sequence
  Description of Artificial Sequence Synthetic oligonucleotide
cauugaagaagauaagagaaaguacugagaaguguuggccauggaacagguag

SEQ ID NO: 1073 - RNA, 53 nt, Artificial Sequence
  Description of Artificial Sequence Synthetic oligonucleotide
  modified_base (41)..(41): 2-amino purine
  misc_feature (41)..(41): n is a, c, g, or u
cauugaagaagauaagagaaaguacugagaaguguuggccnuggaacagguag

SEQ ID NO: 1074 - RNA, 53 nt, Artificial Sequence; identical to SEQ ID NO: 1072
  Description of Artificial Sequence Synthetic oligonucleotide

SEQ ID NO: 1075 - RNA, 45 nt, Artificial Sequence
  Description of Artificial Sequence Synthetic oligonucleotide
gacugagguacuccuuagagaaaggugccacuucuuggcaaagga

SEQ ID NO: 1076 - RNA, 40 nt, Artificial Sequence
  Description of Artificial Sequence Synthetic oligonucleotide
guaggcaugggaggaaaaggugccacuucuuggcaaagga

SEQ ID NO: 1077 - RNA, 50 nt, Artificial Sequence
  Description of Artificial Sequence Synthetic oligonucleotide
cuguccaacacagccccagccuuugagaccucugcccagaguuguucucc

SEQ ID NO: 1078 - DNA, 51 bp, Artificial Sequence
  Description of Artificial Sequence Synthetic oligonucleotide
cuguccaacacagccccagccuuugagaccucuguccagaguuguucucct

SEQ ID NO: 1079 - DNA, 51 bp, Artificial Sequence; identical to SEQ ID NO: 1078
  Description of Artificial Sequence Synthetic oligonucleotide

SEQ ID NO: 1080 - DNA, 51 bp, Artificial Sequence; identical to SEQ ID NO: 1078
  Description of Artificial Sequence Synthetic oligonucleotide

SEQ ID NO: 1081 - DNA, 51 bp, Artificial Sequence
  Description of Artificial Sequence Synthetic oligonucleotide
  modified_base (38)..(38): deoxy 2-amino purine
  misc_feature (38)..(38): n is a, c, g, t or u
cuguccaacacagccccagccuuugagaccucuguccngaguuguucucct

SEQ ID NO: 1082 - DNA, 51 bp, Artificial Sequence
  Description of Artificial Sequence Synthetic oligonucleotide
cuguccaacacagccccagccuuugagaccucuguccagaauuguucucct

SEQ ID NO: 1083 - DNA, 51 bp, Artificial Sequence; identical to SEQ ID NO: 1078
  Description of Artificial Sequence Synthetic oligonucleotide

SEQ ID NO: 1084 - DNA, 51 bp, Artificial Sequence; identical to SEQ ID NO: 1078
  Description of Artificial Sequence Synthetic oligonucleotide

SEQ ID NO: 1085 - DNA, 51 bp, Artificial Sequence; identical to SEQ ID NO: 1078
  Description of Artificial Sequence Synthetic oligonucleotide

SEQ ID NO: 1086 - DNA, 51 bp, Artificial Sequence; identical to SEQ ID NO: 1078
  Description of Artificial Sequence Synthetic oligonucleotide

SEQ ID NO: 1087 - RNA, 50 nt, Artificial Sequence
  Description of Artificial Sequence Synthetic oligonucleotide
cuguccaacacagccccagccuuugagaccucuguccagaguuguucucc

SEQ ID NO: 1088 - RNA, 50 nt, Artificial Sequence; identical to SEQ ID NO: 1087
  Description of Artificial Sequence Synthetic oligonucleotide

SEQ ID NO: 1089 - RNA, 50 nt, Artificial Sequence; identical to SEQ ID NO: 1087
  Description of Artificial Sequence Synthetic oligonucleotide

SEQ ID NO: 1090 - RNA, 50 nt, Artificial Sequence; identical to SEQ ID NO: 1087
  Description of Artificial Sequence Synthetic oligonucleotide

SEQ ID NO: 1091 - RNA, 50 nt, Artificial Sequence; identical to SEQ ID NO: 1087
  Description of Artificial Sequence Synthetic oligonucleotide

SEQ ID NO: 1092 - RNA, 50 nt, Artificial Sequence; identical to SEQ ID NO: 1087
  Description of Artificial Sequence Synthetic oligonucleotide

SEQ ID NO: 1093 - RNA, 50 nt, Artificial Sequence; identical to SEQ ID NO: 1087
  Description of Artificial Sequence Synthetic oligonucleotide

SEQ ID NO: 1094 - RNA, 50 nt, Artificial Sequence; identical to SEQ ID NO: 1087
  Description of Artificial Sequence Synthetic oligonucleotide

SEQ ID NO: 1095 - DNA, 51 bp, Artificial Sequence; identical to SEQ ID NO: 1078
  Description of Artificial Sequence Synthetic oligonucleotide

SEQ ID NO: 1096 - RNA, 43 nt, Artificial Sequence
  Description of Artificial Sequence Synthetic oligonucleotide
acacagcuccagccuuugagaccucugcccagaguuguucucc

SEQ ID NO: 1097 - RNA, 42 nt, Artificial Sequence
  Description of Artificial Sequence Synthetic oligonucleotide
cacagccccagccuuugagaccucuguccagaguuguucucc

SEQ ID NO: 1098 - RNA, 42 nt, Artificial Sequence; identical to SEQ ID NO: 1097
  Description of Artificial Sequence Synthetic oligonucleotide

SEQ ID NO: 1099 - RNA, 42 nt, Artificial Sequence
  Description of Artificial Sequence Synthetic oligonucleotide
cacagccccagccuuugagaccucugcccagaauuguucucc

SEQ ID NO: 1100 - RNA, 45 nt, Artificial Sequence
  Description of Artificial Sequence Synthetic oligonucleotide
gacugagguacuccauagggaaaggcaccacuucuuggcaaagga

SEQ ID NO: 1101 - RNA, 45 nt, Artificial Sequence; identical to SEQ ID NO: 1100
  Description of Artificial Sequence Synthetic oligonucleotide

SEQ ID NO: 1102 - RNA, 45 nt, Artificial Sequence; identical to SEQ ID NO: 1100
  Description of Artificial Sequence Synthetic oligonucleotide

SEQ ID NO: 1103 - RNA, 45 nt, Artificial Sequence; identical to SEQ ID NO: 1075
  Description of Artificial Sequence Synthetic oligonucleotide

SEQ ID NO: 1104 - RNA, 53 nt, Artificial Sequence; identical to SEQ ID NO: 1072
  Description of Artificial Sequence Synthetic oligonucleotide

SEQ ID NO: 1105 - RNA, 53 nt, Artificial Sequence; identical to SEQ ID NO: 1072
  Description of Artificial Sequence Synthetic oligonucleotide

SEQ ID NO: 1106 - RNA, 44 nt, Artificial Sequence
  Description of Artificial Sequence Synthetic oligonucleotide
guggauaguauaacaauaugcumaauguuguuauaguaucccac

SEQ ID NO: 1107 - RNA, 45 nt, Artificial Sequence
  Description of Artificial Sequence Synthetic oligonucleotide
guggasssgsasascaauaugcumaauguugsususgssscccac

SEQ ID NO: 1108 - RNA, 45 nt, Artificial Sequence
  Description of Artificial Sequence Synthetic oligonucleotide
guggaasagsasaacaauaugcumaauguuguususgusucccac

SEQ ID NO: 1109 - RNA, 44 nt, Artificial Sequence
  Description of Artificial Sequence Synthetic oligonucleotide
guggasasuauaacaauaugcumaauguuguuauagyaycccac

SEQ ID NO: 1110 - RNA, 45 nt, Artificial Sequence
  Description of Artificial Sequence Synthetic oligonucleotide
guggasssgssssssssuaugcumaaugssssssssgssscccac

SEQ ID NO: 1111 - RNA, 45 nt, Artificial Sequence
  Description of Artificial Sequence Synthetic oligonucleotide
guggaassgsasascaauaugcumaauguugsususgssucccac

SEQ ID NO: 1112 - RNA, 46 nt, Artificial Sequence
  Description of Artificial Sequence Synthetic oligonucleotide
guggsauaguauaacaauaugcumaauguuguuauaguauscccac

SEQ ID NO: 1113 - RNA, 16 nt, Artificial Sequence
  Description of Artificial Sequence Synthetic oligonucleotide
acgcaaccaagucaua

SEQ ID NO: 1114 - RNA, 16 nt, Artificial Sequence
  Description of Artificial Sequence Synthetic oligonucleotide
gcaaugccaucaccuc

SEQ ID NO: 1115 - RNA, 16 nt, Artificial Sequence
  Description of Artificial Sequence Synthetic oligonucleotide
agggguccacauggca

SEQ ID NO: 1116 - RNA, 16 nt, Artificial Sequence
  Description of Artificial Sequence Synthetic oligonucleotide
ggcuccccaggccccu

SEQ ID NO: 1117 - RNA, 16 nt, Artificial Sequence
  Description of Artificial Sequence Synthetic oligonucleotide
ugccguccaccaggau

SEQ ID NO: 1118 - RNA, 16 nt, Artificial Sequence
  Description of Artificial Sequence Synthetic oligonucleotide
cagauuccagguggga

SEQ ID NO: 1119 - RNA, 16 nt, Artificial Sequence
  Description of Artificial Sequence Synthetic oligonucleotide
ucccugccagaauaga

SEQ ID NO: 1120 - RNA, 16 nt, Artificial Sequence
  Description of Artificial Sequence Synthetic oligonucleotide
cuccgcccaccaaaug

SEQ ID NO: 1121 - RNA, 16 nt, Artificial Sequence
  Description of Artificial Sequence Synthetic oligonucleotide
cccaaaccacaacaga

SEQ ID NO: 1122 - RNA, 16 nt, Artificial Sequence
  Description of Artificial Sequence Synthetic oligonucleotide
acccacccacccaggu

SEQ ID NO: 1123 - RNA, 16 nt, Artificial Sequence
  Description of Artificial Sequence Synthetic oligonucleotide
cugccgccagcuggau

SEQ ID NO: 1124 - RNA, 16 nt, Artificial Sequence
  Description of Artificial Sequence Synthetic oligonucleotide
agggaaccagacaguu

SEQ ID NO: 1125 - RNA, 17 nt, Artificial Sequence
  Description of Artificial Sequence Synthetic oligonucleotide
uucacuuaguguaugcc

SEQ ID NO: 1126 - RNA, 17 nt, Artificial Sequence
  Description of Artificial Sequence Synthetic oligonucleotide
uucacuuaguguaugcc

SEQ ID NO: 1127 - RNA, 17 nt, Artificial Sequence
  Description of Artificial Sequence Synthetic oligonucleotide
accuccaucagugugau

SEQ ID NO: 1128 - RNA, 17 nt, Artificial Sequence
  Description of Artificial Sequence Synthetic oligonucleotide
uuuccucacuguugcaa

SEQ ID NO: 1129 - RNA, 17 nt, Artificial Sequence
  Description of Artificial Sequence Synthetic oligonucleotide
uguguauuugcugugag

SEQ ID NO: 1130 - RNA, 17 nt, Artificial Sequence
  Description of Artificial Sequence Synthetic oligonucleotide
gagguccugggggcgcu

SEQ ID NO: 1131 - RNA, 17 nt, Artificial Sequence
  Description of Artificial Sequence Synthetic oligonucleotide
gaucuucugauggccac

SEQ ID NO: 1132 - RNA, 17 nt, Artificial Sequence
  Description of Artificial Sequence Synthetic oligonucleotide
agccacaacuccgucag

SEQ ID NO: 1133 - RNA, 17 nt, Artificial Sequence
  Description of Artificial Sequence Synthetic oligonucleotide
gauuuucugauagcuac

SEQ ID NO: 1134 - RNA, 17 nt, Artificial Sequence
  Description of Artificial Sequence Synthetic oligonucleotide
ggccacaauucugucag

SEQ ID NO: 1135 - RNA, 17 nt, Artificial Sequence
  Description of Artificial Sequence Synthetic oligonucleotide
gaucuucugauggccac

SEQ ID NO: 1136 - RNA, 17 nt, Artificial Sequence
  Description of Artificial Sequence Synthetic oligonucleotide
ggccacaacuccgucag

SEQ ID NO: 1137 - RNA, 17 nt, Artificial Sequence
  Description of Artificial Sequence Synthetic oligonucleotide
gauuuucugauagcaac

SEQ ID NO: 1138 - RNA, 17 nt, Artificial Sequence
  Description of Artificial Sequence Synthetic oligonucleotide
ggcuacgacucugucaa

SEQ ID NO: 1139 - RNA, 17 nt, Artificial Sequence
  Description of Artificial Sequence Synthetic oligonucleotide
aggccgcgucguggcgg

SEQ ID NO: 1140 - RNA, 19 nt, Artificial Sequence
  Description of Artificial Sequence Synthetic oligonucleotide
ccgcuccucucagcccguc

SEQ ID NO: 1141 - RNA, 17 nt, Artificial Sequence
  Description of Artificial Sequence Synthetic oligonucleotide
acgccacagcuccaacu

SEQ ID NO: 1142 - RNA, 17 nt, Artificial Sequence
  Description of Artificial Sequence Synthetic oligonucleotide
gucucacaauugcucuc

SEQ ID NO: 1143 - RNA, 17 nt, Artificial Sequence
  Description of Artificial Sequence Synthetic oligonucleotide
gaaauacaucagauuug

SEQ ID NO: 1144 - RNA, 17 nt, Artificial Sequence
  Description of Artificial Sequence Synthetic oligonucleotide
aauuagcuucuggccau

SEQ ID NO: 1145 - RNA, 17 nt, Artificial Sequence
  Description of Artificial Sequence Synthetic oligonucleotide
gaucagcuccuggccau

SEQ ID NO: 1146 - RNA, 17 nt, Artificial Sequence
  Description of Artificial Sequence Synthetic oligonucleotide
gaucagcuucuggccau

SEQ ID NO: 1147 - RNA, 17 nt, Artificial Sequence
  Description of Artificial Sequence Synthetic oligonucleotide
gaucagcuucuggccau

SEQ ID NO: 1148 - RNA, 16 nt, Artificial Sequence
  Description of Artificial Sequence Synthetic oligonucleotide
acugccaggcaucagc

SEQ ID NO: 1149 - RNA, 17 nt, Artificial Sequence
  Description of Artificial Sequence Synthetic oligonucleotide
cacugccgggcaucagc

SEQ ID NO: 1150 - RNA, 17 nt, Artificial Sequence
  Description of Artificial Sequence Synthetic oligonucleotide
uccgcccgauccacgau

SEQ ID NO: 1151 - RNA, 17 nt, Artificial Sequence
  Description of Artificial Sequence Synthetic oligonucleotide
ccuuucugucgaugguc

SEQ ID NO: 1152 - RNA, 17 nt, Artificial Sequence
  Description of Artificial Sequence Synthetic oligonucleotide
ccuuucugucgaugguc

SEQ ID NO: 1153 - RNA, 17 nt, Artificial Sequence
  Description of Artificial Sequence Synthetic oligonucleotide
cuugauaauccaguucc

SEQ ID NO: 1154 - RNA, 17 nt, Artificial Sequence
  Description of Artificial Sequence Synthetic oligonucleotide
uuucaggauuuccuccg

SEQ ID NO: 1155 - RNA, 17 nt, Artificial Sequence
  Description of Artificial Sequence Synthetic oligonucleotide
cuucaggauggggcagc

SEQ ID NO: 1156 - RNA, 17 nt, Artificial Sequence
  Description of Artificial Sequence Synthetic oligonucleotide
aggaacaaaccuuuguc

SEQ ID NO: 1157 - RNA, 17 nt, Artificial Sequence
  Description of Artificial Sequence Synthetic oligonucleotide
uuucacaauccaucaac

SEQ ID NO: 1158 - RNA, 17 nt, Artificial Sequence
  Description of Artificial Sequence Synthetic oligonucleotide
cuucacgauccaucaac

SEQ ID NO: 1159 - RNA, 17 nt, Artificial Sequence
  Description of Artificial Sequence Synthetic oligonucleotide
ugggacaaaccccugcc

SEQ ID NO: 1160 - RNA, 17 nt, Artificial Sequence
  Description of Artificial Sequence Synthetic oligonucleotide
cgacuccucuggauguu

SEQ ID NO: 1161 - RNA, 17 nt, Artificial Sequence
  Description of Artificial Sequence Synthetic oligonucleotide
cgacucuucuggauguu

SEQ ID NO: 1162 - RNA, 85 nt, Artificial Sequence
  Description of Artificial Sequence Synthetic oligonucleotide
guggaauaguauaacaauaugcuaaauguuguuauaguaucccacgugcagccagccguccucuagagggcccugaagagggccc

SEQ ID NO: 1163 - RNA, 85 nt, Artificial Sequence
  Description of Artificial Sequence Synthetic oligonucleotide
guggaagaggagaacaauaugcuaaauguuguucucgucucccacgugcagccagccguccucuagagggcccugaagagggccc

SEQ ID NO: 1164 - RNA, 95 nt, Artificial Sequence
  Description of Artificial Sequence Synthetic oligonucleotide
guggucgagaagaggagaacaauaugcuaaauguuguucucgucuccucgaccacgugcagccagccguccucuagagggcccugaagagggccc

SEQ ID NO: 1165 - RNA, 18 nt, Artificial Sequence
  Description of Artificial Sequence Synthetic oligonucleotide
gcaaugccaucaccuccc

SEQ ID NO: 1166 - RNA, 63 nt, Artificial Sequence
  Description of Artificial Sequence Synthetic oligonucleotide
ggugaauaguauaacaauaugcuaaauguuguuauaguauccaccgcaaugccaucaccuccc

SEQ ID NO: 1167 - RNA, 63 nt, Artificial Sequence
  Description of Artificial Sequence Synthetic oligonucleotide
ggugaagaggagaacaauaugcuaaauguuguucucgucuccaccgcaaugccaucaccuccc

SEQ ID NO: 1168 - RNA, 73 nt, Artificial Sequence
  Description of Artificial Sequence Synthetic oligonucleotide
ggugucgagaagaggagaacaauaugcuaaauguuguucucgucuccucgacaccgcaaugccaucaccuccc

SEQ ID NO: 1169 - RNA, 18 nt, Artificial Sequence
  Description of Artificial Sequence Synthetic oligonucleotide
agggguccacauggcaac

SEQ ID NO: 1170 - RNA, 63 nt, Artificial Sequence
  Description of Artificial Sequence Synthetic oligonucleotide
ggugaauaguauaacaauaugcuaaauguuguuauaguauccaccagggguccacauggcaac

SEQ ID NO: 1171 - RNA, 63 nt, Artificial Sequence
  Description of Artificial Sequence Synthetic oligonucleotide
ggugaagaggagaacaauaugcuaaauguuguucucgucuccaccagggguccacauggcaac

SEQ ID NO: 1172 - RNA, 73 nt, Artificial Sequence
  Description of Artificial Sequence Synthetic oligonucleotide
ggugucgagaagaggagaacaauaugcuaaauguuguucucgucuccucgacaccagggguccacauggcaac

SEQ ID NO: 1173 - RNA, 23 nt, Artificial Sequence
  Description of Artificial Sequence Synthetic oligonucleotide
ggugucgagaagaggagaacaau

SEQ ID NO: 1174 - RNA, 18 nt, Artificial Sequence
  Description of Artificial Sequence Synthetic oligonucleotide
ggggugccaagcaguugg

SEQ ID NO: 1175 - RNA, 73 nt, Artificial Sequence
  Description of Artificial Sequence Synthetic oligonucleotide
ggugucgagaagaggagaacaauaugcuaaauguuguucucgucuccucgacaccggggugccaagcaguugg

SEQ ID NO: 1176 - RNA, 18 nt, Artificial Sequence
  Description of Artificial Sequence Synthetic oligonucleotide
ggggugccaagcaguugg

SEQ ID NO: 1177 - RNA, 73 nt, Artificial Sequence
  Description of Artificial Sequence Synthetic oligonucleotide
ggugucgagaagaggagaacaauaugcuaaauguuguucucgucuccucgacaccguuuuuccagacggcagg

SEQ ID NO: 1178 - RNA, 23 nt, Artificial Sequence
  Description of Artificial Sequence Synthetic oligonucleotide
ggugucgagaagaggagaacaau

SEQ ID NO: 1179 - RNA, 29 nt, Artificial Sequence
  Description of Artificial Sequence Synthetic oligonucleotide
ggugucgagaagaggagaacaauaugcua

SEQ ID NO: 1180 - RNA, 73 nt, Artificial Sequence
  Description of Artificial Sequence Synthetic oligonucleotide
ggugucgagaagaggagaacaauaugcuaaauguuguucucgucuccucgacaccccuuucucgucgaugguc

SEQ ID NO: 1181 - RNA, 31 nt, Artificial Sequence
  Description of Artificial Sequence Synthetic oligonucleotide
cauggccccagcagcuucagucccuuucucg

SEQ ID NO: 1182 - DNA, 91 bp, Artificial Sequence
  Description of Artificial Sequence Synthetic oligonucleotide
ggugucgagaagaggagaacaauaugcuaaauguuguucuuccucgacacccauggccccagcagcuucagucccuuucucgucgatggtc

SEQ ID NO: 1183 - DNA, 95 bp, Artificial Sequence
  Description of Artificial Sequence Synthetic oligonucleotide
ggugucgagaagaggagaacaauaugcuaaauguuguucucgucuccucgacacccauggccccagcagcuucagucccuuucucgucgatggtc

SEQ ID NO: 1184 - RNA, 23 nt, Artificial Sequence
  Description of Artificial Sequence Synthetic oligonucleotide
ggugucgagaagaggagaacaau

SEQ ID NO: 1185 - RNA, 63 nt, Artificial Sequence; identical to SEQ ID NO: 1170
  Description of Artificial Sequence Synthetic oligonucleotide

SEQ ID NO: 1186 - RNA, 63 nt, Artificial Sequence; identical to SEQ ID NO: 1171
  Description of Artificial Sequence Synthetic oligonucleotide

SEQ ID NO: 1187 - RNA, 73 nt, Artificial Sequence; identical to SEQ ID NO: 1172
  Description of Artificial Sequence Synthetic oligonucleotide

SEQ ID NO: 1188 - RNA, 64 nt, Artificial Sequence
  Description of Artificial Sequence Synthetic oligonucleotide
ggaccaacugcuuggcaccccuggccaaggucauccaugacaacuuugguaucguggaaggacc

SEQ ID NO: 1189 - RNA, 61 nt, Artificial Sequence
  Description of Artificial Sequence Synthetic oligonucleotide
gggaacuggaucuaucaagacugaguugauuucugugucugaaguguaagugaacacagaa

SEQ ID NO: 1190 - DNA, 61 bp, Artificial Sequence
  Description of Artificial Sequence Synthetic oligonucleotide
ggaccatcgacgagaaagggactgaagctgctggggccatgtttttagaggccatacccat

SEQ ID NO: 1191 - RNA, 22 nt, Artificial Sequence
  Description of Artificial Sequence Synthetic oligonucleotide
ucauuaaacgccagaguccgga

SEQ ID NO: 1192 - RNA, 22 nt, Artificial Sequence
  Description of Artificial Sequence Synthetic oligonucleotide
ucugaauaauccaggaaaagca

SEQ ID NO: 1193 - RNA, 22 nt, Artificial Sequence
  Description of Artificial Sequence Synthetic oligonucleotide
uauaggggugccaagcaguugg

SEQ ID NO: 1194 - RNA, 22 nt, Artificial Sequence
  Description of Artificial Sequence Synthetic oligonucleotide
uaugguuuuuccagacggcagg

SEQ ID NO: 1195 - RNA, 22 nt, Artificial Sequence
  Description of Artificial Sequence Synthetic oligonucleotide
ggugcagauuccaggugggacg

SEQ ID NO: 1196 - RNA, 22 nt, Artificial Sequence
  Description of Artificial Sequence Synthetic oligonucleotide
acagacuuggccacugaguggg

SEQ ID NO: 1197 - RNA, 22 nt, Artificial Sequence
  Description of Artificial Sequence Synthetic oligonucleotide
uaugugucggccacggaacagg

SEQ ID NO: 1198 - RNA, 22 nt, Artificial Sequence
  Description of Artificial Sequence Synthetic oligonucleotide
aauaagggguccacauggcaac

SEQ ID NO: 1199 - RNA, 22 nt, Artificial Sequence
  Description of Artificial Sequence Synthetic oligonucleotide
ucgagcaaugccaucaccuccc

SEQ ID NO: 1200 - RNA, 22 nt, Artificial Sequence
  Description of Artificial Sequence Synthetic oligonucleotide
uauuucccugccagaauagaug

SEQ ID NO: 1201 - RNA, 22 nt, Artificial Sequence
  Description of Artificial Sequence Synthetic oligonucleotide
gaugcuccaaccaccacaaguu

SEQ ID NO: 1202 - RNA, 22 nt, Artificial Sequence
  Description of Artificial Sequence Synthetic oligonucleotide
cgucucuugcccacgccaccag

SEQ ID NO: 1203 - RNA, 22 nt, Artificial Sequence
  Description of Artificial Sequence Synthetic oligonucleotide
gucucuugauacauccaguucc

SEQ ID NO: 1204 - RNA, 22 nt, Artificial Sequence
  Description of Artificial Sequence Synthetic oligonucleotide
cacaugggauucccauugauga

SEQ ID NO: 1205 - RNA, 22 nt, Artificial Sequence
  Description of Artificial Sequence Synthetic oligonucleotide
uaucgaccaaacccguugacuc

SEQ ID NO: 1206 - RNA, 22 nt, Artificial Sequence
  Description of Artificial Sequence Synthetic oligonucleotide
cacgucaugagcccuuccacga

SEQ ID NO: 1207 - RNA, 22 nt, Artificial Sequence
  Description of Artificial Sequence Synthetic oligonucleotide
aacgagggaucccgcuccugga

SEQ ID NO: 1208 - RNA, 22 nt, Artificial Sequence
  Description of Artificial Sequence Synthetic oligonucleotide
gaagaggcugucgucauacuuc

SEQ ID NO: 1209 - RNA, 22 nt, Artificial Sequence
  Description of Artificial Sequence Synthetic oligonucleotide
caagaggucaacgaagggguca

SEQ ID NO: 1210 - RNA, 22 nt, Artificial Sequence
  Description of Artificial Sequence Synthetic oligonucleotide
aacgccaggggcgcuaagcagu

SEQ ID NO: 1211 - RNA, 22 nt, Artificial Sequence
  Description of Artificial Sequence Synthetic oligonucleotide
uacgcauggaccguggucauga

SEQ ID NO: 1212 - RNA, 22 nt, Artificial Sequence
  Description of Artificial Sequence Synthetic oligonucleotide
uacaugacccucuuggcucccc

SEQ ID NO: 1213 - RNA, 22 nt, Artificial Sequence
  Description of Artificial Sequence Synthetic oligonucleotide
gacuagccaaacucguugucau

SEQ ID NO: 1214 - RNA, 22 nt, Artificial Sequence
  Description of Artificial Sequence Synthetic oligonucleotide
agucgccacagcuucccggagg

SEQ ID NO: 1215 - RNA, 22 nt, Artificial Sequence
  Description of Artificial Sequence Synthetic oligonucleotide
uguauauccaccuuaccagagu

SEQ ID NO: 1216 - RNA, 22 nt, Artificial Sequence
  Description of Artificial Sequence Synthetic oligonucleotide
aggaggggucucacuccuugga

SEQ ID NO: 1217 - RNA, 22 nt, Artificial Sequence
  Description of Artificial Sequence Synthetic oligonucleotide
cuaggcaacaacauccacuuua

SEQ ID NO: 1218 - RNA, 22 nt, Artificial Sequence
  Description of Artificial Sequence Synthetic oligonucleotide
ccgagcgccagcagaggcaggg

SEQ ID NO: 1219 - RNA, 22 nt, Artificial Sequence
  Description of Artificial Sequence Synthetic oligonucleotide
uaugguuuuuccagacggcagg

SEQ ID NO: 1220 - RNA, 22 nt, Artificial Sequence
  Description of Artificial Sequence Synthetic oligonucleotide
gaagaggcugucgucauacuuc

SEQ ID NO: 1221 - RNA, 22 nt, Artificial Sequence
  Description of Artificial Sequence Synthetic oligonucleotide
gaagaggcugucgucauacuuc

SEQ ID NO: 1222 - RNA, 22 nt, Artificial Sequence
  Description of Artificial Sequence Synthetic oligonucleotide
uacaugacccucuuggcucccc

SEQ ID NO: 1223 - RNA, 22 nt, Artificial Sequence
  Description of Artificial Sequence Synthetic oligonucleotide
uacaugacccucuuggcucccc

SEQ ID NO: 1224 - RNA, 22 nt, Artificial Sequence
  Description of Artificial Sequence Synthetic oligonucleotide
agucgccacagcuucccggagg

SEQ ID NO: 1225 - RNA, 22 nt, Artificial Sequence
  Description of Artificial Sequence Synthetic oligonucleotide
agucgccacagcuucccggagg

SEQ ID NO: 1226 - RNA, 22 nt, Artificial Sequence
  Description of Artificial Sequence Synthetic oligonucleotide
aggaggggucucacuccuugga

SEQ ID NO: 1227 - RNA, 22 nt, Artificial Sequence
  Description of Artificial Sequence Synthetic oligonucleotide
aggaggggucucacuccuugga

SEQ ID NO: 1228 - DNA, 55 bp, Artificial Sequence
  Description of Artificial Sequence Synthetic oligonucleotide
taatctaggaaaactgagaacagaggccctgaaaaagggccaaattcttccaccc

SEQ ID NO: 1229 - RNA, 70 nt, Artificial Sequence
  Description of Artificial Sequence Synthetic oligonucleotide
aaaccgagggaucauaggggacugaauccaccauucuucucccaaucccugcaacuccuucuuccccugc

SEQ ID NO: 1230 - RNA, 70 nt, Artificial Sequence
  Description of Artificial Sequence Synthetic oligonucleotide
ugaacagcuccucgcccuugcucacuggcagagcccuccagcaucgcgagcaggcgcugccuccuccgcc

SEQ ID NO: 1231 - RNA, 71 nt, Artificial Sequence
  Description of Artificial Sequence Synthetic oligonucleotide
ucucaguccaauguaugguccgagcacaagcucuaaucaaaguccgcggguguagaccgguugccauagga

SEQ ID NO: 1232 - RNA, 71 nt, Artificial Sequence
  Description of Artificial Sequence Synthetic oligonucleotide
acagcuccucgcccuugcucacuggcagagcccuccagcaucgcgagcaggcgcugccuccuccgccgcug

SEQ ID NO: 1233 - RNA, 71 nt, Artificial Sequence
  Description of Artificial Sequence Synthetic oligonucleotide
acagcuccucgcccuugcucacuggcagagcccucaagcaucgcgagcaggcgcugccuccuccgccgcug

SEQ ID NO: 1234 - RNA, 71 nt, Artificial Sequence
  Description of Artificial Sequence Synthetic oligonucleotide
acagcuccucgcccuugcucacuggcagagcccucuagcaucgcgagcaggcgcugccuccuccgccgcug

SEQ ID NO: 1235 - RNA, 71 nt, Artificial Sequence
  Description of Artificial Sequence Synthetic oligonucleotide
acagcuccucgcccuugcucacuggcagagcccucgagcaucgcgagcaggcgcugccuccuccgccgcug

SEQ ID NO: 1236 - RNA, 71 nt, Artificial Sequence
  Description of Artificial Sequence Synthetic oligonucleotide
acagcuccucgcccuugcucacuggcagagcccugcagcaucgcgagcaggcgcugccuccuccgccgcug

SEQ ID NO: 1237 - RNA, 71 nt, Artificial Sequence
  Description of Artificial Sequence Synthetic oligonucleotide
acagcuccucgcccuugcucacuggcagagcccuucagcaucgcgagcaggcgcugccuccuccgccgcug

SEQ ID NO: 1238 - RNA, 71 nt, Artificial Sequence
  Description of Artificial Sequence Synthetic oligonucleotide
acagcuccucgcccuugcucacuggcagagcccuacagcaucgcgagcaggcgcugccuccuccgccgcug

SEQ ID NO: 1239 - RNA, 71 nt, Artificial Sequence
  Description of Artificial Sequence Synthetic oligonucleotide
acagcuccucgcccuugcucacuggcagagcccuccugcaucgcgagcaggcgcugccuccuccgccgcug

SEQ ID NO: 1240 - RNA, 71 nt, Artificial Sequence
  Description of Artificial Sequence Synthetic oligonucleotide
acagcuccucgcccuugcucacuggcagagcccugcugcaucgcgagcaggcgcugccuccuccgccgcug

SEQ ID NO: 1241 - RNA, 71 nt, Artificial Sequence
  Description of Artificial Sequence Synthetic oligonucleotide
acagcuccucgcccuugcucacuggcagagcccuucugcaucgcgagcaggcgcugccuccuccgccgcug

SEQ ID NO: 1242 - RNA, 71 nt, Artificial Sequence
  Description of Artificial Sequence Synthetic oligonucleotide
acagcuccucgcccuugcucacuggcagagcccuacugcaucgcgagcaggcgcugccuccuccgccgcug

SEQ ID NO: 1243 - RNA, 71 nt, Artificial Sequence
  Description of Artificial Sequence Synthetic oligonucleotide
acagcuccucgcccuugcucacuggcagagcccucccgcaucgcgagcaggcgcugccuccuccgccgcug

SEQ ID NO: 1244 - RNA, 71 nt, Artificial Sequence
  Description of Artificial Sequence Synthetic oligonucleotide
acagcuccucgcccuugcucacuggcagagcccugccgcaucgcgagcaggcgcugccuccuccgccgcug

SEQ ID NO: 1245 - RNA, 71 nt, Artificial Sequence
  Description of Artificial Sequence Synthetic oligonucleotide
acagcuccucgcccuugcucacuggcagagcccuuccgcaucgcgagcaggcgcugccuccuccgccgcug

SEQ ID NO: 1246 - RNA, 71 nt, Artificial Sequence
  Description of Artificial Sequence Synthetic oligonucleotide
acagcuccucgcccuugcucacuggcagagcccuaccgcaucgcgagcaggcgcugccuccuccgccgcug

SEQ ID NO: 1247 - RNA, 71 nt, Artificial Sequence
  Description of Artificial Sequence Synthetic oligonucleotide
acagcuccucgcccuugcucacuggcagagcccuccggcaucgcgagcaggcgcugccuccuccgccgcug

SEQ ID NO: 1248 - RNA, 71 nt, Artificial Sequence; identical to SEQ ID NO: 1240
  Description of Artificial Sequence Synthetic oligonucleotide

SEQ ID NO: 1249 - RNA, 71 nt, Artificial Sequence
  Description of Artificial Sequence Synthetic oligonucleotide
acagcuccucgcccuugcucacuggcagagcccuucggcaucgcgagcaggcgcugccuccuccgccgcug

SEQ ID NO: 1250 - RNA, 71 nt, Artificial Sequence
  Description of Artificial Sequence Synthetic oligonucleotide
acagcuccucgcccuugcucacuggcagagcccuacggcaucgcgagcaggcgcugccuccuccgccgcug

SEQ ID NO: 1251 - RNA, 31 nt, Artificial Sequence
  Description of Artificial Sequence Synthetic oligonucleotide
acuggcagagcccuccagcaucgcgagcagg

SEQ ID NO: 1252 - RNA, 51 nt, Artificial Sequence
  Description of Artificial Sequence Synthetic oligonucleotide
gcccuugcucacuggcagagcccuccagcaucgcgagcaggcgcugccucc

SEQ ID NO: 1253 - RNA, 71 nt, Artificial Sequence; identical to SEQ ID NO: 1232
  Description of Artificial Sequence Synthetic oligonucleotide

SEQ ID NO: 1254 - RNA, 91 nt, Artificial Sequence
  Description of Artificial Sequence Synthetic oligonucleotide
accccggugaacagcuccucgcccuugcucacuggcagagcccuccagcaucgcgagcaggcgcugccuccuccgccgcugccuccuccgc

SEQ ID NO: 1255 - RNA, 282 nt, Artificial Sequence
  Description of Artificial Sequence Synthetic polynucleotide
gcucgaccaggaugggcaccaccccggugaacagcuccucgcccuugcucacuggcagagcccuccagcaucgcgagcaggcgcugccuccuccgccgcugccuccuccgccgcugccuccuccgcccugcucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguaca

SEQ ID NO: 1256 - RNA, 151 nt, Artificial Sequence
  Description of Artificial Sequence Synthetic polynucleotide
ucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguaca

SEQ ID NO: 1257 - RNA, 171 nt, Artificial Sequence
  Description of Artificial Sequence Synthetic polynucleotide
gccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccau

SEQ ID NO: 1258 - RNA, 191 nt, Artificial Sequence
  Description of Artificial Sequence Synthetic polynucleotide
ugaacuuguggccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccaugccgccggug

SEQ ID NO: 1259 - RNA, 211 nt, Artificial Sequence
  Description of Artificial Sequence Synthetic polynucleotide
ccggacacgcugaacuuguggccguuuacgucgccguccagcucgaccaggaugggcaccaccccggugaacagcuccucgcccuugcucacuggcagagcccuccagcaucgcgagcaggcgcugccuccuccgccgcugccuccuccgccgcugccuccuccgcccugcagcuuguacagcucguccaugccgccgguggaguggcggc

SEQ ID NO: 1260 - RNA, 31 nt, Artificial Sequence
  Description of Artificial Sequence Synthetic oligonucleotide
gcgaccggggaucuccacagauucuuccggc

SEQ ID NO: 1261 - RNA, 51 nt, Artificial Sequence
  Description of Artificial Sequence Synthetic oligonucleotide
gcucacgguggcgaccggggaucuccacagauucuuccggcguguauaccu

SEQ ID NO: 1262 - RNA, 71 nt, Artificial Sequence
  Description of Artificial Sequence Synthetic oligonucleotide
ccucgcccuugcucacgguggcgaccggggaucuccacagauucuuccggcguguauaccuucugcugccu

SEQ ID NO: 1263 - RNA, 91 nt, Artificial Sequence
  Description of Artificial Sequence Synthetic oligonucleotide
gugaacagcuccucgcccuugcucacgguggcgaccggggaucuccacagauucuuccggcguguauaccuucugcugccuccuccgccgc

SEQ ID NO: 1264 - RNA, 111 nt, Artificial Sequence
  Description of Artificial Sequence Synthetic polynucleotide
caccaccccggugaacagcuccucgcccuugcucacgguggcgaccggggaucuccacagauucuuccggcguguauaccuucugcugccuccuccgccgcugccuccucc

SEQ ID NO: 1265 - RNA, 131 nt, Artificial Sequence
  Description of Artificial Sequence Synthetic polynucleotide
ccaggaugggcaccaccccggugaacagcuccucgcccuugcucacgguggcgaccggggaucuccacagauucuuccggcguguauaccuucugcugccuccuccgccgcugccuccuccgccgcugccu

SEQ ID NO: 1266 - RNA, 151 nt, Artificial Sequence
  Description of Artificial Sequence Synthetic polynucleotide
uccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccu

SEQ ID NO: 1267 - RNA, 171 nt, Artificial Sequence
  Description of Artificial Sequence Synthetic polynucleotide
cggcgacguauccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuugua

SEQ ID NO: 1268 - RNA, 191 nt, Artificial Sequence
  Description of Artificial Sequence Synthetic polynucleotide
uguggccguuuacgucgccguccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuuguacagcucgucc

SEQ ID NO: 1269 - RNA, 211 nt, Artificial Sequence
  Description of Artificial Sequence Synthetic polynucleotide
acgcugaacuuguggccguuuacgucgccguccagcucgaccaggaugggcaccaccccggugaacagcuccucgcccuugcucacgguggcgaccggggaucuccacagauucuuccggcguguauaccuucugcugccuccuccgccgcugccuccuccgccgcugccuccuccgcccugcagcuuguacagcucguccaugccgccgg

SEQ ID NO: 1270 - RNA, 71 nt, Artificial Sequence
  Description of Artificial Sequence Synthetic oligonucleotide
cagcaucgcgagcaggcgcugccuccuccgccgcugccuccuccgccgcugccuccuccgcccugcagcuu

SEQ ID NO: 1271 - RNA, 71 nt, Artificial Sequence
  Description of Artificial Sequence Synthetic oligonucleotide
cccuccagcaucgcgagcaggcgcugccuccuccgccgcugccuccuccgccgcugccuccuccgcccugc

SEQ ID NO: 1272 - RNA, 71 nt, Artificial Sequence
  Description of Artificial Sequence Synthetic oligonucleotide
cagagcccuccagcaucgcgagcaggcgcugccuccuccgccgcugccuccuccgccgcugccuccuccgc

SEQ ID NO: 1273 - RNA, 72 nt, Artificial Sequence
  Description of Artificial Sequence Synthetic oligonucleotide
acuggcagagcccucccagcaucgcgagcaggcgcugccuccuccgccgcugccuccuccgccgcugccucc

SEQ ID NO: 1274 - RNA, 71 nt, Artificial Sequence
  Description of Artificial Sequence Synthetic oligonucleotide
ugcucacuggcagagcccuccagcaucgcgagcaggcgcugccuccuccgccgcugccuccuccgccgcug

SEQ ID NO: 1275 - RNA, 71 nt, Artificial Sequence
  Description of Artificial Sequence Synthetic oligonucleotide
gcccuugcucacuggcagagcccuccagcaucgcgagcaggcgcugccuccuccgccgcugccuccuccgc

SEQ ID NO: 1276 - RNA, 71 nt, Artificial Sequence
  Description of Artificial Sequence Synthetic oligonucleotide
uccucgcccuugcucacuggcagagcccuccagcaucgcgagcaggcgcugccuccuccgccgcugccucc

SEQ ID NO: 1277 - RNA, 71 nt, Artificial Sequence
  Description of Artificial Sequence Synthetic oligonucleotide
ggugaacagcuccucgcccuugcucacuggcagagcccuccagcaucgcgagcaggcgcugccuccuccgc

SEQ ID NO: 1278 - RNA, 71 nt, Artificial Sequence
  Description of Artificial Sequence Synthetic oligonucleotide
accccggugaacagcuccucgcccuugcucacuggcagagcccuccagcaucgcgagcaggcgcugccucc

SEQ ID NO: 1279 - RNA, 71 nt, Artificial Sequence
  Description of Artificial Sequence Synthetic oligonucleotide
gcaccaccccggugaacagcuccucgcccuugcucacuggcagagcccuccagcaucgcgagcaggcgcug

SEQ ID NO: 1280 - RNA, 71 nt, Artificial Sequence
  Description of Artificial Sequence Synthetic oligonucleotide
gaugggcaccaccccggugaacagcuccucgcccuugcucacuggcagagcccuccagcaucgcgagcagg

SEQ ID NO: 1281 - RNA, 71 nt, Artificial Sequence
  Description of Artificial Sequence Synthetic oligonucleotide
accaggaugggcaccaccccggugaacagcuccucgcccuugcucacuggcagagcccuccagcaucgcga

SEQ ID NO: 1282 - RNA, 71 nt, Artificial Sequence
  Description of Artificial Sequence Synthetic oligonucleotide
gcucgaccaggaugggcaccaccccggugaacagcuccucgcccuugcucacuggcagagcccuccagcau

SEQ ID NO: 1283 - RNA, 71 nt, Artificial Sequence
  Description of Artificial Sequence Synthetic oligonucleotide
guccagcucgaccaggaugggcaccaccccggugaacagcuccucgcccuugcucacuggcagagcccucc

SEQ ID NO: 1284 - RNA, 71 nt, Artificial Sequence
  Description of Artificial Sequence Synthetic oligonucleotide
cacagauucuuccggcguguauaccuucugcugccuccuccgccgcugccuccuccgccgcugccuccucc

SEQ ID NO: 1285 - RNA, 71 nt, Artificial Sequence
  Description of Artificial Sequence Synthetic oligonucleotide
aucuccacagauucuuccggcguguauaccuucugcugccuccuccgccgcugccuccuccgccgcugccu

SEQ ID NO: 1286 - RNA, 71 nt, Artificial Sequence
  Description of Artificial Sequence Synthetic oligonucleotide
cggggaucuccacagauucuuccggcguguauaccuucugcugccuccuccgccgcugccuccuccgccgc

SEQ ID NO: 1287 - RNA, 71 nt, Artificial Sequence
  Description of Artificial Sequence Synthetic oligonucleotide
gcgaccggggaucuccacagauucuuccggcguguauaccuucugcugccuccuccgccgcugccuccucc

SEQ ID NO: 1288 - RNA, 71 nt, Artificial Sequence
  Description of Artificial Sequence Synthetic oligonucleotide
cgguggcgaccggggaucuccacagauucuuccggcguguauaccuucugcugccuccuccgccgcugccu

SEQ ID NO: 1289 - RNA, 71 nt, Artificial Sequence
  Description of Artificial Sequence Synthetic oligonucleotide
gcucacgguggcgaccggggaucuccacagauucuuccggcguguauaccuucugcugccuccuccgccgc

SEQ ID NO: 1290 - RNA, 71 nt, Artificial Sequence
  Description of Artificial Sequence Synthetic oligonucleotide
cccuugcucacgguggcgaccggggaucuccacagauucuuccggcguguauaccuucugcugccuccucc

SEQ ID NO: 1291 - RNA, 71 nt, Artificial Sequence
  Description of Artificial Sequence Synthetic oligonucleotide
cagcuccucgcccuugcucacgguggcgaccggggaucuccacagauucuuccggcguguauaccuucugc

SEQ ID NO: 1292 - RNA, 71 nt, Artificial Sequence
  Description of Artificial Sequence Synthetic oligonucleotide
gugaacagcuccucgcccuugcucacgguggcgaccggggaucuccacagauucuuccggcguguauaccu

SEQ ID NO: 1293 - RNA, 71 nt, Artificial Sequence
  Description of Artificial Sequence Synthetic oligonucleotide
ccccggugaacagcuccucgcccuugcucacgguggcgaccggggaucuccacagauucuuccggcgugua

SEQ ID NO: 1294 - RNA, 71 nt, Artificial Sequence
  Description of Artificial Sequence Synthetic oligonucleotide
caccaccccggugaacagcuccucgcccuugcucacgguggcgaccggggaucuccacagauucuuccggc

SEQ ID NO: 1295 - RNA, 71 nt, Artificial Sequence
  Description of Artificial Sequence Synthetic oligonucleotide
augggcaccaccccggugaacagcuccucgcccuugcucacgguggcgaccggggaucuccacagauucuu

SEQ ID NO: 1296 - RNA, 71 nt, Artificial Sequence
  Description of Artificial Sequence Synthetic oligonucleotide
ccaggaugggcaccaccccggugaacagcuccucgcccuugcucacgguggcgaccggggaucuccacaga

SEQ ID NO: 1297 - RNA, 71 nt, Artificial Sequence
  Description of Artificial Sequence Synthetic oligonucleotide
cucgaccaggaugggcaccaccccggugaacagcuccucgcccuugcucacgguggcgaccggggaucucc

SEQ ID NO: 1298 - RNA, 111 nt, Artificial Sequence
  Description of Artificial Sequence Synthetic polynucleotide
gaugggcaccaccccggugaacagcuccucgcccuugcucacuggcagagcccuccagcaucgcgagcaggcgcugccuccuccgcccugcagcuuguacagcucguccau

SEQ ID NO: 1299 - RNA, 111 nt, Artificial Sequence
  Description of Artificial Sequence Synthetic polynucleotide
gaugggcaccaccccggugaacagcuccucgcccuugcucacuggcagagcccugcagcaucgcgagcaggcgcugccuccuccgcccugcagcuuguacagcucguccau

SEQ ID NO: 1300 - RNA, 111 nt, Artificial Sequence
  Description of Artificial Sequence Synthetic polynucleotide
gaugggcaccaccccggugaacagcuccucgcccuugcucacuggcagagcccuucagcaucgcgagcaggcgcugccuccuccgcccugcagcuuguacagcucguccau

SEQ ID NO: 1301 - RNA, 111 nt, Artificial Sequence
  Description of Artificial Sequence Synthetic polynucleotide
gaugggcaccaccccggugaacagcuccucgcccuugcucacuggcagagcccuacagcaucgcgagcaggcgcugccuccuccgcccugcagcuuguacagcucguccau

SEQ ID NO: 1302 - RNA, 111 nt, Artificial Sequence
  Description of Artificial Sequence Synthetic polynucleotide
gaugggcaccaccccggugaacagcuccucgcccuugcucacuggcagagcccuccggcaucgcgagcaggcgcugccuccuccgcccugcagcuuguacagcucguccau

SEQ ID NO: 1303 - RNA, 111 nt, Artificial Sequence
  Description of Artificial Sequence Synthetic polynucleotide
gaugggcaccaccccggugaacagcuccucgcccuugcucacuggcagagcccugcggcaucgcgagcaggcgcugccuccuccgcccugcagcuuguacagcucguccau

SEQ ID NO: 1304 - RNA, 111 nt, Artificial Sequence
  Description of Artificial Sequence Synthetic polynucleotide
gaugggcaccaccccggugaacagcuccucgcccuugcucacuggcagagcccuucggcaucgcgagcaggcgcugccuccuccgcccugcagcuuguacagcucguccau

SEQ ID NO: 1305 - RNA, 111 nt, Artificial Sequence
  Description of Artificial Sequence Synthetic polynucleotide
gaugggcaccaccccggugaacagcuccucgcccuugcucacuggcagagcccuacggcaucgcgagcaggcgcugccuccuccgcccugcagcuuguacagcucguccau

SEQ ID NO: 1306 - RNA, 111 nt, Artificial Sequence
  Description of Artificial Sequence Synthetic polynucleotide
gaugggcaccaccccggugaacagcuccucgcccuugcucacuggcagagcccuccugcaucgcgagcaggcgcugccuccuccgcccugcagcuuguacagcucguccau

SEQ ID NO: 1307 - RNA, 111 nt, Artificial Sequence
  Description of Artificial Sequence Synthetic polynucleotide
gaugggcaccaccccggugaacagcuccucgcccuugcucacuggcagagcccugcugcaucgcgagcaggcgcugccuccuccgcccugcagcuuguacagcucguccau

SEQ ID NO: 1308 - RNA, 111 nt, Artificial Sequence
  Description of Artificial Sequence Synthetic polynucleotide
gaugggcaccaccccggugaacagcuccucgcccuugcucacuggcagagcccuacugcaucgcgagcaggcgcugccuccuccgcccugcagcuuguacagcucguccau

SEQ ID NO: 1309 - RNA, 111 nt, Artificial Sequence
  Description of Artificial Sequence Synthetic polynucleotide
gaugggcaccaccccggugaacagcuccucgcccuugcucacuggcagagcccuucugcaucgcgagcaggcgcugccuccuccgcccugcagcuuguacagcucguccau

SEQ ID NO: 1310 - RNA, 111 nt, Artificial Sequence
  Description of Artificial Sequence Synthetic polynucleotide
gaugggcaccaccccggugaacagcuccucgcccuugcucacuggcagagcccucccgcaucgcgagcaggcgcugccuccuccgcccugcagcuuguacagcucguccau

SEQ ID NO: 1311 - RNA, 111 nt, Artificial Sequence
  Description of Artificial Sequence Synthetic polynucleotide
gaugggcaccaccccggugaacagcuccucgcccuugcucacuggcagagcccugccgcaucgcgagcaggcgcugccuccuccgcccugcagcuuguacagcucguccau

SEQ ID NO: 1312 - RNA, 111 nt, Artificial Sequence
  Description of Artificial Sequence Synthetic polynucleotide
gaugggcaccaccccggugaacagcuccucgcccuugcucacuggcagagcccuuccgcaucgcgagcaggcgcugccuccuccgcccugcagcuuguacagcucguccau

SEQ ID NO: 1313 - RNA, 111 nt, Artificial Sequence
  Description of Artificial Sequence Synthetic polynucleotide
gaugggcaccaccccggugaacagcuccucgcccuugcucacuggcagagcccuaccgcaucgcgagcaggcgcugccuccuccgcccugcagcuuguacagcucguccau

SEQ ID NO: 1314 - RNA, 111 nt, Artificial Sequence
  Description of Artificial Sequence Synthetic polynucleotide
uaccgcuacagccacgcugauuucagcuauaccugcccgguauaaagggacguucacaccgcgauguucucugcuggggaauugcgcgauauucaggauuaaaagaagugc

SEQ ID NO: 1315 - RNA, 151 nt, Artificial Sequence
  Description of Artificial Sequence Synthetic polynucleotide
acuacaguugcuccgauauuuaggcuacgucaauaggcacuaacuuauuggcgcuggugaacggacuuccucucgaguaccagaagaugacuacaaaacuccuuuccauugcgaguaucggagucuggcucaguuuggccagggaggcacu

SEQ ID NO: 1316 - RNA, 51 nt, Artificial Sequence
  Description of Artificial Sequence Synthetic oligonucleotide
cggaagaggguggggccgcgguggccagggagccggcgccgccacgcgcgg

SEQ ID NO: 1317 - RNA, 71 nt, Artificial Sequence
  Description of Artificial Sequence Synthetic oligonucleotide
cagcugaggccggaagaggguggggccgcgguggccagggagccggcgccgccacgcgcggguggggggga

SEQ ID NO: 1318 - RNA, 111 nt, Artificial Sequence
  Description of Artificial Sequence Synthetic polynucleotide
ggaggcgaaagcagcccggacagcugaggccggaagaggguggggccgcgguggccagggagccggcgccgccacgcgcgggugggggggacugggguugcucgcgggcuc

SEQ ID NO: 1319 - RNA, 151 nt, Artificial Sequence
  Description of Artificial Sequence Synthetic polynucleotide
gaggcgcagcauccacaggcggaggcgaaagcagcccggacagcugaggccggaagaggguggggccgcgguggccagggagccggcgccgccacgcgcgggugggggggacugggguugcucgcgggcuccgggcgggcggcgggcgccg

SEQ ID NO: 1320 - RNA, 51 nt, Artificial Sequence
  Description of Artificial Sequence Synthetic oligonucleotide
ucuugccuacgccaccagcuccaaccaccacaaguuuauauucagucauuu

SEQ ID NO: 1321 - RNA, 111 nt, Artificial Sequence
  Description of Artificial Sequence Synthetic polynucleotide
gauucugaauuagcuguaucgucaaggcacucuugccuacgccaccagcuccaaccaccacaaguuuauauucagucauuuucagcaggccucucucccgcaccugggagc

SEQ ID NO: 1322 - RNA, 151 nt, Artificial Sequence
  Description of Artificial Sequence Synthetic polynucleotide
aucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgc

SEQ ID NO: 1323 - RNA, 51 nt, Artificial Sequence
  Description of Artificial Sequence Synthetic oligonucleotide
ucggcaugguaugaaguacuucguccaggagcuggagggcccgguguaagu

SEQ ID NO: 1324 - RNA, 71 nt, Artificial Sequence
  Description of Artificial Sequence Synthetic oligonucleotide
gggucugcaaucggcaugguaugaaguacuucguccaggagcuggagggcccgguguaagugaauuucaau

SEQ ID NO: 1325 - RNA, 111 nt, Artificial Sequence
  Description of Artificial Sequence Synthetic polynucleotide
gaccucagucuaaagguugugggucugcaaucggcaugguaugaaguacuucguccaggagcuggagggcccgguguaagugaauuucaauccagcaagguguuucuuuga

SEQ ID NO: 1326 - RNA, 151 nt, Artificial Sequence
  Description of Artificial Sequence Synthetic polynucleotide
uaagggccccaacgguaaaagaccucagucuaaagguugugggucugcaaucggcaugguaugaaguacuucguccaggagcuggagggcccgguguaagugaauuucaauccagcaagguguuucuuugaugcucugucuuggguaaucc

SEQ ID NO: 1327 - RNA, 51 nt, Artificial Sequence
  Description of Artificial Sequence Synthetic oligonucleotide
ugggggguucggcugccgacaucagcaauugcucugccaccaucucagccc

SEQ ID NO: 1328 - RNA, 71 nt, Artificial Sequence
  Description of Artificial Sequence Synthetic oligonucleotide
agcagggccgugggggguucggcugccgacaucagcaauugcucugccaccaucucagcccauccuccgaa

SEQ ID NO: 1329 - RNA, 111 nt, Artificial Sequence
  Description of Artificial Sequence Synthetic polynucleotide
aguagaaggccaagagccacagcagggccgugggggguucggcugccgacaucagcaauugcucugccaccaucucagcccauccuccgaagugaaugaacaggaaccagc

SEQ ID NO: 1330 - RNA, 151 nt, Artificial Sequence
  Description of Artificial Sequence Synthetic polynucleotide
ccucccaucacgggggccguaguagaaggccaagagccacagcagggccgugggggguucggcugccgacaucagcaauugcucugccaccaucucagcccauccuccgaagugaaugaacaggaaccagcucucaaagggaccuccgcag

SEQ ID NO: 1331 - DNA, 151 bp, Artificial Sequence
  Description of Artificial Sequence Synthetic polynucleotide
gccaaacaccacatgcttgccatctagccaggctgtcttgactgtcgtgatgaagaactgggagccgttggtgtccttgcctgcgttggccatgctcacccagccaggcccgtagtgcttcagtttgaagttctcatcggggaagcgctca

SEQ ID NO: 1332 - DNA, 151 bp, Artificial Sequence
  Description of Artificial Sequence Synthetic polynucleotide
gggagtgggtccgctccaccagatgccagcaccggggccagtgcagctcagagccctgtggcggactacagggcccgcacagacggtcactcaaagaaagatgtccctgtgccctactccttggcgatggcaaagggcttctccacctcga

SEQ ID NO: 1333 - DNA, 151 bp, Artificial Sequence
  Description of Artificial Sequence Synthetic polynucleotide
tgcattttgtaaaatagatactagcagattgtcccaagatgtgtacagctcattctcacagcccagcgagggcacctactccacaaatgcgtggccacaggtcatcacctgtcctgtggccctggcgagcctgatccctcacgccgggcac

SEQ ID NO: 1334 - DNA, 151 bp, Artificial Sequence
  Description of Artificial Sequence Synthetic polynucleotide
gctcattctcacagcccagcgagggcacttactccacaaatgcgtggccacaggtcatcacctgtcctgtggccccggcgagcctgatccctcacgccgggcacccacacggcctgcgtgccttctagacttgagttcgcagctctttaag

SEQ ID NO: 1335 - DNA, 151 bp, Artificial Sequence
  Description of Artificial Sequence Synthetic polynucleotide
tcggccgggccctgggggcggtgggcgctggccaggacgcccaccgtgtggttgctgtccaggacggtcccggcccgcgacacttcggcccagagctgctcctcatccagcagcgccagcagccccatggccgtgagcaccggcttgcgca

SEQ ID NO: 1336 - RNA, 111 nt, Artificial Sequence
  Description of Artificial Sequence Synthetic polynucleotide
ugaccagucuuaagaucuuucuugaccugcaccauaagaacuucuccaaagguaccaaaauacucuuucagguccuguucgguuguuuuccaugggagacccaacacuauu

SEQ ID NO: 1337 - RNA, 111 nt, Artificial Sequence
  Description of Artificial Sequence Synthetic polynucleotide
gaugggcaccaccccggugaacagcuccucgcccuugcucacuggcagagcccucgagcaucgcgagcaggcgcugccuccuccgcccugcagcuuguacagcucguccau

SEQ ID NO: 1338 - RNA, 111 nt, Artificial Sequence
  Description of Artificial Sequence Synthetic polynucleotide
gaugggcaccaccccggugaacagcuccucgcccuugcucacuggcagagcccuggagcaucgcgagcaggcgcugccuccuccgcccugcagcuuguacagcucguccau

SEQ ID NO: 1339 - RNA, 111 nt, Artificial Sequence
  Description of Artificial Sequence Synthetic polynucleotide
gaugggcaccaccccggugaacagcuccucgcccuugcucacuggcagagcccuugagcaucgcgagcaggcgcugccuccuccgcccugcagcuuguacagcucguccau

SEQ ID NO: 1340 - RNA, 111 nt, Artificial Sequence
  Description of Artificial Sequence Synthetic polynucleotide
gaugggcaccaccccggugaacagcuccucgcccuugcucacuggcagagcccuagagcaucgcgagcaggcgcugccuccuccgcccugcagcuuguacagcucguccau

SEQ ID NO: 1341 - RNA, 111 nt, Artificial Sequence
  Description of Artificial Sequence Synthetic polynucleotide
gaugggcaccaccccggugaacagcuccucgcccuugcucacuggcagagcccucgggcaucgcgagcaggcgcugccuccuccgcccugcagcuuguacagcucguccau

SEQ ID NO: 1342 - RNA, 111 nt, Artificial Sequence
  Description of Artificial Sequence Synthetic polynucleotide
gaugggcaccaccccggugaacagcuccucgcccuugcucacuggcagagcccuggggcaucgcgagcaggcgcugccuccuccgcccugcagcuuguacagcucguccau

SEQ ID NO: 1343 - RNA, 111 nt, Artificial Sequence
  Description of Artificial Sequence Synthetic polynucleotide
gaugggcaccaccccggugaacagcuccucgcccuugcucacuggcagagcccuugggcaucgcgagcaggcgcugccuccuccgcccugcagcuuguacagcucguccau

SEQ ID NO: 1344 - RNA, 111 nt, Artificial Sequence
  Description of Artificial Sequence Synthetic polynucleotide
gaugggcaccaccccggugaacagcuccucgcccuugcucacuggcagagcccuagggcaucgcgagcaggcgcugccuccuccgcccugcagcuuguacagcucguccau

SEQ ID NO: 1345 - RNA, 111 nt, Artificial Sequence
  Description of Artificial Sequence Synthetic polynucleotide
gaugggcaccaccccggugaacagcuccucgcccuugcucacuggcagagcccucgugcaucgcgagcaggcgcugccuccuccgcccugcagcuuguacagcucguccau

SEQ ID NO: 1346 - RNA, 111 nt, Artificial Sequence
  Description of Artificial Sequence Synthetic polynucleotide
gaugggcaccaccccggugaacagcuccucgcccuugcucacuggcagagcccuggugcaucgcgagcaggcgcugccuccuccgcccugcagcuuguacagcucguccau

SEQ ID NO: 1347 - RNA, 111 nt, Artificial Sequence
  Description of Artificial Sequence Synthetic polynucleotide
gaugggcaccaccccggugaacagcuccucgcccuugcucacuggcagagcccuagugcaucgcgagcaggcgcugccuccuccgcccugcagcuuguacagcucguccau

SEQ ID NO: 1348 - RNA, 111 nt, Artificial Sequence
  Description of Artificial Sequence Synthetic polynucleotide
gaugggcaccaccccggugaacagcuccucgcccuugcucacuggcagagcccuugugcaucgcgagcaggcgcugccuccuccgcccugcagcuuguacagcucguccau

SEQ ID NO: 1349 - RNA, 111 nt, Artificial Sequence
  Description of Artificial Sequence Synthetic polynucleotide
gaugggcaccaccccggugaacagcuccucgcccuugcucacuggcagagcccucgcgcaucgcgagcaggcgcugccuccuccgcccugcagcuuguacagcucguccau

SEQ ID NO: 1350 - RNA, 111 nt, Artificial Sequence
  Description of Artificial Sequence Synthetic polynucleotide
gaugggcaccaccccggugaacagcuccucgcccuugcucacuggcagagcccuggcgcaucgcgagcaggcgcugccuccuccgcccugcagcuuguacagcucguccau

SEQ ID NO: 1351 - RNA, 111 nt, Artificial Sequence
  Description of Artificial Sequence Synthetic polynucleotide
gaugggcaccaccccggugaacagcuccucgcccuugcucacuggcagagcccuugcgcaucgcgagcaggcgcugccuccuccgcccugcagcuuguacagcucguccau

SEQ ID NO: 1352 - RNA, 111 nt, Artificial Sequence
  Description of Artificial Sequence Synthetic polynucleotide
gaugggcaccaccccggugaacagcuccucgcccuugcucacuggcagagcccuagcgcaucgcgagcaggcgcugccuccuccgcccugcagcuuguacagcucguccau

SEQ ID NO: 1353 - RNA, 111 nt, Artificial Sequence; identical to SEQ ID NO: 1337
  Description of Artificial Sequence Synthetic polynucleotide

SEQ ID NO: 1354 - RNA, 111 nt, Artificial Sequence; identical to SEQ ID NO: 1338
  Description of Artificial Sequence Synthetic polynucleotide

SEQ ID NO: 1355 - RNA, 111 nt, Artificial Sequence; identical to SEQ ID NO: 1339
  Description of Artificial Sequence Synthetic polynucleotide

SEQ ID NO: 1356 - RNA, 111 nt, Artificial Sequence; identical to SEQ ID NO: 1340
  Description of Artificial Sequence Synthetic polynucleotide

SEQ ID NO: 1357 - RNA, 111 nt, Artificial Sequence; identical to SEQ ID NO: 1345
  Description of Artificial Sequence Synthetic polynucleotide

SEQ ID NO: 1358 - RNA, 111 nt, Artificial Sequence; identical to SEQ ID NO: 1341
  Description of Artificial Sequence Synthetic polynucleotide

SEQ ID NO: 1359 - RNA, 111 nt, Artificial Sequence; identical to SEQ ID NO: 1349
  Description of Artificial Sequence Synthetic polynucleotide

SEQ ID NO: 1360 - RNA, 111 nt, Artificial Sequence; identical to SEQ ID NO: 1345
  Description of Artificial Sequence Synthetic polynucleotide

SEQ ID NO: 1361 - RNA, 111 nt, Artificial Sequence; identical to SEQ ID NO: 1346
  Description of Artificial Sequence Synthetic polynucleotide

SEQ ID NO: 1362 - RNA, 111 nt, Artificial Sequence; identical to SEQ ID NO: 1348
  Description of Artificial Sequence Synthetic polynucleotide

SEQ ID NO: 1363 - RNA, 111 nt, Artificial Sequence; identical to SEQ ID NO: 1347
  Description of Artificial Sequence Synthetic polynucleotide

SEQ ID NO: 1364 - RNA, 111 nt, Artificial Sequence; identical to SEQ ID NO: 1337
  Description of Artificial Sequence Synthetic polynucleotide

SEQ ID NO: 1365 - RNA, 111 nt, Artificial Sequence; identical to SEQ ID NO: 1349
  Description of Artificial Sequence Synthetic polynucleotide

SEQ ID NO: 1366 - RNA, 111 nt, Artificial Sequence; identical to SEQ ID NO: 1341
  Description of Artificial Sequence Synthetic polynucleotide

SEQ ID NO: 1367 - RNA, 111 nt, Artificial Sequence
  Description of Artificial Sequence Synthetic polynucleotide
gauucugaauuagcuguaucgucaaggcacucgugccgacgccaccagcuccaaccaccacaaguggagagucagucauuuucagcaggccucucucccgcaccugggagc

SEQ ID NO: 1368 - RNA, 111 nt, Artificial Sequence
  Description of Artificial Sequence Synthetic polynucleotide
gauucugaauuagcuggaucgucaaggcacucgggccgacgccaccagcuccaaccaccacaaguggagagucagucauuuucagcaggccucucucccgcaccggggagc

SEQ ID NO: 1369 - RNA, 111 nt, Artificial Sequence
  Description of Artificial Sequence Synthetic polynucleotide
gggagcagccucuggcauucugggagcuucaucuggaccugggucuucagugaaccauuguucaauaucguccggggacagcaucaaaucauccauugcuugggacggcaa

SEQ ID NO: 1370 - RNA, 111 nt, Artificial Sequence
  Description of Artificial Sequence Synthetic polynucleotide
gggagcagccucuggcauucugggagcuucaucuggaccugggucuucagugaaccauuguucaagaucguccggggacagcaucaaaucauccauugcuugggacggcaa

SEQ ID NO: 1371 - RNA, 111 nt, Artificial Sequence
  Description of Artificial Sequence Synthetic polynucleotide
gggagcagccucuggcagucggggagcuucaucuggaccugggucuucagugaaccauuguucaagaucguccggggacagcaucaaaucauccagugcuugggacggcaa

SEQ ID NO: 1372 - RNA, 111 nt, Artificial Sequence
  Description of Artificial Sequence Synthetic polynucleotide
cauauuacagaauaccuugauagcauccaauuugcauccuugguuagggucaacccaguauucuccacucuugaguucaggauggcagaauuucaggucucugcaguuucu

SEQ ID NO: 1373 - RNA, 111 nt, Artificial Sequence
  Description of Artificial Sequence Synthetic polynucleotide
gugaagauaagccaguccucuaguaacagaaugagcaagacggcaagagcuuacccagucacuuguguggagacuuaaauacuugcauaaagauccauugggauaguacuc

SEQ ID NO: 1374 - RNA, 111 nt, Artificial Sequence
  Description of Artificial Sequence Synthetic polynucleotide
gugaacgucaaacugucggaccaauauggcagaaucuucucucaucucaacuuuccauauccguaucauggaaucauagcauccuguaacuacuagcucucuuacagcugg

SEQ ID NO: 1375 - RNA, 111 nt, Artificial Sequence
  Description of Artificial Sequence Synthetic polynucleotide
gccaaugaucucgugaguuaucucagcagugugagccaucagggugaugacaucccaggcgaucguguggccuccaggagcccagagcaggaaguugaggagaaggugccu

SEQ ID NO: 1376 - RNA, 111 nt, Artificial Sequence
  Description of Artificial Sequence Synthetic polynucleotide
caagacggugaaccacuccauggucuucuugucggcuuucugcacuguguacccccagagcuccguguugccgacauccugggguggcuuccacuccagagccacauuaag

SEQ ID NO: 1377 - RNA, 111 nt, Artificial Sequence
  Description of Artificial Sequence Synthetic polynucleotide
aggauucucuuuugaaguauugcucccccaguggauuggguggcuccauucacuccaaugcugagcacuuccacagaguggguuaaagcggcuccgaacacgaaacgugua

SEQ ID NO: 1378 - RNA, 111 nt, Artificial Sequence
  Description of Artificial Sequence Synthetic polynucleotide
gacgcccaccgugugguugcuguccaggacggucccggccugcgacacuucggcccagagcugcuccucauccagcagcgccagcagccccauggccgugagcaccggcuu

SEQ ID NO: 1379 - RNA, 111 nt, Artificial Sequence
  Description of Artificial Sequence Synthetic polynucleotide
gacgcccaccgugugguugcuguccaggacggucccggccugcgacacuucggcccagagcugcuccucaucugcggggcgggggggggccgucgccgcguggggucguug

SEQ ID NO: 1380 - DNA, 151 bp, Artificial Sequence
  Description of Artificial Sequence Synthetic polynucleotide
gggtgatgggtgctggccaggacacccactgtatgattgctgtccaacacagccccagcctttgagacctctgcccagagttgttctccatccaacagggccatgagccccatgactgtgagtactggctttcgcagcaactgcacatggg

SEQ ID NO: 1381 - DNA, 151 bp, Artificial Sequence
  Description of Artificial Sequence Synthetic polynucleotide
actacagttgctccgatatttaggctacgtcaataggcactaacttattggcgctggtgaacggacttcctctcgagtaccagaagatgactacaaaactcctttccattgcgagtatcggagtctggctcagtttggccagggaggcact

SEQ ID NO: 1382 - DNA, 99 bp, Artificial Sequence
  Description of Artificial Sequence Synthetic oligonucleotide
ctgcagggcggaggaggcagcggcggaggaggcagcggcggaggaggcagcagaaggtatacacgccggaagaatctgtagagatccccggtcgccacc

SEQ ID NO: 1383 - DNA, 81 bp, Artificial Sequence
  Description of Artificial Sequence Synthetic oligonucleotide
ctgcagggcggaggaggcagcggcggaggaggcagcggcggaggaggcagcgcctgctcgcgatgctagagggctctgcca

SEQ ID NO: 1384 - DNA, 51 bp, Artificial Sequence
  Description of Artificial Sequence Synthetic oligonucleotide
ctgcagggcggaggaggcagcgcctgctcgcgatgctagagggctctgcca

SEQ ID NO: 1385 - RNA, 70 nt, Artificial Sequence; identical to SEQ ID NO: 1229
  Description of Artificial Sequence Synthetic oligonucleotide

SEQ ID NO: 1386 - DNA, 111 bp, Artificial Sequence
  Description of Artificial Sequence Synthetic polynucleotide
atggacgagctgtacaagctgcagggcggaggaggcagcgcctgctcgcgatgctatagggctctgccagtgagcaagggcgaggagctgttcaccggggtggtgcccatc

SEQ ID NO: 1387 - RNA, 91 nt, Artificial Sequence
  Description of Artificial Sequence Synthetic oligonucleotide
uagcuguaucgucaaggcacucuugccuacgccaccagcuccaaccaccacaaguuuauauucagucauuuucagcaggccucucucccgc

SEQ ID NO: 1388 - RNA, 111 nt, Artificial Sequence
  Description of Artificial Sequence Synthetic polynucleotide
gauucugaauuagcuguaucgucaaggcacucuugccuacgccaccagcuccaacuaccacaaguuuauauucagucauuuucagcaggccucucucccgcaccugggagc

SEQ ID NO: 1389 - RNA, 131 nt, Artificial Sequence
  Description of Artificial Sequence Synthetic polynucleotide
uccacaaaaugauucugaauuagcuguaucgucaaggcacucuugccuacgccaccagcuccaacuaccacaaguuuauauucagucauuuucagcaggccucucucccgcaccugggagccgcugagccu

SEQ ID NO: 1390 - RNA, 151 nt, Artificial Sequence; identical to SEQ ID NO: 1322
  Description of Artificial Sequence Synthetic polynucleotide

SEQ ID NO: 1391 - RNA, 171 nt, Artificial Sequence
  Description of Artificial Sequence Synthetic polynucleotide
cuauuguuggaucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgccgccgccuuc

SEQ ID NO: 1392 - RNA, 191 nt, Artificial Sequence
  Description of Artificial Sequence Synthetic polynucleotide
uaggaauccucuauuguuggaucauauucguccacaaaaugauucugaauuagcuguaucgucaaggcacucuugccuacgccaccagcuccaaccaccacaaguuuauauucagucauuuucagcaggccucucucccgcaccugggagccgcugagccucuggccccgccgccgccuucagugccugcg

SEQ ID NO: 1393 - RNA, 151 nt, Artificial Sequence; identical to SEQ ID NO: 1319
  Description of Artificial Sequence Synthetic polynucleotide

SEQ ID NO: 1394 - RNA, 151 nt, Artificial Sequence
  Description of Artificial Sequence Synthetic polynucleotide
uccuguagcuaaggccacaaaauuauccacuguuuuuggaacagucuuuccgaagagaccaaagaucacccggcccacaucuucaucuccaauucguaggucaaaauacaccuugacggugacuuugggccccuucuucuucucaucggcc

SEQ ID NO: 1395 - RNA, 151 nt, Artificial Sequence
  Description of Artificial Sequence Synthetic polynucleotide
gcccuggaucaugaaguccuugauuacacgauggaauuugcuguuuuuguagccaaauccuuucucuccuguagccaaggccacaaaauuauccacuguuuuuggaacagucuuuccgaagagaccaaagaucacccggccuacaucuuca

SEQ ID NO: 1396 - RNA, 72 nt, Artificial Sequence
  Description of Artificial Sequence Synthetic oligonucleotide
gcgcaaguuagguuuugucaagaaaggguguaacgcaaccaagucauaguccgccuagaagcauuugcggug

SEQ ID NO: 1397 - RNA, 131 nt, Artificial Sequence
  Description of Artificial Sequence Synthetic polynucleotide
gccaugccaaucucaucuuguuuucugcgcaaguuagguuuugucaagaaaggguguaacgcaaccaagucauaguccgccuagaagcauuugcgguggacgauggaggggccggacucgucauacuccug

SEQ ID NO: 1398 - RNA, 70 nt, Artificial Sequence
  Description of Artificial Sequence Synthetic oligonucleotide
ggacuuccuguaacaacgcaucucauauuuggaaugaccauuaaaaaaacaacaaugugcaaucaaaguc

SEQ ID NO: 1399 - RNA, 71 nt, Artificial Sequence
  Description of Artificial Sequence Synthetic oligonucleotide
caaggugcggcuccggccccuccccucuucaagggguccacauggcaacugugaggaggggagauucagug

SEQ ID NO: 1400 - RNA, 91 nt, Artificial Sequence
  Description of Artificial Sequence Synthetic oligonucleotide
uagcuguaucgucaaggcacucgugccgacgccaccagcuccaaccaccacaaggggagagucagucagggucagcaggccucucucccgc

SEQ ID NO: 1401 - RNA, 91 nt, Artificial Sequence
  Description of Artificial Sequence Synthetic oligonucleotide
uagcuguaucgucaaggcacucuugccgacgccaccagcuccaaccaccacaaguguauagucagucauuuucagcaggccucucucccgc

SEQ ID NO: 1402 - RNA, 91 nt, Artificial Sequence
  Description of Artificial Sequence Synthetic oligonucleotide
uagcuggaucgucaaggcacucgugccgacgccaccagcuccaaccaccacaaggggagaggcagucagggucagcaggccucucucccgc

SEQ ID NO: 1403 - RNA, 111 nt, Artificial Sequence
  Description of Artificial Sequence Synthetic polynucleotide
gauucugaauuagcuguaucgucaaggcacucuugccgacgccaccagcuccaaccaccacaaguguauagucagucauuuucagcaggccucucucccgcaccugggagc

SEQ ID NO: 1404 - RNA, 111 nt, Artificial Sequence; identical to SEQ ID NO: 1367
  Description of Artificial Sequence Synthetic polynucleotide

SEQ ID NO: 1405 - RNA, 111 nt, Artificial Sequence
  Description of Artificial Sequence Synthetic polynucleotide
gauucugaauuagcuguaucgucaaggcacucgugccgacgccaccagcuccaaccaccacaaggggagagucagucagggucagcaggccucucucccgcaccugggagc

SEQ ID NO: 1406 - RNA, 111 nt, Artificial Sequence
  Description of Artificial Sequence Synthetic polynucleotide
gcuccccggugcgggagagaggccugcugacccugacugccucuccccuuguggugguuggagcugguggcgucggcacgagugccuugacgauccagcuaauucagaauc

SEQ ID NO: 1407 - RNA, 14 nt, Artificial Sequence
  Description of Artificial Sequence Synthetic oligonucleotide
gcagagccuccagc

SEQ ID NO: 1408 - RNA, 21 nt, Artificial Sequence
  Description of Artificial Sequence Synthetic oligonucleotide
cucacuggcagagccuccagc

SEQ ID NO: 1409 - RNA, 27 nt, Artificial Sequence
  Description of Artificial Sequence Synthetic oligonucleotide
cccuugcucacuggcagagccuccagc

SEQ ID NO: 1410 - RNA, 33 nt, Artificial Sequence
  Description of Artificial Sequence Synthetic oligonucleotide
cucucgcccuugcucacuggcagagccuccagc

SEQ ID NO: 1411 - RNA, 38 nt, Artificial Sequence
  Description of Artificial Sequence Synthetic oligonucleotide
cucucgcccuugcucacuggcagagccuccagcaucgc

SEQ ID NO: 1412 - RNA, 45 nt, Artificial Sequence
  Description of Artificial Sequence Synthetic oligonucleotide
ugaacagcucucgcccuugcucacuggcagagccuccagcaucgc

SEQ ID NO: 1413 - RNA, 70 nt, Artificial Sequence; identical to SEQ ID NO: 1230
  Description of Artificial Sequence Synthetic oligonucleotide

SEQ ID NO: 1414 - RNA, 30 nt, Artificial Sequence
  Description of Artificial Sequence Synthetic oligonucleotide
acaaauggggacgaggggggcggggcggcc

SEQ ID NO: 1415 - RNA, 20 nt, Artificial Sequence
  Description of Artificial Sequence Synthetic oligonucleotide
cggagagcagagagggagcg

SEQ ID NO: 1416 - DNA, 49 bp, Artificial Sequence
  Description of Artificial Sequence Synthetic oligonucleotide
aaaaaaaaaagatcttgaaactgttttaaggttggccgatcttaaaaaa

SEQ ID NO: 1417 - RNA, 51 nt, Artificial Sequence
  Description of Artificial Sequence Synthetic oligonucleotide
ucauaaucaauuuauuauuuucuuuuauuuuauucacauaauuuuguuuuu

SEQ ID NO: 1418 - DNA, 1962 bp, Homo sapiens
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 1419 - DNA, 70 bp, Artificial Sequence
  GluR2.20.6
  misc_feature: n is any nucleotide base
  misc_feature (46)..(51): n is a, c, g, or t
  misc_feature (53)..(65): n is a, c, g, or t
gtggaatagtataacaatatgctaaatgttgttatagtatcccacnnnnnncnnnnnnnnnnnnnttttt

SEQ ID NO: 1420 - DNA, 195 bp, Artificial Sequence
  GluR2.100.50
  misc_feature: n is any nucleotide
  misc_feature (46)..(95): n is a, c, g, or t
  misc_feature (97)..(145): n is a, c, g, or t
gtggaatagtataacaatatgctaaatgttgttatagtatcccacnnnnnnnnnnnnnnnnnnnnnnnnnnnnnnnnnnnnnnnnnnnnnnnnnncnnnnnnnnnnnnnnnnnnnnnnnnnnnnnnnnnnnnnnnnnnnnnnnnngtggaatagtataacaatatgctaaatgttgttatagtatcccacttttt

SEQ ID NO: 1421 - DNA, 368 bp, Artificial Sequence
  Alu.100.50
  misc_feature: n is any nucleotide
  misc_feature (117)..(166): n is a, c, g, or t
  misc_feature (168)..(216): n is a, c, g, or t
ggccgggcgcggtggctcacgcctgtaatcccagcactttgggaggccgaggcggggagattgcttgagcccaggagttcgagaccagcctgggcaacatagcgagaccccgtctcnnnnnnnnnnnnnnnnnnnnnnnnnnnnnnnnnnnnnnnnnnnnnnnnnncnnnnnnnnnnnnnnnnnnnnnnnnnnnnnnnnnnnnnnnnnnnnnnnnnagccgggcgtggtggcgcgcgcctgtagtcccagctactcgggaggctgaggcaggaggatcgcttgagcccaggagttcgaggctgcagtgagctatgatcgcgccactgcactccagcctgggcgacagagcgagaccctgtctcttttt

SEQ ID NO: 1422 - DNA, 160 bp, Artificial Sequence
  U6+27.100.50
  misc_feature: n is any nucleotide
  misc_feature (34)..(83): n is a, c, g, or t
  misc_feature (85)..(133): n is a, c, g, or t
gtgctcgcttcggcagcacatatactagtcgacnnnnnnnnnnnnnnnnnnnnnnnnnnnnnnnnnnnnnnnnnnnnnnnnnncnnnnnnnnnnnnnnnnnnnnnnnnnnnnnnnnnnnnnnnnnnnnnnnnntctagagcggacttcggtccgcttttt

SEQ ID NO: 1423 - DNA, 283 bp, Artificial Sequence
  Circular.100.50
  misc_feature: n is any nucleotide
  misc_feature (88)..(137): n is a, c, g, or t
  misc_feature (139)..(187): n is a, c, g, or t
gccatcagtcgccggtcccaagcccggataaaatgggagggggcgggaaaccgcctaaccatgccgactgatggcagaaaaaaaaaannnnnnnnnnnnnnnnnnnnnnnnnnnnnnnnnnnnnnnnnnnnnnnnnncnnnnnnnnnnnnnnnnnnnnnnnnnnnnnnnnnnnnnnnnnnnnnnnnnaaaaaaaaaactgccatcagtcggcgtggactgtagaacactgccaatgccggtcccaagcccggataaaagtggagggtacagtccacgcttttt

SEQ ID NO: 1424 - DNA, 283 bp, Artificial Sequence
  Ribozyme.mutant.100.50
  misc_feature: n is any nucleotide
  misc_feature (88)..(137): n is a, c, g, or t
  misc_feature (139)..(187): n is a, c, g, or t
gccatcagtcgccgtgcccaagcccggataaaatgggagggggcgggaaaccgcctaaccatgccgactgatggcagaaaaaaaaaannnnnnnnnnnnnnnnnnnnnnnnnnnnnnnnnnnnnnnnnnnnnnnnnncnnnnnnnnnnnnnnnnnnnnnnnnnnnnnnnnnnnnnnnnnnnnnnnnnaaaaaaaaaactgccatcagtcggcgtggactgtagaacactgccaatgccggtcccgagctcggataaaagtggagggtacagtccacgcttttt

SEQ ID NO: 1425 - DNA, 20 bp, Artificial Sequence
  RAB7A.20.6
tgccgccagctggatttccc

SEQ ID NO: 1426 - DNA, 100 bp, Artificial Sequence
  RAB7A.100.50
tgataaaaggcgtacataattcttgtgtctactgtacagaatactgccgccagctggatttcccaattctgagtaacactctgcaatccaaacagggttc

SEQ ID NO: 1427 - DNA, 200 bp, Artificial Sequence
  RAB7A.200.100
agacagttgtccccctggagagatgaaaagcttgttggctcttaagtctttgataaaaggcgtacataattcttgtgtctactgtacagaatactgccgccagctggatttcccaattctgagtaacactctgcaatccaaacagggttcaaccctccaccttacaggcctgcattacaggacttaaacacataatccaa

SEQ ID NO: 1428 - DNA, 200 bp, Artificial Sequence
  RAB7A.200.100.loops
agacagttgtccccctggagagatgaaaagcttgttggctcttaagtctttgataaaaggcgtacacttgtcttgtgtctactgtacagaatactgccgccagctgctaatcccaattctgagtaacactctgcaatccaaacagggttcaaccctccaccttacaggcctgcattacaggacttaaacacataatccaa

SEQ ID NO: 1429 - DNA, 200 bp, Artificial Sequence
  RAB7A.200.100.loops.interspersed.v1
agacagttgtccccctggagagatgaaatcgatgttggctcttaagtgaaagataaaaggcgtacacttgtcttgtgtctactgtacagaatactgccgccagctgctaatcccaattctgagtatgtgtctgcaatccaaacacccatcaaccctccacctttgtcgcctgcattacaggacttaaacacataatccaa

SEQ ID NO: 1430 - DNA, 200 bp, Artificial Sequence
  RAB7A.200.100.loops.interspersed.v2
agacagttgtccccctggagagatgaaatcgatgttggctcttaatggaaagataaaaggcgtacacttgtcttgtgtctactgtacagaagactgccgccagctgctaatcccaattctgagtatgtgtctgcaatccaaacacccatcaaccctccacctttgtcgcctgcattacaggagaataacacataatccaa

SEQ ID NO: 1431 - DNA, 200 bp, Artificial Sequence
  RAB7A.200.100.loops.interspersed.v3
agacagttgtccccctggagagatgaaatcgatgttggctcttaatggaaagataaaaggcgtacattcaagatgtgtctactgtacagaatactgccgccagctgctaatcccaattctgagtatgtgtctgcaatccaaacacccatcaaccctccacctttgtcgcctgcattacaggagaataacacataatccaa

SEQ ID NO: 1432 - DNA, 100 bp, Artificial Sequence
  Cypridina Luciferase.100.50
atgtaccaggttcctggaactggaatctctttccatagaatgttctaaaccatcctgcggcctctactctgcattcaattacatactgacacattcggca

SEQ ID NO: 1433 - DNA, 200 bp, Artificial Sequence
  Cypridina Luciferase.200.100
ggtgatggacaccttccagtcgccgcccttggttccttgacccaacacgtatgtaccaggttcctggaactggaatctctttccatagaatgttctaaaccatcctgcggcctctactctgcattcaattacatactgacacattcggcaacatgttgttcctggtttattgtcacacagtccatctgatagtatgtctc

SEQ ID NO: 1434 - DNA, 200 bp, Artificial Sequence
  GAPDH.200.100
ggccatccacagtcttctgggtggcagtgatggcatggactgtggtcatgagtccttccacgataccaaagttgtcatggatgaccttggccaggggtgccaagcagttggtggtgcaggaggcattgctgatgatcttgaggctgttgtcatacttctcatggttcacacccatgacgaacatgggggcatcagcagag

SEQ ID NO: 1435 - DNA, 200 bp, Artificial Sequence
  GAPDH.200.100.bulges
ggccatccacagtcttctgggtggcagtgatggcatggactgtggtcatgagtccttccacgagaccaaagtggtcatggatgaccttggccaggggtgccaagcagtgggtggggcaggaggcatggcggaggatcgggaggctgttgtcatacttctcatggttcacacccatgacgaacatgggggcatcagcagag

SEQ ID NO: 1436 - DNA, 200 bp, Artificial Sequence
  GAPDH.200.100.loops
ggccatccacagtcttctgggtggcagtgatggcatggactgtggtcatgagtccttccacgatacgttcgttgtcatggatgaccttggccaggggtgccaagcacaacgtggtgcaggaggcattgctgatgatcttgaggctgttgtcatacttctcatggttcacacccatgacgaacatgggggcatcagcagag

SEQ ID NO: 1437 - DNA, 200 bp, Artificial Sequence
  GAPDH.200.100.loops.interspersed
ggccatccacagtcttctgggtggcagtctacgcatggactgtggtctactgtccttccacgatacgttcgttgtcatggatgaccttggccaggggtgccaagcacaacgtggtgcaggaggcaaacgtgatgatcttgaggcacaagtcatacttctcatgcaagacacccatgacgaacatgggggcatcagcagag

SEQ ID NO: 1438 - DNA, 201 bp, Artificial Sequence
  ALDOA.200.100
gtccgtccttcttgtactgggcacagcgctcagacagcccatccaacccttgggtggtagtctcgccatttgtccctgccagggggaccacgcccttgtccaccttgatgcccacaacaccgcccttggatttgataacttgggggaagggacgcccatcatccgccttctggtagagtgtctcatggaagaggatgacac

SEQ ID NO: 1439 - DNA, 200 bp, Artificial Sequence
  DAXX.200.100
gctcctgtaacctgatgcccacatctcggaaggcatcctgagccatgagctggagctgctgtcgggggaggccaaggctgtgtcgggcagctgccttctccacagcccgaagcacatccccatagtcagggaaggtatcaggccctggcttgttgatgagccgctcaatgcgcctgttaacctctgggtagcgggtgcca

SEQ ID NO: 1440 - DNA, 200 bp, Artificial Sequence
  TARDBP.200.100.loops.interspersed
tgtgtttcatattccgtaaaacgaacaatcggaaacccctttgaatgtggtgtcttaagatctttcaactcctgcaccataagaacttctccaaaggtaccaaaattgagtttcaggtcctgttcccaagtgttccatgggagagggtacactattaaatcggtacagttctggactgctcttgtcactttcactgctga

SEQ ID NO: 1441 - DNA, 200 bp, Artificial Sequence
  SMAD4.200.100.loops.interspersed
ttgtagtccaccatcctgataaggttaacccgcccaacggtaaaagaggagagtctaaaggttgtgccagtgcaatcggcatggtatgaagtacttcgtccaggaggaccagggcccggtgtaagacggtttcaatccagcaagcacattctttgatgctctgagaagggtaatccggtccccagcctttcacaaaactc

SEQ ID NO: 1442 - DNA, 200 bp, Artificial Sequence
  FANCC.200.100.loops.interspersed
gagtctgggctgagggacctggctctgctaaatgtaaaatagatacttcgtgattgtcccaagatgacatcagctcattctcacagcccagcgagggcacctactcgtgtaatgcgtggccacagcagttcacctgtcctgtgggggaggcgagcctgatcccagtggccgggcacccacacggcctgcgtgccttctag

SEQ ID NO: 1443 - DNA, 200 bp, Artificial Sequence
  CTNNB1.200.100.loops.interspersed
aaaagtttacttcaattgttcccataggtttgtcagcttggttagtgacagaggcactttctgagaatggagcccacccctcgagccctctcagcaactccacaggggtttcacaatgcaagttctctgaatacagctgtataatctcagaaaggctgctattatacattaaaacagcaaatgttactcatttgagtatt

SEQ ID NO: 1444 - DNA, 200 bp, Artificial Sequence
  mPCSK9.200.100
atcttcctgaaagacaaatacaccctaaggcctgagaactgaggccttgcagcgagcatcctcaggttctggtacactggagcagctggccagagccagccacagcttaatgtctgatggcagaaagccaagcacaggccccctgaccgtgaaggaggtgcttccatacaccaggaatgggtacccagggtgagacacca

SEQ ID NO: 1445 - DNA, 200 bp, Artificial Sequence
  mIDUA.200.100
actccaggccgctgcggagccttcagggtgatgggtgctggccaggacacccactgtatgattgctgtccaacacagccccagcctttgagacctctgcccagagttgttctccatccaacagggccatgagccccatgactgtgagtactggctttcgcagcaactgcacgtggggtgggtgagtattgttgacctgga

SEQ ID NO: 1446 - DNA, 200 bp, Artificial Sequence
  mIDUA.scrambled.200.100
tgctggttctggccacatcccatccgtgtgagagcacggtatcacgaggcagtggcaagtgaatagccggcctggcatcagcccttggatttgtcaattcccagggcaccattgttgcctactgcgccgaaggtggcagagttatcctgggtacgcacccggactggtgctcggctttacagagccctcggtacccatag

SEQ ID NO: 1447 - DNA, 21 bp, Artificial Sequence
  RAB7A_seq_F primer
cctccctccttgaaggctacc

SEQ ID NO: 1448 - DNA, 24 bp, Artificial Sequence
  RAB7A_seq_R primer
aagctccgctaacctaagaatacc

SEQ ID NO: 1449 - DNA, 20 bp, Artificial Sequence
  GAPDH_seq_F primer
gatgctggcgctgagtacgt

SEQ ID NO: 1450 - DNA, 21 bp, Artificial Sequence
  GAPDH_seq_R primer
caccactgacacgttggcagt

SEQ ID NO: 1451 - DNA, 25 bp, Artificial Sequence
  DAXX_seq_F primer
ctgaccggccgtgtcatagagcagc

SEQ ID NO: 1452 - DNA, 28 bp, Artificial Sequence
  DAXX_seq_R primer
gtgaggtggcagccaaagttgtagatga

SEQ ID NO: 1453 - DNA, 23 bp, Artificial Sequence
  ALDOA_seq_F
gacagctgacgaccgcgtgaacc

SEQ ID NO: 1454 - DNA, 25 bp, Artificial Sequence
  ALDOA_seq_R primer
ccccaatcttcagcacacaacgcca

SEQ ID NO: 1455 - DNA, 19 bp, Artificial Sequence
  CTNNB_Seq_F primer
agggtgggagtggtttagg

SEQ ID NO: 1456 - DNA, 24 bp, Artificial Sequence
  CTNNB_Seq_R primer
ccatcttgtgatccattcttgtgc

SEQ ID NO: 1457 - DNA, 21 bp, Artificial Sequence
  TARDBP_seq_F
ccatcggaagacgatgggacg

SEQ ID NO: 1458 - DNA, 22 bp, Artificial Sequence
  TARDBP_seq_R
cctgaatggcttggggatgaag

SEQ ID NO: 1459 - DNA, 20 bp, Artificial Sequence
  SMAD4_seq_F primer
gcgtgcacctggagatgctg

SEQ ID NO: 1460 - DNA, 34 bp, Artificial Sequence
  SMAD4_seq_R primer
acaggtgaagaattaataagaatgtgtttctcct

SEQ ID NO: 1461 - DNA, 22 bp, Artificial Sequence
  FANCC_seq_F primer
cctgcacaacagctgatcaggc

SEQ ID NO: 1462 - DNA, 30 bp, Artificial Sequence
  FANCC_seq_R primer
ttctttaatggttcatgaccaaattcttgg

SEQ ID NO: 1463 - DNA, 19 bp, Artificial Sequence
  mPCSK9_seq_F primer
ggggagattctgcgtggga

SEQ ID NO: 1464 - DNA, 22 bp, Artificial Sequence
  mPCSK9_seq_R primer
gcccagcctggcattattcagg

SEQ ID NO: 1465 - DNA, 20 bp, Artificial Sequence
  mIDUA_seq_F primer
gagttcaaggatacccctat

SEQ ID NO: 1466 - DNA, 20 bp, Artificial Sequence
  mIDUA_seq_R primer
agtgatggcaccggaagctt

SEQ ID NO: 1467 - DNA, 24 bp, Artificial Sequence
  RAB7A_qPCR_F primer
gtggtattcttaggttagcggagc

SEQ ID NO: 1468 - DNA, 21 bp, Artificial Sequence
  RAB7A_qPCR_R primer
ttgcctgtcaaggaaagtaca

SEQ ID NO: 1469 - DNA, 21 bp, Artificial Sequence
  ALDOA_qPCR_F primer
ttgcctgtcaaggaaagtaca

SEQ ID NO: 1470 - DNA, 21 bp, Artificial Sequence
  ALDOA_qPCR_R primer
gggaacacctccgcttaatag

SEQ ID NO: 1471 - DNA, 22 bp, Artificial Sequence
  DAXX_qPCR_F primer
caggcatggtctcttctacttc

SEQ ID NO: 1472 - DNA, 20 bp, Artificial Sequence
  DAXX_qPCR_R primer
ccctgatcctgtttgcttct

SEQ ID NO: 1473 - DNA, 23 bp, Artificial Sequence
  GAPDH_qPCR_F primer
gtcaaggctgagaacgggaagct

SEQ ID NO: 1474 - DNA, 22 bp, Artificial Sequence
  GAPDH_qPCR_R primer
gccttctccatggtggtgaaga

SEQ ID NO: 1475 - DNA, 21 bp, Artificial Sequence
  B-ACTIN_qPCR_F primer
catgtacgttgctatccaggc

SEQ ID NO: 1476 - DNA, 21 bp, Artificial Sequence
  B-ACTIN_qPCR_R primer
ctccttaatgtcacgcacgat

SEQ ID NO: 1477 - DNA, 24 bp, Artificial Sequence
  SMAD4_qPCR_F primer
tgagagagcaaggttgcacatagg

SEQ ID NO: 1478 - DNA, 22 bp, Artificial Sequence
  SMAD4_qPCR_R primer
acactgacgcaaatcaaagacc

SEQ ID NO: 1479 - DNA, 23 bp, Artificial Sequence
  CTNNB1_qPCR_F primer
cattccattgtttgtgcagctgc

SEQ ID NO: 1480 - DNA, 23 bp, Artificial Sequence
  CTNNB1_qPCR_R primer
tatgtcgccacaccttcattcct

SEQ ID NO: 1481 - DNA, 23 bp, Artificial Sequence
  FANCC_qPCR_F primer
gctgcaagaccctcaagatatcc

SEQ ID NO: 1482 - DNA, 22 bp, Artificial Sequence
  FANCC_qPCR_R primer
tcggctgccgacatcagtaatt

SEQ ID NO: 1483 - DNA, 22 bp, Artificial Sequence
  TARDBP_qPCR_F primer
cgggtaaccgaagatgagaacg

SEQ ID NO: 1484 - DNA, 24 bp, Artificial Sequence
  TARDBP_qPCR_R primer
catgcagaattccttctaccagcc

SEQ ID NO: 1485 - DNA, 26 bp, Artificial Sequence
  mPCSK9_qPCR_F primer
gactctcagtttgtactggagaacca

SEQ ID NO: 1486 - DNA, 22 bp, Artificial Sequence
  mPCSK9_qPCR_R primer
gcccagcctggcattattcagg

SEQ ID NO: 1487 - DNA, 23 bp, Artificial Sequence
  mIDUA_qPCR_F primer
gactggaaagatggctcagtaga

SEQ ID NO: 1488 - DNA, 23 bp, Artificial Sequence
  mIDUA_qPCR_R primer
ccaccaacactaggacaataggc

SEQ ID NO: 1489 - DNA, 21 bp, Artificial Sequence
  mGAPDH_qPCR_F primer
aggtcggtgtgaacggatttg

SEQ ID NO: 1490 - DNA, 23 bp, Artificial Sequence
  mGAPDH_qPCR_R primer
tgtagaccatgtagttgaggtca

SEQ ID NO: 1491 - DNA, 24 bp, Artificial Sequence
  IDUA_circ_check_F primer
ggctgtgttggacagcaatcatac

SEQ ID NO: 1492 - DNA, 21 bp, Artificial Sequence
  IDUA_circ_check_R primer
ttctccatccaacagggccat

SEQ ID NO: 1493 - DNA, 17 bp, Artificial Sequence
  PCSK9_circ_check_F primer
ctctggccagctgctcc

SEQ ID NO: 1494 - DNA, 24 bp, Artificial Sequence
  PCSK9_circ_check_R primer
cttaatgtctgatggcagaaagcc

SEQ ID NO: 1495 - DNA, 32 bp, Artificial Sequence
  RAB7A_circ_check_F primer
cagtattctgtacagtagacacaagaattatg

SEQ ID NO: 1496 - DNA, 28 bp, Artificial Sequence
  RAB7A_circ_check_R primer
gatttcccaattctgagtaacactctgc

SEQ ID NO: 1497 - DNA, 28 bp, Artificial Sequence
  RAB7A_linear_check_F primer
tgataaaaggcgtacataattcttgtgt

SEQ ID NO: 1498 - DNA, 24 bp, Artificial Sequence
  RAB7A_linear_check_R primer
gaaccctgtttggattgcagagtg

SEQ ID NO: 1499 - DNA, 20 bp, Artificial Sequence
  Ligation_stem_F primer
catgccgactgatggcagaa

SEQ ID NO: 1500 - DNA, 17 bp, Artificial Sequence
  Ligation_stem_R primer
cgccgactgatggcagt

SEQ ID NO: 1501 - DNA, 16 bp, Artificial Sequence
  Ribozyme_check_F primer
ggcgggaaaccgccta

SEQ ID NO: 1502 - DNA, 21 bp, Artificial Sequence
  Ribozyme_check_R primer
ggcattggcagtgttctacag

SEQ ID NO: 1503 - DNA, 20 bp, Artificial Sequence
  mRIGI_qPCR_F primer
gaccccacctacatcctcag

SEQ ID NO: 1504 - DNA, 20 bp, Artificial Sequence
  mRIGI_qPCR_R primer
gagtgaggcagcttccattg

SEQ ID NO: 1505 - DNA, 21 bp, Artificial Sequence
  mMDA5_qPCR_F primer
agacacaagtttggcagaagg

SEQ ID NO: 1506 - DNA, 20 bp, Artificial Sequence
  mMDA5_qPCR_R primer
cttcccatggtgcctgaatc

SEQ ID NO: 1507 - DNA, 20 bp, Artificial Sequence
  mOAS1a_qPCR_F primer
attacctccttcccgacacc

SEQ ID NO: 1508 - DNA, 20 bp, Artificial Sequence
  mOAS1a_qPCR_R primer
caaactccacctcctgatgc

SEQ ID NO: 1509 - DNA, 20 bp, Artificial Sequence
  mOASL2_qPCR_F primer
gggaggtcgtcatcagcttc

SEQ ID NO: 1510 - DNA, 20 bp, Artificial Sequence
  mOASL2_qPCR_R primer
cccttttgccctctctgtgg

SEQ ID NO: 1511 - DNA, 23 bp, Artificial Sequence
  mPKR_qPCR_F primer
gatggaaaatcccgaacaaggag

SEQ ID NO: 1512 - DNA, 23 bp, Artificial Sequence
  mPKR_qPCR_R primer
aggcccaaagcaaagatgtccac

SEQ ID NO: 1513 - DNA, 27 bp, Artificial Sequence
  mADAR1_150_qPCR_F primer
ttcaaggaaacgaaagtgaactctggg

SEQ ID NO: 1514 - DNA, 20 bp, Artificial Sequence
  mADAR1_150_qPCR_R primer
tgtgggtcccctgaaccctt

SEQ ID NO: 1515 - DNA, 26 bp, Artificial Sequence
  mADAR1_110_qPCR_F
ccattgattcctgactgaaggtggaa

SEQ ID NO: 1516 - DNA, 19 bp, Artificial Sequence
  mADAR1_110_qPCR_F
cgattcctcggacgctgcc

SEQ ID NO: 1517 - DNA, 21 bp, Artificial Sequence
  mADAR2_qPCR_F primer
gtttcgacagggacgaagtgt

SEQ ID NO: 1518 - DNA, 20 bp, Artificial Sequence
  mADAR2_qPCR_R primer
tggcgtcataccctctagca

SEQ ID NO: 1519 - DNA, 42 bp, Artificial Sequence
  T7_circ_F primer
atgctaatacgactcactatagggccatcagtcgccggtccc

SEQ ID NO: 1520 - DNA, 24 bp, Artificial Sequence
  T7_circ_R
aaaaaagcgtggactgtaccctcc

SEQ ID NO: 1521 - DNA, 57 bp, Artificial Sequence
  T7_circ_RAB7A_sense_F primer
atgctaatacgactcactatagggtttggattatgtgtttaagtcctgtaatgcagg

SEQ ID NO: 1522 - DNA, 49 bp, Artificial Sequence
  circ_RAB7A_sense_R primer
ccatgccgactgatggcagaaaaaaaaaagacagttgtccccctggaga

SEQ ID NO: 1523 - DNA, 47 bp, Artificial Sequence
  T7_circ_GAPDH_sense_F primer
atgctaatacgactcactatagggtttctctgctgatgcccccatgt

SEQ ID NO: 1524 - DNA, 50 bp, Artificial Sequence
  circ_GAPDH_sense_R primer
ccatgccgactgatggcagaaaaaaaaaaggccatccacagtcttctggg

SEQ ID NO: 1525 - DNA, 45 bp, Artificial Sequence
  T7_circ_DAXX_sense_F primer
atgctaatacgactcactatagggttttggcacccgctacccaga

SEQ ID NO: 1526 - DNA, 51 bp, Artificial Sequence
  circ_DAXX_sense_R
ccatgccgactgatggcagaaaaaaaaaagctcctgtaacctgatgcccac

SEQ ID NO: 1527 - DNA, 53 bp, Artificial Sequence
  T7_circ_ALDOA_sense_F primer
atgctaatacgactcactatagggtttgtgtcatcctcttccatgagacactc

SEQ ID NO: 1528 - DNA, 52 bp, Artificial Sequence
  circ_ALDOA_sense_R primer
ccatgccgactgatggcagaaaaaaaaaatccgtccttcttgtactgggcac

SEQ ID NO: 1529 - DNA, 51 bp, Artificial Sequence
  T7_Circ_CTNNB1_Sense_F primer
atgctaatacgactcactatagggtttaatactcaaatgagtaacatttgc

SEQ ID NO: 1530 - DNA, 54 bp, Artificial Sequence
  circ_CTNNB1_Sense_R primer
ccatgccgactgatggcagaaaaaaaaaaagtttacttcaattgttcccatagg

SEQ ID NO: 1531 - DNA, 49 bp, Artificial Sequence
  T7_Circ_SMAD4_Sense_F primer
atgctaatacgactcactatagggttgagttatgtgaaaggctggggac

SEQ ID NO: 1532 - DNA, 53 bp, Artificial Sequence
  circ_SMAD4_Sense_R primer
ccatgccgactgatggcagaaaaaaaaaattgtagtccaccatcctgataagg

SEQ ID NO: 1533 - DNA, 45 bp, Artificial Sequence
  T7_Circ_FANCC_Sense_F primer
atgctaatacgactcactatagggttgtctagaaggcacgcaggc

SEQ ID NO: 1534 - DNA, 49 bp, Artificial Sequence
  circ_FANCC_Sense_R primer
ccatgccgactgatggcagaaaaaaaaaagagtctgggctgagggacct

SEQ ID NO: 1535 - DNA, 49 bp, Artificial Sequence
  T7_Circ_TARDBP_Sense_F primer
atgctaatacgactcactatagggtttcagcagtgaaagtgaaaagagc

SEQ ID NO: 1536 - DNA, 55 bp, Artificial Sequence
  circ_TARDBP_Sense_R primer
ccatgccgactgatggcagaaaaaaaaaatgtgtttcatattccgtaaaacgaac

SEQ ID NO: 1537 - DNA, 47 bp, Artificial Sequence
  T7_circ_mPCSK9_sense_F primer
atgctaatacgactcactatagggtttggtgtctcaccctgggtacc

SEQ ID NO: 1538 - DNA, 58 bp, Artificial Sequence
  circ_mPCSK9_sense_R primer
ccatgccgactgatggcagaaaaaaaaaacttcctgaaagacaaatacaccctaaggc

SEQ ID NO: 1539 - DNA, 51 bp, Artificial Sequence
  T7_circ_mIDUA_sense_F primer
atgctaatacgactcactatagggttttccaggtcaacaatactcacccac

SEQ ID NO: 1540 - DNA, 45 bp, Artificial Sequence
  circ_mIDUA_sense_R primer
ccatgccgactgatggcagaaaaaaaaaaactccaggccgctgcg

SEQ ID NO: 1541 - DNA, 409 bp, Artificial Sequence
  cDNA sequence that interacts with APOBEC
actgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatcc

SEQ ID NO: 1542 - DNA, 16 bp, Artificial Sequence
  AAV-ITR-F primer
cggcctcagtgagcga

SEQ ID NO: 1543 - DNA, 21 bp, Artificial Sequence
  AAV-ITR-R primer
ggaacccctagtgatggagtt

SEQ ID NO: 1544 - DNA, 33 bp, Artificial Sequence
  clip3 pAdapter Sequence
agatcggaagagcacacgtctgaactccagtca

SEQ ID NO: 1545 - DNA, 33 bp, Artificial Sequence
  clip3 pAdapter Sequence
agatcggaagagcgtcgtgtagggaaagagtgt

SEQ ID NO: 1546 - RNA, 21 nt, Artificial Sequence
  IDUA fragment Exon 9
gaacaacucuaggcagagguc

SEQ ID NO: 1547 - RNA, 21 nt, Artificial Sequence
  IDUA fragment Exon 9
gaacaacucugggcagagguc

SEQ ID NO: 1548 - protein, 7 aa, Artificial Sequence
  IDUA fragment
EQLWAEV

(19) United States
(12) Patent Application Publication
(10) Pub. No.: US 2025/0270543 A1
(43) Pub. Date: Aug. 28, 2025

(54) PROGRAMMABLE RNA EDITING IN VIVO VIA RECRUITMENT OF ENDOGENOUS ADARS

(71) Applicant: The Regents of the University of California, Oakland, CA (US)

(72) Inventors: PRASHANT MALI, LA JOLLA, CA (US); Dhruva Katrekar, La Jolla, CA (US); James Yen, La Jolla, CA (US)

(21) Appl. No.: 18/270,730

(22) PCT Filed: Jan. 4, 2022
(86) PCT No.: PCT/US2022/011187

§ 371 Date: Jul. 2, 2023

Related U.S. Application Data
(60) Provisional application No. 63/133,727, filed on Jan. 4, 2021.
(60) Provisional application No. 63/280,605, filed on Nov. 17, 2021.

(51) Int. Cl. C12N 15/11; A61K 48/00; A61P 3/00; C12N 15/86

(52) U.S. Cl. CPC C12N 15/11; A61P 3/00; C12N 15/86; A61K 48/00; C12N 2310/20; C12N 2310/532; C12N 2750/14143

(57) ABSTRACT

Disclosed herein are engineered guide RNAs, constructs for forming engineered guide RNAs, pharmaceutical compositions thereof, methods of making the engineered guide RNAs, and methods of treating or preventing a diseases and disorders of a subject by administering one or more of the engineered guide RNAs or the constructs for forming the engineered guide RNAs.

### CROSS REFERENCE TO RELATED APPLICATIONS

This application is a U.S. National Phase application filed under 35 U.S.C. § 371 and claims priority to International Application No. PCT/US2022/011187, filed Jan. 4, 2022, which application claims priority under 35 U.S.C. § 119(e) to U.S. Provisional Application No. 63/133,727, filed Jan. 4, 2021, and to U.S. Provisional Application No. 63/280,605, filed Nov. 17, 2021, the disclosures of which are incorporated herein by reference for all purposes.

### STATEMENT OF GOVERNMENT SUPPORT

This invention was made with government support under Grant Nos. GM123313, CA222826, and HG009285, awarded by the National Institutes of Health. The government has certain rights in the invention.

### FIELD OF THE INVENTION

The disclosure provides for engineered guide RNAs, pharmaceutical compositions thereof, methods of making the engineered guide RNAs, vectors comprising engineered guide RNAs or precursors thereof, and methods of treating a subject by administering one or more engineered guide RNAs.

### INCORPORATION BY REFERENCE OF SEQUENCE LISTING

Accompanying this filing is a Sequence Listing entitled, “Sequence-Listing_ST25” created on Jan. 4, 2022 and having 393,305 bytes of data, machine formatted on IBM-PC, MS-Windows operating system. The sequence listing is hereby incorporated by reference in its entirety for all purposes.

### SUMMARY

The disclosure provides a circular engineered guide RNA comprising an antisense region with partial complementarity to a region of an IDUA target RNA sequence. In one embodiment, the circular engineered guide RNA is configured to facilitate editing of a base of a target nucleotide in the IDUA target RNA sequence by an RNA editing entity. In another embodiment, the circular engineered guide RNA further comprises an RNA editing entity recruiting domain. In a further embodiment, the RNA editing entity recruiting domain comprises at least about 80% sequence identity to at least about 20 contiguous nucleic acids of: an Alu domain, an APOBEC recruiting domain, or a GluR2 domain. In still a further embodiment, the RNA editing entity recruiting domain comprises at least about 80% sequence identity to at least about 20 contiguous nucleic acids of the Alu domain. In yet a further embodiment, the RNA editing entity recruiting domain comprises at least about 80% sequence identity to the Alu domain. In another embodiment, the RNA editing entity recruiting domain comprises at least about 80% sequence identity to at least about 20 contiguous nucleic acids of the APOBEC recruiting domain. In a further embodiment, the RNA editing entity recruiting domain comprises at least about 80% sequence identity to the APOBEC recruiting domain. In another embodiment, the RNA editing entity recruiting domain comprises at least about 80% sequence identity to at least about 20 contiguous nucleic acids of the GluR2 domain. In a further embodiment, the RNA editing entity recruiting domain comprises at least about 80% sequence identity to the GluR2 domain. In still another or further embodiment of any of the foregoing embodiments, the RNA editing entity recruiting domain recruits an RNA editing entity that, when associated with the circular engineered guide RNA and the IDUA target RNA sequence, performs a chemical transformation on a base of a target nucleotide in the IDUA target RNA sequence, thereby generating an edited IDUA target RNA sequence. In a further embodiment, a protein translated from the edited IDUA target sequence is longer than a protein translated from an unedited IDUA target sequence as demonstrated in an in vitro assay. In another or further embodiment, the RNA editing entity is an endogenous enzyme. In another or further embodiment, the RNA editing entity is a recombinant enzyme. In another or further embodiment of any of the foregoing embodiments, the circular engineered guide RNA comprises at least about 80% sequence identity to the reverse complement of SEQ ID NO: 1418, or at least about 80% sequence identity to 50-200 nucleotides of SEQ ID NO: 1418 containing nucleotides 1204-1206. In another or further embodiment of any of the foregoing embodiments, the antisense region comprises a sequence length from about 20 nucleotides to about 1,000 nucleotides in length. In a further embodiment, the antisense region comprises a sequence length from about 50 nucleotides to about 200 nucleotides in length. In another embodiment, the antisense region comprises a sequence length from about 60 nucleotides to about 100 nucleotides in length. In another or further embodiment, the chemical transformation transforms a stop codon into a sense codon. In another or further embodiment of any of the foregoing embodiments, the circular engineered guide RNA comprising an antisense region of about 100 bp or more has at least about: a 2-fold increase, a 3-fold increase, or a 3.5-fold increase in RNA editing as compared to a comparable linear engineered guide RNA as measured by an in vitro assay. In a further embodiment, the circular engineered guide RNA comprising an antisense region of about 100 bp to about 200 bp has at least about: a 2-fold increase, a 3-fold increase, or a 3.5-fold increase in RNA editing as compared to a comparable linear engineered guide RNA as measured by an in vitro assay. In another or further embodiment of any of the foregoing embodiments, the circular engineered guide RNA does not comprise a G mismatch opposite all non-target adenosines. In another or further embodiment of any of the foregoing embodiments, the circular engineered guide RNA comprises at least one 8, 9, 10, 11 or 12-bp loop. In a further embodiment, the circular engineered guide with the at least one 8-bp loop has decreased hyperediting as compared to a circular engineered guide RNA without the at least one 8-bp loop as measured by an in vitro assay. In another or further embodiment of any of the foregoing embodiments, the circular engineered guide RNA or a linear precursor thereof is genetically encodable. In another or further embodiment of any of the foregoing embodiments, the circular engineered guide RNA or a linear precursor thereof does not have a chemical modification.

The disclosure also provides a nucleic acid encoding a linear precursor of the circular engineered guide RNA of any of the foregoing embodiments, or a vector comprising the nucleic acid. In one embodiment, the nucleic acid comprises two copies of the circular engineered guide RNA. In another or further embodiment, the nucleic acid comprises a U6 promoter downstream of a CMV promoter. In another or further embodiment of any of the foregoing embodiments, the nucleic acid is double stranded.

The disclosure also provides a vector comprising the circular engineered guide RNA of any of the foregoing embodiments or the nucleic acid of any of the foregoing embodiments. In one embodiment, the vector comprises a liposome, a nanoparticle, or any combination thereof. In another embodiment, the vector is a viral vector. In a further embodiment, the viral vector is an adeno-associated virus (AAV) vector. In still a further embodiment, the AAV vector comprises an AAV8 serotype, or a derivative thereof. In another embodiment, the AAV vector comprises an AAV1 serotype, an AAV2 serotype, AAV3 serotype, AAV4 serotype, AAV5 serotype, an AAV6 serotype, AAV7 serotype, an AAV9 serotype, a derivative of any of these, or any combination thereof.

The disclosure also provides an isolated cell that comprises the circular engineered guide RNA, the nucleic acid, or the vector of any for the foregoing embodiments.

The disclosure also provides a pharmaceutical composition comprising the circular engineered guide RNA, the nucleic acid, or the vector of any of the foregoing embodiments, and a pharmaceutically acceptable: excipient, diluent, or carrier wherein optionally the pharmaceutical composition is in unit dose form.

The disclosure also provides a kit comprising the circular engineered guide RNA, the vector, or the pharmaceutical composition of any of the foregoing embodiments, compartmentalized to include one or more containers.

The disclosure provides a method of treating a human in need thereof comprising: administering to the human a vector encoding a circular engineered guide RNA or a linear precursor thereof that comprises an antisense region with complementarity to a region of an IDUA target RNA sequence. In one embodiment, the method further comprises administering an RNA editing entity or a polynucleotide encoding an RNA editing entity to the human in need thereof. In a further embodiment, the RNA editing entity is a recombinant enzyme. In another or further embodiment, the human has or is suspected of having a disease or condition that comprises a Mucopolysaccharidosis type I (MPS I). In still a further embodiment, the disease or condition MPS I comprises Hurler syndrome, Hurler-Scheie syndrome, Scheie syndrome, or any combination thereof.

The disclosure also provides a circular engineered guide RNA comprising an antisense region with partial complementarity to a region of a FANCC, a CTNNB1, a SMAD4, a TARDBP, or any combination thereof, target RNA sequence. In one embodiment, the circular engineered guide RNA is configured to facilitate editing of a base of a target nucleotide in the FANCC, the CTNNB1, the SMAD4, or the TARDBP target RNA sequence by an RNA editing entity. In another embodiment, the circular engineered guide RNA further comprises an RNA editing entity recruiting domain. In another embodiment, the RNA editing entity recruiting domain comprises at least about 80% sequence identity to at least about 20 contiguous nucleic acids of: an Alu domain, an APOBEC recruiting domain, or a GluR2 domain. In another or further embodiment, the RNA editing entity recruiting domain recruits an RNA editing entity that, when associated with the circular engineered guide RNA and the FANCC, the CTNNB1, the SMAD4, or the TARDBP target RNA sequence, performs a chemical transformation on a base of a target nucleotide in the FANCC, the CTNNB1, the SMAD4, or the TARDBP target RNA sequence, thereby generating an edited FANCC, CTNNB1, SMAD4, or TARDBP target RNA sequence. In still a further embodiment, the RNA editing entity is an endogenous enzyme. In another or further embodiment, the RNA editing entity is a recombinant enzyme. In still another or further embodiment of any of the foregoing embodiments, the antisense region comprises a sequence length from about 20 nucleotides to about 1,000 nucleotides in length. In still another or further embodiment of any of the foregoing embodiments, the circular engineered guide RNA comprising an antisense region of about 100 bp or more has at least about: a 2-fold increase, a 3-fold increase, or a 3.5-fold increase in RNA editing as compared to a comparable linear engineered guide RNA as measured by an in vitro assay. In still another or further embodiment of any of the foregoing embodiments, the circular engineered guide RNA does not comprise a G mismatch opposite all non-target adenosines. In still another or further embodiment of any of the foregoing embodiments, the circular engineered guide RNA comprises at least one 8-bp loop. In a further embodiment, the circular engineered guide with the at least one 8-bp loop has decreased hyperediting as compared to a circular engineered guide RNA without the at least one 8-bp loop as measured by an in vitro assay.

The disclosure also provides a nucleic acid encoding a linear precursor of the circular engineered guide RNA of any of the immediately preceding embodiments, or a vector comprising the nucleic acid. In one embodiment, the nucleic acid comprises two copies of the circular engineered guide RNA. In another or further embodiment, the nucleic acid comprises a U6 promoter downstream of a CMV promoter. In still another or further embodiment of any of the foregoing embodiments, the nucleic acid is double stranded. The disclosure also provides a vector comprising the circular engineered guide RNA described immediately above or the nucleic acid as described above. In one embodiment, the vector comprises a liposome, a nanoparticle, or any combination thereof. In another embodiment, the vector is a viral vector. In a further embodiment, the viral vector is an adeno-associated virus (AAV) vector. In still a further embodiment, the AAV vector comprises an AAV8 serotype, or a derivative thereof. In a further embodiment, the AAV vector comprises an AAV1 serotype, an AAV2 serotype, AAV3 serotype, AAV4 serotype, AAV5 serotype, an AAV6 serotype, AAV7 serotype, an AAV9 serotype, a derivative of any of these, or any combination thereof. The disclosure also provides a recombinant or isolated cell or a pharmaceutical composition that comprises the circular engineered guide RNA, nucleic acid or vector described herein. In one embodiment, the pharmaceutical composition comprises a pharmaceutically acceptable excipient, diluent, or carrier wherein optionally the pharmaceutical composition is in unit dose form. The disclosure further comprises a kit comprising the circular engineered guide RNA, the vector, or the pharmaceutical composition and a container.

The disclosure provides a method of treating a human in need thereof comprising: administering to the human a vector encoding a circular engineered guide RNA or a linear precursor thereof that comprises an antisense region with complementarity to a region of a FANCC, a CTNNB1, a SMAD4, a TARDBP, or any combination thereof target RNA sequence. In a further embodiment, the human has or is suspected of having a disease or condition that comprises Fanconi anemia, a colorectal cancer (CRC), a pilomatrixoma (PTR), a medulloblastoma (MDB), an ovarian cancer, a pilomatrixoma, a neurodevelopmental disorder, a hemorrhagic telangiectasia, a juvenile polyposis syndrome, Myhre syndrome, or an amyotrophic lateral sclerosis (ALS).

The disclosure also provides an engineered guide RNA for editing a nucleotide in a target RNA, the engineered guide RNA comprising: an RNA editing entity recruiting domain; a targeting domain that is at least 85% complementary to the target RNA and comprises a modification mismatch and a plurality of off-target-inhibitory mismatches; wherein the RNA editing entity recruiting domain recruits an RNA editing entity that, when associated with the engineered guide RNA, performs a chemical transformation on a base of a nucleotide in the RNA sequence at the modification mismatch, thereby generating an edited RNA sequence, wherein the engineered guide RNA is a closed loop. In one embodiment, the targeting domain comprises a sequence length from about 20 nucleotides to about 1,000 nucleotides in length. In another embodiment, the targeting domain comprises a sequence length of at least about 100 nucleotides in length. In another embodiment, the plurality of off-target-inhibitory mismatches comprise loops of 6-12 bp. In another embodiment, the plurality of off-target-inhibitory mismatches are −5 bp and +30 bp from the modification mismatch on the targeting domain. In another or further embodiment of any of the foregoing embodiments, the modification mismatch comprises an A in the target RNA and a C in the targeting domain. In another or further embodiment of any of the foregoing embodiments, the plurality of off-target-inhibitory mismatches comprise A in the target RNA and a G in the targeting domain. In a further embodiment, the plurality of off-target-inhibitory mismatches comprises mismatches at −5 bp and +30 bp from the modification mismatch and one or more additional off-target-inhibitory mismatches spaced 15 bp from the −5 bp and +30 bp mismatch. In still another embodiment, the plurality of off-target-inhibitory mismatches comprise 8 bp loops along the targeting domain at intervals of 15 bp flanking a 36 bp central region that carries the modification mismatch. In another or further embodiment of any of the foregoing embodiments, the plurality of off-target-inhibitory mismatches reduces bystander adenosine editing compared to a target domain lacking the plurality of off-target-inhibitory mismatches. In a further embodiment, the reduction of bystander adenosine editing is greater than 5%. In another embodiment, the reduction of bystander adenosine editing is greater than 10%. In another embodiment, the reduction of bystander adenosine editing is greater than 20%. In another or further embodiment of any of the foregoing embodiments, the chemical transformation on the base results in at least a partial knockdown of the edited RNA sequence. In a further embodiment, the partial knockdown comprises a reduced level of a protein or fragment thereof expressed from the edited RNA sequence. In still a further embodiment, the reduced level is from about 5% to 100%. In yet a further embodiment, the reduced level is from about 60% to 100%. In another or further embodiment of any of the foregoing embodiments, the partial knockdown or reduced level is determined compared to an otherwise identical unedited RNA sequence as determined in an in vitro assay. In another or further embodiment of any of the foregoing embodiments, the chemical transformation results in a sense codon read as a stop codon. In another or further embodiment of any of the foregoing embodiments, the chemical transformation results in a stop codon read as a sense codon. In another or further embodiment of any of the foregoing embodiments, the chemical transformation results in a first sense codon read as a second sense codon. In another or further embodiment of any of the foregoing embodiments, the chemical transformation results in a first stop codon read as a second stop codon. In another or further embodiment of any of the foregoing embodiments, the engineered guide RNA is configured to form a secondary structure comprising: a stem-loop, a cruciform, a toe hold, a mismatch bulge, or any combination thereof. In another or further embodiment of any of the foregoing embodiments, the RNA editing entity recruiting domain comprises at least about 80% sequence homology to at least about 20 nucleic acids of: an Alu domain, an APOBEC recruiting domain, a GluR2 domain, or a Cas13 recruiting domain. In a further embodiment, the RNA editing entity recruiting domain comprises at least about 80% sequence homology to at least about 20 nucleic acids of the Alu domain. In still a further embodiment, the RNA editing entity recruiting domain comprises at least about 80% sequence homology to the Alu domain. In another embodiment, the RNA editing entity recruiting domain comprises at least about 80% sequence homology to at least about 20 nucleic acids of the APOBEC recruiting domain. In a further embodiment, the RNA editing entity recruiting domain comprises at least about 80% sequence homology to the APOBEC recruiting domain. In another embodiment, the RNA editing entity recruiting domain comprises at least about 80% sequence homology to at least about 20 nucleic acids of the GluR2 recruiting domain. In a further embodiment, the sequence comprises at least about 80% sequence homology to the GluR2 recruiting domain. In another or further embodiment of any of the foregoing embodiments, the RNA editing entity is an endogenous enzyme. In another or further embodiment of any of the foregoing embodiments, the RNA editing entity is a recombinant enzyme. In another or further embodiment of any of the foregoing embodiments, the engineered guide RNA comprises a modified nucleotide base. In a further embodiment, the modification comprises a sugar modification. In another embodiment, a nucleotide of the engineered guide RNA comprises a methyl group, a fluoro group, a methoxyethyl group, an ethyl group, a phosphate group, an amide group, an ester group, or any combination thereof. In another embodiment, the engineered guide RNA comprises a protein coating. In still another embodiment, the engineered guide RNA is genetically encodable. In yet another embodiment, the RNA editing entity is operably linked to the engineered guide RNA. In a further embodiment, a linkage between the engineered guide RNA and the RNA editing entity is a direct or an indirect covalent linkage. In another embodiment, the engineered guide RNA retains a half-life, in an aqueous solution at a physiological pH, that is at least about 4 times longer than a comparable guide RNA that is not circular. In another embodiment, a therapeutically effective amount of the engineered guide RNA dosed to a subject in need thereof is at least about 4 times less than a comparable guide RNA that is not circular on a weight-to-weight basis. In still another embodiment, the targeting domain has complementarity to a region of an IDUA target RNA sequence.

The disclosure also provides a recombinant RNA polynucleotide construct for editing RNA, wherein the construct comprises the following domains: a 5′ ribozyme region; a 5′ ligation sequence adjacent to the 5′ ribozyme region; an antisense/targeting domain comprising an adenosine deaminases acting on RNA (ADAR) guide sequence that is used to edit a targeted mRNA sequence; a 3′ ligation sequence that is adjacent to the antisense domain; and a 3′ ribozyme region, wherein the RNA construct recruits ADARs, wherein the 5′ ribozyme and 3′ ribozyme regions upon autocatalytic cleavage leave termini that can be ligated together by an RNA ligase to yield circular RNA constructs, and wherein the antisense/targeting domain comprises a modification mismatch and a plurality of off-target-inhibitory mismatches. In one embodiment, the antisense/targeting domain comprises a sequence length from about 20 nucleotides to about 1,000 nucleotides in length. In another embodiment, the antisense/targeting domain comprises a sequence length of at least about 100 nucleotides in length. In still another embodiment, the plurality of off-target-inhibitory mismatches comprise loops of 6-12 bp. In yet another embodiment, the plurality of off-target-inhibitory mismatches are −5 bp and +30 bp from the modification mismatch on the targeting domain. In another or further embodiment of any of the foregoing embodiments, the modification mismatch comprises an A in the target RNA and a C in the antisense/targeting domain. In another or further embodiment of any of the foregoing embodiments, the plurality of off-target-inhibitory mismatches comprise A in the target RNA and a G in the antisense/targeting domain. In a furth embodiment, the plurality of off-target-inhibitory mismatches comprises mismatches at −5 bp and +30 bp from the modification mismatch and one or more additional off-target-inhibitory mismatches spaced 15 bp from the −5 bp and +30 bp mismatch. In another embodiment, the plurality of off-target-inhibitory mismatches comprise 8 bp loops along the antisense/targeting domain at intervals of 15 bp flanking a 36 bp central region that carries the modification mismatch. In another or further embodiment of any of the foregoing embodiments, the plurality of off-target-inhibitory mismatches reduces bystander adenosine editing compared to a target domain lacking the plurality of off-target-inhibitory mismatches. In a further embodiment, the reduction of bystander adenosine editing is greater than 5%. In still another embodiment, the reduction of bystander adenosine editing is greater than 10%. In yet another embodiment, the reduction of bystander adenosine editing is greater than 20%. In another or further embodiment of any of the foregoing embodiments, the chemical transformation on the base results in at least a partial knockdown of the edited RNA sequence. In a further embodiment, the partial knockdown comprises a reduced level of a protein or fragment thereof expressed from the edited RNA sequence. In yet another embodiment, the reduced level is from about 5% to 100%. In another embodiment, the reduced level is from about 60% to 100%. In yet another embodiment, the 5′ ribozyme region and the 3′ ribozyme region are twister ribozymes. In another embodiment, the ADAR guide sequence comprises a GluR2 sequence. In another embodiment, the one or more off-target inhibitory mismatches comprises a guanidine base that are mismatched opposite to non-targeted adenine base in the target mRNA sequence. In still yet another embodiment, the targeting mismatch and the one or more off-target inhibitory mismatches form loop structures that are 6 bp to 15 bp in length. The disclosure also provides a method to edit a targeted mRNA sequence with endogenous adenosine deaminases acting on RNA (ADARs), comprising: contacting cells comprising the targeted mRNA sequence with the engineered guide RNA or the RNA construct as described in any of the foregoing embodiments.

### BRIEF DESCRIPTION OF THE DRAWINGS

A better understanding of certain features and advantages of the disclosure will be obtained by reference to the following detailed description that sets forth illustrative embodiments, in which the principles of the disclosure are utilized, and the accompanying drawings of which:

FIG. 1A-D shows engineering circular ADAR recruiting guide RNAs (cadRNAs). (A) A comparison of the RNA editing efficiencies in the 3′ UTR of the RAB7A transcript via various adRNA (ADAR recruiting guide RNAs) designs. Values represent mean+/−SEM (n=3; with respect to the linear.100.50, left-to-right, p=0.7289, p=0.0226, p=0.0019, p=0.0055, p=0.0027, and p=0.0006; unpaired t-test, two-tailed). In the schematics, the bottom strand represents the antisense domain of the adRNA while the target mRNA is the top. The bulge indicates the A-C mismatch between the target mRNA and adRNA. The adRNAs are labelled using the following convention: (domain name).(antisense length).(position of A-C mismatch from 5′ end of the antisense). (B) RNA editing efficiencies achieved 48 hours and 96 hours post transfection of various adRNA designs. Values represent mean+/−SEM (n=3; left-to-right, p=0.0019, p=0.0027, p=0.0006 and p=0.8488, p=0.0014, p=0.0077; unpaired t-test, two-tailed). The 48 hour panel data is reproduced from FIG. 1a. (C) RT-PCR based confirmation of adRNA circularization in cells. (D) The ability of adRNAs to effect RNA editing of the clue transcript was assessed in the presence of an siRNA targeting ADAR1. Values represent mean+/−SEM (n=3; left-to-right, p=0.0002, p=0.0216 and p=0.0001; unpaired t-test, two-tailed). All experiments were carried out in HEK293FT cells.

FIG. 2A-D shows transcriptome-wide and target transcript-level specificity profiles of cadRNAs. (A) (left-panel) 2D histograms comparing the transcriptome-wide A-to-G editing yields observed with a circular adRNA construct (y-axis) to the yields observed with the control sample (x-axis). Each histogram represents the same set of reference sites, where read coverage was at least 10 and at least one putative editing event was detected in at least one sample. N_{sig} is the number of sites with significant changes in editing yield. Points corresponding to such sites are shown with crosses. The on-target editing values obtained via Sanger sequencing for the three samples analyzed via RNA seq are HEK293FT: 0%, circular.100.50: 40.47% and circular 200.100: 43.54% respectively. (right-panel) A comparison of the number of off-targets induced by delivery of circular adRNAs, linear adRNAs, and linear adRNAs with co-delivered ADAR2. All experiments were carried out in HEK293FT cells. (B) Engineered cadRNA designs for reducing bystander editing. The bottom strand represents the antisense domain while the target mRNA is the top strand. The target adenosine is depicted while the mismatch is opposite. Design 1 (cadRNA): Unmodified circular.200.100 antisense. Design 2 (cadRNA.bulges): Antisense bulges created by positioning guanosines opposite bystander edited adenosines. Design 3 (cadRNA.loops): Loops of size 8 bp created at position −5 and +30 relative to the target adenosine. Design 4 (cadRNA.loops.interspersed): Loops of size 8 bp created at position −5 and +30 relative to the target adenosine and additional 8 bp loops added at 15 bp intervals all along the antisense strand. Plots depicting the location and extent of all substitutions in the 200 bp dsRNA stretch (n=1 representative plot shown for each construct, analyzed via CRISPResso2 36). (C) Plot depicts % of perfectly edited reads and those with further A-to-G substitutions (in addition to the target site) in the 200 bp dsRNA stretch formed between the cadRNA and target RNA as observed with the various designs. Substitutions other than A-to-G were not considered for this analysis. Values represent mean %+/−SEM on-target editing in 200 bp long amplicons as quantified by NGS (n=3). All experiments were carried out in HEK293FT cells. (D) Heatmaps of percent editing within a 60 bp window around the target adenosine in the GAPDH transcript and RAB7A transcript as quantified by Sanger sequencing. The positions of adenosines relative to the target adenosine (0) are listed below the heatmap. Values represent mean % editing (n=3 for GAPDH and n=2 for RAB7A). All experiments were carried out in HEK293FT cells.

FIG. 3A-B shows in vitro activity of cadRNAs. (A) Plasmid delivered in situ cadRNA generation: RNA editing efficiencies across various transcripts observed in HEK293FT and K562 cells via plasmid delivered circular.200.100 adRNA, 48 hours post transfections are shown. Values represent mean+/−SEM (n=3). These experiments were carried out using either cadRNA or cadRNA.loops.interspersed from FIG. 2B. Associated changes in expression levels of target transcripts as compared to levels seen in untransfected controls is also shown, 48 hours post transfections (p=0.2599, p=0.0135, p=0.1982, p=0.7871, p=0.0144, p=0.2674, p=0.1168, p=0.7852, p=0.5145; unpaired t-test, two-tailed). (B) In vitro transcribed (IVT) circular adRNA generation: Linear forms of twister ribozyme flanked circular adRNAs were transcribed in vitro using a T7 polymerase, purified using LiCl, and transfected into cells, where they circularize in situ by the endogenous RNA ligase RtcB. RNA editing efficiencies across various transcripts observed in HEK293FT and K562 cells via IVT circular adRNA, 24 hours post transfections are shown. Values represent mean+/−SEM (n=3). Associated levels of IVT and plasmid delivered circular.200.100 adRNA targeting RAB7A measured in transfected HEK293FT cells 24 hours post transfections are also shown. Values represent mean+/−SEM (n=3).

FIG. 4A-H shows in vivo activity of cadRNAs. (A) (i) AAV vectors used for adRNA delivery. (ii) Schematic of the in vivo experiment. (B) In vivo RNA editing efficiencies of the mPCSK9 transcript in mice livers via systemic delivery of U6 transcribed linear (U6+27) and genetically encoded circular adRNAs packaged in AAV8. Values represent mean+/−SEM (n=3; p=0.0002; unpaired t-test, two-tailed). (C) Relative expression levels of circular adRNAs. Values represent mean+/−SEM (n=3; p=0.0305; unpaired t-test, two-tailed). (D) mPCSK9 transcript levels relative to GAPDH. Values represent mean+/−SEM (n=3; p=0.6179, p=0.6125, p=0.9323; unpaired t-test, two-tailed). (E) Schematic of the IDUA-W392X mRNA, and RNA editing experiment (SEQ ID NOs:1546-1548). (F) In vivo UAG-to-UGG RNA editing efficiencies of the IDUA transcript in mice livers via systemic delivery of genetically encoded circular adRNAs packaged in AAV8. Values represent mean+/−SEM (n=3). (G) IDUA transcript levels relative to GAPDH. Values represent mean+/−SEM (n=3; p=0.1185, p=0.3815, p=0.0042; unpaired t-test, two-tailed). (H) GAG content in mice livers of AAV8-scrambled.2x.circular.200.100 and AAV8-IDUA.2x.circular.200.100 injected IDUA-W392X mice. Wild type C57BL/6J mice were included as controls. Values represent mean+/−SEM (n=3; p=0.0285; unpaired t-test, two-tailed).

FIG. 5A-D shows characterization of genetically encoded cadRNAs. (A) RNA editing efficiencies achieved 48 hours and 96 hours post transfection of circular.200.100 and ribozyme.mutant.200.100 plasmids. Ribozyme.mutant.200.100 was created by substituting two residues in both twister ribozymes (P3 ribozyme: residue 15 G to U and residue 16 U to G; P1 ribozyme: residue 22 A to G and residue 26 C to U) of the construct circular.200.100 37,38. Values represent mean+/−SEM (n=3; p=0.0021, p=0.0112; unpaired t-test, two-tailed). (B) Schematic representation of various products detected by inward and outward binding primers used for quantification. The outward binding primers selectively amplify the cadRNA. The inward binding primers amplify uncleaved and cleaved-unligated fractions in addition to cadRNA. Values represent mean+/−SEM (n=3). (C) Cells transfected with circular.200.100 and ribozyme.mutant.200.100 plasmids were treated with actinomycin D for 1, 6 and 16 hours starting at 24 hours post transfections. qPCRs were carried out using inward binding primers from panel (b) and expression levels were normalized to untreated samples. (D) Levels of circular.100.50 and linear.100.50 adRNA were measured in the nucleus and cytoplasm. GFP transfected cells were included as controls. U1 snRNA and GAPDH were used to normalize for the nuclear and cytoplasmic compartments respectively. Relative U1 snRNA and GAPDH levels seen in the nuclear vs cytoplasmic fractions were consistent with other work. Values represent mean+/−SEM (n=3). All experiments were carried out in HEK293FT cells.

FIG. 6 shows characterization of IVT synthesized cadRNAs. qPCRs were carried out on cDNA synthesized from IVT-circular.200.100 adRNA and IVT-ribozyme.mutant.200.100 adRNA using primers binding to the ligation stem and ribozyme sequence. n.d.: not detected. Values represent mean+/−SEM (n=3).

FIG. 7 shows in vivo specificity of cadRNAs. 2D histograms comparing the transcriptome-wide A-to-G editing yields observed with an AAV delivered construct (y-axis) to the yields observed with the control AAV construct (x-axis). Each histogram represents the same set of reference sites, where read coverage was at least 10 and at least one putative editing event was detected in at least one sample. Nsig is the number of sites with significant changes in editing yield. Points corresponding to such sites are shown with crosses. The on-target editing efficiency values obtained in the RNA seq are highly inflated due to a large number of reads coming from the cadRNAs mapping onto the target and thus have been omitted from the 2D histograms. The on-target editing values obtained via Sanger sequencing for the four samples analyzed by RNA seq were mCherry-M1: 0%, mCherry-M2: 0%, 2x.circular.200.100-M1: 42.94% and 2x.circular.200.100-M2: 41.32% respectively. M1 and M2 refer to injected mouse 1 and 2.

FIG. 8A-C shows transcriptomic changes associated with in vivo cadRNA expression. (A) qPCRs were carried out on IFN-inducible genes involved in sensing of dsRNA 2 weeks and 8 weeks post AAV injections. Values represent mean+/−SEM (n=3; p-values for 2-week long experiment, 2x.circular.200.100 vs mCherry, for genes from left to right p=0.0721, p=0.0353, p=0.8082, p=0.0748, p=0.0303; p-values for 8-week long experiment, 2x.circular.200.100 vs mCherry, for genes from left to right p=0.7276, p=0.6020, p=0.3838, p=0.3491, p=0.2746; unpaired t-test, two-tailed). (B) qPCRs were carried out on ADAR variants 2 weeks and 8 weeks post AAV injections. Values represent mean+/−SEM (n=3; p-values for 2-week long experiment, 2x.circular.200.100 vs. mCherry, for ADAR variants from left to right p=0.3165, p=0.1885, p=0.2815; p-values for 8-week long experiment, 2x.circular.200.100 vs. mCherry, for genes from left to right p=0.8150, p=0.1440, p=0.9532; unpaired t-test, two-tailed). (C) Transcriptome-wide differentially expressed genes in the two groups: 2x.circular.200.100 vs. mCherry (black dots).

FIG. 9 provides for Table 13.

FIG. 10 shows curbing bystander editing of the RAB7A transcript. Histograms of percent A-to-G editing within a 200 bp window around the target adenosine in the RAB7A transcript as quantified by Sanger sequencing. The target adenosine is located at position 0. The dsRNA stretch formed between the antisense and the target are shown below each histogram. Design 1 (cadRNA): Unmodified circular.200.100 antisense, in addition to the A-C mismatch at position 0, two mismatches are seen at positions +66 and +91 that were created to avoid a stretch of poly Us to allow for transcription from a U6 promoter. Design 2 (cadRNA.loops.interspersed.v1): Loops of size 8 bp created at position −5 and +30 relative to the target adenosine and additional 8 bp loops added at 15 bp intervals along the antisense strand. Design 3 (cadRNA.loops.interspersed.v2): As compared to v1, a G-mismatch was positioned opposite a highly edited A (at position +9), an additional 8 bp loop was added at position −81 and the loop at position +49 was changed to a 12 bp loop. Design 4 (cadRNA.loops.interspersed.v3): As compared to v1, the 8 bp loop at +30 was changed to a 12 bp loop starting at position +27, one additional 8 bp loop was added at position −81 and the loop at position +49 was changed to a 12 bp loop. Values represent mean % editing (n=2). All experiments were carried out in HEK293FT cells.

FIG. 11 provides IDUA mRNA sequence (SEQ ID NO:1418).

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in their entirety.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers may be modified by the term “about”. The term “about” when used to describe the invention, in connection with percentages means ±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The terms “adenine”, “guanine”, “cytosine”, “thymine”, “uracil” and “hypoxanthine” (the nucleobase in inosine) as used herein refer to the nucleobases as such.

The terms “adenosine”, “guanosine”, “cytidine”, “thymidine”, “uridine” and “inosine”, refer to the nucleobases linked to the (deoxy)ribosyl sugar.

The term “adeno-associated virus” or “AAV” as used herein refers to a member of the class of viruses associated with this name and belonging to the genus depend parvovirus, family Parvoviridae. Multiple serotypes of this virus can be suitable for gene delivery. In some cases, serotypes can infect cells from various tissue types. Examples of AAV serotypes are AAV1, AAV2, AAV3, AAV4, AAV5, AAV6, AAV7, AAV8, AAV9, AAV10, and AAV11. Non-limiting exemplary serotypes useful for the purposes disclosed herein include any of the 11 serotypes, e.g., AAV2 and AAV8.

The term “Adenosine Deaminase acting on RNA” or “ADAR” as used herein refers to an adenosine deaminase that can convert adenosines (A) to inosines (I) in an RNA sequence. ADAR1 and ADAR2 are two exemplary species of ADAR that are involved in mRNA editing in vivo. Non-limiting exemplary sequences for ADAR1 may be found under the following reference numbers from different databases: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof. Related orthologs and homologs can be readily identified using various sequence search tools and databases.

The term “adRNA” stands for ADAR recruiting RNA. The terms “cadRNA” or “circ adRNA” stand for circular ADAR recruiting guide RNA. As used herein, circular guide RNAs can be referred to as circular ADAR recruiting guide RNAs (cadRNAs).

The term “Alu domain” can refer to a sequence obtained from the Alu transposable element (“Alu element”). In some cases, the Alu element can be about 300 base pairs in length. An Alu element typically comprise a structure: cruciform-polyA5-TAC-polyA6-cruciform-polyA tail, wherein both cruciform domains are similar in nucleotide sequence. An “Alu domain” can comprise a cruciform portion of the Alu element. In some embodiments, two Alu domains comprising cruciform structures are linked by a sequence complementary to a target RNA sequence.

As used herein, the term “circularized” and/or “circular” used in the context of a nucleic acid molecule (e.g., an engineered guide RNA) can generally refer to a nucleic acid molecule that can be represented as a polynucleotide sequence in a circular 2-dimensional format with one nucleotide after the other wherein the represented polynucleotide is circular or a closed loop. In some embodiments, a circular nucleic acid molecule does not comprise a 5′ reducing hydroxyl, a 3′ reducing hydroxyl, or both capable of being exposed to a solvent

The term “contacting” can mean direct or indirect binding or interaction between two or more entities. An example of direct interaction is binding. An example of an indirect interaction is where one entity acts upon an intermediary molecule, which in turn acts upon the second referenced entity. Contacting as used herein includes in solution, in solid phase, in vitro, ex vivo, in a cell and in vivo. In one embodiment, contacting can occur between a guide RNA and an RNA editing entity. Contacting in vivo can be referred to as administering, or administration.

The term “deficiency” as used herein can refer to lower than normal (physiologically acceptable) levels of a particular agent. In context of a protein, a deficiency can refer to lower than normal levels of the full-length protein.

As used herein the term “domain” refers to a particular region of a larger construct such that the domain is contained in or is part of the larger construct. With respect to nucleic acids a domain can refer to a coding sequence found in a larger construct containing multiple coding sequences.

The term “encode” as it is applied to polynucleotides can refer to a polynucleotide which is said to “encode” a polypeptide if, in its native state or when manipulated, it can be transcribed and/or translated to produce the mRNA for the polypeptide and/or a fragment thereof. The antisense strand is the complement of such a nucleic acid, and the encoding sequence can be deduced therefrom.

An “engineered polynucleotide” or “engineered guide RNA” are used interchangeably with circular guide RNA. An engineered polynucleotide can comprise a recombinant polynucleotide of DNA or RNA or a hybrid DNA/RNA construct. The engineered polynucleotide can give rise to a guide RNA and more particularly can give rise to a circular guide RNA.

The terms “equivalent” or “biological equivalent” are used interchangeably when referring to a particular molecule, biological or cellular material having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology can refer to a percent (%) identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein, such particular peptide, polypeptide or nucleic acid sequence can be determined conventionally using computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, a sequence of the disclosure) and a subject sequence, also referred to as a global sequence alignment, can be determined using the FASTDB computer program. In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

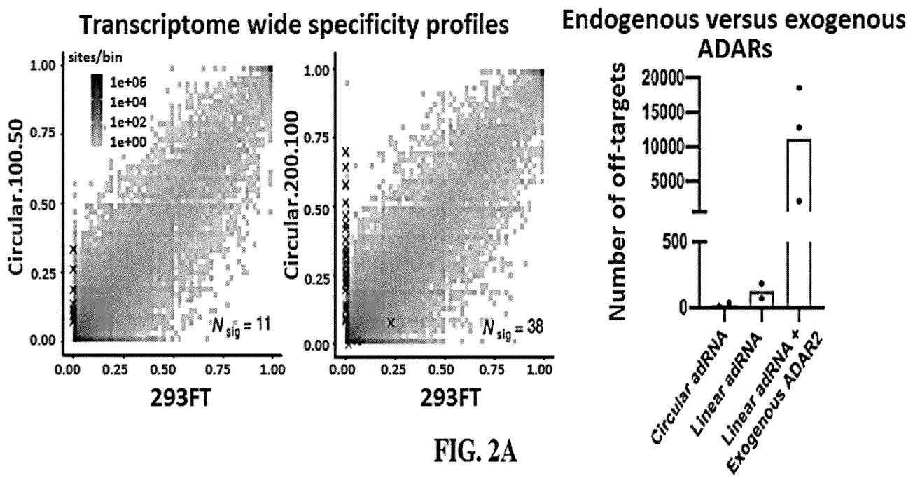

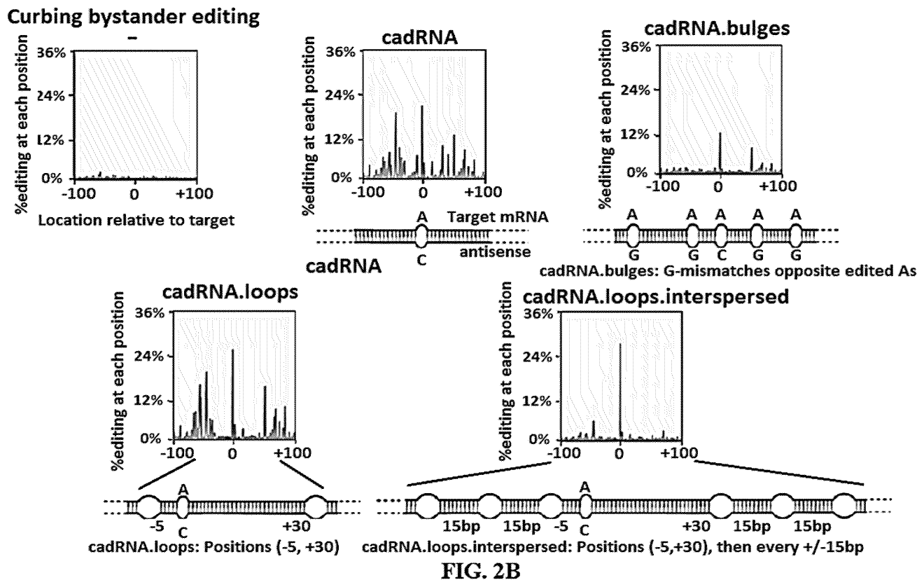

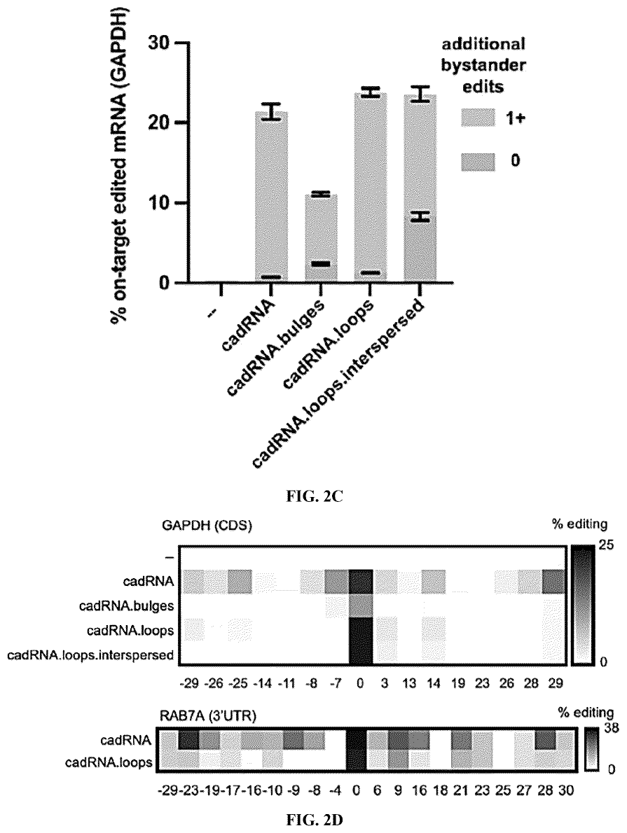

As used herein, “interspersed loops” or “interspersed loops in gRNA” refers to engineered mismatches that form bulges or loops when a gRNA interacts with its corresponding target RNA. The interspersed loops are engineered to increase target specificity, wherein each side of the gRNA (5′ and 3′) of the engineered mismatches are complementary to the target RNA to be chemically altered. For example, in certain embodiments, the mismatch forms the interspersed loops/bulges occurs at −5 and +30 from the site to be chemically modified and then every 15 bp 5′ or 3′ from the −5 and +30 sites. In still another embodiment, a circular antisense guide RNA comprises a plurality of loops/bulges generated between the gRNA and the target RNA that are created by positioning guanosine mismatches opposite hyperedited adenosines in the target RNA strand. In some embodiment, the loop/bulges are created following a pattern of . . . , −35, −20, −5, 0, +30, +45, +60, . . . etc., wherein 0 is the site of desired chemical modification. Schematic representations of the foregoing are provided in FIG. 2B.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells, or tissues.

The term “lentivirus” as used herein refers to a member of the class of viruses associated with this name and belonging to the genus lentivirus, family Retroviridae. While some lentiviruses can cause diseases, other lentivirus can be suitable for gene delivery.

“Messenger RNA” or “mRNA” is a nucleic acid molecule that is transcribed from DNA and then processed to remove non-coding sections known as introns. In some cases, the resulting mRNA is exported from the nucleus (or another locus where the DNA is present) and translated into a protein. The term “pre-mRNA” can refer to the strand prior to processing to remove non-coding sections.

The term “mutation” as used herein, can refer to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” can refer to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation can indicate that a tissue or sample is benign. As an alternative, absence of a mutation may not indicate that a tissue or sample is benign. Methods as described herein can comprise identifying a presence of a mutation in a sample.

The term “off-target-inhibitory mismatch” refers to a loop or bulge in a targeting domain (antisense domain or region) of a targeting RNA comprising a “G” opposite a non-targeted “A” in a target RNA. Typically, the off-target-inhibitory mismatches are located at −5 bp from the targeted “A” to be modified (modification mismatch) and then optionally about every 15 bp 5′ from the modification mismatch, and +30 bp from the modification mismatch and then about every 15 bp 3′ from the +30 off-target inhibitory mismatch. These off-target-inhibitory mismatches reduce off target modifications of “A” in the target RNA.

The terms “polynucleotide” and “oligonucleotide” are used interchangeably and refer to a polymeric form of nucleotides of any length, either deoxyribonucleotides or ribonucleotides or analogs or combinations thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form. In some embodiments, a polynucleotide can include both RNA and DNA nucleotides.

The term “polynucleotide sequence” can be the alphabetical representation of a polynucleotide molecule. This alphabetical representation can be input into databases in a computer having a central processing unit and used for bioinformatics applications such as functional genomics and homology searching. In any alphabetic representation, the disclosure contemplates both RNA and DNA (wherein “T” is replaced with “U” or vice-a-versa).

The term “recruiting domain” refers to a polynucleotide sequence that can bind to or recruit one or more RNA editing entities. Exemplary recruiting domains can be an Alu domain, an APOBEC recruiting domain, a GluR2 domain, a Cas13 recruiting domain or any combination thereof.

The term “RNA editing entity” refers to a biological molecule that can cause a chemical modification of a nucleotide to change the nucleotide to a different nucleotide. In some embodiments, an RNA editing entity can be recruited to a particular site in a polynucleotide to cause a change in the nucleic acid sequence at a desired site. Examples of RNA editing entities include APOBEC protein (e.g., APOBEC1, APOBEC2, APOBEC3A, APOBEC3B, APOBEC3C, APOBEC3E, APOBEC3F, APOBEC3G, APOBEC3H, or APOBEC4 protein) or an ADAR protein (e.g., ADAR1, ADAR2, or ADAR3 protein).

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient.

A “targeting domain” or “antisense region” refers to a polynucleotide sequence that can be at least partially complementary to a target RNA in a cell. The targeting domain is typically not 100% identical to the target RNA, but rather has mismatch(es) at one or more site where a chemical reaction is desired to modify the target RNA sequence. A targeting domain includes the complementary RNA antisense sequence to the target RNA as well as DNA sequence that encode (upon transcription) the antisense RNA sequence that is complementary to the RNA target sequence. The targeting domain is typically sufficiently complementary to the target RNA sequence to hybridize under biological condition to the target RNA sequence. In some instances, the targeting domain will comprise a plurality of off-target-inhibitory mismatches.

“Transfer ribonucleic acid” or “tRNA” is a nucleic acid molecule that helps translate mRNA to protein. tRNA have a distinctive folded structure, comprising three hairpin loops; one of these loops comprises a “stem” portion that encodes an anticodon. The anticodon recognizes the corresponding codon on the mRNA. Each tRNA is “charged with” an amino acid corresponding to the mRNA codon; this “charging” is accomplished by the enzyme tRNA synthetase. Upon tRNA recognition of the codon corresponding to its anticodon, the tRNA transfers the amino acid with which it is charged to the growing amino acid chain to form a polypeptide or protein. Endogenous tRNA can be charged by endogenous tRNA synthetase. Accordingly, endogenous tRNA are typically charged with canonical amino acids. Orthogonal tRNA, derived from an external source, require a corresponding orthogonal tRNA synthetase. Such orthogonal tRNAs may be charged with both canonical and non-canonical amino acids. In some embodiments, the amino acid with which the tRNA is charged may be detectably labeled to enable detection in vivo. Techniques for labeling include, but are not limited to, click chemistry wherein an azide/alkyne containing unnatural amino acid is added by the orthogonal tRNA/synthetase pair and, thus, can be detected using alkyne/azide comprising fluorophore or other such molecule.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder. Also, the terms “treat”, “treating” and “treatment” include preventing or delaying the onset of the disease or disorder symptoms, and/or lessening the severity or frequency of symptoms of the disease or disorder.

As used herein, the term “vector” can refer to a nucleic acid construct deigned for transfer between different hosts, including but not limited to a plasmid, a virus, a cosmid, a phage, a bacterial artificial chromosome (BAC), a yeast artificial chromosome (YAC), etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves. Alphavirus vectors, such as Semliki Forest virus-based vectors and Sindbis virus-based vectors, have also been developed for use in gene therapy and immunotherapy. In aspects where gene transfer is mediated by a retroviral vector, a vector construct can refer to the polynucleotide comprising the retroviral genome or part thereof, and a gene of interest.

Adenosine to inosine (A-to-I) RNA editing is a post-transcriptional RNA modification catalyzed by Adenosine Deaminases acting on RNA (ADAR) enzymes. ADARs edit double stranded RNA (dsRNA), predominantly in non-coding regions such as Alu repetitive elements while also editing sites in coding regions, leading to alterations in protein function. The structural similarity between inosine and guanosine accounts for the translation and splicing machinery recognizing the edited base as guanosine, thereby making ADARs tools for altering protein sequences. ADAR enzymes can be used for site-specific RNA editing by recruiting them to a target RNA sequence, using engineered ADAR recruiting RNAs (adRNAs), both in vitro and in vivo. In some cases, editing can rely on exogenously expressed ADAR enzymes and their variants. In some cases, a limitation of using exogenous enzyme overexpression is its propensity to introduce large number of off-target A-to-I edits across the transcriptome. A potential solution to this problem is the engineering of adRNAs to enable recruitment of endogenous ADARs which are expressed across a variety of different cell types. In some cases, using a long antisense RNA of length 100 bp suffices to recruit endogenous ADARs and these long antisense RNA are both genetically encodable and chemically synthesizable. The use of both genetically encodable long antisense RNA as well as chemically modified antisense oligonucleotides enabled highly transcript specific RNA editing. Additionally, chemically modified antisense oligonucleotides can be expensive to synthesize. On the contrary, genetically encodable adRNA can be delivered as DNA, and transcribed by the cell itself via an H1, U6 or similar promoter or be delivered as RNA when synthesized by in vitro transcription. The use of genetically encodable adRNA can be cheaper and more convenient than chemically modified antisense oligonucleotides.

In some cases, a hurdle in the RNA editing space can be guide stability. An adRNA may be present for extended periods of time in order to successfully recruit endogenous ADARs, but single stranded RNAs may have a half-life of about 30 minutes or less in mammalian cells. This may be due to their susceptibility to exonucleases that may degrade single stranded RNA from the 5′ or 3′ ends. Modifications may be made to a guide RNA to increase guide stability. As described herein, forming a circular guide RNA may be one type of modification to enhance guide RNA stability. Circularization may prevent exposed ends of a guide RNA from being degraded and may increase the half-life of a guide RNA, such as in vivo or in vitro. In some cases, a circular guide RNA may prevent one or more exposed ends from hydrolytic degradation. In some cases, a circular guide RNA may increase a half-life of the guide RNA as compared to a comparable guide RNA that is not circular. In some cases, forming a circular guide RNA may increase a half-life of a guide RNA when delivered in vivo, such as to a subject, as compared to a comparable guide RNA that is not circular. In some cases, forming a circular guide RNA may reduce an amount (such as a therapeutically effective amount) of the guide RNA dosed to a subject as compared to a comparable guide RNA that is not circular. In some cases, forming a circular guide RNA may enhance efficiency of editing, may reduce off target editing, or a combination thereof as compared to a comparable guide RNA that is not circular. In some cases, a circular guide RNA herein may have reduced hyperediting (e.g., off target editing of non-target adenosine). In some cases, a circular guide RNA comprising one or more loops may have decreased hyperediting as compared to a circular engineered guide RNA without a least one 8-bp loop as measured by an in vivo assay.

Circular guide RNAs may provide various benefits as compared to non-circular guide RNAs. Circular guides may provide greater stability, improved recruitment of RNA editing entities (such as endogenous RNA editing enzymes), longer half-lives, or any combination thereof as compared to a comparable guide RNA that is not circular. Circular guide RNA may provide one or more of these improved qualities and may retain genetic encodability as compared guide RNAs comprising other types of modifications designed to improve guide stability—such as chemical modifications or sugar additions. Circular guide RNAs may be capable of being genetically encoded, capable of being delivered by a vector, and retain improved stability. A circular engineered guide RNA may be less susceptible to hydrolytic degradation than an mRNA naturally present in a human cell. A circular engineered guide RNA may also retain a substantially similar secondary structure as a substantially similar engineered guide RNA that is not circular. In some cases, an encoded engineered guild RNA can be codon optimized.

An aspect of the disclosure provides for engineered guide RNAs, vectors comprising engineered guide RNAs, compositions, and pharmaceutical compositions for RNA editing. Any of the above or as described herein can be configured for an A (adenosine) to I (inosine) edit, a C (cytosine) to T (thymine) edit, or a combination thereof. In some cases, an A to I edit can be interpreted or read as a C to U mutation. In some cases, upon editing of an A to an I, the I can be interpreted or read by cellular machinery as a G. Engineered guide RNAs, vectors comprising engineered guide RNAs, compositions, and pharmaceutical compositions as described herein can provide enhanced editing efficiencies as compared to native systems, reduced off-target editing, enhanced stability or in vivo half-lives, or any combination thereof.

An aspect of the disclosure provides for a vector. The vector can comprise a nucleic acid with a polynucleotide sequence encoding (i) an RNA editing entity recruiting domain, or (ii) a targeting domain complementary to at least a portion of a target RNA, or (iii) optionally more than one of either domain (i) and/or (ii), or (iv) any combination thereof. In some cases, the vector can be administered to a subject, such as a subject in need thereof. In some cases, the vector can be administered as part of a pharmaceutical composition to a subject, such as a subject in need thereof. In some cases, the polynucleotide sequence encodes for a circular guide RNA or a linear precursor thereof.

An aspect of the disclosure provides for a non-naturally occurring RNA. In some cases, a non-naturally occurring RNA can refer to an engineered RNA, for example, an engineered guide RNA. In some instances, an engineered RNA can refer to a non-naturally occurring RNA. The non-naturally occurring RNA can comprise (i) an RNA editing entity recruiting domain, or (ii) a targeting domain complementary to at least a portion of a target RNA, or (iii) optionally more than one of either domain (i) and/or (ii), or (iv) any combination thereof. In some cases, the non-naturally occurring RNA is circular. In some cases, the non-naturally occurring RNA does not comprise (lacks) an exposed end or a single stranded end. In some cases, the non-naturally occurring RNA can be administered to a subject, such as a subject in need thereof. In some cases, the non-naturally occurring RNA can be administered as part of a pharmaceutical composition to a subject, such as a subject in need thereof. In some cases, the non-naturally occurring RNA can be formulated in a vector for administration. The vector can comprise a viral vector, a liposome, a nanoparticle, or any combination thereof. In some cases, the non-naturally occurring RNA can comprise at least one base, at least one sugar, more than one of either, or a combination thereof having a modification, such as a chemical modification.

Two-dimensional shape or secondary structure of a domain can influence efficiency of editing, off target effects, or a combination thereof as compared to a nucleic acid that can form a different two-dimension shape or secondary structure. Therefore, an aspect of the disclosure includes modifying nucleic acids such that two dimensional shapes can be advantageously designed to enhance efficiency of editing and reduce off target effects. Modifications to a sequence comprising a naturally occurring recruiting domains can also enhance editing efficiency and reduce off target effects. Therefore, an aspect of the disclosure includes modifying nucleic acids such that a sequence (such as a synthetic sequence) can be advantageously designed to enhance efficiency of editing and reduce off target effects. Modifications can include altering a length of a domain (such as extending a length), altering a native sequence that results in a change in secondary structure, adding a chemical modification, or any combination thereof. Nucleic acids as described herein can provide these advantages. Modifications can include providing the guide RNA in a circular form. Modifications can include forming a circular guide RNA to remove one or more exposed ends or one or more single stranded ends. Circularization of a guide RNA may permit the guide RNA to retain a secondary structure, such as a stem loop or cruciform.

An engineered guide RNA herein may be circular. An engineered guide RNA may not comprise a 5′ reducing hydroxyl capable of being exposed to a solvent. An engineered guide RNA may not comprise a 5′ reducing hydroxyl, 3′ reducing hydroxyl, or both capable of being exposed to a solvent. A circular engineered guide RNA may comprise a recruiting domain, a targeting domain (an antisense region), or both. The circular engineered guide RNA may recruit an RNA editing entity, such as an enzyme, to edit a base of an RNA sequence. A circular engineered guide RNA may be pre-strained. A circular engineered guide RNA may comprise a decreased level of entropy.

In some embodiments, an engineered polynucleotide may not comprise (lacks) a 5′ reducing hydroxyl, a 3′ reducing hydroxyl, or both, capable of being exposed to a solvent. In some cases, each 5′ hydroxyl, and each 3′ hydroxyl may be independently bonded to a phosphorous by a covalent oxygen phosphorous bond. In some instances, the phosphorous may be contained in a phosphodiester group.

An engineered guide RNA can comprise one or more domains, such as 1, 2, 3, 4, 5 or more domains. In some cases, an engineered guide RNA can comprise a recruiting domain, a targeting domain, more than one of either, or a combination thereof. In some cases, an engineered guide RNA can comprise a targeting domain and a recruiting domain. In some cases, an engineered guide RNA can comprise a targeting domain and two recruiting domains. In some cases, a circular engineered guide RNA can comprise 1, 2, 3, 4, 5, or more different targeting domains. In some cases, a circular engineered guide RNA can comprise 1, 2, 3, 4, 5, or more identical targeting domains.

A domain can form a two-dimensional shape or secondary structure. For example, an antisense region, a recruiting domain or a combination thereof can form a secondary structure that can comprise a linear region, a cruciform or portion thereof, a toe hold, a stem loop, or any combination thereof. The domain itself can form a substantially linear two-dimensional structure. The domain can form a secondary structure that can comprise a cruciform. The domain can form a secondary structure that can comprise a stem loop. The domain can form a secondary structure that can comprise a toehold.

In some cases, a targeting domain (an antisense region) can be positioned adjacent to a recruiting domain, including immediately adjacent or adjacent to but separated by a number of nucleotides (e.g., 1, 2, 3, 4, 5, 6, 7, 8, 9, 10, 15, 20, 25, 30 or more nucleotides). In some cases, a targeting domain can be flanked by two recruiting domains. In some cases, two or more recruiting domains can be adjacent to one another.

A circular engineered guide RNA may comprise a recruiting domain, such as an RNA editing entity recruiting domain that may recruit an RNA editing entity to perform a chemical transformation on a base in an RNA sequence. The recruiting domain may recruit an endogenous RNA editing entity or an exogenous RNA editing entity. In some aspects, a circular engineered guide RNA may not comprise a separate recruiting domain, or may not comprise a recruiting domain. The RNA editing entity may be an enzyme, such as an endogenous enzyme or a recombinant enzyme. The enzyme may perform the edit to the base. The circular engineered guide RNA may also comprise a targeting domain.

In some cases, a recruiting domain can comprise at least about: 80%, 85%, 90%, or 95% sequence homology to at least about: 15, 20, 25, 30, or 35 nucleic acids of an Alu domain. In some cases, at least a portion of a recruiting domain can comprise at least about 80% sequence homology to an Alu domain encoding sequence. In some cases, at least a portion of a recruiting domain can comprise at least about 85% sequence homology to an Alu domain encoding sequence. In some cases, at least a portion of a recruiting domain can comprise at least about 90% sequence homology to an Alu domain encoding sequence. In some cases, at least a portion of a recruiting domain can comprise at least about 95% sequence homology to an Alu domain encoding sequence. In some cases, the Alu domain encoding sequence can be a non-naturally occurring sequence. In some cases, the Alu domain encoding sequence can comprise a modified portion. In some cases, the Alu domain encoding sequence can comprise a portion of a naturally occurring Alu domain sequence.

In some cases, a recruiting domain can comprise at least about: 80%, 85%, 90%, or 95% sequence homology to at least about: 15, 20, 25, 30, or 35 nucleic acids of an APOBEC recruiting domain. In some cases, at least a portion of a recruiting domain can comprise at least about 80% sequence homology to an APOBEC recruiting domain encoding sequence. In some cases, at least a portion of a recruiting domain can comprise at least about 85% sequence homology to an APOBEC recruiting domain encoding sequence. In some cases, at least a portion of a recruiting domain can comprise at least about 90% sequence homology to an APOBEC recruiting domain encoding sequence. In some cases, at least a portion of a recruiting domain can comprise at least about 95% sequence homology to an APOBEC recruiting domain encoding sequence. In some cases, the APOBEC recruiting domain encoding sequence can be a non-naturally occurring sequence. In some cases, the APOBEC recruiting domain encoding sequence can comprise a modified portion. In some cases, the APOBEC recruiting domain encoding sequence can comprise a portion of a naturally occurring APOBEC recruiting domain sequence.

In some cases, a recruiting domain can comprise at least about: 80%, 85%, 90%, or 95% sequence homology to at least about: 15, 20, 25, 30, or 35 nucleic acids of an GluR2 domain. In some cases, at least a portion of a recruiting domain can comprise at least about 80% sequence homology to a GluR2 domain encoding sequence. In some cases, at least a portion of a recruiting domain can comprise at least about 85% sequence homology to a GluR2 domain encoding sequence. In some cases, at least a portion of a recruiting domain can comprise at least about 90% sequence homology to a GluR2 domain encoding sequence. In some cases, at least a portion of a recruiting domain can comprise at least about 95% sequence homology to a GluR2 domain encoding sequence. In some cases, the GluR2 domain encoding sequence can be a non-naturally occurring sequence. In some cases, the GluR2 domain encoding sequence can comprise a modified portion. In some cases, the GluR2 domain encoding sequence can comprise a portion of a naturally occurring GluR2 domain sequence.

In some cases, a recruiting domain can comprise at least about: 80%, 85%, 90%, or 95% sequence homology to at least about: 15, 20, 25, 30, or 35 nucleic acids of a Cas13 recruiting domain. The Cas13 recruiting domain may be a Cas13a recruiting domain, a Cas13b recruiting domain, a Cas13c recruiting domain, or a Cas 13d recruiting domain. In some cases, at least a portion of a recruiting domain can comprise at least about 80% sequence homology to a Cas13 recruiting domain encoding sequence. In some cases, at least a portion of a recruiting domain can comprise at least about 85% sequence homology to a Cas13 recruiting domain encoding sequence. In some cases, at least a portion of a recruiting domain can comprise at least about 90% sequence homology to a Cas13 recruiting domain encoding sequence. In some cases, at least a portion of a recruiting domain can comprise at least about 95% sequence homology to a Cas13 recruiting domain encoding sequence. In some cases, the Cas13 recruiting domain encoding sequence can be a non-naturally occurring sequence. In some cases, the Cas13 recruiting domain encoding sequence can comprise a modified portion. In some cases, the Cas13 recruiting domain encoding sequence can comprise a portion of a naturally occurring Cas13 recruiting domain sequence.

An engineered polynucleotide (e.g., an engineered guide RNA) may comprise a targeting domain that may be at least partially complementary to a target RNA. In some cases, the engineered guide RNA can comprise a backbone comprising a plurality of sugar and phosphate moieties covalently linked together. In some cases, the backbone may not comprise (lacks) a 5′ reducing hydroxyl, a 3′ reducing hydroxyl, or both, capable of being exposed to a solvent. In some cases, the engineered guide RNA may have an RNA editing entity recruiting domain. The RNA editing entity recruiting domain may be configured to interact with an RNA editing entity, such as, for example, ADAR1 or ADAR2. In some cases, the engineered guide RNA may not have (lacks) an RNA editing entity recruiting domain.

In some cases, a circular engineered polynucleotide can comprise a targeting domain (an antisense region). In some cases, the targeting domain may be configured to at least partially associate with a coding region of a target RNA. In some cases, a targeting domain can be at least partially complementary to a target RNA. In some cases, a targeting domain with at least partial complementarity can comprise a polynucleotide sequence with at least about 80% sequence homology to a reverse complement of the target RNA. In some instances, a targeting domain with at least partial complementarity can comprise a polynucleotide sequence with at least about 70%, at least about 80%, or at least about 90% sequence homology to the reverse complement of the target RNA. In some instances, a targeting domain can comprise a sequence with at least about 70%, at least about 80%, or at least about 90% complementarity to at least a portion of the target RNA. In some cases, the targeting domain can at least partially bind to a target RNA that may be implemented in a disease or condition. The association of the targeting domain and the target RNA may facilitate an edit of a base by an RNA editing entity such as ADAR1, ADAR2, APOBEC, or a combination thereof. In some cases, a circular engineered polynucleotide may further comprise an RNA editing entity recruiting domain. In some cases, an edit of a base may be a chemical transformation of a base. In some embodiments, the target RNA can comprise a nonsense mutation, a missense mutation, or both. In some cases, a targeting domain can comprise at least a single nucleotide that may be mismatched to the target RNA. In some instances, the mismatched nucleotide on the targeting domain can be adjacent to two nucleotides, one on each side of the mismatched nucleotide, which may be complementary to the target RNA.

In some embodiments, a circular engineered guide RNA can comprise an antisense region with partial complementarity to a region of a target RNA sequence. In some cases, a target RNA sequence can comprise a transcript of ALDOA, DAXX, IDUA, FANCC, CTNNB1, SMAD4, or TARDBP. In some instances, the circular engineered guide RNA can be configured to facilitate editing of a target nucleotide in a target RNA sequence by an RNA editing entity. In some cases, a circular engineered guide RNA can further comprise an RNA editing entity recruiting domain. In some instances, an RNA editing recruiting domain can comprise at least about 80% sequence identity to at least about 20 contiguous nucleic acids of: an Alu domain (Seq ID NO: 1421), an APOBEC recruiting domain (SEQ ID NO:1541 or a fragment thereof), or a GluR2 domain (Seq ID NO: 1419 and 1420). In some cases, the RNA editing entity recruiting domain can recruit an RNA editing entity that, when associated with the circular engineered guide RNA and the target RNA sequence, performs a chemical transformation on a base of a target nucleotide in the target RNA sequence, thereby generating an edited target RNA sequence. In some cases, a protein translated from the edited target sequence is longer than a protein translated from an unedited target sequence as demonstrated in an in vitro assay. In some cases, a chemical transformation can transform a stop codon into a sense codon. In some cases, a chemical transformation can edit a missense or a nonsense mutation. In some cases, a protein translated from the edited target sequence is longer than a protein translated from an unedited target sequence as demonstrated in an in vitro assay. In some cases, the circular engineered guide RNA comprises at least about: 75%, 80%, 85%, 90%, 91%, 92%, 93%, 94%, 95%, 96%, 97%, 98%, 99%, or 100%, sequence identity to 50-200 nucleotides of SEQ ID NO: 1418 containing nucleotides 1204-1206. In some cases, the circular engineered guide RNA comprises at least about: 75%, 80%, 85%, 90%, 91%, 92%, 93%, 94%, 95%, 96%, 97%, 98%, 99%, or 100%, sequence identity to 50-200 nucleotides of SEQ ID NO: 1438-1445. In some embodiments, the antisense region can comprise a sequence length from about: 20 nucleotides to about 1000 nucleotides, 50 nucleotides to about 200 nucleotides, or 60 nucleotides to about 100 nucleotides. In some cases, the circular engineered guide RNA comprising an antisense region of about: 100 bp or more or about 100 bp to about 200 bp has at least about: a 2-fold increase, a 3-fold increase, or a 3.5-fold increase in RNA editing as compared to a comparable linear engineered guide RNA as measured by an in vitro assay. In some cases, the circular engineered guide RNA does not comprise a G mismatch opposite all non-target adenosines. In some cases, the circular engineered guide RNA comprises 1, 2, 3, 4, 5, 6, or more mismatched guanines opposite all non-target adenosines. In some cases, the circular engineered guide RNA comprises at least one 8-bp loop. In some cases, the circular engineered guide RNA comprises 1, 2, 3, 4, 5, 6 or more 8-bp loops. In some cases, a circular engineered guide with an 8-bp loop can have decreased hyperediting as compared to a circular engineered guide RNA without the at least one 8-bp loop as measured by an in vitro assay.

In some embodiments, a chemical transformation, such as a chemical transformation by an RNA editing entity, may comprise an edit of a base. In some embodiments, a chemical transformation, such as an edit of a base may result in an increased level of a protein or fragment thereof after translation of a target RNA with the chemical transformation, relative to an otherwise comparable target RNA lacking the chemical transformation. In some cases, an increased level can be from about: 5% to about 100%, 10% to about 50%, 25% to about 75%, or from about 40% to about 90%. In some embodiments, a chemical transformation can result in a decreased level of a protein or fragment thereof after translation of a target RNA with the chemical transformation, relative to an otherwise comparable target RNA lacking the chemical transformation. In some cases, a decreased level can be from about: 5% to about 99%, 10% to about 50%, 25% to about 75%, or from about 40% to about 90%. In some embodiments, a chemical transformation can result in an increased length of a protein or fragment thereof, an increased functionality of a protein or fragment thereof, increased stability of a protein or fragment thereof, or any combination thereof after translation of the target RNA with the edit of the base, relative to a translated protein of an otherwise comparable target RNA lacking the edit. In some cases, an increased length can be from about: 5% to about 100%, 2% to about 10%, 10% to about 25%, 25% to about 50%, 40% to about 80%, or about 75% to about 150%. In some cases, the increased length of a protein or a fragment thereof can be over 100%. In some cases, the increased stability can be an increased half-life of the protein or fragment thereof. In some cases, the increased half-life can be at least about: 2×, 3×, 4×, 5×, 6×, 7×, 8×, 9×, or 10× greater than to a translated protein of an otherwise comparable target RNA lacking the edit. In some cases, increased functionality can comprise a protein or fragment thereof, such as an enzyme that may increase the speed of a reaction, increase the Vₘₐₓ, or both. In some cases, increased functionality may comprise a protein (e.g., an enzyme) or fragment thereof, encoded by a target RNA with the edit of the base, comprising a lower energy of activation as compared to a translated protein of an otherwise comparable target RNA lacking the edit.

The chemical transformation on the base may include editing one or more bases of the targeted RNA sequence. The chemical transformation on a base may edit a sense codon to a stop codon, a stop codon to a sense codon, a first sense codon to a second sense codon, or a first stop codon to a second stop codon. In some cases, the chemical transformation can covert a sense codon specifying a first amino acid into a second sense codon specifying a second amino acid. In some cases, the first amino acid can flank a protease cleavage site.

In some embodiments, RNA editing may be determined in an in vitro assay by transfecting a target RNA and an engineered polynucleotide designed to target the target RNA into the same cell. The target RNA may be sequenced to identify editing by the engineered polynucleotide. In some cases, transfecting a target RNA into a primary cell line can comprise transfecting a plasmid encoding for the target RNA into a primary cell line. In some instances, transfecting an engineered polynucleotide into a primary cell line can comprise transfecting a plasmid that encodes for an engineered polynucleotide into a primary cell line. In some cases, the percent RNA editing of a target RNA can be determined at different time points (e.g., 24 hours, 48 hours, 96 hours) after transfection with a guide RNA or engineered polynucleotide by reverse transcribing the target RNA to cDNA then using Sanger sequencing to determine the percent RNA editing of a target RNA. In some cases, the cDNA can be amplified prior to sequencing by polymerase chain reaction. Sanger traces from Sanger sequencing can be analyzed to assess the editing efficiency of guide RNAs. In some cases, an isolated cell can comprise an engineered guide described herein. In some cases, a cell can be a primary cell. In some cases, a primary cell or a cell can be a neuron, a photoreceptor cell (e.g., a S cone cell, a L cone cell, a M cone cell, a rod cell), a retinal pigment epithelium cell, a glia cell (e.g., an astrocyte, an oligodendrocyte, a microglia), a muscle cell (e.g., a myoblast, a myotube), a hepatocyte, a lung epithelial cell, or a fibroblast (e.g., dermal fibroblast). In some cases, a cell can be a horizontal cell, a ganglion cell, or a bipolar cell. In some cases, a cell line can be a mammalian cell line, such as HEK293T, NCI-60, MCF-7, HL-60, RD, LHCN differentiated, LHCN undifferentiated, Saos-2, CHO, or HeLa cells. In some cases, a cell line can be an insect cell line, such as Sf9.

In some instances, a polynucleotide sequence can share about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence homology to a sequence described herein. In some instances, the length of any sequence recited herein can be truncated to about: 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%, of the original sequence.

In some instances, a targeting domain (an antisense region) can have a sequence length of from about: 20 nucleotides to about 1,000 nucleotides, 10 nucleotides to about 100 nucleotides, 50 nucleotides to about 200 nucleotides, 60 nucleotides to about 100 nucleotides, 100 nucleotides to about 200 nucleotides, 50 nucleotides to about 500 nucleotides or about 400 nucleotides to about 1000 nucleotides in length. In some instances, a targeting domain of an engineered polynucleotide, or a construct for forming an engineered polynucleotide, can comprise a polynucleotide sequence with at least about: 70%, 75%, 80%, 85%, 90%, or 95% homology to any one of the polynucleotides in SEQ ID NOs:1-1417 (See also, Tables 1-12). In some instances, the sequences in Tables 1-12 can at least in part encode for the targeting domain of an engineered polynucleotide, or a construct for forming an engineered polynucleotide. In some instances, in Tables 1-12, a T (thymine) can be substituted with a U (uracil) in a polynucleotide. In some instances, in Tables 1-12, all Ts can be substituted with Us in a polynucleotide. In some instances, the sequences in Tables 1-12 can at least in part encode for a targeting domain of an engineered polynucleotide and will comprise a “C” opposite an “A” in the target RNA to be chemically modified and may further comprise one or more (e.g., 2, 3, 4, 5, 6, 7, 8, 9, 10 or more) “G” nucleotides opposite non-targeted “A” nucleotides in the target RNA. In some instances, a targeting domain of a construct for forming an engineered polynucleotide, or an engineered polynucleotide, can comprise a polynucleotide sequence with at least about: 70%, 75%, 80%, 85%, 90%, or 95% sequence length to any one of the polynucleotides in Tables 1-12. In some instances, a targeting domain of a construct for forming an engineered polynucleotide, or an engineered polynucleotide, can comprise a polynucleotide sequence with at least about: 70%, 75%, 80%, 85%, 90%, or 95% sequence length to any one of the polynucleotides in Tables 1-12 and a polynucleotide sequence with at least about: 70%, 75%, 80%, 85%, 90%, or 95% homology to any one of the polynucleotides in Tables 1-12. In some instances, an engineered polynucleotide can comprise a polynucleotide sequence with at least about 80% sequence homology to any one of the polynucleotides in Table 1. In some instances, an engineered polynucleotide can comprise a polynucleotide sequence with at least about 80% sequence homology to any one of the polynucleotides in Table 2. In some instances, an engineered polynucleotide can comprise a polynucleotide sequence at least about 80% sequence homology to any one of the polynucleotides in Table 3. In some instances, an engineered polynucleotide can comprise a polynucleotide sequence with at least about 80% sequence homology to any one of the polynucleotides in Table 4. In some instances, an engineered polynucleotide can comprise a polynucleotide sequence at least about 80% sequence homology to any one of the polynucleotides in Table 5. In some instances, an engineered polynucleotide can comprise a polynucleotide sequence with at least about 80% sequence homology to any one of the polynucleotides in Table 6. In some instances, an engineered polynucleotide can comprise a polynucleotide sequence at least about 80% sequence homology to any one of the polynucleotides in Table 7. In some instances, an engineered polynucleotide can comprise a polynucleotide sequence with at least about 80% sequence homology to any one of the polynucleotides in Table 8. In some instances, an engineered polynucleotide can comprise a polynucleotide sequence with at least about 80% sequence homology to any one of the polynucleotides in Table 9. In some instances, an engineered polynucleotide can comprise a polynucleotide sequence with at least about 80% sequence homology to the polynucleotide in Table 10. In some instances, an engineered polynucleotide can comprise a polynucleotide sequence with at least about 80% sequence homology to any one of the polynucleotides in Table 11. In some instances, an engineered polynucleotide can comprise a polynucleotide sequence with at least about 80% sequence homology to any one of the polynucleotides in Table 12.

TABLE 1
(it will be recognized that the sequences below
can be RNA or DNA (T can be U or vice-a-versa
unless methylation clearly indicates otherwise).
For example, a DNA sequence can be expressed
from a vector to produce RNA)

| SEQ ID | |
|---|---|
| NO. | Sequence 5′ -> 3 |
| 1 | G*G*G*C*A*C*A*A*G*G*G*C*A*C*A*G*A*C*T*T |
| 2 | G*G*C*A*C*A*A*G*G*G*C*A*C*A*G*A*C*T*T*C |
| 3 | G*C*A*C*A*A*G*G*G*C*A*C*A*G*A*C*T*T*C*C |
| 4 | C*A*C*A*A*G*G*G*C*A*C*A*G*A*C*T*T*C*C*A |
| 5 | A*C*A*A*G*G*G*C*A*C*A*G*A*C*T*T*C*C*A*A |
| 6 | C*A*A*G*G*G*C*A*C*A*G*A*C*T*T*C*C*A*A*A |
| 7 | mG*mG*mG*mC*mA*C*A*A*G*G*G*C*A*C*A*G*A*C*T*T |
| 8 | mG*mG*mC*mA*mC*A*A*G*G*G*C*A*C*A*G*A*C*T*T*C |
| 9 | mG*mC*mA*mU*mA*A*G*G*G*C*A*C*A*G*A*C*T*T*C*C |
| 10 | mC*mA*mC*mA*mA*G*G*G*C*A*C*A*G*A*C*T*T*C*C*A |
| 11 | mA*mC*mA*mA*mG*G*G*C*A*C*A*G*A*C*T*T*C*C*A*A |
| 12 | mC*mA*mA*mG*mG*G*C*A*C*A*G*A*C*T*T*C*C*A*A*A |
| 13 | mG*mG*mG*mC*mA*C*A*A*G*G*G*C*A*C*A*mG*mA*mC*mU*mU |
| 14 | mG*mG*mC*mA*mC*A*A*G*G*G*C*A*C*A*G*mA*mC*mU*mU*mC |
| 15 | mG*mC*mA*mC*mA*A*G*G*G*C*A*C*A*G*A*mC*mU*mU*mC*mC |
| 16 | mC*mA*mU*mA*mA*G*G*G*C*A*C*A*G*A*C*mU*mU*mC*mC*mA |
| 17 | mA*mC*mA*mA*mG*G*G*C*A*C*A*G*A*C*T*mU*mC*mC*mA*mA |
| 18 | mC*mA*mA*mG*mG*G*C*A*C*A*G*A*C*T*T*mC*mC*mA*mA*mA |
| 19 | mG*mC*mA*mC*mA*mA*mG*mG*G*C*A*C*A*G*A*mC*mU*mU*mC*mC |
| 20 | mC*mA*mC*mA*mA*mG*mG*G*C*A*C*A*G*A*mC*mU*mU*mC*mC*mA |
| 21 | mA*mC*mA*mA*mG*mG*G*C*A*C*A*G*A*mC*mU*mU*mC*mC*mA*mA |
| 22 | mC*mA*mA*mG*mG*G*C*A*C*A*G*A*mC*mU*mU*mC*mC*mA*mA*mA |
| 23 | mGmCmAmCmAmAmGmG*G*C*A*C*A*G*A*mCmUmUmCmC |
| 24 | mCmAmCmAmAmGmG*G*C*A*C*A*G*A*mCmUmUmCmCmA |
| 25 | mAmCmAmAmGmG*G*C*A*C*A*G*A*mCmUmUmCmCmAmA |
| 26 | mCmAmAmGmG*G*C*A*C*A*G*A*mCmUmUmCmCmAmAmA |
| 27 | mGmGmGmCmA*C*A*A*G*G*G*C*A*C*A*mGmAmCmUmU |
| 28 | mGmGmCmAmC*A*A*G*G*G*C*A*C*A*G*mAmCmUmUmC |
| 29 | mGmCmAmCmA*A*G*G*G*C*A*C*A*G*A*mCmUmUmCmC |
| 30 | mCmAmCmAmA*G*G*G*C*A*C*A*G*A*C*mUmUmCmCmA |
| 31 | mAmCmAmAmG*G*G*C*A*C*A*G*A*C*T*mUmCmCmAmA |
| 32 | mCmAmAmGmG*G*C*A*C*A*G*A*C*T*T*mCmCmAmAmA |
| 33 | G*SG*SG*SC*SA*SC*SA*SA*SG*SG*SG*SC*SA*SC*RA*SG*SA*SC* |
| | ST*ST |
| 34 | G*SG*SC*SA*SC*SA*SA*SG*SG*SG*SC*SA*SC*RA*SG*SA*SC*ST* |
| | ST*SC |
| 35 | G*SC*SA*SC*SA*SA*SG*SG*SG*SC*SA*SC*RA*SG*SA*SC*ST*ST* |
| | SC*SC |
| 36 | C*SA*SC*SA*SA*SG*SG*SG*SC*SA*SC*RA*SG*SA*SC*ST*ST*SC* |
| | SC*SA |
| 37 | A*SC*SA*SA*SG*SG*SG*SC*SA*SC*RA*SG*SA*SC*ST*ST*SC*SC* |
| | SA*SA |
| 38 | C*SA*SA*SG*SG*SG*SC*SA*SC*RA*SG*SA*SC*ST*ST*SC*SC*SA* |
| | SA*SA |
| 39 | mG*SmG*SmC*SmA*SmC*SA*SA*SG*SG*SG*SC*SA*SC*RA*SG*SmA* |
| | SmC*SmU*SmU*SmC |
| 40 | mG*RmG*RmC*RmA*RmC*SA*SA*SG*SG*SG*SC*SA*SC*RA*SG*SmA* |
| | RmC*RmU*RmU*RmC |
| 41 | mGmGmCmAmC*SA*SA*SG*SG*SG*SC*SA*SC*RA*SG*SmAmCmUmUmC |
| 42 | mG*SmG*SmC*SmA*SmC*SmA*SmA*SmG*SG*SG*SC*SA*SC*RA*SG* |
| | SA*SC*ST*ST*SC |
| 43 | mG*RmG*RmC*RmA*RmU*RmA*RmA*RmG*SG*SG*SC*SA*SC*RA*SG* |
| | SA*SC*ST*ST*SC |
| 44 | mGmGmCmAmCmAmAmG*SG*SG*SC*SA*SC*RA*SG*SA*SC*ST*ST*SC |
| 45 | mG*RmGmCmAmC*SA*SA*SG*SG*SG*SC*SA*SC*RA*SG*SmAmCmUmU* |
| | RmC |
| 46 | mG*SmGmCmAmC*SA*SA*SG*SG*SG*SC*SA*SC*RA*SG*SmAmCmUmU* |
| | SmC |
| 47 | G*SC*SA*SG*SG*SG*SC*SA*SC*SA*SA*SG*SG*SG*SC*SA*SC*RA* |
| | SG*SA |
| 48 | C*SA*SG*SG*SG*SC*SA*SC*SA*SA*SG*SG*SG*SC*SA*SC*RA*SG* |
| | SA*SC |
| 49 | A*SG*SG*SG*SC*SA*SC*SA*SA*SG*SG*SG*SC*SA*SC*RA*SG*SA* |
| | SC*ST |
| 50 | A*SA*SG*SG*SG*SC*SA*SC*RA*SG*SA*SC*ST*ST*SC*SC*SA*SA* |
| | SA*SG |
| 51 | A*SG*SG*SG*SC*SA*SC*RA*SG*SA*SC*ST*ST*SC*SC*SA*SA*SA* |
| | SG*SG |
| 52 | G*SG*SG*SC*SA*SC*RA*SG*SA*SC*ST*ST*SC*SC*SA*SA*SA*SG* |
| | SG*SC |
| 53 | mG*mG*mC*mA*mC*A*A*G*G*G*C*A*C*A*G*mA*mC*mU*BrdU*mC |
| 54 | mG*mG*mC*mA*mC*A*A*G*G*G*C*A*C*A*G*mA*mC*BrdU*BrdU*mC |
| 55 | G*G*G*C*A*C*A*A*G*G*G*C*d2AP*C*A*G*A*C*T*T |
| 56 | G*G*C*A*C*A*A*G*G*G*C*d2AP*C*A*G*A*C*T*T*C |
| 57 | G*C*A*C*A*A*G*G*G*C*d2AP*C*A*G*A*C*T*T*C*C |
| 58 | G*G*G*C*A*C*A*A*G*G*G*C*dDAP*C*A*G*A*C*T*T |
| 59 | G*G*C*A*C*A*A*G*G*G*C*dDAP*C*A*G*A*C*T*T*C |
| 60 | G*C*A*C*A*A*G*G*G*C*dDAP*C*A*G*A*C*T*T*C*C |
| 61 | G*SmGmCmAmC*SA*SA*SG*SG*SG*SC*SA*SC*RA*SG*SmAmCmUmU* |
| | SC |
| 62 | G*mGmCmAmC*A*A*G*G*G*C*A*C*A*G*mAmCmUmU*C |
| 63 | mG*mGmCmAmC*A*A*G*G*G*C*A*C*A*G*mAmCmUmU*mC |
| 64 | Geo*Geom5CeoAeom5Ceo*A*A*G*G*G*C*A*C*A*G*Aeom5CeoTeo |
| | Teo*m5Ceo |
| 65 | G*A*G*C*A*G*C*T*G*C*A*A*C*C*T*G*G*C*A*A |
| 66 | A*G*C*A*G*C*T*G*C*A*A*C*C*T*G*G*C*A*A*C |
| 67 | G*C*A*G*C*T*G*C*A*A*C*C*T*G*G*C*A*A*C*A |
| 68 | C*A*G*C*T*G*C*A*A*C*C*T*G*G*C*A*A*C*A*A |
| 69 | A*G*C*T*G*C*A*A*C*C*T*G*G*C*A*A*C*A*A*C |
| 70 | G*C*T*G*C*A*A*C*C*T*G*G*C*A*A*C*A*A*C*C |
| 71 | mG*mA*mG*mC*mA*G*C*T*G*C*A*A*C*C*T*G*G*C*A*A |
| 72 | mA*mG*mC*mA*mG*C*T*G*C*A*A*C*C*T*G*G*C*A*A*C |
| 73 | mG*mC*mA*mG*mC*T*G*C*A*A*C*C*T*G*G*C*A*A*C*A |
| 74 | mC*mA*mG*mC*mU*G*C*A*A*C*C*T*G*G*C*A*A*C*A*A |
| 75 | mA*mG*mC*mU*mG*C*A*A*C*C*T*G*G*C*A*A*C*A*A*C |
| 76 | mG*mC*mU*mG*mC*A*A*C*C*T*G*G*C*A*A*C*A*A*C*C |
| 77 | mG*mA*mG*mC*mA*G*C*T*G*C*A*A*C*C*T*mG*mG*mC*mA*mA |
| 78 | mA*mG*mC*mA*mG*C*T*G*C*A*A*C*C*T*G*mG*mC*mA*mA*mC |
| 79 | mG*mC*mA*mG*mC*T*G*C*A*A*C*C*T*G*G*mC*mA*mA*mC*mA |
| 80 | mC*mA*mG*mC*mU*G*C*A*A*C*C*T*G*G*C*mA*mA*mC*mA*mA |
| 81 | mA*mG*mC*mU*mG*C*A*A*C*C*T*G*G*C*A*mA*mC*mA*mA*mC |
| 82 | mG*mC*mU*mG*mG*A*A*C*C*T*G*G*C*A*A*mC*mA*mA*mC*mC |
| 83 | mG*mA*mG*mC*mA*mG*mC*T*G*C*A*A*C*C*mU*mG*mG*mC*mA*mA |
| 84 | mGmAmGmCmAmGmC*T*G*C*A*A*C*C*mUmGmGmCmAmA |
| 85 | mA*mG*mC*mA*mG*mC*T*G*C*A*A*C*C*T*G*mG*mC*mA*mA*mC |
| 86 | mAmGmCmAmGmC*T*G*C*A*A*C*C*T*G*mGmCmAmAmC |
| 87 | mG*mG*mA*mG*mC*T*G*C*A*A*C*C*mG*mG*mG*mC*mA*mA*mC*mA |
| 88 | mGmCmAmGmC*T*G*C*A*A*C*C*mUmGmGmCmAmAmCmA |
| 89 | mGmAmGmCmA*G*C*T*G*C*A*A*C*C*T*mGmGmCmAmA |
| 90 | mAmGmCmAmG*C*T*G*C*A*A*C*C*T*G*mGmCmAmAmC |
| 91 | mGmCmAmGmC*T*G*C*A*A*C*C*T*G*G*mCmAmAmCmA |
| 92 | mCmAmGmCmU*G*C*A*A*C*C*T*G*G*C*mAmAmCmAmA |
| 93 | mAmGmCmUmG*C*A*A*C*C*T*G*G*C*A*mAmCmAmAmC |
| 94 | mGmCmUmGmC*A*A*C*C*T*G*G*C*A*A*mCmAmAmCmC |
| 95 | G*SA*SG*SC*SA*SG*SC*ST*SG*SC*SA*RA*SC*SC*ST*SG*SG*SC* |
| | SA*SA |
| 96 | A*SG*SC*SA*SG*SC*Sr4SG*SC*SA*RA*SC*SC*ST*SG*SG*SC*SA* |
| | SA*SC |
| 97 | G*SC*SA*SG*SC*ST*SG*SC*SA*RA*SC*SC*ST*SG*SG*SC*SA*SA* |
| | SC*SA |
| 98 | C*SA*SG*SC*ST*SG*SC*SA*RA*SC*SC*ST*SG*SG*SC*SA*SA*SC* |
| | SA*SA |
| 99 | A*SG*SC*ST*SG*SC*SA*RA*SC*SC*ST*SG*SG*SC*SA*SA*SC*SA* |
| | SA*SC |
| 100 | G*SC*ST*SG*SC*SA*RA*SC*SC*ST*SG*SG*SC*SA*SA*SC*SA*SA* |
| | SC*SC |
| 101 | G*G*G*C*C*A*A*C*A*G*C*C*A*G*C*C*T*G*C*A |
| 102 | G*G*C*C*A*A*C*A*G*C*C*A*G*C*C*T*G*C*A*G |
| 103 | G*C*C*A*A*C*A*G*C*C*A*G*C*C*T*G*T*A*G*G |
| 104 | C*C*A*A*C*A*G*C*C*A*G*C*C*T*G*C*A*G*G*A |
| 105 | C*A*A*C*A*G*C*C*A*G*C*C*T*G*C*A*G*G*A*G |
| 106 | A*A*C*A*G*C*T*A*G*C*C*T*G*C*A*G*G*A*G*G |
| 107 | mG*mG*mG*mC*mC*A*A*C*A*G*C*C*A*G*C*C*T*G*C*A |
| 108 | mG*mG*mC*mC*mA*A*C*A*G*C*C*A*G*C*C*T*G*C*A*G |
| 109 | mG*mC*mC*mA*mA*C*A*G*C*C*A*G*C*C*T*G*C*A*G*G |
| 110 | mC*mC*mA*mA*mC*A*G*C*C*A*G*C*C*T*G*C*A*G*G*A |
| 111 | mC*mA*mA*mC*mA*G*C*C*A*G*C*C*T*G*C*A*G*G*A*G |
| 112 | mA*mA*mC*mA*mG*T*C*A*G*C*C*T*G*C*A*G*G*A*G*G |
| 113 | mG*mG*mG*mC*mC*A*A*C*A*G*C*C*A*G*C*mC*mU*mG*mC*mA |
| 114 | mG*mG*mC*mC*mA*A*C*A*G*C*C*A*G*C*C*mU*mG*mC*mA*mG |
| 115 | mG*mC*mC*mA*mA*C*A*G*C*C*A*G*C*C*T*mG*mU*mA*mG*mG |
| 116 | mC*mC*mA*mA*mC*A*G*C*C*A*G*C*C*T*G*mC*mA*mG*mG*mA |
| 117 | mC*mA*mA*mU*mA*G*C*C*A*G*C*C*T*G*C*mA*mG*mG*mA*mG |
| 118 | mA*mA*mC*mA*mG*C*C*A*G*C*C*T*G*C*A*mG*mG*mA*mG*mG |
| 119 | mG*mG*mG*mC*mC*mA*mA*C*A*G*C*C*A*G*mC*mC*mU*mG*mC*mA |
| 120 | mGmGmGmCmCmAmA*C*A*G*C*C*A*G*mCmCmUmGmCmA |
| 121 | mG*mG*mC*mC*mA*mA*C*A*G*C*C*A*G*C*C*mU*mG*mC*mA*mG |
| 122 | mGmGmCmCmAmA*C*A*G*C*C*A*G*C*C*mUmGmCmAmG |
| 123 | mG*mC*mC*mA*mA*C*A*G*C*C*A*G*mC*mC*mU*mG*mC*mA*mG*mG |
| 124 | mGmCmCmAmA*C*A*G*C*C*A*G*mCmCmUmGmCmAmGmG |
| 125 | mGmGmGmCmC*A*A*C*A*G*C*C*A*G*C*mCmUmGmCmA |
| 126 | mGmGmCmCmA*A*C*A*G*C*C*A*G*C*C*mUmGmCmAmG |
| 127 | mGmCmCmAmA*C*A*G*C*C*A*G*C*C*T*mGmCmAmGmG |
| 128 | mCmCmAmAmC*A*G*C*C*A*G*C*C*T*G*mCmAmGmGmA |
| 129 | mCmAmAmCmA*G*C*C*A*G*C*C*T*G*C*mAmGmGmAmG |
| 130 | mAmAmCmAmG*C*C*A*G*C*C*T*G*C*A*mGmGmAmGmG |
| 131 | G*SG*SG*SC*SC*SA*SA*SC*SA*SG*SC*RC*SA*SG*SC*SC*ST*SG* |
| | SC*SA |
| 132 | G*SG*SC*SC*SA*SA*SC*SA*SG*SC*RC*SA*SG*SC*SC*ST*SG*SC* |
| | SA*SG |
| 133 | G*SC*SC*SA*SA*SC*SA*SG*SC*RC*SA*SG*SC*SC*ST*SG*SC*SA* |
| | SG*SG |
| 134 | C*SC*SA*SA*SC*SA*SG*SC*RC*SA*SG*SC*SC*ST*SG*SC*SA*SG* |
| | SG*SA |
| 135 | C*SA*SA*SC*SA*SG*SC*RC*SA*SG*SC*SC*ST*SG*SC*SA*SG*SG* |
| | SA*SG |
| 136 | A*SA*SC*SA*SG*SC*RC*SA*SG*SC*SC*ST*SG*SC*SA*SG*SG*SA* |
| | SG*SG |
| 137 | A*T*T*A*A*T*A*A*A*T*T*G*T*C*A*T*C*A*C*C |
| 138 | A*ST*ST*SA*SA*ST*SA*SA*SA*ST*ST*SG*ST*SC*RA*ST*SC*SA* |
| | SC*SC |
| 139 | A*ST*ST*SA*SA*ST*SA*SA*SA*ST*ST*SG*ST*SC*SA*RT*SC*SA* |
| | SC*SC |
| 140 | mA*SmU*SmU*SmA*SmA*SmU*SA*SA*SA*ST*ST*SG*ST*SC*RA*ST* |
| | SmC*SmA*SmC*SmC |
| 141 | mA*RmU*RmU*RmA*RmA*RmU*SA*SA*SA*ST*ST*SG*ST*SC*RA*ST* |
| | SmC*RmA*RmC*RmC |
| 142 | mA*SmU*SmU*SmA*SmA*SmU*SmA*SmA*SA*ST*ST*SG*ST*SC*RA* |
| | ST*SC*SA*SC*SC |
| 143 | mA*RmU*RmU*RmA*RmA*RmU*RmA*RmA*SA*ST*ST*SG*ST*SC*RA* |
| | ST*SC*SA*SC*SC |
| 144 | mAmUmUmAmAmUmAmA*SA*ST*ST*SG*ST*SC*RA*ST*SC*SA*SC*SC |
| 145 | mAmUmUmAmAmU*SA*SA*SA*ST*ST*SG*ST*SC*RA*ST*SmCmAmCmC |
| 146 | mA*SmUmUmAmAmU*SA*SA*SA*ST*ST*SG*ST*SC*RA*ST*SmCmAmC* |
| | SmC |
| 147 | mA*RmUmUmAmAmU*SA*SA*SA*ST*ST*SG*ST*SC*RA*ST*SmCmAmC* |
| | RmC |
| 148 | A*SmUmUmAmAmU*SA*SA*SA*ST*ST*SG*ST*SC*RA*ST*SmCmAmC* |
| | SC |
| 149 | A*mUmUmAmAmU*A*A*A*T*T*G*T*C*A*T*mCmAmC*C |
| 150 | T*G*T*C*A*T*C*A*C*C*A*G*A*A*A*mA*mA*mG*mU*mC |
| 151 | mU*T*G*T*C*A*T*C*A*C*C*A*G*A*A*mA*mA*mA*mG*mU |
| 152 | T*T*G*T*C*A*T*C*A*C*C*A*G*A*A*mA*mA*mA*mG*mU |
| 153 | mA*mU*T*G*T*C*A*T*C*A*C*C*A*G*A*mA*mA*mA*mA*mG |
| 154 | mA*T*T*G*T*C*A*T*C*A*C*C*A*G*A*mA*mA*mA*mA*mG |
| 155 | mA*mA*mU*T*G*T*C*A*T*C*A*C*C*A*G*mA*mA*mA*mA*mA |
| 156 | mA*mA*T*T*G*T*C*A*T*C*A*C*C*A*G*mA*mA*mA*mA*mA |
| 157 | mA*mA*mA*T*T*G*T*C*A*T*C*A*C*C*A*mG*mA*mA*mA*mA |
| 158 | mA*mA*mA*mU*T*G*T*C*A*T*C*A*C*C*A*mG*mA*mA*mA*mA |
| 159 | mil*mA*mA*mA*mU*T*G*T*C*A*T*C*A*C*C*A*mG*mA*mA*mA |
| 160 | mil*mA*mA*mA*mU*T*G*T*C*A*T*C*A*C*C*mA*mG*mA*mA*mA |
| 161 | mA*mU*mA*mA*mA*T*T*G*T*C*A*T*C*A*C*C*mA*mG*mA*mA |
| 162 | mA*mU*mA*mA*mA*T*T*G*T*C*A*T*C*A*C*mC*mA*mG*mA*mA |
| 163 | mA*mA*mU*mA*mA*A*T*T*G*T*C*A*T*C*A*C*C*mA*mG*mA |
| 164 | mA*mA*mU*mA*mA*A*T*T*G*T*C*A*T*C*A*C*mC*mA*mG*mA |
| 165 | mA*mA*mU*mA*mA*A*T*T*G*T*C*A*T*C*A*mC*mC*mA*mG*mA |
| 166 | mU*mA*mA*mU*mA*A*A*T*T*G*T*C*A*T*C*A*C*C*mA*mG |
| 167 | mU*mA*mA*mU*mA*A*A*T*T*G*T*C*A*T*C*A*C*mC*mA*mG |
| 168 | mU*mA*mA*mU*mA*A*A*T*T*G*T*C*A*T*C*A*mC*mC*mA*mG |
| 169 | mU*mA*mA*mU*mA*A*A*T*T*G*T*C*A*T*C*mA*mC*mC*mA*mG |
| 170 | mU*mU*mA*mA*mU*A*A*A*T*T*G*T*C*A*T*C*A*C*C*mA |
| 171 | mU*mU*mA*mA*mU*A*A*A*T*T*G*T*C*A*T*C*A*C*mC*mA |
| 172 | mU*mU*mA*mA*mU*A*A*A*T*T*G*T*C*A*T*C*A*mC*mC*mA |
| 173 | mU*mU*mA*mA*mU*A*A*A*T*T*G*T*C*A*T*C*mA*mC*mC*mA |
| 174 | mA*mU*mU*mA*mA*T*A*A*A*T*T*G*T*C*A*T*C*A*C*C |
| 175 | mA*mU*mU*mA*mA*T*A*A*A*T*T*G*T*C*A*T*C*A*C*mC |
| 176 | mA*mU*mU*mA*mA*T*A*A*A*T*T*G*T*C*A*T*C*A*mC*mC |
| 177 | mA*mU*mU*mA*mA*T*A*A*A*T*T*G*T*C*A*T*C*mA*mC*mC |
| 178 | mU*mA*mU*mU*mA*A*T*A*A*A*T*T*G*T*C*A*T*C*A*C |
| 179 | mU*mA*mU*mU*mA*A*T*A*A*A*T*T*G*T*C*A*T*C*A*mC |
| 180 | mU*mA*mU*mU*mA*A*T*A*A*A*T*T*G*T*C*A*T*C*mA*mC |
| 181 | mC*mU*mA*mU*mU*A*A*T*A*A*A*T*T*G*T*C*A*T*C*A |
| 182 | mC*mU*mA*mU*mU*A*A*T*A*A*A*T*T*G*T*C*A*T*C*mA |
| 183 | mA*mC*mU*mA*mU*T*A*A*T*A*A*A*T*T*G*T*C*A*T*C |
| 184 | T*G*T*C*A*T*C*A*C*C*A*G*A*A*A*mAmAmGmU*mC |
| 185 | mU*T*G*T*C*A*T*C*A*C*C*A*G*A*A*mAmAmAmG*mU |
| 186 | T*T*G*T*C*A*T*C*A*C*C*A*G*A*A*mAmAmAmG*mU |
| 187 | mA*mU*T*G*T*C*A*T*C*A*C*C*A*G*A*mAmAmAmA*mG |
| 188 | mA*T*T*G*T*C*A*T*C*A*C*C*A*G*A*mAmAmAmA*mG |
| 189 | mA*mAmU*T*G*T*C*A*T*C*A*C*C*A*G*mAmAmAmA*mA |
| 190 | mA*mA*T*T*G*T*C*A*T*C*A*C*C*A*G*mAmAmAmA*mA |
| 191 | mA*mAmA*T*T*G*T*C*A*T*C*A*C*C*A*mGmAmAmA*mA |
| 192 | mA*mAmAmU*T*G*T*C*A*T*C*A*C*C*A*mGmAmAmA*mA |
| 193 | mU*mAmAmAmU*T*G*T*C*A*T*C*A*C*C*A*mGmAmA*mA |
| 194 | mU*mAmAmAmU*T*G*T*C*A*T*C*A*C*C*mAmGmAmA*mA |
| 195 | mA*mUmAmAmA*T*T*G*T*C*A*T*C*A*C*C*mAmGmA*mA |
| 196 | mA*mUmAmAmA*T*T*G*T*C*A*T*C*A*C*mCmAmGmA*mA |
| 197 | mA*mAmUmAmA*A*T*T*G*T*C*A*T*C*A*C*C*mAmG*mA |
| 198 | mA*mAmUmAmA*A*T*T*G*T*C*A*T*C*A*C*mCmAmG*mA |
| 199 | mA*mAmUmAmA*A*T*T*G*T*C*A*T*C*A*mCmCmAmG*mA |
| 200 | mU*mAmAmUmA*A*A*T*T*G*T*C*A*T*C*A*C*C*mA*mG |
| 201 | mU*mAmAmUmA*A*A*T*T*G*T*C*A*T*C*A*C*mCmA*mG |
| 202 | mU*mAmAmUmA*A*A*T*T*G*T*C*A*T*C*A*mCmCmA*mG |
| 203 | mU*mAmAmUmA*A*A*T*T*G*T*C*A*T*C*mAmCmCmA*mG |
| 204 | mUmUmAmAmU*A*A*A*T*T*G*T*C*A*T*C*A*C*C*mA |
| 205 | mU*mUmAmAmU*A*A*A*T*T*G*T*C*A*T*C*A*C*mC*mA |
| 206 | mU*mUmAmAmU*A*A*A*T*T*G*T*C*A*T*C*A*mCmC*mA |
| 207 | mU*mUmAmAmU*A*A*A*T*T*G*T*C*A*T*C*mAmCmC*mA |
| 208 | mA*mUmUmAmA*T*A*A*A*T*T*G*T*C*A*T*C*A*C*C |
| 209 | mA*mUmUmAmA*T*A*A*A*T*T*G*T*C*A*T*C*A*C*mC |
| 210 | mA*mUmUmAmA*T*A*A*A*T*T*G*T*C*A*T*C*A*mC*mC |
| 211 | mA*mUmUmAmA*T*A*A*A*T*T*G*T*C*A*T*C*mAmC*mC |
| 212 | mU*mAmUmUmA*A*T*A*A*A*T*T*G*T*C*A*T*C*A*C |
| 213 | mU*mAmUmUmA*A*T*A*A*A*T*T*G*T*C*A*T*C*A*mC |
| 214 | mU*mAmUmUmA*A*T*A*A*A*T*T*G*T*C*A*T*C*mA*mC |
| 215 | mC*mUmAmUmU*A*A*T*A*A*A*T*T*G*T*C*A*T*C*A |
| 216 | mC*mUmAmUmU*A*A*T*A*A*A*T*T*G*T*C*A*T*C*mA |
| 217 | mA*mCmUmAmU*T*A*A*T*A*A*A*T*T*G*T*C*A*T*C |
| 218 | G*SG*SG*SC*SA*SC*SA*SA*SG*SG*SG*SC*SA*SC*RA*SG*SA*SC* |
| | ST*ST |
| 219 | G*SG*SC*SA*SC*SA*SA*SG*SG*SG*SC*SA*SC*RA*SG*SA*SC*ST* |
| | ST*SC |
| 220 | G*SC*SA*SC*SA*SA*SG*SG*SG*SC*SA*SC*RA*SG*SA*SC*ST*ST* |
| | SC*SC |
| 221 | C*SA*SC*SA*SA*SG*SG*SG*SC*SA*SC*RA*SG*SA*SC*ST*ST*SC* |
| | SC*SA |
| 222 | A*SC*SA*SA*SG*SG*SG*SC*SA*SC*RA*SG*SA*SC*ST*ST*SC*SC* |
| | SA*SA |
| 223 | C*SA*SA*SG*SG*SC*SC*SA*SC*RA*SG*SA*SC*ST*ST*SC*SC*SA* |
| | SA*SA |
| 224 | mG*SmG*SmC*SmA*SmC*SA*SA*SG*SG*SG*SC*SA*SC*RA*SG*SmA* |
| | SmC*SmU*SmU*SmC |
| 225 | mG*RmG*RmC*RmA*RmC*SA*SA*SG*SG*SG*SC*SA*SC*RA*SG*SmA* |
| | RmC*RmU*RmU*RmC |
| 226 | mGmGmCmAmC*SA*SA*SG*SG*SG*SC*SA*SC*RA*SG*SmAmCmUmUmC |
| 227 | mG*SmG*SmC*SmA*SmC*SmA*SmA*SmG*SG*SG*SC*SA*SC*RA*SG* |
| | SA*SC*ST*ST*SC |
| 228 | mG*RmG*RmC*RmA*RmC*RmA*RmA*RmG*SG*SG*SC*SA*SC*RA*SG* |
| | SA*SC*ST*ST*SC |
| 229 | mGmGmCmAmCmAmAmG*SG*SG*SC*SA*SC*RA*SG*SA*SC*ST*ST*SC |
| 230 | mG*RmGmCmAmC*SA*SA*SG*SG*SG*SC*SA*SC*RA*SG*SmAmCmUmU* |
| | RmC |
| 231 | mG*SmGmCmAmC*SA*SA*SG*SG*SG*SC*SA*SC*RA*SG*SmAmCmUmU* |
| | SmC |
| 232 | G*SC*SA*SG*SG*SG*SC*SA*SC*SA*SA*SG*SG*SG*SC*SA*SC*RA* |
| | SG*SA |
| 233 | C*SA*SG*SG*SG*SC*SA*SC*SA*SA*SG*SG*SG*SC*SA*SC*RA*SG* |
| | SA*SC |
| 234 | A*SG*SG*SG*SC*SA*SC*SA*SA*SG*SG*SG*SC*SA*SC*RA*SG*SA* |
| | SC*ST |
| 235 | A*SA*SG*SG*SG*SC*SA*SC*RA*SG*SA*SC*ST*ST*SC*SC*SA*SA* |
| | SA*SG |
| 236 | A*SG*SG*SG*SC*SA*SC*RA*SG*SA*SC*ST*ST*SC*SC*SA*SA*SA* |
| | SG*SG |
| 237 | G*SG*SG*SC*SA*SC*RA*SG*SA*SC*ST*ST*SC*SC*SA*SA*SA*SG* |
| | SG*SC |
| 238 | G*SmGmCmAmC*SA*SA*SG*SG*SG*SC*SA*SC*RA*SG*SmAmCmUmU* |
| | SC |
| 239 | G*SG*SG*SC*SC*SA*SA*SC*SA*SG*SC*RC*SA*SG*SC*SC*ST*SG* |
| | SC*SA |
| 240 | G*SG*SC*SC*SA*SA*SC*SA*SG*SC*RC*SA*SG*SC*SC*ST*SG*SC* |
| | SA*SG |
| 241 | G*SC*SC*SA*SA*SC*SA*SG*SC*RC*SA*SG*SC*SC*ST*SG*SC*SA* |
| | SG*SG |
| 242 | C*SC*SA*SA*SC*SA*SG*SC*RC*SA*SG*SC*SC*ST*SG*SC*SA*SG* |
| | SG*SA |
| 243 | C*SA*SA*SC*SA*SG*SC*RC*SA*SG*SC*SC*ST*SG*SC*SA*SG*SG* |
| | SA*SG |
| 244 | A*SA*SC*SA*SG*SC*RC*SA*SG*SC*SC*ST*SG*SC*SA*SG*SG*SA* |
| | SG*SG |
| 245 | G*SA*SG*SC*SA*SG*SC*ST*SG*SC*SA*RA*SC*SC*ST*SG*SG*SC* |
| | SA*SA |
| 246 | A*SG*SC*SA*SG*SC*ST*SG*SC*SA*RA*SC*SC*ST*SG*SG*SC*SA* |
| | SA*SC |
| 247 | G*SC*SA*SG*SC*ST*SG*SC*SA*RS*SC*SC*ST*SG*SG*SC*SA*SA* |
| | SC*SA |
| 248 | C*SA*SG*SC*ST*SG*SC*SA*RA*SC*SC*ST*SG*SG*SC*SA*SA*SC* |
| | SA*SA |
| 249 | A*SG*SC*ST*SG*SC*SA*RA*SC*SC*ST*SG*SG*SC*SA*SA*SC*SA* |
| | SA*SC |
| 250 | G*SC*ST*SG*SC*SA*RA*SC*SC*ST*SG*SG*SC*SA*SA*SC*SA*SA* |
| | SC*SC |
| 251 | A*T*T*A*A*T*A*A*A*T*T*G*T*C*A*T*C*A*C*C |
| 252 | A*ST*ST*SA*SA*ST*SA*SA*SA*ST*ST*SG*ST*SC*RA*ST*SC*SA* |
| | SC*SC |
| 253 | A*ST*ST*SA*SA*ST*SA*SA*SA*ST*ST*SG*ST*SC*SA*RT*SC*SA* |
| | SC*SC |
| 254 | mA*SmU*SmU*SmA*SmA*SmU*SA*SA*SA*ST*ST*SG*ST*SC*RA*ST* |
| | SmC*SmA*SmC*SmC |
| 255 | mA*RmU*RmU*RmA*RmA*RmU*SA*SA*SA*ST*ST*SG*ST*SC*RA*ST* |
| | SmC*RmA*RmC*RmC |
| 256 | mA*SmU*SmU*SmA*SmA*SmU*SmA*SmA*SA*ST*ST*SG*ST*SC*RA* |
| | ST*SC*SA*SC*SC |
| 257 | mA*RmU*RmU*RmA*RmA*RmU*RmA*RmA*SA*ST*ST*SG*ST*SC*RA* |
| | ST*SC*SA*SC*SC |
| 258 | mAmUmUmAmAmUmAmA*SA*ST*ST*SG*ST*SC*RA*ST*SC*SA*SC*SC |
| 259 | mAmUmUmAmAmU*SA*SA*SA*ST*ST*SG*ST*SC*RA*ST*SmCmAmCmC |
| 260 | mA*SmUmUmAmAmU*SA*SA*SA*ST*ST*SG*ST*SC*RA*ST*SmCmAmC* |
| | SmC |
| 261 | mA*RmUmUmAmAmU*SA*SA*SA*ST*ST*SG*ST*SC*RA*ST*SmCmAmC* |
| | RmC |
| 262 | A*SmUmUmAmAmU*SA*SA*SA*ST*ST*SG*ST*SC*RA*ST*SmCmAmC* |
| | SC |

In Table 1; “*” represents a stereorandom phosphorothioate linkage; “*S” represents an Sp phosphorothioate linkage; “*R” represents an Rp phosphorothioate linkage; all non-labeled linkage is a natural phosphate linkage; “in” preceding a base represents 2′-OMe; “d2AP” represents a 2-amino purine; “dDAP” represents a 2,6-diamino purine; “eo” following a base represents 2′-MOE; and “BrdU” represents Bromodeoxyuridine.

TABLE 2

| SEQ | |
|---|---|
| ID | |
| NO. | Sequence 5′→3′ |
| 263 | m5C*m5C*G*T*m5C*G*m5C*m5C*m5C*T*T*m5C*A*G*m5C*A*m5C*G* |
| | m5C*A |
| 264 | m5C*Sm5C*SG*ST*Sm5C*SG*Sm5C*Sm5C*Sm5C*ST*ST*Sm5C*RA*SG* |
| | Sm5C*SA*Sm5C*SG*Sm5C*SA |
| 265 | m5Ceo*m5Ceo*Geo*Teo*m5Ceo*G*m5C*m5C*m5C*T*T*m5C*A*G*m5C*A |
| | eo*m5Ceo*Geo*m5Ceo*Aeo |
| 266 | m5Ceo*Sm5Ceo*SGeo*STeo*Sm5Ceo*SG*Sm5C*Sm5C*Sm5C*ST*ST*Sm5 |
| | C*SA*SG*Sm5C*SAeo*Sm5Ceo*SGeo*Sm5Ceo*SAeo |
| 267 | m5Ceo*Sm5Ceo*SGeo*STeo*Sm5Ceo*SG*Sm5C*Sm5C*Sm5C*ST*ST*Sm5 |
| | C*RA*SG*Sm5C*SAeo*Sm5Ceo*SGeo*Sm5Ceo*SAeo |
| 268 | m5Ceo*Rm5Ceo*RGeo*RTeo*Rm5Ceo*RG*Sm5C*Sm5C*Sm5C*5T*ST*Sm5 |
| | C*RA*SG*Sm5C*SAeo*Rm5Ceo*RGeo*Rm5Ceo*RAeo |
| 269 | Geo*Teo*m5Ceo*m5Ceo*m5Ceo*T*G*A*A*G*A*T*G*T*m5C*Aeo*Aeo*Te |
| | o*Geo*m5Ceo |
| 270 | Geo*RTeo*Rm5Ceo*Rm5Ceo*Rm5Ceo*RT*RG*RA*RA*RG*RA*RT*RG*RT |
| | *Rm5C*RAeo*RAeo*RTeo*RGeo*Rm5Ceo |
| 271 | Geo*STeo*Sm5Ceo*Sm5Ceo*Sm5Ceo*ST*SG*SA*SA*SG*SA*ST*SG*ST*S |
| | m5C*SAeo*SAeo*STeo*SGeo*Sm5Ceo |
| 272 | Geo*RTeo*Rm5Ceo*Rm5Ceo*Rm5Ceo*RT*SG*SA*SA*SG*SA*ST*SG*ST* |
| | Sm5C*SAeo*RAeo*RTeo*RGeo*Rm5Ceo |
| 273 | Geo*STeo*Sm5Ceo*Sm5Ceo*Sm5Ceo*ST*RG*RA*RA*RG*RA*RT*RG*RT* |
| | Rm5C*RAeo*SAeo*STeo*SGeo*Sm5Ceo |
| 274 | mG*mG*mC*mA*mC*A*A*G*G*G*C*A*C*A*G*mA*mC*mU*mU*mC |
| 275 | mG*SmG*SmC*SmA*SmC*SA*SA*SG*SG*SG*SC*SA*SC*RA*SG*SmA*S |
| | mC*SmU*SmU*SmC |
| 276 | mG*RmG*RmC*RmA*RmC*SA*SA*SG*SG*SG*SC*SA*SC*RA*SG*SmA* |
| | RmC*RmU*RmU*RmC |
| 277 | mGmGmCmAmC*SA*SA*SG*SG*SG*SC*SA*SC*RA*SG*SmAmCmUmUm |
| | C |
| 278 | mG*RmGmCmAmC*SA*SA*SG*SG*SG*SC*SA*SC*RA*SG*SmAmCmUm |
| | U*RmC |
| 279 | mG*SmGmCmAmC*SA*SA*SG*SG*SG*SC*SA*SC*RA*SG*SmAmCmUm |
| | U*SmC |
| 280 | G*SmGmCmAmC*SA*SA*SG*SG*SG*SC*SA*SC*RA*SG*SmAmCmUmU* |
| | SC |
| 281 | G*mGmCmAmC*A*A*G*G*G*C*A*C*A*G*mAmCmUmU*C |
| 282 | mG*mGmCmAmC*A*A*G*G*G*C*A*C*A*G*mAmCmUmU*mC |
| 283 | Geo*Geom5CeoAeom5Ceo*A*A*G*G*G*C*A*C*A*G*Aeom5CeoTeoTeo*m |
| | 5Ceo |
| 284 | mU*mC*mA*mA*mG*mG*mA*mA*mG*mA*mU*mG*mG*mC*mA*mU*mU |
| | *mU*mC*mU |
| 285 | mU*SmC*SmA*SmA*SmG*SmG*SmA*SmA*SmG*SmA*SmU*SmG*SmG*S |
| | mC*SmA*SmU*SmU*SmU*SmC*SmU |
| 286 | mU*RmC*RmA*RmA*RmG*RmG*RmA*RmA*RmG*RmA*RmU*RmG*RmG |
| | *RmC*RmA*RmU*RmU*RmU*RmC*RmU |
| 287 | mU*SmC*RmA*SmA*RmG*SmG*RmA*SmA*RmG*SmA*RmU*SmG*RmG* |
| | SmC*RmA*SmU*RmU*SmU*RmC*SmU |
| 288 | mU*RmC*RmA*RmA*SmG*SmG*SmA*SmA*SmG*SmA*SmU*SmG*SmG* |
| | SmC*SmA*SmU*SmU*RmU*RmC*RmU |
| 289 | mU*SmC*SmA*SmA*RmG*RmG*RmA*RmA*RmG*RmA*RmU*RmG*mG* |
| | RmC*RmA*RmU*RmU*SmU*SmC*SmU |
| 290 | mU*RmC*RmA*RmA*RmG*RmG*SmA*SmA*RmG*SmA*SmU*RmG*SmG* |
| | SmC*RmA*RmU*RmU*RmU*RmC*RmU |
| 291 | mU*SmC*SmA*SmA*SmG*SmG*RmA*RmA*SmG*RmA*RmU*SmG*RmG* |
| | RmC*SmA*SmU*SmU*SmU*SmC*SmU |
| 292 | mU*RmC*RmA*RmA*SmG*SmG*RmA*RmA*SmG*RmA*RmU*RmG*SmG |
| | *RmC*RmA*SmU*SmU*RmU*RmC*RmU |
| 293 | mU*SmC*SmA*SmA*RmG*RmG*SmA*SmA*RmG*SmA*SmU*SmG*RmG* |
| | SmC*SmA*RmU*RmU*SmU*SmC*SmU |
| 294 | mU*SmC*RmA*RmA*RmG*RmG*RmA*RmA*RmG*RmA*RmU*RmG*RmG |
| | *RmC*RmA*RmU*RmU*RmU*RmC*SmU |
| 295 | mU*RmC*SmA*SmA*SmG*SmG*SmA*SmA*SmG*SmA*SmU*SmG*SmG*S |
| | mC*SmA*SmU*SmU*SmU*SmC*RmU |
| 296 | mU*SmC*RmA*SmA*SmG*RmG*RmA*SmA*SmG*RmA*SmU*SmG*RmG* |
| | RmC*RmA*SmU*SmU*SmU*SmC*RmU |
| 297 | mU*RmC*SmA*RmA*RmG*SmG*SmA*RmA*RmG*SmA*RmU*RmG*SmG* |
| | SmC*SmA*RmU*RmU*RmU*RmC*SmU |
| 298 | mU*SmC*SmA*RmA*RmG*RmG*RmA*RmA*RmG*RmA*RmU*SmG*RmG |
| | *RmC*SmA*RmU*SmU*SmU*SmC*SmU |
| 299 | mU*RmC*RmA*SmA*SmG*SmG*SmA*SmA*SmG*SmA*SmU*RmG*SmG* |
| | SmC*RmA*SmU*RmU*RmU*RmC*RmU |
| 300 | T*C*A*A*G*G*A*A*G*A*T*G*G*C*A*T*T*T*C*T |
| 301 | mUmCmAmAmGmGmAmAmGmAmUmGmGmCmAmUmUmUmCmU |
| 302 | T*RC*RA*RA*RG*RG*RA*RA*RG*RA*RT*RG*RG*RC*RA*RT*RT*RT*R |
| | C*RT |
| 303 | T*SC*SA*SA*SG*SG*SA*SA*SG*SA*ST*SG*SG*SC*SA*ST*ST*ST*SC*S |
| | T |
| 304 | T*SC*SA*SA*SG*SmGmAmAmGmAmUmGmGmCA*ST*ST*ST*SC*ST |
| 305 | mUmCmAmAG*SG*SA*SmAG*SA*ST*SG*SmGC*SA*ST*SmUmUmCmU |
| 306 | T*SmCA*SmAG*SmGA*SmAG*SmAT*SmGG*SmCA*SmUT*SmUC*SmU |
| 307 | mUC*SmAA*SmGG*SmAA*SmGA*SmUG*SmGC*SmAT*SmUT*SmCmU |
| 308 | T*SC*SmAmAG*SG*SmAmAG*SA*ST*SmGmGC*SA*SmUmUT*SC*SmU |
| 309 | T*SC*SA*SmAmGmGA*SA*SmGmAmUG*SG*SmCmAmUT*ST*SC*SmU |
| 310 | T*SC*SA*SA*SmGmGmAmAG*SA*ST*SmGmGmCmAT*ST*ST*SC*SmU |
| 311 | T*SC*SA*SmAG*SG*SA*SmAG*SA*ST*SmGG*SC*SA*SmUT*ST*SC*Sm |
| | U |
| 312 | mUmCmAmAG*SG*SA*SA*SG*SmAmUmGmGmCA*ST*ST*ST*SC*SmU |
| 313 | T*SC*SmAmAmGmGmAmAmGmAT*SmGmGC*SmAT*ST*ST*SC*SmU |
| 314 | T*C*A*A*G*mGmAmAmGmAmUmGmGmCA*T*T*T*C*T |
| 315 | mUmCmAmAG*G*A*mAG*A*T*G*mGC*A*T*mUmUmCmU |
| 316 | T*mCA*mAG*mGA*mAG*mAT*mGG*mCA*mUT*mUC*mU |
| 317 | mUC*mAA*mGG*mAA*mGA*mUG*mGC*mAT*mUT*mCmU |
| 318 | T*C*mAmAG*G*mAmAG*A*T*mGmGC*A*mUmUT*C*mU |
| 319 | T*C*A*mAmGmGA*A*mGmAmUG*G*mCmAmUT*T*C*mU |
| 320 | T*C*A*A*mGmGmAmAG*A*T*mGmGmCmAT*T*T*C*mU |
| 321 | T*C*A*mAG*G*A*mAG*A*T*mGG*C*A*mUT*T*C*mU |
| 322 | mUmCmAmAG*G*A*A*G*mAmUmGmGmCA*T*T*T*C*mU |
| 323 | T*C*mAmAmGmGmAmAmGmAT*mGmGC*mAT*T*T*C*mU |
| 324 | fU*fC*fA*fA*fG*fG*fA*fA*fG*fA*fU*fG*fG*fC*fA*fU*fU*fU*fC*fU |
| 325 | fU*fC*mA*mA*mG*mG*mA*mA*mG*mA*fU*mG*mG*fC*mA*fU*fU*fU*f |
| | C*fU |
| 326 | mU*mC*fA*fA*fG*fG*fA*fA*fG*fA*mU*fG*fG*mC*fA*mU*mU*mU*mC* |
| | mU |
| 327 | mU*fC*mA*fA*mG*fG*mA*fA*mG*fA*mU*fG*mG*fC*mA*fU*mU*fU*m |
| | C*fU |
| 328 | mU*mC*mA*mA*mG*mG*fA*fA*fG*fA*fU*fG*fG*fC*mA*mU*mU*mU*m |
| | C*mU |
| 329 | fU*fC*fA*fA*fG*fG*mA*mA*mG*mA*mU*mG*mG*mC*fA*fU*fU*fU*fC* |
| | fU |
| 330 | mU*fC*mA*mA*fG*fG*mA*mA*fG*mA*mU*fG*fG*fC*mA*mU*mU*mU*f |
| | C*mU |
| 331 | fU*mC*fA*fA*mG*mG*fA*fA*mG*fA*fU*mG*mG*mC*fA*fU*fU*fU*mC* |
| | fU |
| 332 | mUmCmAmAmGmG*SmA*SmA*RmG*SmA*SmU*RmG*SmG*SmC*RmAm |
| | UmUmUmCmU |
| 333 | mUmCmAmAmGmG*RmA*RmA*SmG*RmA*RmU*SmG*RmG*RmC*SmAm |
| | UmUmUmCmU |
| 334 | mU*SmCmAmAmGmG*SmA*SmA*RmG*SmA*SmU*RmG*SmG*SmC*RmA |
| | mUmUmUmC*SmU |
| 335 | mU*RmCmAmAmGmG*RmA*RmA*SmG*RmA*RmU*SmG*RmG*RmC*Sm |
| | AmUmUmUmC*RmU |
| 336 | mU*RmCmAmAmGmG*SmA*SmA*RmG*SmA*SmU*RmG*SmG*SmC*RmA |
| | mUmUmUmC*RmU |
| 337 | mU*SmCmAmAmGmG*RmA*RmA*SmG*RmA*RmU*SmG*RmG*RmC*Sm |
| | AmUmUmUmC*SmU |
| 338 | mU*SmC*SmA*SmA*SmG*SmG*SmA*SmA*RmG*SmA*SmU*RmG*SmG*S |
| | mC*RmA*SmU*SmU*SmU*SmC*SmU |
| 339 | mU*SmC*RmA*SmA*SmG*RmG*SmA*SmA*RmG*SmA*SmU*RmG*SmG* |
| | SmC*RmA*SmU*SmU*RmU*SmC*SmU |
| 340 | mU*RmC*SmA*RmA*RmG*SmG*RmA*RmA*SmG*RmA*RmU*SmG*RmG |
| | *RmC*SmA*RmU*RmU*SmU*RmC*RmU |
| 341 | mUmCmAmAmGmG*SmA*SmAmG*SmA*SmUmG*SmG*SmCmAmUmUmU |
| | mCmU |
| 342 | mU*SmCmAmAmGmG*SmA*SmAmG*SmA*SmUmG*SmG*SmCmAmUmU |
| | mUmC*SmU |
| 343 | mU*SmCmAmAmGmGmAmAmGmAmUmGmGmCmAmUmUmUmC*SmU |
| 344 | mU*SmC*SmAmAmGmGmAmAmGmAmU*SmGmGmC*SmAmU*SmU*SmU |
| | *SmC*SmU |
| 345 | mU*SmC*SmA*SmA*SmG*SmGmAmAmGmAmUmGmGmCmA*SmU*SmU* |
| | SmU*SmC*SmU |
| 346 | mU*RmC*RmA*RmA*RmG*RmGmAmAmGmAmUmGmGmCmA*RmU*Rm |
| | U*RmU*RmC*RmU |
| 347 | mU*SmC*SmA*SmA*SmG*SmG*RmA*RmA*RmG*RmA*RmU*RmG*RmG* |
| | RmC*RA*SmU*SmU*SmU*SmC*SmU |
| 348 | mU*RmC*RmA*RmA*RmG*RmG*SmA*SmA*SmG*SmA*SmU*SmG*SmG* |
| | SmC*SmA*RmU*RmU*RmU*RmC*RmU |
| 349 | mU*SmC*SmA*SmA*SmG*SmG*SmA*SmA*SmG*SmA*SmU*RmG*RmG* |
| | RmC*RA*RmU*RmU*RmU*RmC*RmU |
| 350 | mU*RmC*RmA*RmA*RmG*RmG*RmA*RmA*RmG*RmA*RmU*SmG*SmG |
| | *SmC*SmA*SmU*SmU*SmU*SmC*SmU |
| 351 | mU*RmC*RmA*RmA*RmG*SmG*SmA*RmA*SmG*SmA*RmU*SmG*SmG* |
| | RmC*RmA*RmU*RmU*RmU*RmC*RmU |
| 352 | mU*RmC*RmA*RmA*RmG*RmG*RmA*SmA*SmG*RmA*SmU*SmG*RmG |
| | *SmC*SmA*RmU*RmU*RmU*RmC*RmU |
| 353 | fU*SfC*SfA*SfA*SfG*SfG*SfA*SfA*SfG*SfA*SfU*SfG*SfG*SfC*SfA*SfU* |
| | SfU*SfU*SfC*SfU |
| 354 | fU*RfC*RfA*RfA*RfG*RfG*RfA*RfA*RfG*RfA*RfU*RfG*RfG*RfC*RfA*R |
| | fU*RfU*RfU*RfC*RfU |
| 355 | fU*RfC*RfA*RfA*RfG*RfG*SfA*SfA*RfG*SfA*SfU*RfG*SfG*SfC*RfA*Rf |
| | U*RfU*RfU*RfC*RfU |
| 356 | fU*SfC*RfA*RfA*RfG*RfG*RfA*RfA*RfG*RfA*RfU*RfG*RfG*RfC*RfA*Rf |
| | U*RfU*RfU*RfC*SfU |
| 357 | fU*SfC*SfA*RfA*RfG*RfG*RfA*RfA*RfG*RfA*RfU*SfG*RfG*RfC*SfA*Rf |
| | U*SfU*SfU*SfC*SfU |
| 358 | fU*SfC*SmA*SmA*SmG*SmG*SmA*SmA*SmG*SmA*SfU*SmG*SmG*SfC* |
| | SmA*SfU*SfU*SfU*SfC*SfU |
| 359 | fU*RfC*RmA*RmA*RmG*RmG*RmA*RmA*RmG*RmA*RfU*RmG*RmG*R |
| | fC*RmA*RfU*RfU*RfU*RfC*RfU |
| 360 | fU*RfC*RmA*RmA*RmG*RmG*SmA*SmA*RmG*SmA*SfU*RmG*SmG*Sf |
| | C*RmA*RfU*RfU*RfU*RfC*RfU |
| 361 | fU*SfC*RmA*RmA*RmG*RmG*RmA*RmA*RmG*RmA*RfU*RmG*RmG*R |
| | fC*RmA*RfU*RfU*RfU*RfC*SfU |
| 362 | fU*SfC*SmA*RmA*RmG*RmG*RmA*RmA*RmG*RmA*RfU*SmG*RmG*Rf |
| | C*SmA*RfU*SfU*SfU*SfC*SfU |
| 363 | mU*SmC*SfA*SfA*SfG*SfG*SfA*SfA*SfG*SfA*SmU*SfG*SfG*SmC*SfA*S |
| | mU*SmU*SmU*SmC*SmU |
| 364 | mU*RmC*RfA*RfA*RfG*RfG*RfA*RfA*RfG*RfA*RmU*RfG*RfG*RmC*Rf |
| | A*RmU*RmU*RmU*RmC*RmU |
| 365 | mU*RmC*RfA*RfA*RfG*RfG*SfA*SfA*RfG*SfA*SmU*RfG*SfG*SmC*RfA |
| | *RmU*RmU*RmU*RmC*RmU |
| 366 | mU*SmC*RfA*RfA*RfG*RfG*RfA*RfA*RfG*RfA*RmU*RfG*RfG*RmC*Rf |
| | A*RmU*RmU*RmU*RmC*SmU |
| 367 | mU*SmC*SfA*RfA*RfG*RfG*RfA*RfA*RfG*RfA*RmU*SfG*RfG*RmC*SfA |
| | *RmU*SmU*SmU*SmC*SmU |
| 368 | mU*SfC*SmA*SfA*SmG*SfG*SmA*SfA*SmG*SfA*SmU*SfG*SmG*SfC*Sm |
| | A*SfU*SmU*SfU*SmC*SfU |
| 369 | mU*RfC*RmA*RfA*RmG*RfG*RmA*RfA*RmG*RfA*RmU*RfG*RmG*RfC* |
| | RmA*RfU*RmU*RfU*RmC*RfU |
| 370 | mU*RfC*RmA*RfA*RmG*RfG*SmA*SfA*RmG*SfA*SmU*RfG*SmG*SfC*R |
| | mA*RfU*RmU*RfU*RmC*RfU |
| 371 | mU*SfC*RmA*RfA*RmG*RfG*RmA*RfA*RmG*RfA*RmU*RfG*RmG*RfC* |
| | RmA*RfU*RmU*RfU*RmC*SfU |
| 372 | mU*SfC*SmA*RfA*RmG*RfG*RmA*RfA*RmG*RfA*RmU*SfG*RmG*RfC* |
| | SmA*RfU*SmU*SfU*SmC*SfU |
| 373 | mU*SmC*SmA*SmA*SmG*SmG*SfA*SfA*SfG*SfA*SfU*SfG*SfG*SfC*Sm |
| | A*SmU*SmU*SmU*SmC*SmU |
| 374 | mU*RmC*RmA*RmA*RmG*RmG*RfA*RfA*RfG*RfA*RfU*RfG*RfG*RfC* |
| | RmA*RmU*RmU*RmU*RmC*RmU |
| 375 | mU*RmC*RmA*RmA*RmG*RmG*SfA*SfA*RfG*SfA*SfU*RfG*SfG*SfC*R |
| | mA*RmU*RmU*RmU*RmC*RmU |
| 376 | mU*SmC*RmA*RmA*RmG*RmG*RfA*RfA*RfG*RfA*RfU*RfG*RfG*RfC* |
| | RmA*RmU*RmU*RmU*RmC*SmU |
| 377 | mU*SmC*SmA*RmA*RmG*RmG*RfA*RfA*RfG*RfA*RfU*SfG*RfG*RfC*S |
| | mA*RmU*SmU*SmU*SmC*SmU |
| 378 | fU*SfC*SfA*SfA*SfG*SfG*SmA*SmA*SmG*SmA*SmU*SmG*SmG*SmC*Sf |
| | A*SfU*SfU*SfU*SfC*SfU |
| 379 | fU*RfC*RfA*RfA*RfG*RfG*RmA*RmA*RmG*RmA*RmU*RmG*RmG*RmC |
| | *RfA*RfU*RfU*RfU*RfC*RfU |
| 380 | fU*RfC*RfA*RfA*RfG*RfG*SmA*SmA*RmG*SmA*SmU*RmG*SmG*SmC* |
| | RfA*RfU*RfU*RfU*RfC*RfU |
| 381 | fU*SfC*RfA*RfA*RfG*RfG*RmA*RmA*RmG*RmA*RmU*RmG*RmG*RmC |
| | *RfA*RfU*RfU*RfU*RfC*SfU |
| 382 | fU*SfC*SfA*RfA*RfG*RfG*RmA*RmA*RmG*RmA*RmU*SmG*RmG*RmC |
| | *SfA*RfU*SfU*SfU*SfC*SfU |
| 383 | mU*SfC*SmA*SmA*SfG*SfG*SmA*SmA*SfG*SmA*SmU*SfG*SfG*SfC*Sm |
| | A*SmU*SmU*SmU*SfC*SmU |
| 384 | mU*RfC*RmA*RmA*RfG*RfG*RmA*RmA*RfG*RmA*RmU*RfG*RfG*RfC* |
| | RmA*RmU*RmU*RmU*RfC*RmU |
| 385 | mU*RfC*RmA*RmA*RfG*RfG*SmA*SmA*RfG*SmA*SmU*RfG*SfG*SfC*R |
| | mA*RmU*RmU*RmU*RfC*RmU |
| 386 | mU*SfC*RmA*RmA*RfG*RfG*RmA*RmA*RfG*RmA*RmU*RfG*RfG*RfC* |
| | RmA*RmU*RmU*RmU*RfC*SmU |
| 387 | mU*SfC*SmA*RmA*RfG*RfG*RmA*RmA*RfG*RmA*RmU*SfG*RfG*RfC* |
| | SmA*RmU*SmU*SmU*SfC*SmU |
| 388 | fU*SmC*SfA*SfA*SmG*SmG*SfA*SfA*SmG*SfA*SfU*SmG*SmG*SmC*Sf |
| | A*SfU*SfU*SfU*SmC*SfU |
| 389 | fU*RmC*RfA*RfA*RmG*RmG*RfA*RfA*RmG*RfA*RfU*RmG*RmG*RmC* |
| | RfA*RfU*RfU*RfU*RmC*RfU |
| 390 | fU*RmC*RfA*RfA*RmG*RmG*SfA*SfA*RmG*SfA*SfU*RmG*SmG*SmC*R |
| | fA*RfU*RfU*RfU*RmC*RfU |
| 391 | fU*SmC*RfA*RfA*RmG*RmG*RfA*RfA*RmG*RfA*RfU*RmG*RmG*RmC* |
| | RfA*RfU*RfU*RfU*RmC*SfU |
| 392 | fU*SmC*SfA*RfA*RmG*RmG*RfA*RfA*RmG*RfA*RfU*SmG*RmG*RmC* |
| | SfA*RfU*SfU*SfU*SmC*SfU |
| 393 | mU*SmC*SmA*SmA*SmG*SmG*SmA*SmA*SmG*SmA*SmU*SmG*SmG*S |
| | mC*SmA*SmU*SmU*SmU*SmC*SmU |
| 394 | mU*mC*mA*mA*mG*mG*mA*mA*mG |
| | *mA*mU*mG*mG*mC*mA*mU*mU*mU*mC*mU |
| 395 | mG*mG*mC*mC*mA*mA*mA*mC*mC |
| | *mU*mC*mG*mG*mC*mU*mU*mA*mC*mC*mU |
| 396 | mC*mU*mC*mC*mA*mA*mC*mA*mU |
| | *mC*mA*mA*mG*mG*mA*mA*mG*m |
| | A*mU*mG*mG*mC*mA*mU*mU*mU*mC*mU*mA*mG |
| 397 | mA*mC*mC*mA*mG*mA*mG*mU*mA |
| | *mA*mC*mA*mG*mU*mC*mU*mG*mA*mG*mU*mA*mG*mG*mA*mG |
| 398 | mC*mA*mC*mC*mA*mG*mA*mG*mU |
| | *mA*mA*mC*mA*mG*mU*mC*mU*mG*mA*mG*mU*mA*mG*mG*mA |
| 399 | mU*mC*mA*mC*mC*mA*mG*mA*mG |
| | *mU*mA*mA*mC*mA*mG*mU*mC*mU*mG*mA*mG*mU*mA*mG*mG |
| 400 | mG*mU*mC*mA*mC*mC*mA*mG*mA |
| | *mG*mU*mA*mA*mC*mA*mG*mU*mC*mU*mG*mA*mG*mU*mA*mG |
| 401 | mG*mU*mU*mG*mU*mG*mU*mC*mA*mC*mC*mA*mG*mA*mG*mU*mA |
| | *mA*mC*mA*mG*mU*mC*mU*mG |
| 402 | mG*mG*mU*mU*mG*mU*mG*mU*mC*mA*mC*mC*mA*mG*mA*mG*mU |
| | *mA*mA*mC*mA*mG*mU*mC*mU |
| 403 | mA*mG*mG*mU*mU*mG*mU*mG*mU*mC*mA*mC*mC*mA*mG*mA*mG |
| | *mU*mA*mA*mC*mA*mG*mU*mC |
| 404 | mC*mA*mG*mG*mU*mU*mG*mU*mG*mU*mC*mA*mC*mC*mA*mG*mA |
| | *mG*mU*mA*mA*mC*mA*mG*mU |
| 405 | mA*mC*mA*mG*mG*mU*mU*mG*mU*mG*mU*mC*mA*mC*mC*mA*mG |
| | *mA*mG*mU*mA*mA*mC*mA*mG |
| 406 | mC*mC*mA*mC*mA*mG*mG*mU*mU*mG*mU*mG*mU*mC*mA*mC*mC |
| | *mA*mG*mA*mG*mU*mA*mA*mC |
| 407 | mA*mC*mC*mA*mC*mA*mG*mG*mU*mU*mG*mU*mG*mU*mC*mA*mC |
| | *mC*mA*mG*mA*mG*mU*mA*mA |
| 408 | mA*mA*mC*mC*mA*mC*mA*mG*mG*mU*mU*mG*mU*mG*mU*mC*mA |
| | *mC*mC*mA*mG*mA*mG*mU*mA |
| 409 | mU*mA*mA*mC*mC*mA*mC*mA*mG*mG*mU*mU*mG*mU*mG*mU*mC |
| | *mA*mC*mC*mA*mG*mA*mG*mU |
| 410 | mG*mU*mA*mA*mC*mC*mA*mC*mA*mG*mG*mU*mU*mG*mU*mG*mU |
| | *mC*mA*mC*mC*mA*mG*mA*mG |
| 411 | mA*mG*mU*mA*mA*mC*mC*mA*mC*mA*mG*mG*mU*mU*mG*mU*mG |
| | *mU*mC*mA*mC*mC*mA*mG*mA |
| 412 | mU*mA*mG*mU*mA*mA*mC*mC*mA*mC*mA*mG*mG*mU*mU*mG*mU |
| | *mG*mU*mC*mA*mC*mC*mA*mG |
| 413 | mU*mU*mA*mG*mU*mA*mA*mC*mC*mA*mC*mA*mG*mG*mU*mU*mG |
| | *mU*mG*mU*mC*mA*mC*mC*mA |
| 414 | mC*mU*mU*mA*mG*mU*mA*mA*mC*mC*mA*mC*mA*mG*mG*mU*mU |
| | *mG*mU*mG*mU*mC*mA*mC*mC |
| 415 | mC*mC*mU*mU*mA*mG*mU*mA*mA*mC*mC*mA*mC*mA*mG*mG*mU |
| | *mU*mG*mU*mG*mU*mC*mA*mC |
| 416 | mU*mC*mC*mU*mU*mA*mG*mU*mA*mA*mC*mC*mA*mC*mA*mG*mG |
| | *mU*mU*mG*mU*mG*mU*mC*mA |
| 417 | mG*mU*mU*mU*mC*mC*mU*mU*mA*mG*mU*mA*mA*mC*mC*mA*mC |
| | *mA*mG*mG*mU*mU*mG*mU*mG |
| 418 | mA*mG*mU*mU*mU*mC*mC*mU*mU*mA*mG*mU*mA*mA*mC*mC*mA |
| | *mC*mA*mG*mG*mU*mU*mG*mU |
| 419 | mC*mA*mG*mU*mU*mU*mC*mC*mU*mU*mA*mG*mU*mA*mA*mC*mC |
| | *mA*mC*mA*mG*mG*mU*mU*mG |
| 420 | mG*mC*mA*mG*mU*mU*mU*mC*mC*mU*mU*mA*mG*mU*mA*mA*mC |
| | *mC*mA*mC*mA*mG*mG*mU*mU |
| 421 | mG*mG*mC*mA*mG*mU*mU*mU*mC*mC*mU*mU*mA*mG*mU*mA*mA |
| | *mC*mC*mA*mC*mA*mG*mG*mU |
| 422 | mU*mG*mG*mC*mA*mG*mU*mU*mU*mC*mC*mU*mU*mA*mG*mU*mA |
| | *mA*mC*mC*mA*mC*mA*mG*mG |
| 423 | mA*mU*mG*mG*mC*mA*mG*mU*mU*mU*mC*mC*mU*mU*mA*mG*mU |
| | *mA*mA*mC*mC*mA*mC*mA*mG |
| 424 | mA*mG*mA*mU*mG*mG*mC*mA*mG*mU*mU*mU*mC*mC*mU*mU*mA |
| | *mG*mU*mA*mA*mC*mC*mA*mC |
| 425 | mG*mA*mG*mA*mU*mG*mG*mC*mA*mG*mU*mU*mU*mC*mC*mU*mU |
| | *mA*mG*mU*mA*mA*mC*mC*mA |
| 426 | mG*mG*mA*mG*mA*mU*mG*mG*mC*mA*mG*mU*mU*mU*mC*mC*mU |
| | *mU*mA*mG*mU*mA*mA*mC*mC |
| 427 | mU*mG*mG*mA*mG*mA*mU*mG*mG*mC*mA*mG*mU*mU*mU*mC*mC |
| | *mU*mU*mA*mG*mU*mA*mA*mC |
| 428 | mU*mU*mG*mG*mA*mG*mA*mU*mG*mG*mC*mA*mG*mU*mU*mU*mC |
| | *mC*mU*mU*mA*mG*mU*mA*mA |
| 429 | mU*mU*mU*mG*mG*mA*mG*mA*mU*mG*mG*mC*mA*mG*mU*mU*mU |
| | *mC*mC*mU*mU*mA*mG*mU*mA |
| 430 | mA*mG*mU*mU*mU*mG*mG*mA*mG*mA*mU*mG*mG*mC*mA*mG*mU |
| | *mU*mU*mC*mC*mU*mU*mA*mG |
| 431 | mU*mA*mG*mU*mU*mU*mG*mG*mA*mG*mA*mU*mG*mG*mC*mA*mG |
| | *mU*mU*mU*mC*mC*mU*mU*mA |
| 432 | mC*mU*mA*mG*mU*mU*mU*mG*mG*mA*mG*mA*mU*mG*mG*mC*mA |
| | *mG*mU*mU*mU*mC*mC*mU*mU |
| 433 | mU*mC*mU*mA*mG*mU*mU*mU*mG*mG*mA*mG*mA*mU*mG*mG*mC |
| | *mA*mG*mU*mU*mU*mC*mC*mU |
| 434 | mU*mU*mC*mU*mA*mG*mU*mU*mU*mG*mG*mA*mG*mA*mU*mG*mG |
| | *mC*mA*mG*mU*mU*mU*mC*mC |
| 435 | mC*mA*mU*mU*mU*mC*mU*mA*mG*mU*mU*mU*mG*mG*mA*mG*mA |
| | *mU*mG*mG*mC*mA*mG*mU*mU |
| 436 | mG*mC*mA*mU*mU*mU*mC*mU*mA*mG*mU*mU*mU*mG*mG*mA*mG |
| | *mA*mU*mG*mG*mC*mA*mG*mU |
| 437 | mA*mU*mG*mG*mC*mA*mU*mU*mU*mC*mU*mA*mG*mU*mU*mU*mG |
| | *mG*mA*mG*mA*mU*mG*mG*mC |
| 438 | mG*mA*mA*mG*mA*mU*mG*mG*mC*mA*mU*mU*mU*mC*mU*mA*mG |
| | *mU*mU*mU*mG*mG*mA*mG*mA |
| 439 | mA*mG*mG*mA*mA*mG*mA*mU*mG*mG*mC*mA*mU*mU*mU*mC*mU |
| | *mA*mG*mU*mU*mU*mG*mG*mA |
| 440 | mA*mA*mG*mG*mA*mA*mG*mA*mU*mG*mG*mC*mA*mU*mU*mU*mC |
| | *mU*mA*mG*mU*mU*mU*mG*mG |
| 441 | mC*mA*mA*mG*mG*mA*mA*mG*mA*mU*mG*mG*mC*mA*mU*mU*mU |
| | *mC*mU*mA*mG*mU*mU*mU*mG |
| 442 | mC*mA*mU*mC*mA*mA*mG*mG*mA*mA*mG*mA*mU*mG*mG*mC*mA |
| | *mU*mU*mU*mC*mU*mA*mG*mU |
| 443 | mA*mC*mA*mU*mC*mA*mA*mG*mG*mA*mA*mG*mA*mU*mG*mG*mC |
| | *mA*mU*mU*mU*mC*mU*mA*mG |
| 444 | mA*mA*mC*mA*mU*mC*mA*mA*mG*mG*mA*mA*mG*mA*mU*mG*mG |
| | *mC*mA*mU*mU*mU*mC*mU*mA |
| 445 | mC*mA*mA*mC*mA*mU*mC*mA*mA*mG*mG*mA*mA*mG*mA*mU*mG |
| | *mG*mC*mA*mU*mU*mU*mC*mU |
| 446 | mC*mU*mC*mC*mA*mA*mC*mA*mU*mC*mA*mA*mG*mG*mA*mA*mG |
| | *mA*mU*mG*mG*mC*mA*mU*mU |
| 447 | mA*mC*mC*mU*mC*mC*mA*mA*mC*mA*mU*mC*mA*mA*mG*mG*mA |
| | *mA*mG*mA*mU*mG*mG*mC*mA |
| 448 | mG*mU*mA*mC*mC*mU*mC*mC*mA*mA*mC*mA*mU*mC*mA*mA*mG |
| | *mG*mA*mA*mG*mA*mU*mG*mG |
| 449 | mA*mG*mG*mU*mA*mC*mC*mU*mC*mC*mA*mA*mC*mA*mU*mC*mA |
| | *mA*mG*mG*mA*mA*mG*mA*mU |
| 450 | mA*mG*mA*mG*mC*mA*mG*mG*mU*mA*mC*mC*mU*mC*mC*mA*mA |
| | *mC*mA*mU*mC*mA*mA*mG*mG |
| 451 | mC*mA*mG*mA*mG*mC*mA*mG*mG*mU*mA*mC*mC*mU*mC*mC*mA |
| | *mA*mC*mA*mU*mC*mA*mA*mG |
| 452 | mC*mU*mG*mC*mC*mA*mG*mA*mG*mC*mA*mG*mG*mU*mA*mC*mC |
| | *mU*mC*mC*mA*mA*mC*mA*mU |
| 453 | mU*mC*mU*mG*mC*mC*mA*mG*mA*mG*mC*mA*mG*mG*mU*mA*mC |
| | *mC*mU*mC*mC*mA*mA*mC*mA |
| 454 | mA*mU*mC*mU*mG*mC*mC*mA*mG*mA*mG*mC*mA*mG*mG*mU*mA |
| | *mC*mC*mU*mC*mC*mA*mA*mC |
| 455 | mA*mA*mU*mC*mU*mG*mC*mC*mA*mG*mA*mG*mC*mA*mG*mG*mU |
| | *mA*mC*mC*mU*mC*mC*mA*mA |
| 456 | mA*mA*mA*mU*mC*mU*mG*mC*mC*mA*mG*mA*mG*mC*mA*mG*mG |
| | *mU*mA*mC*mC*mU*mC*mC*mA |
| 457 | mG*mA*mA*mA*mU*mC*mU*mG*mC*mC*mA*mG*mA*mG*mC*mA*mG |
| | *mG*mU*mA*mC*mC*mU*mC*mC |
| 458 | mU*mG*mA*mA*mA*mU*mC*mU*mG*mC*mC*mA*mG*mA*mG*mC*mA |
| | *mG*mG*mU*mA*mC*mC*mU*mC |
| 459 | mU*mU*mG*mA*mA*mA*mU*mC*mU*mG*mC*mC*mA*mG*mA*mG*mC |
| | *mA*mG*mG*mU*mA*mC*mC*mU |
| 460 | mC*mC*mC*mG*mG*mU*mU*mG*mA*mA*mA*mU*mC*mU*mG*mC*mC |
| | *mA*mG*mA*mG*mC*mA*mG*mG |
| 461 | mC*mC*mA*mA*mG*mC*mC*mC*mG*mG*mU*mU*mG*mA*mA*mA*mU |
| | *mC*mU*mG*mC*mC*mA*mG*mA |
| 462 | mU*mC*mC*mA*mA*mG*mC*mC*mC*mG*mG*mU*mU*mG*mA*mA*mA |
| | *mU*mC*mU*mG*mC*mC*mA*mG |
| 463 | mG*mU*mC*mC*mA*mA*mG*mC*mC*mC*mG*mG*mU*mU*mG*mA*mA |
| | *mA*mU*mC*mU*mG*mC*mC*mA |
| 464 | mU*mC*mU*mG*mU*mC*mC*mA*mA*mG*mC*mC*mC*mG*mG*mU*mU |
| | *mG*mA*mA*mA*mU*mC*mU*mG |
| 465 | mU*mU*mC*mU*mG*mU*mC*mC*mA*mA*mG*mC*mC*mC*mG*mG*mU |
| | *mU*mG*mA*mA*mA*mU*mC*mU |
| 466 | mG*mU*mU*mC*mU*mG*mU*mC*mC*mA*mA*mG*mC*mC*mC*mG*mG |
| | *mU*mU*mG*mA*mA*mA*mU*mC |
| 467 | mA*mG*mU*mU*mC*mU*mG*mU*mC*mC*mA*mA'mG*mC*mC*mC*mG* |
| | mG*mU*mU*mG*mA*mA*mA*mU |
| 468 | mA*mA*mG*mU*mU*mC*mU*mG*mU*mC*mC*mA*mA*mG*mC*mC*mC |
| | *mG*mG*mU*mU*mG*mA*mA*mA |
| 469 | mU*mA*mA*mG*mU*mU*mC*mU*mG*mU*mC*mC*mA*mA*mG*mC*mC |
| | *mC*mG*mG*mU*mU*mG*mA*mA |
| 470 | mG*mU*mA*mA*mG*mU*mU*mC*mU*mG*mU*mC*mC*mA*mA*mG*mC |
| | *mC*mC*mG*mG*mU*mU*mG*mA |
| 471 | mG*mG*mU*mA*mA*mG*mU*mU*mC*mU*mG*mU*mC*mC*mA*mA*mG |
| | *mC*mC*mC*mG*mG*mU*mU*mG |
| 472 | mC*mG*mG*mU*mA*mA*mG*mU*mU*mC*mU*mG*mU*mC*mC*mA*mA |
| | *mG*mC*mC*mC*mG*mG*mU*mU |
| 473 | mU*mC*mG*mG*mU*mA*mA*mG*mU*mU*mC*mU*mG*mU*mC*mC*mA |
| | *mA*mG*mC*mC*mC*mG*mG*mU |
| 474 | mG*mU*mC*mG*mG*mU*mA*mA*mG*mU*mU*mC*mU*mG*mU*mC*mC |
| | *mA*mA*mG*mC*mC*mC*mG*mG |
| 475 | mA*mG*mU*mC*mG*mG*mU*mA*mA*mG*mU*mU*mC*mU*mG*mU*mC |
| | *mC*mA*mA*mG*mC*mC*mC*mG |
| 476 | mC*mA*mG*mU*mC*mG*mG*mU*mA*mA*mG*mU*mU*mC*mU*mG*mU |
| | *mC*mC*mA*mA*mG*mC*mC*mC |
| 477 | mA*mA*mA*mG*mC*mC*mA*mG*mU*mC*mG*mG*mU*mA*mA*mG*mU |
| | *mU*mC*mU*mG*mU*mC*mC*mA |
| 478 | mG*mA*mA*mA*mG*mC*mC*mA*mG*mU*mC*mG*mG*mU*mA*mA*mG |
| | *mU*mU*mC*mU*mG*mU*mC*mC |
| 479 | mG*mU*mC*mA*mC*mC*mC*mA*mC*mC*mA*mU*mC*mA*mC*mC*mC* |
| | mU*mC*mU*mG*mU*mG*mA*mU |
| 480 | mG*mG*mU*mC*mA*mC*mC*mC*mA |
| | *mC*mC*mA*mU*mC*mA*mC*mC*mC*mU*mC*mU*mG*mU*mG*mA |
| 481 | mA*mA*mG*mG*mU*mC*mA*mC*mC*mC*mA*mC*mC*mA*mU*mC*mA |
| | *mC*mC*mC*mU*mC*mU*mG*mU |
| 482 | mC*mA*mA*mG*mG*mU*mC*mA*mC*mC*mC*mA*mC*mC*mA*mU*mC |
| | *mA*mC*mC*mC*mU*mC*mU*mG |
| 483 | mU*mC*mA*mA*mG*mG*mU*mC*mA*mC*mC*mC*mA*mC*mC*mA*mU |
| | *mC*mA*mC*mC*mC*mU*mC*mU |
| 484 | mC*mU*mC*mA*mA*mG*mG*mU*mC*mA*mC*mC*mC*mA*mC*mC*mA |
| | *mU*mC*mA*mC*mC*mC*mU*mC |
| 485 | mC*mU*mU*mG*mA*mU*mC*mA*mA*mG*mC*mA*mG*mA*mG*mA*mA |
| | *mA*mG*mC*mC*mA*mG*mU*mC |
| 486 | mA*mU*mA*mA*mC*mU*mU*mG*mA*mU*mC*mA*mA*mG*mC*mA*mG |
| | *mA*mG*mA*mA*mA*mG*mC*mC |
| 487 | mA*mG*mU*mA*mA*mC*mA*mG*mU*mC*mU*mG*mA*mG*mU*mA*mG |
| | *mG*mA*mG |
| 488 | mG*mA*mG*mU*mA*mA*mC*mA*mG*mU*mC*mU*mG*mA*mG*mU*mA |
| | *mG*mG*mA |
| 489 | mA*mG*mA*mG*mU*mA*mA*mC*mA*mG*mU*mC*mU*mG*mA*mG*mU |
| | *mA*mG*mG |
| 490 | mC*mA*mG*mA*mG*mU*mA*mA*mC*mA*mG*mU*mC*mU*mG*mA*mG |
| | *mU*mA*mG |
| 491 | mG*mU*mC*mA*mC*mC*mA*mG*mA*mG*mU*mA*mA*mC*mA*mG*mU |
| | *mC*mU*mG |
| 492 | mU*mG*mU*mC*mA*mC*mC*mA*mG*mA*mG*mU*mA*mA*mC*mA*mG |
| | *mU*mC*mU |
| 493 | mG*mU*mG*mU*mC*mA*mC*mC*mA*mG*mA*mG*mU*mA*mA*mC*mA |
| | *mG*mU*mC |
| 494 | mU*mG*mU*mG*mU*mC*mA*mC*mC*mA*mG*mA*mG*mU*mA*mA*mC |
| | *mA*mG*mU |
| 495 | mU*mU*mG*mU*mG*mU*mC*mA*mC*mC*mA*mG*mA*mG*mU*mA*mA |
| | *mC*mA*mG |
| 496 | mG*mG*mU*mU*mG*mU*mG*mU*mC*mA*mC*mC*mA*mG*mA*mG*mU |
| | *mA*mA*mC |
| 497 | mA*mG*mG*mU*mU*mG*mU*mG*mU*mC*mA*mC*mC*mA*mG*mA*mG |
| | *mU*mA*mA |
| 498 | mC*mA*mG*mG*mU*mU*mG*mU*mG*mU*mC*mA*mC*mC*mA*mG*mA |
| | *mG*mU*mA |
| 499 | mA*mC*mA*mG*mG*mU*mU*mG*mU*mG*mU*mC*mA*mC*mC*mA*mG |
| | *mA*mG*mU |
| 500 | mC*mA*mC*mA*mG*mG*mU*mU*mG*mU*mG*mU*mC*mA*mC*mC*mA |
| | *mG*mA*mG |
| 501 | mC*mC*mA*mC*mA*mG*mG*mU*mU*mG*mU*mG*mU*mC*mA*mC*mC |
| | *mA*mG*mA |
| 502 | mA*mC*mC*mA*mC*mA*mG*mG*mU*mU*mG*mU*mG*mU*mC*mA*mC |
| | *mC*mA*mG |
| 503 | mA*mA*mC*mC*mA*mC*mA*mG*mG*mU*mU*mG*mU*mG*mU*mC*mA |
| | *mC*mC*mA |
| 504 | mU*mA*mA*mC*mC*mA*mC*mA*mG*mG*mU*mU*mG*mU*mG*mU*mC |
| | *mA*mC*mC |
| 505 | mG*mU*mA*mA*mC*mC*mA*mC*mA*mG*mG*mU*mU*mG*mU*mG*mU |
| | *mC*mA*mC |
| 506 | mA*mG*mU*mA*mA*mC*mC*mA*mC*mA*mG*mG*mU*mU*mG*mU*mG |
| | *mU*mC*mA |
| 507 | mC*mU*mU*mA*mG*mU*mA*mA*mC*mC*mA*mC*mA*mG*mG*mU*mU |
| | *mG*mU*mG |
| 508 | mC*mC*mU*mU*mA*mG*mU*mA*mA*mC*mC*mA*mC*mA*mG*mG*mU |
| | *mU*mG*mU |
| 509 | mU*mC*mC*mU*mU*mA*mG*mU*mA*mA*mC*mC*mA*mC*mA*mG*mG |
| | *mU*mU*mG |
| 510 | mU*mU*mC*mC*mU*mU*mA*mG*mU*mA*mA*mC*mC*mA*mC*mA*mG |
| | *mG*mU*mU |
| 511 | mU*mU*mU*mC*mC*mU*mU*mA*mG*mU*mA*mA*mC*mC*mA*mC*mA |
| | *mG*mG*mU |
| 512 | mG*mU*mU*mU*mC*mC*mU*mU*mA*mG*mU*mA*mA*mC*mC*mA*mC |
| | *mA*mG*mG |
| 513 | mA*mG*mU*mU*mU*mC*mC*mU*mU*mA*mG*mU*mA*mA*mC*mC*mA |
| | *mC*mA*mG |
| 514 | mG*mC*mA*mG*mU*mU*mU*mC*mC*mU*mU*mA*mG*mU*mA*mA*mC |
| | *mC*mA*mC |
| 515 | mG*mG*mC*mA*mG*mU*mU*mU*mC*mC*mU*mU*mA*mG*mU*mA*mA |
| | *mC*mC*mA |
| 516 | mU*mG*mG*mC*mA*mG*mU*mU*mU*mC*mC*mU*mU*mA*mG*mU*mA |
| | *mA*mC*mC |
| 517 | mA*mU*mG*mG*mC*mA*mG*mU*mU*mU*mC*mC*mU*mU*mA*mG*mU |
| | *mA*mA*mC |
| 518 | mG*mA*mU*mG*mG*mC*mA*mG*mU*mU*mU*mC*mC*mU*mU*mA*mG |
| | *mU*mA*mA |
| 519 | mA*mG*mA*mU*mG*mG*mC*mA*mG*mU*mU*mU*mC*mC*mU*mU*mA |
| | *mG*mU*mA |
| 520 | mG*mG*mA*mG*mA*mU*mG*mG*mC*mA*mG*mU*mU*mU*mC*mC*mU |
| | *mU*mA*mG |
| 521 | mU*mG*mG*mA*mG*mA*mU*mG*mG*mC*mA*mG*mU*mU*mU*mC*mC |
| | *mU*mU*mA |
| 522 | mU*mU*mG*mG*mA*mG*mA*mU*mG*mG*mC*mA*mG*mU*mU*mU*mC |
| | *mC*mU*mU |
| 523 | mU*mU*mU*mG*mG*mA*mG*mA*mU*mG*mG*mC*mA*mG*mU*mU*mU |
| | *mC*mC*mU |
| 524 | mG*mU*mU*mU*mG*mG*mA*mG*mA*mU*mG*mG*mC*mA*mG*mU*mU |
| | *mU*mC*mC |
| 525 | mC*mU*mA*mG*mU*mU*mU*mG*mG*mA*mG*mA*mU*mG*mG*mC*mA |
| | *mG*mU*mU |
| 526 | mU*mC*mU*mA*mG*mU*mU*mU*mG*mG*mA*mG*mA*mU*mG*mG*mC |
| | *mA*mG*mU |
| 527 | mA*mU*mU*mU*mC*mU*mA*mG*mU*mU*mU*mG*mG*mA*mG*mA*mU |
| | *mG*mG*mC |
| 528 | mU*mG*mG*mC*mA*mU*mU*mU*mC*mU*mA*mG*mU*mU*mU*mG*mG |
| | *mA*mG*mA |
| 529 | mG*mA*mU*mG*mG*mC*mA*mU*mU*mU*mC*mU*mA*mG*mU*mU*mU |
| | *mG*mG*mA |
| 530 | mA*mG*mA*mU*mG*mG*mC*mA*mU*mU*mU*mC*mU*mA*mG*mU*mU |
| | *mU*mG*mG |
| 531 | mA*mA*mG*mA*mU*mG*mG*mC*mA*mU*mU*mU*mC*mU*mA*mG*mU |
| | *mU*mU*mG |
| 532 | mA*mG*mG*mA*mA*mG*mA*mU*mG*mG*mC*mA*mU*mU*mU*mC*mU |
| | *mA*mG*mU |
| 533 | mA*mA*mG*mG*mA*mA*mG*mA*mU*mG*mG*mC*mA*mU*mU*mU*mC |
| | *mU*mA*mG |
| 534 | mC*mA*mA*mG*mG*mA*mA*mG*mA*mU*mG*mG*mC*mA*mU*mU*mU |
| | *mC*mU*mA |
| 535 | mU*mC*mA*mA*mG*mG*mA*mA*mG*mA*mU*mG*mG*mC*mA*mU*mU |
| | *mU*mC*mU |
| 536 | mA*mC*mA*mU*mC*mA*mA*mG*mG*mA*mA*mG*mA*mU*mG*mG*mC |
| | *mA*mU*mU |
| 537 | mC*mA*mA*mC*mA*mU*mC*mA*mA*mG*mG*mA*mA*mG*mA*mU*mG |
| | *mG*mC*mA |
| 538 | mU*mC*mC*mA*mA*mC*mA*mU*mC*mA*mA*mG*mG*mA*mA*mG*mA |
| | *mU*mG*mG |
| 539 | mC*mC*mU*mC*mC*mA*mA*mC*mA*mU*mC*mA*mA*mG*mG*mA*mA |
| | *mG*mA*mU |
| 540 | mA*mG*mG*mU*mA*mC*mC*mU*mC*mC*mA*mA*mC*mA*mU*mC*mA |
| | *mA*mG*mG |
| 541 | mC*mA*mG*mG*mU*mA*mC*mC*mU*mC*mC*mA*mA*mC*mA*mU*mC |
| | *mA*mA*mG |
| 542 | mA*mG*mA*mG*mC*mA*mG*mG*mU*mA*mC*mC*mU*mC*mC*mA*mA |
| | *mC*mA*mU |
| 543 | mC*mA*mG*mA*mG*mC*mA*mG*mG*mU*mA*mC*mC*mU*mC*mC*mA |
| | *mA*mC*mA |
| 544 | mC*mC*mA*mG*mA*mG*mC*mA*mG*mG*mU*mA*mC*mC*mU*mC*mC |
| | *mA*mA*mC |
| 545 | mG*mC*mC*mA*mG*mA*mG*mC*mA*mG*mG*mU*mA*mC*mC*mU*mC |
| | *mC*mA*mA |
| 546 | mU*mG*mC*mC*mA*mG*mA*mG*mC*mA*mG*mG*mU*mA*mC*mC*mU |
| | *mC*mC*mA |
| 547 | mC*mU*mG*mC*mC*mA*mG*mA*mG*mC*mA*mG*mG*mU*mA*mC*mC |
| | *mU*mC*mC |
| 548 | mU*mC*mU*mG*mC*mC*mA*mG*mA*mG*mC*mA*mG*mG*mU*mA*mC |
| | *mC*mU*mC |
| 549 | mA*mU*mC*mU*mG*mC*mC*mA*mG*mA*mG*mC*mA*mG*mG*mU*mA |
| | *mC*mC*mU |
| 550 | mU*mU*mG*mA*mA*mA*mU*mC*mU*mG*mC*mC*mA*mG*mA*mG*mC |
| | *mA*mG*mG |
| 551 | mC*mC*mC*mG*mG*mU*mU*mG*mA*mA*mA*mU*mC*mU*mG*mC*mC |
| | *mA*mG*mA |
| 552 | mG*mC*mC*mC*mG*mG*mU*mU*mG*mA*mA*mA*mU*mC*mU*mG*mC |
| | *mC*mA*mG |
| 553 | mA*mG*mC*mC*mC*mG*mG*mU*mU*mG*mA*mA*mA*mU*mC*mU*mG |
| | *mC*mC*mA |
| 554 | mC*mC*mA*mA*mG*mC*mC*mC*mG*mG*mU*mU*mG*mA*mA*mA*mU |
| | *mC*mU*mG |
| 555 | mU*mC*mC*mA*mA*mG*mC*mC*mC*mG*mG*mU*mU*mG*mA*mA*mA |
| | *mU*mC*mU |
| 556 | mG*mU*mC*mC*mA*mA*mG*mC*mC*mC*mG*mG*mU*mU*mG*mA*mA |
| | *mA*mU*mC |
| 557 | mU*mG*mU*mC*mC*mA*mA*mG*mC*mC*mC*mG*mG*mU*mU*mG*mA |
| | *mA*mA*mU |
| 558 | mC*mU*mG*mU*mC*mC*mA*mA*mG*mC*mC*mC*mG*mG*mU*mU*mG |
| | *mA*mA*mA |
| 559 | mU*mC*mU*mG*mU*mC*mC*mA*mA*mG*mC*mC*mC*mG*mG*mU*mU |
| | *mG*mA*mA |
| 560 | mU*mU*mC*mU*mG*mU*mC*mC*mA*mA*mG*mC*mC*mC*mG*mG*mU |
| | *mU*mG*mA |
| 561 | mG*mU*mU*mC*mU*mG*mU*mC*mC*mA*mA*mG*mC*mC*mC*mG*mG |
| | *mU*mU*mG |
| 562 | mA*mG*mU*mU*mC*mU*mG*mU*mC*mC*mA*mA*mG*mC*mC*mC*mG |
| | *mG*mU*mU |
| 563 | mA*mA*mG*mU*mU*mC*mU*mG*mU*mC*mC*mA*mA*mG*mC*mC*mC |
| | *mG*mG*mU |
| 564 | mU*mA*mA*mG*mU*mU*mC*mU*mG*mU*mC*mC*mA*mA*mG*mC*mC |
| | *mC*mG*mG |
| 565 | mG*mU*mA*mA*mG*mU*mU*mC*mU*mG*mU*mC*mC*mA*mA*mG*mC |
| | *mC*mC*mG |
| 566 | mG*mG*mU*mA*mA*mG*mU*mU*mC*mU*mG*mU*mC*mC*mA*mA*mG |
| | *mC*mC*mC |
| 567 | mC*mA*mG*mU*mC*mG*mG*mU*mA*mA*mG*mU*mU*mC*mU*mG*mU |
| | *mC*mC*mA |
| 568 | mC*mC*mA*mG*mU*mC*mG*mG*mU*mA*mA*mG*mU*mU*mC*mU*mG |
| | *mU*mC*mC |
| 569 | mC*mC*mA*mC*mC*mA*mU*mC*mA*mC*mC*mC*mU*mC*mU*mG*mU |
| | *mG*mA*mU |
| 570 | mC*mC*mC*mA*mC*mC*mA*mU*mC*mA*mC*mC*mC*mU*mC*mU*mG* |
| | mU*mG*mA |
| 571 | mC*mA*mC*mC*mC*mA*mC*mC*mA*mU*mC*mA*mC*mC*mC*mU*mC* |
| | mU*mG*mU |
| 572 | mU*mC*mA*mC*mC*mC*mA*mC*mC*mA*mU*mC*mA*mC*mC*mC*mU* |
| | mC*mU*mG |
| 573 | mG*mU*mC*mA*mC*mC*mC*mA*mC*mC*mA*mU*mC*mA*mC*mC*mC* |
| | mU*mC*mU |
| 574 | mG*mG*mU*mC*mA*mC*mC*mC*mA*mC*mC*mA*mU*mC*mA*mC*mC |
| | *mC*mU*mC |
| 575 | mU*mC*mA*mA*mG*mC*mA*mG*mA*mG*mA*mA*mA*mG*mC*mC*mA |
| | *mG*mU*mC |
| 576 | mU*mU*mG*mA*mU*mC*mA*mA*mG*mC*mA*mG*mA*mG*mA*mA*mA |
| | *mG*mC*mC |
| 577 | mC*mA*mA*mA*mG*mA*mA*mG*mA*mU*mG*mG*mC*mA*mU*mU*mU |
| | *mC*mU*mA*mG*mU*mU*mU*mG |
| 578 | mG*mC*mA*mA*mA*mG*mA*mA*mG*mA*mU*mG*mG*mC*mA*mU*mU |
| | *mU*mC*mU |
| 579 | fG*fC*fA*fA*fA*fG*mA*mA*mG*mA*mU*mG*mG*mC*fA*fU*fU*f |
| | U*fC*fU |
| 580 | fU*fC*fA*fA*fG*mG*mA*mA*mG*mA*mU*mG*mG*mC*mA*fU*fU*f |
| | U*fC*fU |
| 581 | fU*fC*fA*fA*mG*mG*mA*mA*mG*mA*mU*mG*mG*mC*mA*mU*fU*f |
| | U*fC*fU |
| 582 | fU*fC*fA*mA*mG*mG*mA*mA*mG*mA*mU*mG*mG*mC*mA*mU*mU*f |
| | U*fC*fU |
| 583 | fU*fC*mA*mA*mG*mG*mA*mA*mG*mA*mU*mG*mG*mC*mA*mU*mU* |
| | mU*fC*fU |
| 584 | fU*mC*mA*mA*mG*mG*mA*mA*mG*mA*mU*mG*mG*mC*mA*mU*mU* |
| | mU*mC*fU |
| 585 | fU*fC*fA*fA*fG*fGmA*mA*mG*mA*mU*mG*mG*mCfA*fU*fU*fU*fC*fU |
| 586 | fU*fC*fA*fA*fGfGmA*mA*mG*mA*mU*mG*mG*mCfAfU*fU*fU*fC*fU |
| 587 | fU*fC*fA*fAfGfGmA*mA*mG*mA*mU*mG*mG*mCfAfUfU*fU*fC*fU |
| 588 | fU*fC*fAfAfGfGmA*mA*mG*mA*mU*mG*mG*mCfAfUfUfU*fC*fU |
| 589 | fU*fCfAfAfGfGmA*mA*mG*mA*mU*mG*mG*mCfAfUfUfUfC*fU |
| 590 | fUfCfAfAfGfGmA*mA*mG*mA*mU*mG*mG*mCfAfUfUfUfCfU |
| 591 | fU*fC*fA*fA*fG*fG*A*fA*fG*fA*mU*mG*mG*mC*mA*mU*mU*m |
| | U*mC*mU |
| 592 | mU*mC*mA*mA*mG*mG*mA*mA*mG*mA*fU*fG*fG*fC*fA*fU*fU*f |
| | U*fC*fU |
| 593 | fU*fC*fA*fA*fG*fG*mA*mA*mG*mA*mU*mG*mG*mC*mA*mU*mU*m |
| | U*mC*mU |
| 594 | mU*mC*mA*mA*mG*mG*mA*mA*mG*mA*mU*mG*mG*mC*fA*fU*fU*f |
| | U*fC*fU |
| 595 | mU*RmC*RmA*RmA*RmG*RmG*RmA*RmA*RmG*RmA*RmU*RmG*RmG |
| | *RmC*RmA*RmU*RmU*RmU*RmC*RmU |
| 596 | mU*SmC*RmA*SmA*RmG*SmG*RmA*SmA*RmG*SmA*RmU*SmG*RmG* |
| | SmC*RmA*SmU*RmU*SmU*RmC*SmU |
| 597 | mU*RmC*RmA*RmA*SmG*SmG*SmA*SmA*SmG*SmA*SmU*SmG*SmG* |
| | SmC*SmA*SmU*SmU*RmU*RmC*RmU |
| 598 | mU*SmC*SmA*SmA*RmG*RmG*RmA*RmA*RmG*RmA*RmU*RmG*RmG |
| | *RmC*RmA*RmU*RmU*SmU*SmC*SmU |
| 599 | mU*RmC*RmA*RmA*RmG*RmG*SmA*SmA*RmG*SmA*SmU*RmG*SmG* |
| | SmC*RmA*RmU*RmU*RmU*RmC*RmU |
| 600 | mU*SmC*SmA*SmA*SmG*SmG*RmA*RmA*SmG*RmA*RmU*SmG*RmG* |
| | RmC*SmA*SmU*SmU*SmU*SmC*SmU |
| 601 | mU*RmC*RmA*RmA*SmG*SmG*RmA*RmA*SmG*RmA*RmU*RmG*SmG |
| | *RmC*RmA*SmU*SmU*RmU*RmC*RmU |
| 602 | mU*SmC*SmA*SmA*RmG*RmG*SmA*SmA*RmG*SmA*SmU*SmG*RmG* |
| | SmC*SmA*RmU*RmU*SmU*SmC*SmU |
| 603 | mU*SmC*RmA*RmA*RmG*RmG*RmA*RmA*RmG*RmA*RmU*RmG*RmG |
| | *RmC*RmA*RmU*RmU*RmU*RmC*SmU |
| 604 | mU*RmC*SmA*SmA*SmG*SmG*SmA*SmA*SmG*SmA*SmU*SmG*SmG*S |
| | mC*SmA*SmU*SmU*SmU*SmC*RmU |
| 605 | mU*SmC*RmA*SmA*SmG*RmG*RmA*SmA*SmG*RmA*SmU*SmG*RmG* |
| | RmC*RmA*SmU*SmU*SmU*SmC*RmU |
| 606 | mU*RmC*SmA*RmA*RmG*SmG*SmA*RmA*RmG*SmA*RmU*RmG*SmG* |
| | SmC*SmA*RmU*RmU*RmU*RmC*SmU |
| 607 | mU*SmC*SmA*RmA*RmG*RmG*RmA*RmA*RmG*RmA*RmU*SmG*RmG |
| | *RmC*SmA*RmU*SmU*SmU*SmC*SmU |
| 608 | mU*RmC*RmA*SmA*SmG*SmG*SmA*SmA*SmG*SmA*SmU*RmG*SmG* |
| | SmC*RmA*SmU*RmU*RmU*RmC*RmU |
| 609 | fG*fG*fC*fC*fA*fA*fA*fC*fC*fU*fC*fG*fG*fC*fU*fU*fA*fC*fC*fU |
| 610 | mG*mG*fC*fC*mA*mA*mA*fC*fC*fU*fC*mG*mG*fC*fU*fU*mA*fC*fC*f |
| | U |
| 611 | fG*fG*mC*mC*fA*fA*fA*mC*mC*mU*mC*fG*fG*mC*mU*mU*fA*mC*m |
| | C*mU |
| 612 | mG*fG*mC*fC*mA*fA*mA*fC*mC*fU*mC*fG*mG*fC*mU*fU*mA*fC*mC |
| | *fU |
| 613 | mG*mG*mC*mC*mA*mA*fA*fC*fC*fU*fC*fG*fG*fC*mU*mU*mA*mC*m |
| | C*mU |
| 614 | fG*fG*fC*fC*fA*fA*mA*mC*mC*mU*mC*mG*mG*mC*fU*fU*fA*fC*fC*f |
| | U |
| 615 | fG*fG*fC*fC*mA*mA*mA*fC*fC*mU*fC*fG*fG*fC*mU*mU*mA*fC*fC*m |
| | U |
| 616 | mG*mG*mC*mC*fA*fA*fA*mC*mC*fU*mC*mG*mG*mC*fU*fU*fA*mC*m |
| | C*fU |
| 617 | fU*fC*fA*fA*fG*fG*fA*fA*fG*fA*fU*fG*fG*fC*fA*fU*fU*fU*fC*fU |
| 618 | fU*fC*mA*mA*mG*mG*mA*mA*mG*mA*fU*mG*mG*fC*mA*fU*fU*fU*f |
| | C*fU |
| 619 | mU*mC*fA*fA*fG*fG*fA*fA*fG*fA*mU*fG*fG*mC*fA*mU*mU*mU*mC* |
| | mU |
| 620 | mU*fC*mA*fA*mG*G*mA*fA*mG*fA*mU*fG*mG*fC*mA*fU*mU*fU*mC |
| | *fU |
| 621 | mU*mC*mA*mA*mG*mG*fA*fA*fG*fA*fU*fG*fG*fC*mA*mU*mU*mU*m |
| | C*mU |
| 622 | fU*fC*fA*fA*fG*fG*mA*mA*mG*mA*mU*mG*mG*mC*fA*fU*fU*fU*fC*f |
| | U |
| 623 | mU*fC*mA*mA*fG*fG*mA*mA*fG*mA*mU*fG*fG*fC*mA*mU*mU*mU*f |
| | C*mU |
| 624 | fU*mC*fA*fA*mG*mG*fA*fA*mG*fA*fU*mG*mG*mC*fA*fU*fU*fU*mC*f |
| | U |
| 625 | G*G*C*C*A*A*A*C*C*T*C*G*G*C*T*T*A*C*C*T |
| 626 | mGmGmCmCmAmAmAmCmCmUmCmGmGmCmUmUmAmCmCmU |
| 627 | G*RG*RC*RC*RA*RA*RA*RC*RC*RT*RC*RG*RG*RC*RT*RT*RA*RC*R |
| | C*RT |
| 628 | G*SG*SC*SC*SA*SA*SA*SC*SC*ST*SC*SG*SG*SC*ST*ST*SA*SC*SC*S |
| | T |
| 629 | G*SG*SC*SC*SA*SmAmAmCmCmUmCmGmGmCT*ST*SA*SC*SC*ST |
| 630 | mGmGmCmCA*SA*SA*SmCC*ST*SC*SG*SmGC*ST*ST*SmAmCmCmU |
| 631 | G*SmGC*SmCA*SmAA*SmCC*SmUC*SmGG*SmCT*SmUA*SmCC*SmU |
| 632 | mGG*SmCC*SmAA*SmAC*SmCT*SmCG*SmGC*SmUT*SmAC*SmCmU |
| 633 | G*SG*SmCmCA*SA*SmAmCC*ST*SC*SmGmGC*ST*SmUmAC*SC*SmU |
| 634 | G*SG*SC*SmCmAmAA*SC*SmCmUmCG*SG*SmCmUmUA*SC*SC*SmU |
| 635 | G*SG*SC*SC*SmAmAmAmCC*ST*SC*SmGmGmCmUT*SA*SC*SC*SmU |
| 636 | G*SG*SC*SmCA*SA*SA*SmCC*ST*SC*SmGG*SC*ST*SmUA*SC*SC* |
| | SmU |
| 637 | mGmGmCmCA*SA*SA*SC*SC*SmUmCmGmGmCT*ST*SA*SC*SC*SmU |
| 638 | G*G*C*C*A*mAmAmCmCmUmCmGmGmCT*T*A*C*C*T |
| 639 | mGmGmCmCA*A*A*mCC*T*C*G*mGC*T*T*mAmCmCmU |
| 640 | G*mGC*mCA*mAA*mCC*mUC*mGG*mCT*mUA*mCC*mU |
| 641 | mGG*mCC*mAA*mAC*mCT*mCG*mGC*mUT*mAC*mCmU |
| 642 | G*G*mCmCA*A*mAmCmCT*C*mGmGC*T*mUmAC*C*mU |
| 643 | G*G*C*mCmAmAA*C*mCmUmCG*G*mCmUmUA*C*C*mU |
| 644 | G*G*C*C*mAmAmAmCC*T*C*mGmGmCmUT*A*C*C*mU |
| 645 | G*G*C*mCA*A*A*mCC*T*C*mGG*C*T*mUA*C*C*mU |
| 646 | mGmGmCmCA*A*A*C*C*mUmCmGmGmCT*T*A*C*C*mU |
| 647 | G*G*mCmCmAmAmAmCmCmUC*mGmGC*mUT*A*C*C*mU |
| 648 | mG*mG*mC*mC*mA*mAmAmCmCmUm |
| | CmGmGmCmU*mU*mA*mC*mC*mU |
| 649 | mGmGmCmCmA*mA*mA*mCmC*mU*m |
| | C*mG*mGmC*mU*mU*mAmCmCmU |
| 650 | mG*mGmC*mCmA*mAmA*mCmC*mUm |
| | C*mGmG*mCmU*mUmA*mCmC*mU |
| 651 | mGmG*mCmC*mAmA*mAmC*mCmU*m |
| | CmG*mGmC*mUmU*mAmC*mCmU |
| 652 | mG*mG*mCmCmA*mA*mAmCmCmU*m |
| | C*mGmGmC*mU*mUmAmC*mC*mU |
| 653 | mG*mG*mC*mCmAmAmA*mC*mCmUm |
| | CmG*mG*mCmUmUmA*mC*mC*mU |
| 654 | mG*mG*mC*mC*mAmAmAmCmC*mU* |
| | mC*mGmGmCmUmU*mA*mC*mC*mU |
| 655 | mG*mG*mC*mCmA*mA*mA*mCmC*mU*mC*mGmG*mC*mU*mUmA*mC |
| | *mC*mU |
| 656 | mGmGmCmCmA*mA*mA*mC*mC*mUmCmGmGmCmU*mU*mA*mC*mC* |
| | mU |
| 657 | mG*mG*mCmCmAmAmAmCmCmUmC*m |
| | GmGmC*mUmU*mA*mC*mC*mU |
| 658 | LOO1*mG*mG*mC*mC*mA*mA*mA*mC*mC*mU*mC*mG*mG*mC*mU*m |
| | U*mA*mC*mC*mU |
| 659 | LOO1*mG*mG*mC*mC*mA*mA*mA*mC*mC*mU*mC*mG*mG*mC*mU*m |
| | U*mA*mC*mC*mU*mG*mA*mA*mA*m |
| | G*SG*SmCmCmAmAmAmCmCmUC*Sm |
| 660 | G*SG*SmCmCmAmAmAmCmCmUC*SmGmGC*SmUT*SA*SC*SC*SmU |
| 661 | T*C*A*A*G*G*A*A*G*A*T*G*G*C*A*T*T*T*C*T |
| 662 | mUmCmAmAmGmGmAmAmGmAmUmGmGmCmAmUmUmUmCmU |
| 663 | T*RC*RA*RA*RG*RG*RA*RA*RG*RA*RT*RG*RG*RC*RA*RT*RT*RT |
| | *RC*RT |
| 664 | T*SC*SA*SA*SG*SG*SA*SA*SG*SA*ST*SG*SG*SC*SA*ST*ST*ST |
| | *SC*ST |
| 665 | T*SC*SA*SA*SG*SmGmAmAmGmAmUmGmGmCA*ST*ST*ST*SC*ST |
| 666 | mUmCmAmAG*SG*SA*SmAG*SA*ST*SG*SmGC*SA*ST*SmUmUmCmU |
| 667 | T*SmCA*SmAG*SmGA*SmAG*SmAT*SmGG*SmCA*SmUT*SmUC*SmU |
| 668 | mUC*SmAA*SmGG*SmAA*SmGA*SmUG*SmGC*SmAT*SmUT*SmCmU |
| 669 | T*SC*SmAmAG*SG*SmAmAG*SA*ST*SmGmGC*SA*SmUmUT*SC*SmU |
| 670 | T*SC*SA*SmAmGmGA*SA*SmGmAmUG*SG*SmCmAmUT*ST*SC*SmU |
| 671 | T*SC*SA*SA*SmGmGmAmAG*SA*ST*SmGmGmCmAT*ST*ST*SC*SmU |
| 672 | T*SC*SA*SmAG*SG*SA*SmAG*SA*ST*SmGG*SC*SA*SmUT*ST*SC* |
| | SmU |
| 673 | mUmCmAmAG*SG*SA*SA*SG*SmAmUmGmGmCA*ST*ST*ST*SC*SmU |
| 674 | T*SC*SmAmAmGmGmAmAmGmAT*SmGmGC*SmAT*ST*ST*SC*SmU |
| 675 | T*C*A*A*G*mGmAmAmGmAmUmGmGmCA*T*T*T*C*T |
| 676 | mUmCmAmAG*G*A*mAG*A*T*G*mGC*A*T*mUmUmCmU |
| 677 | T*mCA*mAG*mGA*mAG*mAT*mGG*mCA*mUT*mUC*mU |
| 678 | mUC*mAA*mGG*mAA*mGA*mUG*mGC*mAT*mUT*mCmU |
| 679 | T*C*mAmAG*G*mAmAG*A*T*mGmGC*A*mUmUT*C*mU |
| 680 | T*C*A*mAmGmGA*A*mGmAmUG*G*mCmAmUT*T*C*mU |
| 681 | T*C*A*A*mGmGmAmAG*A*T*mGmGmCmAT*T*T*C*mU |
| 682 | T*C*A*mAG*G*A*mAG*A*T*mGG*C*A*mUT*T*C*mU |
| 683 | mUmCmAmAG*G*A*A*G*mAmUmGmGmCA*T*T*T*C*mU |
| 684 | T*C*mAmAmGmGmAmAmGmAT*mGmGC*mAT*T*T*C*mU |
| 685 | mU*mC*mA*mA*mG*mGmAmAmGmAmUmGmGmCmA*mU*mU*mU*mC* |
| | mU |
| 686 | mUmCmAmAmG*mG*mA*mAmG*mA*mU*mG*mGmC*mA*mU*mUmUmC |
| | mU |
| 687 | mU*mCmA*mAmG*mGmA*mAmG*mAmU*mGmG*mCmA*mUmU*mUmC* |
| | mU |
| 688 | mUmC*mAmA*mGmG*mAmA*mGmA*mUmG*mGmC*mAmU*mUmU*mC |
| | mU |
| 689 | mU*mC*mAmAmG*mG*mAmAmG*mA*mU*mGmGmC*mA*mUmUmU*mC |
| | *mU |
| 690 | mU*mC*mA*mAmGmGmA*mA*mGmAmUmG*mG*mCmAmUmU*mU*mC* |
| | mU |
| 691 | mU*mC*mA*mA*mGmGmAmAmG*mA*mU*mGmGmCmAmU*mU*mU*mC |
| | *mU |
| 692 | mU*mC*mA*mAmG*mG*mA*mAmG*mA*mU*mGmG*mC*mA*mUmU*mU |
| | *mC*mU |
| 693 | mUmCmAmAmG*mG*mA*mA*mG*mAmUmGmGmCmA*mU*mU*mU*mC* |
| | mU |
| 694 | mU*mC*mAmAmGmGmAmAmGmAmU*mGmGmC*mAmU*mU*mU*mC*m |
| | U |
| 695 | rArGrArArArUrGrCrCrArUrCrUrUrCrCrUrUrGrA |
| 696 | mU*SmC*SmA*RmA*RmG*RmG*RmA*RmA*RmG*RmA*RmU*RmG*RmG |
| | *RmC*RmA*RmU*RmU*RmU*SmC*SmU |
| 697 | mU*SmC*SmA*SmA*SmG*RmG*RmA*RmA*RmG*RmA*RmU*RmG*RmG |
| | *RmC*RmA*RmU*SmU*SmU*SmC*SmU |
| 698 | mU*SmC*SmA*SmA*SmG*SmG*RmA*RmA*RmG*RmA*RmU*RmG*RmG* |
| | RmC*RmA*SmU*SmU*SmU*SmC*SmU |
| 699 | mU*SmCmAmAmGmGmAmAmGmAmUmGmGmCmAmUmUmUmC*SmU |
| 700 | mU*SmC*SmAmAmGmGmAmAmGmAmU |
| | mGmGmCmAmUmUmU*SmC*SmU |
| 701 | mU*SmC*SmA*SmAmGmGmAmAmGmA |
| | mUmGmGmCmAmUmU*SmU*SmC*SmU |
| 702 | mU*SmC*SmA*SmA*SmGmGmAmAmG |
| | mAmUmGmGmCmAmU*SmU*SmU*SmC*SmU |
| 703 | mU*SmC*SmA*SmA*SmG*SmGmAmA |
| | mGmAmUmGmGmCmA*SmU*SmU*SmU*SmC*SmU |
| 704 | mU*mCmAmAmGmGmAmAmGmAmUmGmGmCmAmUmUmUmC*mU |
| 705 | mU*mC*mAmAmGmGmAmAmGmAmUmGmGmCmAmUmUmU*mC*mU |
| 706 | mU*mC*mA*mAmGmGmAmAmGmAmUmGmGmCmAmUmU*mU*mC*mU |
| 707 | mU*mC*mA*mA*mGmGmAmAmGmAmU |
| | mGmGmCmAmU*mU*mU*mC*mU |
| 708 | mU*mC*mA*mA*mG*mGmAmAmGmAm |
| | UmGmGmCmA*mU*mU*mU*mC*mU |
| 709 | fU*fC*fA*fA*fG*fG*mAmAmGmAmUmGmGmC*fA*fU*fU*fU*fC*fU |
| 710 | fU*fC*fA*fA*fG*mGmAmAmGmAmUmGmGmCmA*fU*fU*fU*fC*fU |
| 711 | fU*fC*fA*fA*mGmGmAmAmGmAmUmGmGmCmAmU*fU*fU*fC*fU |
| 712 | fU*fC*fA*mAmGmGmAmAmGmAmUmGmGmCmAmUmU*fU*fC*fU |
| 713 | fU*fC*mAmAmGmGmAmAmGmAmUmGmGmCmAmUmUmU*fC*fU |
| 714 | fU*mCmAmAmGmGmAmAmGmAmUmGmGmCmAmUmUmUmC*fU |
| 715 | fU*SfC*SfA*SfA*SfG*SfG*SmAmAmGmAmUmGmGmC*SfA*SfU*SfU*SfU |
| | *SfC*SfU |
| 716 | fU*SfC*SfA*SfA*SfG*SmGmAmAmGmAmUmGmGmCmA*SfU*SfU*SfU |
| | *SfC*SfU |
| 717 | fU*SfC*SfA*SfA*SmGmGmAmAmGmAmUmGmGmCmAmU*SfU*SfU*SfC |
| | *SfU |
| 718 | fU*SfC*SfA*SmAmGmGmAmAmGmA |
| | mUmGmGmCmAmUmU*SfU*SfC*SfU |
| 719 | fU*SfC*SmAmAmGmGmAmAmGmAmUmGmGmCmAmUmUmU*SfC*SfU |
| 720 | fU*SmCmAmAmGmGmAmAmGmAmUmGmGmCmAmUmUmUmC*SfU |
| 721 | fU*SfC*SfA*SfA*SfG*SfG*SmA*RmA*RmG*RmA*RmU*RmG*RmG*RmC* |
| | SfA*SfU*SfU*SfU*SfC*SfU |
| 722 | fU*SfC*SfA*SfA*SfG*SmG*RmA*RmA*RmG*RmA*RmU*RmG*RmG*RmC |
| | *RmA*SfU*SfU*SfU*SfC*SfU |
| 723 | fU*SfC*SfA*SfA*SmG*RmG*RmA*RmA*RmG*RmA*RmU*RmG*RmG*Rm |
| | C*RmA*RmU*SfU*SfU*SfC*SfU |
| 724 | fU*SfC*SfA*SmA*RmG*RmG*RmA*RmA*RmG*RmA*RmU*RmG*RmG*R |
| | mC*RmA*RmU*RmU*SfU*SfC*SfU |
| 725 | fU*SfC*SmA*RmA*RmG*RmG*RmA*RmA*RmG*RmA*RmU*RmG*RmG* |
| | RmC*RmA*RmU*RmU*RmU*SfC*SfU |
| 726 | fU*SmC*RmA*RmA*RmG*RmG*RmA*RmA*RmG*RmA*RmU*RmG*RmG* |
| | RmC*RmA*RmU*RmU*RmU*RmC*SfU |
| 727 | fU*SfC*SfA*SfA*SfG*SfG*SfA*SfA*RmG*RmA*RmU*RmG*RmG*SfC*SfA |
| | *SfU*SfU*SfU*SfC*SfU |
| 728 | fU*SfC*SfA*SfA*SfG*SfG*SfA*SfA*SmG*RmA*RmU*RmG*SfG*SfC*SfA* |
| | SfU*SfU*SfU*SfC*SfU |
| 729 | fU*SfC*SfA*SfA*SfG*SfG*SfA*SfA*SfG*SmA*RmU*SfG*SfG*SfC*SfA*Sf |
| | U*SfU*SfU*SfC*SfU |
| 730 | fU*SfC*SfA*SfA*SfG*SfG*SfA*SfAmGmAmUmGmG*SfC*SfA*SfU*SfU*Sf |
| | U*SfC*SfU |
| 731 | fU*SfC*SfA*SfA*SfG*SfG*SfA*SfA*SmGmAmUmG*SfG*SfC*SfA*SfU*SfU |
| | *SfU*SfC*SfU |
| 732 | fU*SfC*SfA*SfA*SfG*SfG*SfA*SfA*SfG*SmAmU*SfG*SfG*SfC*SfA*SfU* |
| | SfU*SfU*SfC*SfU |
| 733 | fU*SfC*SfA*SfA*StG*SfG*SfA*mA*mG*mA*mU*mG*mG*fC*SfA*SfU*SfU |
| | *SfU*SfC*SfU |
| 734 | mU*SmC*SmA*SmA*SmG*SmG*SmA*RmA*RmG*RmA*RmU*RmG*RmG* |
| | RmC*SmA*SmU*SmU*SmU*SmC*SmU |
| 735 | mU*SmC*SmA*SmA*SmG*SmG*SmA*SmA*RmG*RmA*RmU*RmG*RmG* |
| | SmC*SmA*SmU*SmU*SmU*SmC*SmU |
| 736 | mU*SmC*SmA*SmA*SmG*SmG*SmA*SmA*SmG*RmA*RmU*RmG*SmG* |
| | SmC*SmA*SmU*SmU*SmU*SmC*SmU |
| 737 | mU*SmC*SmA*SmA*SmG*SmG*SmA*SmA*SmG*SmA*RmU*SmG*SmG*S |
| | mC*SmA*SmU*SmU*SmU*SmC*SmU |
| 738 | mU*SmC*SmA*SmA*SmG*SmG*SmA |
| | *mA*mG*mA*mU*mG*mG*mC*SmA*SmU*SmU*SmU*SmC*SmU |
| 739 | LOO1*mU*mC*mA*mA*mG*mG*mA* |
| | mA*mG*mA*mU*mG*mG*mC*mA*mU*mU*mU*mC*mU |
| 740 | Mod013L001*mU*mC*mA*mA*mG* |
| | mG*mA*mA*mG*mA*mU*mG*mG*mC*mA*mU*mU*mU*mC*mU |
| 741 | Mod014L001*mU*mC*mA*mA*mG* |
| | mG*mA*mA*mG*mA*mU*mG*mG*mC*mA*mU*mU*mU*mC*mU |
| 742 | Mod005L001*mU*mC*mA*mA*mG* |
| | mG*mA*mA*mG*mA*mU*mG*mG*mC*mA*mU*mU*mU*mC*mU |
| 743 | Mod015L001*mU*mC*mA*mA*mG* |
| | mG*mA*mA*mG*mA*mU*mG*mG*mC*mA*mU*mU*mU*mC*mU |
| 744 | Mod016L001*mU*mC*mA*mA*mG* |
| | mG*mA*mA*mG*mA*mU*mG*mG*mC*mA*mU*mU*mU*mC*mU |
| 745 | Mod017L001*mU*mC*mA*mA*mG* |
| | mG*mA*mA*mG*mA*mU*mG*mG*mC*mA*mU*mU*mU*mC*mU |
| 746 | Mod018L001*mU*mC*mA*mA*mG*mG*mA*mA*mG*mA*mU*mG*mG*mC |
| | *mA*mU*mU*mU*mC*mU |
| 747 | Mod019L001*mU*mC*mA*mA*mG*mG*mA*mA*mG*mA*mU*mG*mG*mC |
| | *mA*mU*mU*mU*mC*mU |
| 748 | Mod006L001*mU*mC*mA*mA*mG*mG*mA*mA*mG*mA*mU*mG*mG*mC |
| | *mA*mU*mU*mU*mC*mU |
| 749 | Mod020L001*mU*mC*mA*mA*mG*mG*mA*mA*mG*mA*mU*mG*mG*mC |
| | *mA*mU*mU*mU*mC*mU |
| 750 | Mod021*mU*mC*mA*mA*mG*mG*mA*mA*mG*mA*mU*mG*mG*mC*mA |
| | *mU*mU*mU*mC*mU |
| 751 | mU*mC*mA*mA*mG*mG*mAmAmGmAmUmGmGmC*mA*mU*mU*mU*m |
| | C*mU |
| 752 | mU*mC*mA*mA*mG*mG*mA*mAmGmAmUmGmG*mC*mA*mU*mU*mU* |
| | mC*mU |
| 753 | mU*mC*mA*mA*mG*mG*mA*mA*mGmAmUmG*mG*mC*mA*mU*mU*m |
| | U*mC*mU |
| 754 | mU*mC*mA*mA*mG*mG*mA*mA*mG*mAmU*mG*mG*mC*mA*mU*mU* |
| | mU*mC*mU |
| 755 | mU*SmC*SmA*SmA*SmG*SmG*SmAmAmGmAmUmGmGmC*SmA*SmU*S |
| | mU*SmU*SmC*SmU |
| 756 | mU*SmC*SmA*SmA*SmG*SmG*SmA*SmAmGmAmUmGmG*SmC*SmA*S |
| | mU*SmU*SmU*SmC*SmU |
| 757 | mU*SmC*SmA*SmA*SmG*SmG*SmA*SmA*SmGmAmUmG*SmG*SmC*Sm |
| | A*SmU*SmU*SmU*SmC*SmU |
| 758 | mU*SmC*SmA*SmA*SmG*SmG*SmA*SmA*SmG*SmAmU*SmG*SmG*Sm |
| | C*SmA*SmU*SmU*SmU*SmC*SmU |
| 759 | fU*fC*fA*fA*fG*fG*fA*mAmGmAmUmGmG*fC*fA*fU*fU*fU*fU*fU |
| 760 | fU*fC*fA*fA*fG*fG*fA*fA*mGmAmUmG*fG*fC*fA*fU*fU*fU*fC*fU |
| 761 | fU*fC*fA*fA*fG*fG*fA*fA*fG*mAmU*fG*fG*fC*fA*fU*fU*fU*fC*fU |
| 762 | fU*SfC*SfA*SfA*SfG*SfG*SmAmAmGmA*RmUmGmGmC*SfA*SfU*SfU*Sf |
| | U*SfC*SfU |
| 763 | fU*SfC*SfA*SfA*SfG*SfG*SmAmAmG*RmA*RmU*RmGmGmC*SfA*SfU*S |
| | fU*SfU*SfC*SfU |
| 764 | fU*SfC*SfA*SfA*SfG*SfG*SmAmA*RmG*RmA*RmU*RmG*RmGmC*SfA* |
| | SfU*SfU*SfU*SfC*SfU |
| 765 | fU*SfC*SfA*SfA*SfG*SfG*SmAmAmGmAfU*SmGmG*SfC*SfA*SfU*SfU*S |
| | fU*SfC*SfU |
| 766 | fU*SfC*SfA*SfA*SmG*SmG*SfAfAmGmAfU*SmGmG*SfC*SfA*SfU*SfU*S |
| | fU*SfC*SfU |
| 767 | fU*SfC*SfA*SfA*SfG*SfG*SmA*RmA*RmGmAmUmG*RmG*RmC*SfA*SfU |
| | *SfU*SfU*SfC*SfU |
| 768 | fU*SfC*SfA*SfA*SfG*SfG*SmA*RmAmGmAmUmGmG*RmC*SfA*SfU*SfU |
| | *SfU*SfC*SfU |
| 769 | fU*SfC*SfA*SfA*SfG*SfG*SmA*SmA*SmGmAmUmG*SmG*SmC*SfA*SfU |
| | *SfU*SfU*SfC*SfU |
| 770 | fU*SfC*SfA*SfA*SfG*SfG*SmA*SmAmGmAmUmGmG*SmC*SfA*SfU*SfU |
| | *SfU*SfC*SfU |
| 771 | fU*SfC*SfA*SfA*SfG*SfG*SmA*SmA*SmG*SmA*SmU*SmG*SmG*SmC*Sf |
| | A*SfU*SfU*SfU*SfC*SfU |
| 772 | fU*SfC*SfA*SfA*SfG*SfG*SmA*RmA*RmG*RmA*RfU*SmG*RmG*SfC*Sf |
| | A*SfU*SfU*SfU*SfC*SfU |
| 773 | fU*SfC*SfA*SfA*SmG*SmG*SfA*RfA*RmG*RmA*RfU*SmG*RmG*SfC*Sf |
| | A*SfU*SfU*SfU*SfC*SfU |
| 774 | fU*SfC*SfA*SfA*SfG*SfG*SfA*SmAmGmAmUmGmG*SfC*SfA*SfU*SfU*S |
| | fU*SfC*SfU |
| 775 | fU*SfC*SfA*SfA*SfG*SfG*SfA*SmA*RmG*RmA*RmU*RmG*RmG*SfC*Sf |
| | A*SfU*SfU*SfU*SfC*SfU |
| 776 | mU*SmC*SmA*SfA*SfG*SfG*SmA*RmA*RmG*RmA*RmU*RmG*RmG*Rm |
| | C*SfA*SfU*SfU*SmU*SmC*SmU |
| 777 | mU*SmC*SmA*SfA*SfG*SfG*SfA*SmA*RmG*RmA*RmU*RmG*RmG*SfC* |
| | SfA*SfU*SfU*SmU*SmC*SmU |
| 778 | mU*SmC*SmA*SfA*SfG*SfG*SfA*SfA*SmG*RmA*RmU*RmG*SfG*SfC*Sf |
| | A*SfU*SfU*SmU*SmC*SmU |
| 779 | mU*SmC*SmA*SfA*SfG*SfG*SfA*SfA*SfG*SmA*RmU*StG*SfG*SfC*SfA* |
| | SfU*SfU*SmU*SmC*SmU |
| 780 | mU*SmC*SmA*SfA*SfG*SfG*SfA*SfA*SmGmAmUmG*SfG*SfC*SfA*SfU* |
| | SfU*SmU*SmC*SmU |
| 781 | mU*SmC*SmA*SfA*SfG*SfG*SfA*SfA*SfG*SmAmU*SfG*SfG*SfC*SfA*Sf |
| | U*SfU*SmU*SmC*SmU |
| 782 | fU*fC*fA*fA*fG*fG*fA*fA*mG*mA*mU*mG*mG*fC*fA*fU*fU*fU*fC*fU |
| 783 | fU*fC*fA*fA*fG*fC*fA*fA*mG*mA*mU*mG*fG*fC*fA*fU*fU*fU*fC*fU |
| 784 | fU*fC*fA*fA*fG*fG*fA*fA*fG*mA*mU*fG*fc*fA*fU*fU*fU*fC*fU |
| 785 | fU*fC*fA*fA*fG*fG*fA*mA*mG*mA*mU*mG*mG*fC*fA*fU*fU*f |
| | U*fC*fU |
| 786 | mU*mC*mA*fA*fG*fG*mA*mA*mG*mA*mU*mG*mG*mC*fA*fU*fU*mU* |
| | mC*mU |
| 787 | mU*mC*mA*fA*fG*fG*fA*mA*mG*mA*mU*mG*mG*fC*fA*fU*fU*m |
| | U*mC*mU |
| 788 | mU*mC*mA*fA*fG*fG*fA*fA*mG*mA*mU*mG*fG*fC*fA*fU*fU*m |
| | U*mC*mU |
| 789 | mU*mC*mA*fA*fG*fG*fA*fA*fC*mA*mU*fG*fG*fC*fA*fU*fU*m |
| | U*mC*mU |
| 790 | mU*mC*mA*fA*fG*fG*fA*fA*mGmAmUmG*fG*fC*fA*fU*fU*mU*m |
| | C*mU |
| 791 | mU*mC*mA*fA*fG*fG*fA*fA*fG*mAmU*fG*fG*fC*fA*fU*fU*mU |
| | *mC*mU |
| 792 | Mod024L001*mU*mC*mA*mA*mG*mG*mA*mA*mG*mA*mU*mG*mG*mC |
| | *mA*mU*mU*mU*mC*mU |
| 793 | Mod026L001*mU*mC*mA*mA*mG*mG*mA*mA*mG*mA*mU*mG*mG*mC |
| | *mA*mU*mU*mU*mC*mU |
| 794 | fU*fC*fA*fA*fG*fG*mA*mA*mG*mA*BrdU*mG*mG*mC*fA*fU*fU |
| | *fU*fC*fU |
| 795 | fU*fC*fA*fA*fG*fG*fA*fA*fG*mA*BrdU*fG*fC*fC*fA*fU*fU*fU*fC*fU |
| 796 | mU*mC*mA*mA*mG*mG*mA*mA*mG*mA*BrdU*mG*mG*mC*mA*mU*m |
| | U*mU*mC*mU |
| 797 | fU*SfC*SfA*SfA*SfG*SfG*SfA*SfA*SmGmABrdUmG*SfG*SfC*SfA*SfU*Sf |
| | U*SfU*SfC*SfU |
| 798 | fU*fC*fA*fA*fG*fG*fA*fA*mGmABrdUmG*fG*fC*fA*fC*fU*fU*fC*fU |
| 799 | fU*SfC*SfA*SfA*SfG*SfG*SmAmAmGmABrdUmGmGmC*SfA*SfU*SfU*Sf |
| | U*SfC*SfU |
| 800 | fU*fC*fA*fA*fG*fG*mAmAmGmABrdUmGmGmC*fA*fU*fU*fU*fC*fU |
| 801 | LOO1*fU*SfC*SfA*SfA*SfG*SfG*SmAmAmGmAmUmGmGmC*SfA*SfU*Sf |
| | U*SfU*SfC*SfU |
| 802 | Mod015L001*fU*SfC*SfA*SfA*SfG*SfG*SmAmAmGmAmUmGmGmC*SfA* |
| | SfU*SfU*SfU*SfC*SfU |
| 803 | Mod006L001*fU*SfC*SfA*SfA*SfG*SfG*SmAmAmGmAmUmGmGmC*SfA* |
| | SfU*SfU*SfU*SfC*SfU |
| 804 | LOO1*fU*SfC*SfA*SfA*SfG*Sf |
| | G*SfA*SfA*SmGmAmUmG*SfG*SfC*SfA*SfU*SfU*SfU*SfC*SfU |
| 805 | Mod015L001*fU*SfC*SfA*SfA*SfG*SfG*SfA*SfA*SmGmAmUmG*SfG*SfC |
| | *SfA*SfU*SfU*SfU*SfC*SfU |
| 806 | Mod006L001*fU*SfC*SfA*SfA*SfG*SfG*SfA*SfA*SmGmAmUmG*SfG*SfC |
| | *SfA*SfU*SfU*SfU*SfC*SfU |
| 807 | fU*SfC*SfA*SfA*SfG*SfG*SfA*SfA*SmGmAmUmGmG*SfC*SfA*SfU*SfU* |
| | SfU*SfC*SfU |
| 808 | LOO1*fU*fC*fA*fA*fG*fG*mA*mA*mG*mA*mU*mG*mG*mC*fA*fU*fU*f |
| | U*fC*fU |
| 809 | Mod015L001*fU*fC*fA*fA*fG*fG*mA*mA*mG*mA*mU*mG*mG*mC*fA*f |
| | U*fU*fU*fC*fU |
| 810 | Mod006L001*fU*fC*fA*fA*fG*fG*mA*mA*mG*mA*mU*mG*mG*mC*fA*f |
| | U*fU*fU*fC*fU |
| 811 | Mod020L001*fU*fC*fA*fA*fG*fG*mA*mA*mG*mA*mU*mG*mG*mC*fA*f |
| | U*fU*fU*fC*fU |
| 812 | Mod019L001*fU*fC*fA*fA*fG*fG*mA*mA*mG*mA*mU*mG*mG*mC*fA*f |
| | U*fU*fU*fC*fU |
| 813 | LOO1*fU*fC*fA*fA*fG*fG*fA*fA*mGmAmUmG*fG*fC*fA*fU*fU |
| | *fU*fC*fU |
| 814 | Mod015L001*fU*fC*fA*fA*fG*fG*fA*fA*mGmAmUmG*fG*fC*fA*fU*fU*f |
| | U*fC*fU |
| 815 | Mod006L001*fU*fC*fA*fA*fG*fG*fA*fA*mGmAmUmG*fG*fC*fA*fU*fU*f |
| | U*fC*fU |
| 816 | Mod020L001*fU*ft*fA*fA*fG*fG*fA*fA*mGmAmUmG*fG*fC*fA |
| | *fU*fU*fU*fC*fU |
| 817 | Mod019L001*fU*ft*fA*fA*fG*fG*fA*fA*mGmAmUmG*fG*fC*fA |
| | *fU*fU*fU*fC*fU |
| 818 | fU*fC*fA*fA*fG*fG*mAmAmGmA*mUmGmGmC*fA*fU*fU*fU*fC*fU |
| 819 | fU*fC*fA*fA*fG*fG*mAmAmG*mA*mU*mGmGmC*fA*fU*fU*fU*fC*fU |
| 820 | fU*fC*fA*fA*fG*fG*mAmA*mG*mA*mU*mG*mGmC*fA*fU*fU*fU*fC*fU |
| 821 | fU*fC*fA*fA*fG*fG*mA*mA*mGmAmUmG*mG*mC*fA*fU*fU*fU*fC*fU |
| 822 | fU*fC*fA*fA*fG*fG*mA*mAmGmAmUmGmG*mC*fA*fU*fU*fU*fC*fU |
| 823 | fU*fC*fA*fA*fG*fG*mA*mA*mGmAmUmG*mG*mC*fA*fU*fU*fU*fC*fU |
| 824 | fU*fC*fA*fA*fG*PG*mA*mAmGmAmUmGmG*mC*fA*fU*fU*fU*fC*fU |
| 825 | fU*fC*fA*fA*fG*fG*mAmAmGmAfU*mGmG*fC*fA*fU*fU*fU*fC*fU |
| 826 | fU*fC*fA*fA*fG*fG*mA*mA*mG*mA*fU*mG*mG*fC*fA*fU*fU*fU*fC*fU |
| 827 | fU*fC*fA*fA*mG*mG*fAfAmGmAfU*mGmG*fC*fA*fU*fU*fU*fC*fU |
| 828 | fU*fC*fA*fA*mG*mG*fA*fA*mG*mA*fU*mG*mG*fC*fA*fU*fU*fU*fC*fU |
| 829 | fU*fC*fA*fA*fG*fG*mA*mA*mG*mA*fU*mG*mG*fC*fA*fU*fU*fU*fC*fU |
| 830 | fU*fC*fA*fA*mG*mG*fA*fA*mG*mA*fU*mG*mG*fC*fA*fU*fU*fU*fC*fU |
| 831 | fU*fC*fA*fA*mG*mG*fA*fA*mGmAmUmG*mG*fC*fA*fU*fU*fU*fC*fU |
| 832 | fU*fC*fA*fA*mG*mG*mA*mA*mGmAfUmG*mG*fC*fA*fU*fU*fU*fC*fU |
| 833 | fU*fC*fA*fA*mG*mG*fA*fA*mG*mA*mU*mG*mG*fC*fA*fU*fU*f |
| | U*fC*fU |
| 834 | fU*fC*fA*fA*mG*mG*mA*mA*mG*mA*fU*mG*mG*fC*fA*fU*fU*f |
| | U*fC*fU |
| 835 | fU*fC*fA*fA*mG*mG*fAfAmGmA*fU*mGmG*fC*fA*fU*fU*fU*fC*fU |
| 836 | fU*fC*fA*fA*mG*mG*fA*fA*mG*mA*fU*mG*mG*fC*fA*fU*fU*fU*fC*fU |
| 837 | fU*fC*fA*fA*mG*mG*fA*fA*mG*fA*fU*mG*mG*fC*fA*fU*fU*fU*fC*fU |
| 838 | fU*fC*fA*fA*mG*mG*fA*fA*mGmAmUmGmG*fC*fA*fU*fU*fU*fC*fU |
| 839 | fU*fC*fA*fA*mG*mG*fA*fA*mGmAfU*mGmG*fC*fA*fU*fU*fU*fC*fU |
| 840 | fU*fC*fA*fA*mG*mG*mA*mA*mGmAfU*mGmG*fC*fA*fU*fU*fU*fC*fU |
| 841 | fU*SfC*SfA*SfA*SfG*SfG*SmAmAmGmAmU:mGmGmC*SfA*SfU*SfU*SfU |
| | *SfC*SfU |
| 842 | fU*SfC*SfA*SfA*SfG*SfG*SmAmA:mGmAmUmG:mGmC*SfA*SfU*SfU*Sf |
| | U*SfC*SfU |
| 843 | fU*SfC*SfA*SfA*SfG*SfG*SmA:mAmG:mAmUmGmG:mC*SfA*SfU*SfU*Sf |
| | U*SfC*SfU |
| 844 | fU*SfC*SfA*SfA*SfG*SfG*SmA:mAmGmAmU:mGmG:mC*SfA*SfU*SfU*Sf |
| | U*SfC*SfU |
| 845 | fU*SfC*SfA*SfA*fG:fG:mAmAmGmAmU:mGmGmC*SfA*SfU*SfU*S |
| | fU*SfC*SfU |
| 846 | fU*SfC*SfA*SfA*mG:mG:mAmAmGmAmU:mGmGmC*SfA*SfU*SfU*SfU*Sf |
| | C*SfU |
| 847 | fU*SfC*SfA*SfA*SfG*SfG*SfA*SmAmGmAmU:mGmG*SfC*SfA*SfU*SfU*S |
| | fU*SfC*SfU |
| 848 | fU*SfC*SfA*SfA*fG:fG:fA*SmAmGmAmU:mGmG*SfC*SfA*SfU*SfU*SfU*S |
| | fC*SfU |
| 849 | fU*SfC*SfA*SfA*mG:mG:fA*Sm |
| | AmGmAmU:mGmG*SfC*SfA*SfU*SfU*SfU*SfC*SfU |
| 850 | fU*SfC*SfA*SfA*SfG*SfG*SfA*SfA*SmGmAmU:mG*SfG*SfC*SfA*SfU*Sf |
| | U*SfU*SfC*SfU |
| 851 | fU*SfC*SfA*SfA*SfG*SfG*SfA*SfA*SmG:mA:mU:mG*SfG*SPC*SfA*SfU*S |
| | fU*SfU*SfC*SfU |
| 852 | fU*SfC*SfA*SfA*SfG*SfG*SfA*SfA*SmG:mAmU:mG*SfG*SfC*SfA*SfU*Sf |
| | U*SfU*SfC*SfU |
| 853 | fU*SfC*SfA*SfA*fG:fG:fA*SfA*SmGmAmU:mG*SfG*SfC*SfA*SfU*SfU*Sf |
| | U*SfC*SfU |
| 854 | fU*SfC*SfA*SfA*mG:mG:fA*SfA*SmGmAmU:mG*SfG*SfC*SfA*SfU*SfU*S |
| | fU*SfC*SfU |
| 855 | Mod015L001mU*mC*mA*mA*mG*mG*mA*mA*mG*mA*mU*mG*mG*mC* |
| | mA*mU*mU*mU*mC*mU |
| 856 | Mod019L001mU*mC*mA*mA*mG*mG*mA*mA*mG*mA*mU*mG*mG*mC* |
| | mA*mU*mU*mU*mC*mU |
| 857 | Mod020L001mU*mC*mA*mA*mG*mG*mA*mA*mG*mA*mU*mG*mG*mC* |
| | mA*mU*mU*mU*mC*mU |
| 858 | Mod015L001:mU*mC*mA*mA*mG*mG*mA*mA*mG*mA*mU*mG*mG*mC |
| | *mA*mU*mU*mU*mC*mU |
| 859 | Mod019L001:mU*mC*mA*mA*mG*mG*mA*mA*mG*mA*U*mG*mG*mC |
| | *mA*mU*mU*mU*mC*mU |
| 860 | Mod020L001:mU*mC*mA*mA*mG*mG*mA*mA*mG*mA*mU*mG*mG*mC |
| | *mA*mU*mU*mU*mC*mU |
| 861 | fU*SfC*SfA*SfA*SfG:fG:mAmAmGmAmU:mGmGmC*SfA*SfU*SfU* |
| | SfU*SfC*SfU |
| 862 | fU*SfC*SfA*SfA*SmG:mG:mAmAmGmAmU:mGmGmC*SfA*SfU*SfU*SfU* |
| | SfC*SfU |
| 863 | fU*SfC*SfA*SfA*SfG:fG:fA*SmAmGmAmU:mGmG*SfC*SfA*SfU*SfU*SfU* |
| | SfC*SfU |
| 864 | fU*SfC*SfA*SfA*SmG:mG:fA*SmAmGmAmU:mGmG*StC*SfA*SfU*SfU*Sf |
| | U*SfC*SfU |
| 865 | fU*SfC*SfA*SfA*SfG:fG:fA*SfA*SmGmAmU:mG*SPG*SfC*SfA*SfU*SfU*S |
| | fU*SfC*SfU |
| 866 | fU*SPC*SfA*SfA*SmG:mG:fA*SfA*SmGmAmU:mG*SfG*SfC*SfA*SfU*SfU |
| | *SfU*SfC*SfU |
| 867 | fU*SfC*SfA*SfA*SfG*SfG*SfA*SfA*SfG*SmAmUmG*SfG*SfC*SfA*SfU*Sf |
| | U*SfU*SPC*SfU |
| 868 | fU*fC*fA*fA*fG*fG*fA*fA*fG*mAmUmG*fG*fC*fA*fU*fU*fU*fC*fU |
| 869 | fU*SfC*SfA*SfA*SfG*SfG*SmAfA*SmGfA*SmUfG*SmGfC*SfA*SfU*SfU*S |
| | fU*SfC*SfU |
| 870 | fU*SfC*SfA*SfA*SfG*SfG*SfA*SfA*SmGfA*SmUPG*SmG*SfC*SfA*SfU*Sf |
| | U*SfU*SfC*SfU |
| 871 | LOO1mU*mC*mA*mA*mG*mG*mA*mA*mG*mA*mU*mG*mG*mC*mA*m |
| | U*mU*mU*mC*mU |
| 872 | LOO1fU*SfC*SfA*SfA*SfG*SfG*SfA*SfA*SfG*SmAmU*SfG*SfG*SfC*SfA* |
| | SfU*SfU*SfU*SfC*SfU |
| 873 | Mod013L001mU*mC*mA*mA*mG*mG*mA*mA*mG*mA*mU*mG*mG*mC* |
| | mA*mU*mU*mU*mC*mU |
| 874 | Mod013L001fU*SfC*SfA*SfA*SfG*SfG*SfA*SfA*SfG*SmAmU*SfG*SfG*Sf |
| | C*SfA*SfU*SfU*SfU*SfC*SfU |
| 875 | Mod014L001fU*SfC*SfA*SfA*SfG*SfG*SfA*SfA*SfG*SmAmU*SfG*SfG*Sf |
| | C*SfA*SfU*SfU*SfU*SfC*SfU |
| 876 | Mod005L001fU*SfC*SfA*SfA*SfG*SfG*SfA*SfA*SfG*SmAmU*SfG*SfG*Sf |
| | C*SfA*SfU*SfU*SfU*SfC*SfU |
| 877 | Mod015L001fU*SfC*SfA*SfA*SfG*SfG*SfA*SfA*SfG*SmAmU*SfG*SfG*Sf |
| | C*SfA*SfU*SfU*SfU*SfC*SfU |
| 878 | Mod020L001fU*SfC*SfA*SfA*SfG*SfG*SfA*SfA*SfG*SmAmU*SfG*SfG*Sf |
| | C*SfA*SfU*SfU*SfU*SfC*SfU |
| 879 | Mod027L001fU*SfC*SfA*SfA*SfG*SfG*SfA*SfA*SfG*SmAmU*SfG*SfG*Sf |
| | C*SfA*SfU*SfU*SfU*SfC*SfU |
| 880 | Mod029L001fU*SfC*SfA*SfA*SfG*SfG*SfA*SfA*SfG*SmAmU*SfG*SfG*Sf |
| | C*SfA*sfU*SfU*SfU*SfC*SfU |
| 881 | fU*SfC*SfA*SfA*SfG*SfGfA*SmAfG*SmAfU*SmGfGfC*SfA*SfU*SfU*SfU* |
| | SfC*SfU |
| 882 | fU*SfC*SfA*SfA*SfG*SfG*SfA*SfAfG*SmAfU*SmG*SmG*SfC*SfA*SfU*Sf |
| | U*SfU*SfC*SfU |
| 883 | fU*SfC*SfA*SfA*SfG*SfG*SfA*SfA*SfG*SmAfU*SmG*SfG*SfC*SfA*SfU* |
| | SfU*SfU*SfC*SfU |
| 884 | fU*SfC*SfA*SfA*SfG*SfG*SfA*SfA*SmGmAfU*SmGmG*SfC*SfA*SfU*SfU |
| | *SfU*SfC*SfU |
| 885 | fU*SfC*SfA*SfA*SfG*SfG*SfA*SfA*SfG*SmAfU*SmGfG*SfC*SU*SfC*SfU |
| 886 | fU*SfC*SfA*SfA*SfG*mAmAmGmAfU*SmGmG*SfC*SfA*SfU*SfU* |
| | SfU*SfC*SfU |
| 887 | fU*SfC*SfA*SfA*SfG*SfG*SmA*SmA*SmG*SmA*SfU*SmG*SmG*SfC*SfA |
| | *SfU*SfU*SfU*SfC*SfU |
| 888 | fU*SfC*SfA*SfA*SfG*SfG*SmAfA*SmGfA*SfUfG*SmGfC*SfA*SfU*SfU*Sf |
| | U*SfC*SfU |
| 889 | fU*SfC*SfA*SfA*SfG*SfG*SmAfA*SmGfA*SfUmGmGfC*SfA*SfU*SfU*SfU |
| | *SfC*SfU |
| 890 | fU*SfC*SfA*SfA*SfG*SfG*SmAfA*SmGfA*SfU*SmGmGfC*SfA*SfU*SfU*S |
| | fU*SfC*SfU |
| 891 | fU*SfC*SfA*SfA*SfG*SfG*SmAfA*SmGmA*SfU*SmGmGfC*SfA*SfU*SfU* |
| | SfU*SfC*SfU |
| 892 | fU*SfC*SfA*SfA*SfG*SfG*SmAfA*SmGfAfU*SmGmGfC*SfA*SfU*SfU*SfU |
| | *SfC*SfU |
| 893 | fU*SfC*SfA*SfA*SfG*SfG*SmAfA*SmGmAfU*SmGmGfC*SfA*SfU*SfU*Sf |
| | U*SfC*SfU |
| 894 | fU*SfC*SfA*SfA*SfG*SfG*SmAfA*SmGfA*SfU*SmGmGfC*SfAfU*SfU*SfU |
| | *SfC*SfU |
| 895 | fU*SfC*SfA*SfA*SfG*SfG*SmAfA*SmGmA*SfU*SmGmGfC*SfAfU*SfU*Sf |
| | U*SfC*SfU |
| 896 | fU*SfC*SfA*SfA*SfG*SfG*SmAfA*SmGfAfU*SmGmGfC*SmA*SfU*SfU*Sf |
| | U*SfC*SfU |
| 897 | fU*SfC*SfA*SfA*SfG*SfG*SmAfA*SmGmAfU*SmGmGfC*SmA*SfU*SfU*S |
| | fU*SfC*SfU |
| 898 | fU*SfC*SfA*SfA*SfG*SfG*SmAfA*SmGfAfU*SmGmGfC*SmAfU*SfU*SfU* |
| | SfC*SfU |
| 899 | fU*SfC*SfA*SfA*SfG*SfG*SmAfA*SmGmAfU*SmGmGfC*SmAfU*SfU*SfU |
| | *SfC*SfU |
| 900 | fU*SfC*SfA*SfA*SfG*SfG*SmAfA*SmGfAfU*SmGmGfC*SfAfU*SfU*SfU*S |
| | fC*SfU |
| 901 | fU*SfC*SfA*SfA*SfG*SfG*SmAfA*SmGmAfU*SmGmGfC*SfAfU*SfU*SfU* |
| | SfC*SfU |
| 902 | fU*fC*fA*fA*fG*fG*mAfA*mGfA*mUfG*mGfC*fA*fU*fU*fU*fC*fU |
| 903 | Mod030fU*fC*fA*fA*fG*fG*mAfA*mGfA*mUfG*mGfC*fA*fU*fU |
| | *fU*fC*fU |
| 904 | Mod031fU*fC*fA*fA*fG*fG*mAfA*mGfA*mUfG*mGfC*fA*fU*fU |
| | *fU*fC*fU |
| 905 | Mod032fU*fC*fA*fA*fG*fG*mAfA*mGfA*mUfG*mGfC*fA*fU*fU |
| | *fU*fC*fU |
| 906 | Mod033fU*fC*fA*fA*fG*fG*mAfA*mGfA*mUfG*mGfC*fA*fU*fU |
| | *fU*fC*fU |
| 907 | Mod013L001fU*SfC*SfA*SfA*SfG*SfG*SmAfA*SmGmA*SfU*SmGmGfC*Sf |
| | A*SfU*SfU*SfU*SfC*SfU |
| 908 | Mod005L001fU*SfC*SfA*SfA*SfG*SfG*SmAfA*SmGmA*SfU*SmGmGfC*Sf |
| | A*SfU*SfU*SfU*SfC*SfU |
| 909 | Mod015L001fU*SfC*SfA*SfA*SfG*SfG*SmAfA*SmGmA*SfU*SmGmGfC*Sf |
| | A*SfU*SfU*SfU*SfC*SfU |
| 910 | Mod020L001fU*SfC*SfA*SfA*SfG*SfG*SmAfA*SmGmA*SfU*SmGmGfC*Sf |
| | A*SfU*SfU*SfU*SfC*SfU |
| 911 | Mod027L001fU*SfC*SfA*SfA*SfG*SfG*SmAfA*SmGmA*SfU*SmGmGfC*Sf |
| | A*SfU*SfU*SfU*SfC*SfU |
| 912 | Mod029L001fU*SfC*SfA*SfA*SfG*SfG*SmAfA*SmGmA*SfU*SmGmGfC*Sf |
| | A*SfU*SfU*SfU*SfC*SfU |
| 913 | Mod030fU*SfC*SfA*SfA*SfG*SfG*SmAfA*SmGmA*SfU*SmGmGfC*SfA*Sf |
| | U*SfU*SfU*SfC*SfU |
| 914 | Mod032fU*SfC*SfA*SfA*SfG*SfG*SmAfA*SmGmA*SfU*SmGmGfC*SfA*Sf |
| | U*SfU*SfU*SfC*SfU |
| 915 | Mod033fU*SfC*SfA*SfA*SfG*SfG*SmAfA*SmGmA*SfU*SmGmGfC*SfA*Sf |
| | U*SfU*SfU*SfC*SfU |
| 916 | Mod020L001*fU*SfC*SfA*SfA*SfG*SfG*SmAfA*SmGmA*SfU*SmGmGfC*S |
| | fA*SfU*SfU*SfU*SfC* |
| 917 | Mod005L001*fU*SfC*SfA*SfA*SfG*SfG*SmAfA*SmGmA*SfU*SmGmGfC*S |
| | fA*SfU*SfU*SfU*SfC*SfU |
| 918 | Mod014L001fU*SfC*SfA*SfA*SfG*SfG*SmAfA*SmGmA*SfU*SmGmGfC*Sf |
| | A*SfU*SfU*SfU*SfC*SfU |
| 919 | Mod030*fU*SfC*SfA*SfA*SfG*SfG*SmAfA*SmGmA*SfU*SmGmGfC*SfA*S |
| | fU*SfU*SfU*SfC*SfU |
| 920 | Mod032*fU*SfC*SfA*SfA*SfG*SfG*SmAfA*SmGmA*SfU*SmGmGfC*SfA*S |
| | fU*SfU*SfU*SfC*SfU |
| 921 | Mod033*fU*SfC*SfA*SfA*SfG*SfG*SmAfA*SmGmA*SfU*SmGmGfC*SfA*S |
| | fU*SfU*SfU*SfC*SfU |
| 922 | Mod033*fU*fC*fA*fA*fG*fG*mAfA*mGfA*mUfG*mGfC*fA*fU*fU*fU*fC*f |
| | U |
| 923 | Mod020L001fU*fC*fA*fA*fG*fG*mAfA*mGfA*mUfG*mGfC*fA*fU*fU*fU* |
| | fC*fU |
| 924 | Mod020L001*fU*fC*fA*fA*fG*fG*mAfA*mGfA*mUfG*mGfC*fA*fU*fU*fU |
| | *fC*fU |
| 925 | LOO1*fU*SfC*SfA*SfA*SfG*SfG*SmAfA*SmGmA*SfU*SmGmGfC*SfA*SfU |
| | *SfU*SfU*SfC*SfU |
| 926 | LOO1fU*SfC*SfA*SfA*SfG*SfG*SmAfA*SmGmA*SfU*SmGmGfC*SfA*SfU* |
| | SfU*SfU*SfC*SfU |
| 927 | rGrCrCrArArCrUrGrGrGrArGrCrUrGrGrArGrCrGrCrArCrCrArArCrCrArG |
| 928 | LOO1*fU*fC*fA*fA*fG*fG*mAfA*mGfA*mUfG*mGfC*fA*fU*fU*fU*fC*fU |
| 929 | LOO1fU*fC*fA*fA*fG*fG*mAfA*mGfA*mUfG*mGfC*fA*fU*fU*fU*fC*fU |
| 930 | Mod015L001*fU*fC*fA*fA*fG*fG*mAfA*mGfA*mUfG*mGfC*fA*fU*fU*fU |
| | *fC*fU |
| 931 | Mod015L001fU*fC*fA*fA*fG*fG*mAfA*mGfA*mUfG*mGfC*fA*fU*fU*fU* |
| | fC*fU |
| 932 | fC*fC*fU*fU*fC*fC*mCfU*GmAfA*mGmGfU*fU*fC*fC*fU*fC*fC |
| 933 | LOO1fC*fC*fU*fU*fC*fC*mCfU*GmAfA*mGmGfU*fU*fC*fC*fU*fC*fC |
| 934 | Mod020L001fC*fC*fU*fU*fC*fC*mCfU*GmAfA*mGmGfU*fU*fC*fC*fU*fC |
| | *fC |
| 935 | fC*fC*fU*fU*fC*fC*mCfU*mGmAfA*mGmGfU*fU*fC*fC*fU*fC*fC |
| 936 | LOO1fC*fC*fU*fU*fC*fC*mCfU*mGmAfA*mGmGfU*fU*fC*fC*fU*fC*fC |
| 937 | Mod020L001fC*fC*fU*fU*fC*fC*mCfU*mGmAfA*mGmGfU*fU*fC*fC*fU*f |
| | C*fC |
| 938 | Mod015L001*fU*SPC*SfA*SfA*SfG*SfG*SmAfA*SmGmA*SfU*SmGmGfC* |
| | SfA*SfU*SfU*SfU*SfC*SfU |
| 939 | Mod015L001*SfU*SfC*SfA*SfA*SfG*SfG*SmAfA*SmGmA*SfU*SmGmGfC* |
| | SfA*SfU*SfU*SfU*SfC*SfU |
| 940 | L001*SfU*SfC*SfA*SfA*SfG*SfG*SmAfA*SmGmA*SfU*SmGmGfC*SfA*Sf |
| | U*SfU*SfU*SfC* |
| 941 | fU*SfC*SfA*SfA*SfG*SfG*SmAfA*SmGmA*SfU*SmGmGfC*SfA*SfU*SfU* |
| | SfU*SfC |
| 942 | fU*SfC*SfA*SfA*SfG*SfG*SmAfA*SmGmA*SfU*SmGmGfC*SfA*S |
| | fU*SfU*SfU |
| 943 | fC*SfA*SfA*SfG*SfG*SmAfA*SmGmA*SfU*SmGmGfC*SfA*SfU*SfU*SfU* |
| | SfC*SfU |
| 944 | Mod020L001mG*mG*mC*mC*mA*mASmA*mC*mC*mU*mC*mG*mG*mC* |
| | mU*mU*mA*mC*mC*mU |
| 945 | Mod015L001mG*mG*mC*mC*mA*mA*mA*mC*mC*mU*mC*mG*mG*mC* |
| | mU*mU*mA*mC*mC*mU |
| 946 | fU*fU*fC*fU*fG*fU*mA*mA*mG*mG*mU*mU*mU*mU*fU*fA*fU*f |
| | G*fU*fG |
| 947 | fA*fU*fU*fU*rC*fU*mG*mU*mA*mA*mG*mG*mU*mU*fU*fU*fU*f |
| | A*fU*fG |
| 948 | fC*fC*fA*fU*fU*fU*mC*mU*mG*mU*mA*mA*mG*mG*fU*fU*fU*f |
| | U*fU*fA |
| 949 | fA*fU*fC*fC*fA*fU*mU*mU*mC*mU*mG*mU*mA*mA*fG*fG*fU*f |
| | U*fU*fU |
| 950 | fC*fA*fU*fC*fC*fA*mU*mU*mU*mC*mU*mG*mU*mA*fA*fG*fG*f |
| | U*fU*fU |
| 951 | fC*fC*fA*fU*fC*fC*mA*mU*mU*mU*mC*mU*mG*mU*fA*fA*fG*f |
| | G*fU*fU |
| 952 | fG*fC*fC*fA*fU*fC*mC*mA*mU*mU*mU*mC*mU*mG*fU*fA*fA*f |
| | G*fG*fU |
| 953 | fA*fG*fC*fC*fA*fU*mC*mC*mA*mU*mU*mU*mC*mU*fG*fU*fA*f |
| | A*fG*fG |
| 954 | fC*fA*fG*fC*fC*fA*mU*mC*mC*mA*mU*mU*mU*mC*fU*fG*fU*f |
| | A*fA*fG |
| 955 | fU*fC*fA*fG*fC*fC*mA*mU*mC*mC*mA*mU*mU*mU*fC*fU*fG*f |
| | U*fA*fA |
| 956 | fU*fU*fC*fA*fG*fC*mC*mA*mU*mC*mC*mA*mU*mU*fU*fC*fU*f |
| | G*fU*fA |
| 957 | fC*fU*fU*fC*fA*fC*mC*mC*mA*mU*mC*mC*mA*mU*fU*fU*fC*f |
| | U*fG*fU |
| 958 | fA*fC*fU*fU*fC*fA*mG*mC*mC*mA*mU*mC*mC*mA*fU*fU*fU*f |
| | C*fU*fG |
| 959 | fA*fA*fC*fU*fU*fC*mA*mG*mC*mC*mA*mU*mC*mC*fA*fU*fU*f |
| | U*fC*fU |
| 960 | fC*fA*fA*fC*fU*fU*mC*mA*mG*mC*mC*mA*mU*mC*fC*fA*fU*f |
| | U*fU*fC |
| 961 | fU*fC*fA*fA*fC*fU*mU*mC*mA*mG*mC*mC*mA*mU*fC*fC*fA*f |
| | U*fU*fU |
| 962 | fC*fC*fA*fG*fG*fG*mC*mA*mG*mG*mC*mC*mA*mU*fU*fC*fC*f |
| | U*fC*fU |
| 963 | fC*fC*fC*fA*fG*fG*mG*mC*mA*mG*mG*mC*mC*mA*fU*fU*fC*f |
| | C*fU*fC |
| 964 | fC*fC*fC*fC*fA*fG*mG*mG*mC*mA*mG*mG*mC*mC*fA*fU*fU*f |
| | C*fC*fU |
| 965 | fC*fC*fC*fC*fC*fA*mG*mG*mG*mC*mA*mG*mG*mC*fC*fA*fU*f |
| | U*fC*fC |
| 966 | fU*fC*fc*fC*fC*fC*mA*mG*mG*mG*mC*mA*mG*mG*fC*fC*fA*f |
| | U*fU*fC |
| 967 | fA*fU*fC*fC*fC*fC*mC*mA*mG*mG*mG*mC*mA*mG*fG*fC*fC*f |
| | A*fU*fU |
| 968 | fC*fA*fU*fC*fC*fC*mC*mC*mA*mG*mG*mG*mC*mA*fG*fG*fC*f |
| | C*fA*fU |
| 969 | fG*fC*fA*fU*fC*fC*mC*mC*mC*mA*mG*mG*mG*mC*fA*fG*fG*f |
| | C*fC*fA |
| 970 | fA*fG*fC*fA*fU*fC*mC*mC*mC*mC*mA*mG*mG*mG*fC*fA*fG*f |
| | G*fC*fC |
| 971 | fC*fA*fG*fC*fA*fU*mC*mC*mC*mC*mC*mA*mG*mG*fG*fC*fA*f |
| | G*fG*fC |
| 972 | fU*fC*fA*fG*fC*fA*mU*mC*mC*mC*mC*mC*mA*mG*fG*fG*fC*f |
| | A*fG*fG |
| 973 | fU*fU*fC*fA*fG*fC*mA*mU*mC*mC*mC*mC*mC*mA*fG*fG*fG*f |
| | C*fA*fG |
| 974 | fU*fU*fU*fC*fA*fG*mC*mA*mU*mC*mC*mC*mC*mC*fA*fG*fG*f |
| | G*fC*fA |
| 975 | fA*fU*fU*fU*fC*fA*mG*mC*mA*mU*mC*mC*mC*mC*fC*fA*fG*f |
| | G*fG*fC |
| 976 | fG*fA*fU*fU*fU*fC*mA*mG*mC*mA*mU*mC*mC*mC*fC*fC*fA*f |
| | G*fG*fG |
| 977 | fG*fG*fA*fU*fU*fU*mC*mA*mG*mC*mA*mU*mC*mC*ft*fC*fC*f |
| | A*fG*fG |
| 978 | fA*fG*fG*fA*fU*fU*mU*mC*mA*mG*mC*mA*mU*mC*fC*fC*fC*f |
| | C*fA*fG |
| 979 | fC*fA*fG*fG*fA*fU*mU*mU*mC*mA*mG*mC*mA*mU*fC*fC*fC*f |
| | C*fC*fA |
| 980 | fU*fC*fA*fG*fG*fA*mU*mU*mU*mC*mA*mG*mC*mA*fU*fC*fC*f |
| | C*fC*fC |
| 981 | fU*fU*fC*fA*fG*fG*mA*mU*mU*mU*mC*mA*mG*mC*fA*fU*fC*f |
| | C*fC*fC |
| 982 | fU*fU*fU*fC*fA*fG*mG*mA*mU*mU*mU*mC*mA*mG*fC*fA*fU*f |
| | C*fC*fC |
| 983 | fU*fU*fU*fU*fC*fA*mG*mG*mA*mU*mU*mU*mC*mA*fG*fC*fA*f |
| | U*fC*fC |
| 984 | fU*fU*fU*fU*fU*fC*mA*mG*mG*mA*mU*mU*mU*mC*fA*fG*fC*f |
| | A*fU*fC |
| 985 | fU*fU*fU*fU*fU*fU*mC*mA*mG*mG*mA*mU*mU*mU*fC*fA*fG*f |
| | C*fA*fU |
| 986 | fG*fU*fU*fU*fU*fU*mU*mC*mA*mG*mG*mA*mU*mU*fU*fC*fA*f |
| | G*fC*fA |
| 987 | fU*fG*fU*fU*fU*fU*mU*mU*mC*mA*mG*mG*mA*mU*fU*fU*fC*f |
| | A*fG*fC |
| 988 | fC*fU*fG*fU*fU*fU*mU*mU*mU*mC*mA*mG*mG*mA*fU*fU*fU*f |
| | C*fA*fG |
| 989 | fG*fC*fU*fG*fU*fU*mU*mU*mU*mU*mC*mA*mG*mG*fA*fU*fU*f |
| | U*fC*fA |
| 990 | fA*fG*fC*fU*fG*fU*mU*mU*mU*mU*mU*mC*mA*mG*fG*fA*fU*f |
| | U*fU*fC |
| 991 | fG*fA*fG*fC*fU*fG*mU*mU*mU*mU*mU*mU*mC*mA*fG*fG*fA*f |
| | U*fU*fU |
| 992 | fU*fG*fA*fG*fC*fU*mG*mU*mU*mU*mU*mU*mU*mC*fA*fG*fG*f |
| | A*fU*fU |
| 993 | fU*fU*fG*fA*fG*fC*mU*mG*mU*mU*mU*mU*mU*mU*fC*fA*fG*f |
| | G*fA*fU |
| 994 | fU*fU*fU*fG*fA*fG*mC*mU*mG*mU*mU*mU*mU*mU*fU*fC*fA*f |
| | G*fG*fA |
| 995 | fG*fU*fU*fU*fG*fA*mG*mC*mU*mG*mU*mU*mU*mU*fU*fU*fC*f |
| | A*fG*fG |
| 996 | fU*fU*fG*fU*fU*fU*mG*mA*mG*mC*mU*mG*mU*mU*fU*fU*fU*f |
| | U*fC*fA |
| 997 | fC*fA*fU*fU*fG*fU*mU*mU*mG*mA*mG*mC*mU*mG*fU*fU*fU*f |
| | U*fU*fU |
| 998 | fG*fC*fA*fU*fU*fG*mU*mU*mU*mG*mA*mG*mC*mU*fG*fU*fU*f |
| | U*fU*fU |
| 999 | fU*fG*fC*fA*fU*fU*mG*mU*mU*mU*mG*mA*mG*mC*fU*fG*fU*f |
| | U*fU*fU |
| 1000 | fC*fU*fG*fC*fA*fU*mU*mG*mU*mU*mU*mG*mA*mG*fC*fU*fG*f |
| | U*fU*fU |
| 1001 | fU*fC*fU*fG*fC*fA*mU*mU*mG*mU*mU*mU*mG*mA*fG*fC*fU*f |
| | G*fU*fU |
| 1002 | fC*fU*fC*fU*fG*fC*mA*mU*mU*mG*mU*mU*mU*mG*fA*fG*fC*f |
| | U*fG*fU |
| 1003 | fA*fC*fU*fC*fU*fG*mC*mA*mU*mU*mG*mU*mU*mU*fG*fA*fG*f |
| | C*fU*fG |
| 1004 | fU*fA*fC*fU*fC*fU*mG*mC*mA*mU*mU*mG*mU*mU*fU*fG*fA*f |
| | G*fC*fU |
| 1005 | fU*fU*fA*fC*fU*fC*mU*mG*mC*mA*mU*mU*mG*mU*fU*fU*fG*f |
| | A*fG*fC |
| 1006 | fC*fU*fU*fA*fC*fU*mC*mU*mG*mC*mA*mU*mU*mG*fU*fU*fU*f |
| | G*fA*fG |
| 1007 | fU*fC*fU*fU*fA*fC*mU*mC*mU*mG*mC*mA*mU*mU*fG*fU*fU*f |
| | U*fG*fA |
| 1008 | fA*fU*fC*fU*fU*fA*mC*mU*mC*mU*mG*mC*mA*mU*fU*fG*fU*f |
| | U*fU*fG |
| 1009 | fA*fA*fU*fC*fU*fU*mA*mC*mU*mC*mU*mG*mC*mA*fU*fU*fG*f |
| | U*fU*fU |
| 1010 | fC*fA*fA*fA*fU*fC*mU*mU*mA*mC*mU*mC*mU*mG*fC*fA*fU*f |
| | U*fG*fU |
| 1011 | fG*fA*fU*fA*fC*fA*mA*mA*mU*mC*mU*mU*mA*mC*fU*fC*fU*f |
| | G*fC*fA |
| 1012 | Geo*Geo*Geo*Teo*m5Ceo*A*G*C*T*G*C*C*A*A*T*Geo*m5Ceo* |
| | Teo*Aeo*Geo |
| 1013 | Mod030Geo*Geo*Geo*Teo*m5Ceo*A*G*C*T*G*C*C*A*A*T*Geo*m5Ceo*T |
| | eo*Aeo*Geo |
| 1014 | Mod031Geo*Geo*Geo*Teo*m5Ceo*A*G*C*T*G*C*C*A*A*T*Geo*m5Ceo*T |
| | eo*Aeo*Geo |
| 1015 | Mod032Geo*Geo*Geo*Teo*m5Ceo*A*G*C*T*G*C*C*A*A*T*Geo*m5Ceo*T |
| | eo*Aeo*Geo |
| 1016 | Mod033Geo*Geo*Geo*Teo*m5Ceo*A*G*C*T*G*C*C*A*A*T*Geo*m5Ceo*T |
| | eo*Aeo*Geo |

In Table 2; “*” represents a stereorandom phosphorothioate linkage; “*S” represents an Sp phosphorothioate linkage; “*R” represents an Rp phosphorothioate linkage; all non-labeled linkage is a natural phosphate linkage; “m” preceding a base represents 2′-OMe; and “eo” following a base represents 2′-MOE. F represents a fluorinated nucleoside. LOO1 represents a C6 PO (phosphodiester) or PS (phosphorothioate) linker. Mod represents a modification attached to the nucleic acid: Lauric (in Mod013), Myristic (in Mod014), Palmitic (in Mod005), Stearic (in Mod015), Oleic (in Mod016), Linoleic (in Mod017), alpha-Linoleinc (in Mod018), gamma-Linolenic (in Mod019), DHA (in Mod006), Turbinaric (in Mod020), Dilinoleic (in Mod021), TriGlcNAc (in Mod024), TrialphaMannose (in Mod026), MonoSulfonamide (in Mod 027), TriSulfonamide (in Mod029), Lauric (in Mod030), Myristic (in Mod031), Palmitic (in Mod032), and Stearic (in Mod033): Lauric acid (for Mod013), Myristic acid (for Mod014), Palmitic acid (for Mod005), Stearic acid (for Mod015), Oleic acid (for Mod016), Linoleic acid (for Mod017), alpha-Linolenic acid (for Mod018), gamma-Linolenic acid (for Mod019), docosahexaenoic acid (for Mod006), Turbinaric acid (for Mod020), alcohol for Dilinoleyl (for Mod021), acid for TriGlcNAc (for Mod024), acid for TrialphaMannose (for Mod026), acid for MonoSulfonamide (for Mod 027), acid for TriSulfonamide (for Mod029), Lauryl alcohol (for Mod030), Myristyl alcohol (for Mod031), Palmityl alcohol (for Mod032), and Stearyl alcohol (for Mod033), respectively, conjugated to oligonucleotide chains through amide groups, C6 amino linker, phosphodiester linkage (PO), and/or phosphorothioate linkage (PS).

TABLE 3

| SEQ | |
|---|---|
| ID | |
| NO. | Sequence 5′→3′ |
| 1017 | GUGGAAUAGUAUAACAAUAUgcuaaAUGUUGUUAUAGUAUCCCACACU |
| | GAGCAAUGCcGUAGUCAG*C*A*A*U |
| 1018 | GUGGAAUAGUAUAACAAUAUgcuaaAUGUUGUUAUAGUAUCCCACGUA |
| | CUGAGCAAUGCcGUAGUCAGCAA*U*C*U*U |
| 1019 | GGAAUAGUAUAACAAUAUgcuaaAUGUUGUUAUAGUAUCCCACUGAGC |
| | AAUGCcGUAGUCAG*C*A*A*U |
| 1020 | GGAAUAGUAUAACAAUAUgcuaaAUGUUGUUAUAGUAUCCCGUACUGA |
| | GCAAUGCcGUAGUCAGCAA*U*C*U*U |
| 1021 | cgcgcgttttcgcgcgGCUGAACCACUGCAC |
| 1022 | cgcgcgttttcgcgcgGAGAUACUCACAAUU |
| 1023 | cgcgcgttttcgcgcgCGUUGACCUCCACUC |
| 1024 | rGrGrArArUrArGrUrArUrArArCrArArUrArUrgrcrurararArUrGrUrUrGrUrUrAr |
| | UrArGrUrArUrCrCrCmC*mA*mG*mU*mCmCmCmUmUmUmCrUrCrGmUmC |
| | mGmAmUmGmG*mU*mC*mA*mG |
| 1025 | mG*mG*mA*mA*mU*mA*mG*mU*mA*mU*mA*mA*mC*mA*mA*mU*mA |
| | *mU*mG*mC*mU*mA*mA*mA*mU*mG*mU*mU*mG*mU*mU*mA*mU*m |
| | A*mG*mU*mA*mU*mC*mC*mCmC*mA*mG*mU*mCmCmCmUmUmUmCr |
| | UrCrGmUmCmGmAmUmGmG*mU*mC*mA*mG |
| 1026 | unmodified |
| | GUGUUGGCCAUGGAACAUAUAACAAUAUgcuaaAUGUUGUUAUA |
| 1027 | 2′OMe-PS |
| | UAUAACAAUAUgcuaaAUGUUGUUAUAGUGUUGGCCAUGGAACA |
| 1028 | 2′Ome-PS |
| | GUGUUGGCCAUGGAACAUAUAACAAUAUgcuaaAUGUUGUUAUA |
| 1029 | unmodified |
| | GUGUUGGCCAUGGAACAAUAGUAUAACAAUAUgcuaaAUGUUGUU |
| | AUAGUAU |
| 1030 | unmodified |
| | GGAAUAGUAUAACAAUAUgcuaaAUGUUGUUAUAGUAUCCCGUGU |
| | UGGCCAUGGAACA |
| 1031 | unmodified |
| | GUGUUGGCCAUGGAACAGGAAUAGUAUAACAAUAUgcuaaAUGUU |
| | GUUAUAGUAUCCC |
| 1032 | 2′OMe- |
| | PSGGAAUAGUAUAACAAUAUgcuaaAUGUUGUUAUAGUAUCCCGUGUU |
| | GGCCAUGGAACA |

In Table 3; “R” represents no modification; “in” represents 2′O-Me, “PS represents phosphorothioate linkages, and “*” represents phosphorothioate linkage.

TABLE 4

| SEQ ID | | |
|---|---|---|
| NO. | Sequence 5′→3′ | Base modifications |
| 1033 | u*c*a*g*ucccuuucUCGucgauggucagc*a*c*a*g | |
| 1034 | u*c*a*g*ucccuuucuCGucgauggucagc*a*c*a*g | |
| 1035 | u*c*a*g*ucccuuucuCGUcgauggucagc*a*c*a*g | |
| 1036 | u*c*a*g*ucccuuucucgucgauggucagc*a*c*a*g | |
| 1037 | u*c*a*g*ucccuuucU*CGucgauggucagc*a*c*a*g | |
| 1038 | u*c*a*g*ucccuuuc[TCG]ucgauggucagc*a*c*a*g | |
| 1039 | u*c*a*g*ucccuuuc(ucg)ucgauggucagc*a*c*a*g | |
| 1040 | u*c*a*g*ucccuuuc{U}C{G}ucgauggucagc*a*c*a* | |
| | g | |
| 1041 | u*c*a*g*ucccuuuc{U}X{G}ucgauggucagc*a*c*a* | X: 5-Methylcytidine |
| | g | |
| 1042 | u*c*a*g*ucccuuucUCZucgauggucagc*a*c*a*g | Z: Inosine |
| 1043 | u*c*a*g*ucccuuucUXZucgauggucagc*a*c*a*g | X: 5-Methylcytidine, |
| | | Z: Inosine |
| 1044 | u*c*a*g*ucccuuucYXZucgauggucagc*a*c*a*g | Y: Pseudouridine, |
| | | X: 5-Methylcytidine, |
| | | Z: Inosine |
| 1045 | u*c*a*g*ucccuuucYXZucgauggucagc*a*c*a*g | Y: Pseudouridine, |
| | | X: 5-Methylcytidine, |
| | | Z: Inosine |
| 1046 | u*c*a*g*ucccuuucYXZucgauggucagc*a*c*a*g | Y: 5-Methyluridine, |
| | | X: 5-Methylcytidine, |
| | | Z: Inosine |
| 1047 | u*c*a*g*ucccuuucYXZucgauggucagc*a*c*a*g | Y: Thienouridine, |
| | | X: 5-Methylcytidine, |
| | | Z: Inosine |
| 1048 | u*c*a*g*ucccuuucYXZucgauggucagc*a*c*a*g | Y: 2,6-diaminopurine, |
| | | X: 5-Methylcytidine, |
| | | Z: Inosine |
| 1049 | u*c*a*g*ucccuuucUXZucgauggucagc*a*c*a*g | X: Pyrrolocytidine, |
| | | Z: Inosine |
| 1050 | u*c*a*g*ucccuuucYXZucgauggucagc*a*c*a*g | Y: Pseudouridine, |
| | | X: Pyrrolocytidine, |
| | | Z: Inosine |
| 1051 | u*c*a*g*ucccuuucYXZucgauggucagc*a*c*a*g | Y: Thienouridine, |
| | | X: Pyrrolocytidine, |
| | | Z: Inosine |
| 1052 | u*c*a*g*ucccuuucYXZucgauggucagc*a*c*a*g | Y: Pseudouridine, |
| | | X: 5-Methylcytidine, |
| | | Z: Thienoguanosine |
| 1053 | u*c*a*g*ucccuuucYXZucgauggucagc*a*c*a*g | Y: Pseudouridine, |
| | | X: Pyrrolocytidine, |
| | | Z: Thienoguanosine |
| 1054 | u*c*a*g*ucccuuucU′C′Gvucgauggucagc*a*c*a*g | |
| 1055 | u*c*a*g*ucccuuucU″C″G″ucgauggucagc*a*c*a*g | |
| 1056 | u*c*a*g*ucccuuucUACAGAucgauggucagc*a*c*a* | |
| | g | |
| 1057 | u*c*a*g*ucccuuucU#C#G#ucgauggucagc*a*c*a*g | |
| 1058 | u*c*a*g*ucccuuucYXZucgauggucagc*a*c*a*g | Y: 5-Methoxyuridine, |
| | | X: 5-Methylcytidine, |
| | | Z: I nosine |
| 1059 | u*c*a*g*ucccuuucYXZucgauggucagc*a*c*a*g | Y: dihydrouridine, |
| | | X: 5-Methylcytidine, |
| | | Z: Inosine |
| 1060 | u*c*a*g*ucccuuucYXZucgauggucagc*a*c*a*g | Y: Pseudouridine, |
| | | X: 5- |
| | | Hydroxymethylcytidine, |
| | | Z: Inosine |
| 1061 | u*c*a*g*ucccuuucYXZucgauggucagc*a*c*a*g | Y: Pseudouridine, |
| | | X: 5-Methylcytidine, |
| | | Z: 7-Methylguanosine |
| 1062 | u*c*a*g*ucccuuucYXZucgauggucagc*a*c*a*g | Y: Pseudouridine, |
| | | X: 5-Methylcytidine, |
| | | Z: 7-deazaguanosine |
| 1063 | u*c*a*g*ucccuuucYXZucgauggucagc*a*c*a*g | Y: Pseudouridine, |
| | | X: 5-Methylcytidine, |
| | | Z: 8-aza-7- |
| | | deazagunaosine |
| 1064 | u*c*a*g*ucccuuucYXZucgauggucagc*a*c*a*g | Y: Pseudouridine, |
| | | X: 5-Methylcytidine, |
| | | Z: 7-aminomethy 1-7- |
| | | deazaguanosine |
| 1065 | c*c*u*g*cgacacuucggcCCAgagcugcucc*u*c*a*u | |
| 1066 | c*c*u*g*cgacacuucggcXXYgagcugcucc*u*c*a*u | X: 5-Methylcytidine, |
| | | Y: 7-Methyladenosine |
| 1067 | c*c*u*g*cgacacuucggcXXYgagcugcucc*u*c*a*u | X: 5-Methylcytidine, |
| | | Y: 8-Methyladenosine |
| 1068 | c*c*u*g*cgacacuucggcXXYgagcugcucc*u*c*a*u | X: 5-Methylcytidine, |
| | | Y: 3-deazaadenosine |
| 1069 | c*c*u*g*cgacacuucggcXXYgagcugcucc*u*c*a*u | X: 5-Methylcytidine, |
| | | Y: 7-deazaadenosine |
| 1070 | c*c*u*g*cgacacuucggcXXYgagcugcucc*u*c*a*u | X: 5-Methylcytidine, |
| | | Y: 8-azidoadenosine |
| 1071 | c*c*u*g*cgacacuucggcXXYgagcugcucc*u*c*a*u | X: 5-Methylcytidine, |
| | | Y: Inosine |
| 1072 | c*a*u*u*gaagaagauaagagaaaguacugagaaguguuggCCAuggaacag*g*u*a*g | |
| 1073 | c*a*u*u*gaagaagauaagagaaaguacugagaaguguuggC | Z: 2-aminopurine |
| | CZuggaacag*g*u*a*g | |
| 1074 | c*a*u*u*gaagaagauaagagaaaguacugagaaguguugg[C]C[A]uggaacag*g* | |
| | u*a*g | |
| 1075 | g*a*c*u*gagguacuccuuagagaaaggug[CCA]cuucuug | |
| | gcaa*a*g*g*a | |
| 1076 | g*u*a*g*gcaugggaggaaaaggugCCAcuucuuggcaa*a | |
| | *g*g*a | |
| 1077 | c*u*g*u*ccaacacagccccagccuuugagaccucugcCCAg | |
| | aguuguu*c*u*c*c | |
| 1078 | c*u*g*u*ccaacacagccccagccuuugagaccucuguCC[A]gaguuguu*c*u*c* | |
| | cidT | |
| 1079 | c*u*g*u*ccaacacagccccagccuuugagaccucugucC[A]gaguuguu*c*u*c* | |
| | cidT | |
| 1080 | c*u*g*u*ccaacacagccccagccuuugagaccucuguXC[A] | X: 5-Methylcytidine |
| | gaguuguu*c*u*c*c idT | |
| 1081 | c*u*g*u*ccaacacagccccagccuuugagaccucugucC[X] | X: deoxy 2-aminopurine |
| | gaguuguu*c*u*c*c idT | |
| 1082 | c*u*g*u*ccaacacagccccagccuuugagaccucugucC[Agaauuguu*c*u*c* | |
| | cidT | |
| 1083 | c*u*g*u*ccaacacagccccagccuuugagaccucugucC[AGA]guuguu*c*u*c* | |
| | cidT | |
| 1084 | c*u*g*u*ccaacacagccccagccuuugagaccucuguc*C*[A]*G*A*guuguu*c*u | |
| | *c*cidT | |
| 1085 | c*u*g*u*ccaacacagccccagccuuugagaccucuguc*C*[A]*gaguuguu*c*u*c | |
| | *cidT | |
| 1086 | c*u*g*u*ccaacacagccccagccuuugagaccucug*u*C*C*A*g*aguuguu*c*u* | |
| | c*cidT | |
| 1087 | c*u*g*u*ccaacacagccccagccuuugagaccucuguc(caga)guuguu*c*u*c*c | |
| 1088 | c*u*g*u*ccaacacagccccagccuuugagaccucugu[CCA]gaguuguu*c*u*c*c | |
| 1089 | c*u*g*u*c*c*a*a*c*a*c*a*gccccagccuuugagaccucugu[CCA]gaguuguu* | |
| | c*u*c*c | |
| 1090 | c*u*g*u*ccaacacagccc*c*a*g*c*c*u*u*u*g*a*gaccucugu[CCA]gaguug | |
| | uu*c*u*c*c | |
| 1091 | c*u*g*u*c*c*a*a*c*a*c*a*gccc*c*a*g*c*c*u*u*u*g*a*gaccucugu | |
| | [CCA]gaguuguu*c*u*c*c | |
| 1092 | c*u*g*u*ccaacacagccccagccuuugagaccucuguc[CA]gaguuguu*c*u*c*c | |
| 1093 | c*u*g*u*ccaacacagccccagccuuugagaccucugu[C)(c)[A]gaguuguu*c*u* | |
| | c*c | |
| 1094 | c*u*g*u*ccaacacagccccagccuuugagaccucugu[C*C*A*]gaguuguu*c*u* | |
| | c*c | |
| 1095 | c*u*g*u*ccaacacagccccagccuuugagaccucugu[CCA]gaguuguu*c*u*c* | |
| | cidT | |
| 1096 | a*c*a*c*a*G*cuc*c*a*g*c*c*u*u*u*G*A*gaccu*c*u*g*cCCAGaguu*g* | |
| | u*u*c*u*c*c | |
| 1097 | c*a*c*a*gccccagccuuugagaccucugu[CCA]gaguugu | |
| | u*c*u*c*c | |
| 1098 | c*a*c*a*g*c*c*c*c*a*g*c*c*u*u*u*g*agaccucugu[CCA]gaguuguu*c* | |
| | u*c*c | |
| 1099 | c*a*c*a*gccc*c*a*g*c*c*u*u*u*g*a*gaccucugcc*[C*A]*gaauuguu*c* | |
| | u*c*c | |
| 1100 | g*a*c*u*gagguacuccauagggaaaggcacC[A]cuucuuggcaa*a*g*g*a | |
| 1101 | g*a*c*u*gagguacuccauagggaaaggcacC[ACU]ucuuggcaa*a*g*g*a | |
| 1102 | g*a*c*u*gagguacuccauagggaaaggcacC[A]<cu>ucuuggcaa*a*g*g*a | |

In Table 4, specific YXZ base modifications are mentioned in the third column. Lower case nucleotides are RNA and 2′-O-methyl modified. Upper case nucleotides are RNA, except for bracketed [NNN] nucleotides, which is DNA. Lower case nucleotides depicted as (nnn) are 2′-fluoro RNA modified nucleotides. Lower case nucleotides depicted as <nnn> are 2′-NH2 RNA modified nucleotides. Nucleotides depicted as {N} are Unlocked Nucleic Acid (UJNA). “idT” indicates a 3′ inverted T modification which enhances the resistance to degradation and also blocks the 3′-terminus of AON from extension during PCR amplification. “*” represents phosphorothioate linkages; “′”=3′-methylenephosphonate linkages; “″” represents 5′-methylenephosphonate linkages; “Λ” represents 3′-phosphoroamidate linkages; and “#” represents 2′-5′ phosphodiester linkages.

TABLE 5

| SEQ | |
|---|---|
| ID | |
| NO. | Sequence 5′→3′ |
| 1103 | g*a*c*u*gagguacuccuuagagaaaggugCCAcuucuuggcaa*a*g*g*a- |
| 1104 | mC*mA*mU*mU*mGmAmAmGmAmAmGmAmUmAmAmGmAmGmAmAm |
| | AmGmUmAmCmUmGmAmGmAmAmGmUmGmUmUmGmGCCAmUmGmG |
| | mAmAmCmAmG*mG*mU*mA*mG |
| 1105 | cauugaagaa gauaagagaa aguacugaga aguguuggcc auggaacagg uag |

In Table 5; RNA is depicted by A, C, G, or U; DNA is depicted by dA, dC, dG, or dT; 2′-Ome is depicted by mA, mC, mG, or mU; PMO (Phosphorodiamidate morpholino oligomers) are depicted by pA, pC, pG, or pT; and Phosphorothioate is depicted by “*”.

TABLE 6

| SEQ | |
|---|---|
| ID NO. | Sequence 5′→3′ |
| 1106 | GUGGAUAGUAUAACAAUAUGCUMAAUGUUGUUAUAGUAUCCCAC |
| 1107 | GUGGASSSGSASASCAAUAUGCUMAAUGUUGSUSUSGSSSCCCAC |
| 1108 | GUGGAASAGSASAACAAUAUGCUMAAUGUUGUUSUSGUSUCCCAC |
| 1109 | GUGGASASUAUAACAAUAUGCUMAAUGUUGUUAUAGYAYCCCAC |
| 1110 | GUGGASSSGSSSSSSSSUAUGCUMAAUGSSSSSSSSGSSSCCCAC |
| 1111 | GUGGAASSGSASASCAAUAUGCUMAAUGUUGSUSUSGSSUCCCAC |
| 1112 | GUGGSAUAGUAUAACAAUAUGCUMAAUGUUGUUAUAGUAUCCCAC |
| 1113 | ACGCAACCAAGUCAUA |
| 1114 | GCAAUGCCAUCACCUC |
| 1115 | AGGGGUCCACAUGGCA |
| 1116 | GGCUCCCCAGGCCCCU |
| 1117 | UGCCGUCCACCAGGAU |
| 1118 | CAGAUUCCAGGUGGGA |
| 1119 | UCCCUGCCAGAAUAGA |
| 1120 | CUCCGCCCACCAAAUG |
| 1121 | CCCAAACCACAACAGA |
| 1122 | ACCCACCCACCCAGGU |
| 1123 | CUGCCGCCAGCUGGAU |
| 1124 | AGGGAACCAGACAGUU |

In Table 6; “S” can be G or C, “Y” can be C or T; and “M” can be A or C.

TABLE 7

| SEQ ID NO. | Sequence 5′→3′ |
|---|---|
| 1125 | U*U*C*A*C*U*UcAG*U*G*U*As*Us*Gs*Cs*C* |
| 1126 | U*U*C*A*C*U*UcAG*U*G*U*As*Us*Gs*Cs*C* |
| 1127 | A*C*C*U*C*C*AcUC*A*G*U*Gs*Us*Gs*As*U* |
| 1128 | U*U*U*C*C*U*CcAC*U*G*U*Us*Gs*Cs*As*A* |
| 1129 | U*G*U*G*U*A*UcUU*G*C*U*Gs*Us*Gs*As*G* |
| 1130 | G*A*G*G*U*C*CcUG*G*G*G*Gs*Cs*Gs*Cs*U* |
| 1131 | G*A*U*C*U*U*CcUG*A*U*G*Gs*Cs*Cs*As*C* |
| 1132 | A*G*C*C*A*C*AcAC*U*C*C*Gs*Us*Cs*As*G* |
| 1133 | G*A*U*U*U*U*CcUG*A*U*A*Gs*Cs*Us*As*C* |
| 1134 | G*G*C*C*A*C*AcAU*U*C*U*Gs*Us*Cs*As*G* |
| 1135 | G*A*U*C*U*U*CcUG*A*U*G*Gs*Cs*Cs*As*C* |
| 1136 | G*G*C*C*A*C*AcAC*U*C*C*Gs*Us*Cs*As*G* |
| 1137 | G*A*U*U*U*U*CcUG*A*U*A*Gs*Cs*As*As*C* |
| 1138 | G*G*C*U*A*C*GcAC*U*C*U*Gs*Us*Cs*As*A* |
| 1139 | A*G*G*C*C*G*CcGU*C*G*U*Gs*Gs*Cs*Gs*G* |
| 1140 | C*C*G*C*U*C*CcUCcUC*A*G*C*Cs*Cs*Gs*Us*C* |
| 1141 | A*C*G*C*C*A*CCAG*C*U*C*Cs*As*As*Cs*U* |
| 1142 | G*U*C*U*C*A*CCAA*U*U*G*Cs*Us*Cs*Us*C* |
| 1143 | G*A*A*A*U*A*CcAU*C*A*G*As*Us*Us*Us*G* |
| 1144 | A*A*U*U*A*G*CcUU*C*U*G*Gs*Cs*Cs*As*U* |
| 1145 | G*A*U*C*A*G*CcUC*C*U*G*Gs*Cs*Cs*As*U* |
| 1146 | G*A*U*C*A*G*CcUU*C*U*G*Gs*Cs*Cs*As*U* |
| 1147 | G*A*U*C*A*G*CcUU*C*U*G*Gs*Cs*Cs*As*U* |
| 1148 | *A*C*U*G*C*CcAG*G*C*A*Us*Cs*As*Gs*C* |
| 1149 | C*A*C*U*G*C*CcGG*G*C*A*Us*Cs*As*Gs*C* |
| 1150 | U*C*C*G*C*C*CcGA*U*C*C*As*Cs*Gs*As*U* |
| 1151 | C*C*U*U*U*C*UcGU*C*G*A*Us*Gs*Gs*Us*C* |
| 1152 | C*C*U*U*U*C*U*cGU*C*G*A*Us*Gs*Gs*Us*C* |
| 1153 | C*U*U*G*A*U*AcAU*C*C*A*Gs*Us*Us*Cs*C* |
| 1154 | U*U*U*C*A*G*GcAU*U*U*C*Cs*Us*Cs*Cs*G* |
| 1155 | C*U*U*C*A*G*GcAU*G*G*G*Gs*Cs*As*Gs*C* |
| 1156 | A*G*G*A*A*C*AcAA*C*C*U*Us*Us*Gs*Us*C* |
| 1157 | U*U*U*C*A*C*AcAU*C*C*A*Us*Cs*As*As*C* |
| 1158 | C*U*U*C*A*C*GcAU*C*C*A*Us*Cs*As*As*C* |
| 1159 | U*G*G*G*A*C*AcAA*C*C*C*Cs*Us*Gs*Cs*C* |
| 1160 | C*G*A*C*U*C*CcUC*U*G*G*As*Us*Gs*Us*U* |
| 1161 | C*G*A*C*U*C*UcUC*U*G*G*As*Us*Gs*Us*U* |

In Table 7; Nₐ and N_{b} can form a mismatch, in some cases where Nₐ is adenosine and N_{b} is cytidine; N_{c} and N_{d} form a mismatch, in some cases wherein N_{c} and N_{d} are guanosine; “Gs” is a guanosine comprising a phosphorothioate group; “Gsl” is an LNA guanosine comprising a phosphorothioate group; and wherein an asterisk (*) indicates a modification of the nucleotide at the 2 carbon atom, in some cases with 2′-hydrogen (2′-cleoxy), 2′-0-methyl, 2′-0-methoxyethyl or 2′-fluoro; “A” is an adenosine nucleotide or a variant thereof, in some cases an adenosine ribonucleotide, an adenosine deoxynucleotide, a modified adenosine ribonucleotide or a modified adenosine deoxynucleotide; “C” is a cytidine nucleotide or a variant thereof, for example a cytidine ribonucleotide, a cytidine deoxynucleotide, a modified cytidine ribonucleotide or a modified cytidine deoxynucleotide; “G” is a guanosine nucleotide or a variant thereof, for example a guanosine ribonucleotide, a guanosine deoxynucleotide, a modified guanosine ribonucleotide or a modified guanosine deoxynucleotide; “U” is an uridine nucleotide or a variant thereof, for example a uridine ribonucleotide, a uridine deoxynucleotide, a modified uridine ribonucleotide, or a modified uridine deoxynucleotide; “A”, “C”, “G” or “U” is a nucleotide, in some cases a ribonucleotide or a deoxynucleotide as defined above, further comprising a phosphorothioate group; wherein an asterisk (*) indicates a chemical modification of the preceding nucleotide at the 2′ carbon atom, for example with 2′-hydrogen (2′-deoxy), 2′-0-methyl, 2′-0-methoxyethyl or 2′-fluoro; and wherein a lower case letter c indicates the position corresponding to a nucleotide, for example an adenosine or a cytidine, for example an adenosine, to be edited in the target sequence and wherein c represents a cytidine nucleotide or a variant thereof, a deoxycytidine nucleotide or a variant thereof, or an abasic site.

TABLE 8

| SEQ | |
|---|---|
| ID | |
| NO. | Sequence 5′→3′ |
| 1162 | (GUGGAAUAGUAUAACAAUAUGCUAAAUGUUGUUAUAGUAUCCCACG |
| | UGCAGCCAGCCGUCCUCUAGAGGGCCCUGAAGAGGGCCC) |
| 1163 | (GUGGAAGAGGAGAACAAUAUGCUAAAUGUUGUUCUCGUCUCCCACG |
| | UGCAGCCAGCCGUCCUCUAGAGGGCCCUGAAGAGGGCCC) |
| 1164 | (GUGGUCGAGAAGAGGAGAACAAUAUGCUAAAUGUUGUUCUCGUCUC |
| | CUCGACCACGUGCAGCCAGCCGUCCUCUAGAGGGCCCUGAAGAGGGC |
| | CC) |
| 1165 | [GCAAUG](CCA)[UCAC][C*][U][C][C*][C] |
| 1166 | (GGUGAAUAGUAUAACAAUAUGCUAAAUGUUGUUAUAGUAUCCACC) |
| | [GCAAUG](CCA)[UCAC\|[C][U*][C*][C*][C] |
| 1167 | (GGUGAAGAGGAGAACAAUAUGCUAAAUGUUGUUCUCGUCUCCACC) |
| | [GCAAUG](CCA)[UCAC][C][U*][C*][C*][C] |
| 1168 | (GGUGUCGAGAAGAGGAGAACAAUAUGCUAAAUGUUGUUCUCGUCUC |
| | CUCGACACC)[GCAAUG](CCA)[UCAC\|C*\|U*\|[C][C][C] |
| 1169 | [AGGGGU](CCA)[CAUG][G][C*][A][A][C] |
| 1170 | (GGUGAAUAGUAUAACAAUAUGCUAAAUGUUGUUAUAGUAUCCACC) |
| | [AGGGGU](CCA)[CAUG][G*][C*][A][A][C] |
| 1171 | (GGUGAAGAGGAGAACAAUAUGCUAAAUGUUGUUCUCGUCUCCACC) |
| | [AGGGGU](CCA)[CAUG][G*][C*][A*][A*][C] |
| 1172 | (GGUGUCGAGAAGAGGAGAACAAUAUGCUAAAUGUUGUUCUCGUCUC |
| | CUCGACACC)[AGGGGU](CCA)[CAUG][G*][C*][A*][A*][C] |
| 1173 | [G*][G*][U](G)[U][C](GAGAAGAGGAGAA)[C](AA)[U] |
| 1174 | [GGGGUG](CCA)[AGCA][G*][U*][U*][G*][G] |
| 1175 | (GGUGUCGAGAAGAGGAGAACAAUAUGCUAAAUGUUGUUCUCGUCUC |
| | CUCGACACC)[GGGGUG](CCA)[AGCA][G*][U*][U*][G][G] |
| 1176 | [GGGGUG](CCA)[AGCA][G][U][U*][G*][G] |
| 1177 | (GGUGUCGAGAAGAGGAGAACAAUAUGCUAAAUGUUGUUCUCGUCUC |
| | CUCGACACC)[GUUUUU](CCA)[GACG][G*][C*][A*][G*][G] |
| 1178 | [G]*[G]*[U](G)[U][C](GAGAAGAGGAGAA)[C](AA)[U] |
| 1179 | [G]*[G]*[U](GUCGAGAAGAGGAGAACAAUAUGCUA |
| 1180 | (GGUGUCGAGAAGAGGAGAACAAUAUGCUAAAUGUUGUUCUCGUCUC |
| | CUCGACACC)[CCUUUC](UCG)[UCGA][U*][G*][G*][U*][C] |
| 1181 | (CAUGGCCCCAGCAGCUUCAGUC)[C]{C}[UUUC](UCG) |
| 1182 | [G*][G*][U](G)[U][C](GAGAAGAGGAGAA)[C](AA)[U](A)[U](G)[C][U](AAA) |
| | [U](G)[U][U](G)[U][U][C][U][U][C][C][U][C}[GACACCCAUGGCCCCAGC |
| | AGCUUCAGUC)[C]{C}[UUUC](UCG)[UCGA]{T*}[G*][G*]{T*}[C] |
| 1183 | [G*][G*][U](G)[U][C](GAGAAGAGGAGAA)[C](AA)[U](A)[U](G)[C][U](AAA) |
| | [U](G)[U][U](G)[U][U][C][U][C](G)[U][C][U][C][C][U][C](GACACCCAUG |
| | GCCCCAGCAGCUUCAGUC)[C]{C}[UUUCU](CG)[UCGA]{T*}[G*][G*]{T*} |
| | [C] |
| 1184 | [G*][G*][U](G)[U][C](GAGAAGAGGAGAA)[C](AA)[U] |
| 1185 | (GGUGAAUAGUAUAACAAUAUGCUAAAUGUUGUUAUAGUAUCCACCA |
| | GGGGUCCACAUGGCAAC) |
| 1186 | (GGUGAAGAGGAGAACAAUAUGCUAAAUGUUGUUCUCGUCUCCACCA |
| | GGGGUCCACAUGGCAAC) |
| 1187 | (GGUGUCGAGAAGAGGAGAACAAUAUGCUAAAUGUUGUUCUCGUCUC |
| | CUCGACACCAGGGGUCCACAUGGCAAC) |
| 1188 | (GGACCAACUGCUUGGCACCCCUGGCCAAGGUCAUCCAUGACAACUUU |
| | GGUAUCGUGGAAGGACC) |
| 1189 | (GGGAACUGGAUCUAUCAAGACUGAGUUGAUUUCUGUGUCUGAAGUG |
| | UAAGUGAACACAGAA) |
| 1190 | (GGACCATCGACGAGAAAGGGACTGAAGCTGCTGGGGCCATGTTTTTAG |
| | AGGCCATACCCAT) |

In Table 8; a base in parentheses e.g. “(N)” depicts an RNA base; a letter in square brackets e.g. “[N]” depicts a 2′-OMe RNA base; “*” depicts a Phosphorothioate linkage; a base in curly brackets e.g. “{N}” depicts an LNA base; “c” is a cytidine nucleotide or a variant thereof, a deoxycytidine nucleotide or a variant thereof, or an abasic site, at the position corresponding to a nucleotide, in some cases for example an adenosine or a cytidine, in some cases for example an adenosine, to be edited in the target sequence.

TABLE 9

| SEQ ID NO. | Sequence 5′→3′ |
|---|---|
| 1191 | UsCsAUUAAACGCCAGAGUCsCsGsGsA |
| 1192 | UsCsUGAAUAAUCCAGGAAASAsGsCsA |
| 1193 | UsAsUAGGGGUGCCAAGCAGsUsUsGsG |
| 1194 | UsAsUGGUUUUUCCAGACGGsCsAsGsG |
| 1195 | GsGsUGCAGAUUCCAGGUGGSGsAsCsG |
| 1196 | AsCsAGACUUGGCCACUGAGSUsGsGSG |
| 1197 | UsAsUGUGUCGGCCACGGAAsCsAsGsG |
| 1198 | AsASUAAGGGGUCCACAUGGsCsAsAsC |
| 1199 | UsCsGAGCAAUGCCAUCACCsUsCsCsC |
| 1200 | UsAsUUUCCCUGCCAGAAUAsGsAsUsG |
| 1201 | GsAsUGCUCCAACCACCACASASGsUsU |
| 1202 | CsGsUCUCUUGCCCACGCCAsCsCsAsG |
| 1203 | GsUsCUCUUGAUACAUCCAGsUsUsCsC |
| 1204 | CsAsCAUGGGAUUCCCAUUGsAsUsGsA |
| 1205 | UsAsUCGACCAAACCCGUUGsAsCsUsC |
| 1206 | CsAsCGUCAUGAGCCCUUCCsAsCsGsA |
| 1207 | AsAsCGAGGGAUCCCGCUCCsUsGsGsA |
| 1208 | GsAsAGAGGCUGUCGUCAUAsCsUsUsC |
| 1209 | CsAsAGAGGUCAACGAAGGGsGsUsCsA |
| 1210 | AsAsCGCCAGGGGCGCUAAGsCsAsGsU |
| 1211 | UsAsCGCAUGGACCGUGGUCsAsUsGsA |
| 1212 | UsASCAUGACCCUCUUGGCUSCsCsCsC |
| 1213 | GsAsCUAGCCAAACUCGUUGsUsCsAsU |
| 1214 | AsGsUCGCCACAGCUUCCCGsGsAsGsG |
| 1215 | UsGsUAUAUCCACCUUACCASGsAsGsU |
| 1216 | AsGsGAGGGGUCUCACUCCUsUsGsGsA |
| 1217 | CsUsAGGCAACAACAUCCACsUsUsUsA |
| 1218 | CsCsGAGCGCCAGCAGAGGCSAsGsGsG |
| 1219 | UsAsUGGUUUUUCCAGACGGsCsAsGsG |
| 1220 | GsAsAGAGGCUGUCGUCAUAsCsUsUsC |
| 1221 | GsAsAGAGGCUGUCGUCAUAsCsUsUsC |
| 1222 | UsAsCAUGACCCUCUUGGCUSCsCsCsC |
| 1223 | UsASCAUGACCCUCUUGGCUSCsCsCsC |
| 1224 | AsGsUCGCCACAGCUUCCCGsGsAsGsG |
| 1225 | AsGsUCGCCACAGCUUCCCGsGsAsGsG |
| 1226 | AsGsGAGGGGUCUCACUCCUsUsGsGsA |
| 1227 | AsGsGAGGGGUCUCACUCCUsUsGsGsA |

In Table 9; nucleotides highlighted in bold are unmodified and are placed opposite the triplet with the target adenosine in the middle. Nucleotides highlighted in italic are modified with 2-O-methylation, 2′-fluorinated nucleotides are grayed out. The backbone contains terminal phosphorothioate linkages as indicated by “s”. The first three nucleotides at the 5′-end are not complementary to the mRNA substrate, but serve as linker sequence between gRNA and SNAP-tag.

TABLE 10

| SEQ ID NO. | Sequence 5′→3′ |
|---|---|
| 1228 | TAATCTAGGAAAACTGAGAACAGAGGCCCTGAAAAA |
| | GGGCCAAATTCTTCCACCC |

TABLE 11

| SEQ | |
|---|---|
| ID | |
| NO. | Sequence 5′→3′ |
| 1229 | AAACCGAGGGAUCAUAGGGGACUGAAUCCACCAUUCUUCUCCCAAU |
| | CCCUGCAACUCCUUCUUCCCCUGC |
| 1230 | UGAACAGCUCCUCGCCCUUGCUCACUGGCAGAGCCCUCCAGCAUCGC |
| | GAGCAGGCGCUGCCUCCUCCGCC |
| 1231 | UCUCAGUCCAAUGUAUGGUCCGAGCACAAGCUCUAAUCAAAGUCCG |
| | CGGGUGUAGACCGGUUGCCAUAGGA |
| 1232 | ACAGCUCCUCGCCCUUGCUCACUGGCAGAGCCCUCCAGCAUCGCGAG |
| | CAGGCGCUGCCUCCUCCGCCGCUG |
| 1233 | ACAGCUCCUCGCCCUUGCUCACUGGCAGAGCCCUCAAGCAUCGCGAG |
| | CAGGCGCUGCCUCCUCCGCCGCUG |
| 1234 | ACAGCUCCUCGCCCUUGCUCACUGGCAGAGCCCUCUAGCAUCGCGAG |
| | CAGGCGCUGCCUCCUCCGCCGCUG |
| 1235 | ACAGCUCCUCGCCCUUGCUCACUGGCAGAGCCCUCGAGCAUCGCGAG |
| | CAGGCGCUGCCUCCUCCGCCGCUG |
| 1236 | ACAGCUCCUCGCCCUUGCUCACUGGCAGAGCCCUGCAGCAUCGCGAG |
| | CAGGCGCUGCCUCCUCCGCCGCUG |
| 1237 | ACAGCUCCUCGCCCUUGCUCACUGGCAGAGCCCUUCAGCAUCGCGAG |
| | CAGGCGCUGCCUCCUCCGCCGCUG |
| 1238 | ACAGCUCCUCGCCCUUGCUCACUGGCAGAGCCCUACAGCAUCGCGAG |
| | CAGGCGCUGCCUCCUCCGCCGCUG |
| 1239 | ACAGCUCCUCGCCCUUGCUCACUGGCAGAGCCCUCCUGCAUCGCGAG |
| | CAGGCGCUGCCUCCUCCGCCGCUG |
| 1240 | ACAGCUCCUCGCCCUUGCUCACUGGCAGAGCCCUGCUGCAUCGCGAG |
| | CAGGCGCUGCCUCCUCCGCCGCUG |
| 1241 | ACAGCUCCUCGCCCUUGCUCACUGGCAGAGCCCUUCUGCAUCGCGAG |
| | CAGGCGCUGCCUCCUCCGCCGCUG |
| 1242 | ACAGCUCCUCGCCCUUGCUCACUGGCAGAGCCCUACUGCAUCGCGAG |
| | CAGGCGCUGCCUCCUCCGCCGCUG |
| 1243 | ACAGCUCCUCGCCCUUGCUCACUGGCAGAGCCCUCCCGCAUCGCGAG |
| | CAGGCGCUGCCUCCUCCGCCGCUG |
| 1244 | ACAGCUCCUCGCCCUUGCUCACUGGCAGAGCCCUGCCGCAUCGCGAG |
| | CAGGCGCUGCCUCCUCCGCCGCUG |
| 1245 | ACAGCUCCUCGCCCUUGCUCACUGGCAGAGCCCUUCCGCAUCGCGAG |
| | CAGGCGCUGCCUCCUCCGCCGCUG |
| 1246 | ACAGCUCCUCGCCCUUGCUCACUGGCAGAGCCCUACCGCAUCGCGAG |
| | CAGGCGCUGCCUCCUCCGCCGCUG |
| 1247 | ACAGCUCCUCGCCCUUGCUCACUGGCAGAGCCCUCCGGCAUCGCGAG |
| | CAGGCGCUGCCUCCUCCGCCGCUG |
| 1248 | ACAGCUCCUCGCCCUUGCUCACUGGCAGAGCCCUGCUGCAUCGCGAG |
| | CAGGCGCUGCCUCCUCCGCCGCUG |
| 1249 | ACAGCUCCUCGCCCUUGCUCACUGGCAGAGCCCUUCGGCAUCGCGAG |
| | CAGGCGCUGCCUCCUCCGCCGCUG |
| 1250 | ACAGCUCCUCGCCCUUGCUCACUGGCAGAGCCCUACGGCAUCGCGAG |
| | CAGGCGCUGCCUCCUCCGCCGCUG |
| 1251 | ACUGGCAGAGCCCUCCAGCAUCGCGAGCAGG |
| 1252 | GCCCUUGCUCACUGGCAGAGCCCUCCAGCAUCGCGAGCAGGCGCUGC |
| | CUCC |
| 1253 | ACAGCUCCUCGCCCUUGCUCACUGGCAGAGCCCUCCAGCAUCGCGAG |
| | CAGGCGCUGCCUCCUCCGCCGCUG |
| 1254 | ACCCCGGUGAACAGCUCCUCGCCCUUGCUCACUGGCAGAGCCCUCCA |
| | GCAUCGCGAGCAGGCGCUGCCUCCUCCGCCGCUGCCUCCUCCGC |
| 1255 | GCUCGACCAGGAUGGGCACCACCCCGGUGAACAGCUCCUCGCCCUUG |
| | CUCACUGGCAGAGCCCUCCAGCAUCGCGAGCAGGCGCUGCCUCCUCC |
| | GCCGCUGCCUCCUCCGCCGCUGCCUCCUCCGCCCUGCUCGCCGUCCA |
| | GCUCGACCAGGAUGGGCACCACCCCGGUGAACAGCUCCUCGCCCUUG |
| | CUCACUGGCAGAGCCCUCCAGCAUCGCGAGCAGGCGCUGCCUCCUCC |
| | GCCGCUGCCUCCUCCGCCGCUGCCUCCUCCGCCCUGCAGCUUGUACA |
| 1256 | UCGCCGUCCAGCUCGACCAGGAUGGGCACCACCCCGGUGAACAGCUC |
| | CUCGCCCUUGCUCACUGGCAGAGCCCUCCAGCAUCGCGAGCAGGCGC |
| | UGCCUCCUCCGCCGCUGCCUCCUCCGCCGCUGCCUCCUCCGCCCUGC |
| | AGCUUGUACA |
| 1257 | GCCGUUUACGUCGCCGUCCAGCUCGACCAGGAUGGGCACCACCCCGG |
| | UGAACAGCUCCUCGCCCUUGCUCACUGGCAGAGCCCUCCAGCAUCGC |
| | GAGCAGGCGCUGCCUCCUCCGCCGCUGCCUCCUCCGCCGCUGCCUCC |
| | UCCGCCCUGCAGCUUGUACAGCUCGUCCAU |
| 1258 | UGAACUUGUGGCCGUUUACGUCGCCGUCCAGCUCGACCAGGAUGGG |
| | CACCACCCCGGUGAACAGCUCCUCGCCCUUGCUCACUGGCAGAGCCC |
| | UCCAGCAUCGCGAGCAGGCGCUGCCUCCUCCGCCGCUGCCUCCUCCG |
| | CCGCUGCCUCCUCCGCCCUGCAGCUUGUACAGCUCGUCCAUGCCGCC |
| | GGUG |
| 1259 | CCGGACACGCUGAACUUGUGGCCGUUUACGUCGCCGUCCAGCUCGAC |
| | CAGGAUGGGCACCACCCCGGUGAACAGCUCCUCGCCCUUGCUCACUG |
| | GCAGAGCCCUCCAGCAUCGCGAGCAGGCGCUGCCUCCUCCGCCGCUG |
| | CCUCCUCCGCCGCUGCCUCCUCCGCCCUGCAGCUUGUACAGCUCGUC |
| | CAUGCCGCCGGUGGAGUGGCGGC |
| 1260 | GCGACCGGGGAUCUCCACAGAUUCUUCCGGC |
| 1261 | GCUCACGGUGGCGACCGGGGAUCUCCACAGAUUCUUCCGGCGUGUA |
| | UACCU |
| 1262 | CCUCGCCCUUGCUCACGGUGGCGACCGGGGAUCUCCACAGAUUCUUC |
| | CGGCGUGUAUACCUUCUGCUGCCU |
| 1263 | GUGAACAGCUCCUCGCCCUUGCUCACGGUGGCGACCGGGGAUCUCCA |
| | CAGAUUCUUCCGGCGUGUAUACCUUCUGCUGCCUCCUCCGCCGC |
| 1264 | CACCACCCCGGUGAACAGCUCCUCGCCCUUGCUCACGGUGGCGACCG |
| | GGGAUCUCCACAGAUUCUUCCGGCGUGUAUACCUUCUGCUGCCUCCU |
| | CCGCCGCUGCCUCCUCC |
| 1265 | CCAGGAUGGGCACCACCCCGGUGAACAGCUCCUCGCCCUUGCUCACG |
| | GUGGCGACCGGGGAUCUCCACAGAUUCUUCCGGCGUGUAUACCUUC |
| | UGCUGCCUCCUCCGCCGCUGCCUCCUCCGCCGCUGCCU |
| 1266 | UCCAGCUCGACCAGGAUGGGCACCACCCCGGUGAACAGCUCCUCGCC |
| | CUUGCUCACGGUGGCGACCGGGGAUCUCCACAGAUUCUUCCGGCGU |
| | GUAUACCUUCUGCUGCCUCCUCCGCCGCUGCCUCCUCCGCCGCUGCC |
| | UCCUCCGCCCU |
| 1267 | CGGCGACGUAUCCAGCUCGACCAGGAUGGGCACCACCCCGGUGAACA |
| | GCUCCUCGCCCUUGCUCACGGUGGCGACCGGGGAUCUCCACAGAUUC |
| | UUCCGGCGUGUAUACCUUCUGCUGCCUCCUCCGCCGCUGCCUCCUCC |
| | GCCGCUGCCUCCUCCGCCCUGCAGCUUGUA |
| 1268 | UGUGGCCGUUUACGUCGCCGUCCAGCUCGACCAGGAUGGGCACCACC |
| | CCGGUGAACAGCUCCUCGCCCUUGCUCACGGUGGCGACCGGGGAUCU |
| | CCACAGAUUCUUCCGGCGUGUAUACCUUCUGCUGCCUCCUCCGCCGC |
| | UGCCUCCUCCGCCGCUGCCUCCUCCGCCCUGCAGCUUGUACAGCUCG |
| | UCC |
| 1269 | ACGCUGAACUUGUGGCCGUUUACGUCGCCGUCCAGCUCGACCAGGA |
| | UGGGCACCACCCCGGUGAACAGCUCCUCGCCCUUGCUCACGGUGGCG |
| | ACCGGGGAUCUCCACAGAUUCUUCCGGCGUGUAUACCUUCUGCUGCC |
| | UCCUCCGCCGCUGCCUCCUCCGCCGCUGCCUCCUCCGCCCUGCAGCU |
| | UGUACAGCUCGUCCAUGCCGCCGG |
| 1270 | CAGCAUCGCGAGCAGGCGCUGCCUCCUCCGCCGCUGCCUCCUCCGCC |
| | GCUGCCUCCUCCGCCCUGCAGCUU |
| 1271 | CCCUCCAGCAUCGCGAGCAGGCGCUGCCUCCUCCGCCGCUGCCUCCU |
| | CCGCCGCUGCCUCCUCCGCCCUGC |
| 1272 | CAGAGCCCUCCAGCAUCGCGAGCAGGCGCUGCCUCCUCCGCCGCUGC |
| | CUCCUCCGCCGCUGCCUCCUCCGC |
| 1273 | ACUGGCAGAGCCCUCCCAGCAUCGCGAGCAGGCGCUGCCUCCUCCGC |
| | CGCUGCCUCCUCCGCCGCUGCCUCC |
| 1274 | UGCUCACUGGCAGAGCCCUCCAGCAUCGCGAGCAGGCGCUGCCUCCU |
| | CCGCCGCUGCCUCCUCCGCCGCUG |
| 1275 | GCCCUUGCUCACUGGCAGAGCCCUCCAGCAUCGCGAGCAGGCGCUGC |
| | CUCCUCCGCCGCUGCCUCCUCCGC |
| 1276 | UCCUCGCCCUUGCUCACUGGCAGAGCCCUCCAGCAUCGCGAGCAGGC |
| | GCUGCCUCCUCCGCCGCUGCCUCC |
| 1277 | GGUGAACAGCUCCUCGCCCUUGCUCACUGGCAGAGCCCUCCAGCAUC |
| | GCGAGCAGGCGCUGCCUCCUCCGC |
| 1278 | ACCCCGGUGAACAGCUCCUCGCCCUUGCUCACUGGCAGAGCCCUCCA |
| | GCAUCGCGAGCAGGCGCUGCCUCC |
| 1279 | GCACCACCCCGGUGAACAGCUCCUCGCCCUUGCUCACUGGCAGAGCC |
| | CUCCAGCAUCGCGAGCAGGCGCUG |
| 1280 | GAUGGGCACCACCCCGGUGAACAGCUCCUCGCCCUUGCUCACUGGCA |
| | GAGCCCUCCAGCAUCGCGAGCAGG |
| 1281 | ACCAGGAUGGGCACCACCCCGGUGAACAGCUCCUCGCCCUUGCUCAC |
| | UGGCAGAGCCCUCCAGCAUCGCGA |
| 1282 | GCUCGACCAGGAUGGGCACCACCCCGGUGAACAGCUCCUCGCCCUUG |
| | CUCACUGGCAGAGCCCUCCAGCAU |
| 1283 | GUCCAGCUCGACCAGGAUGGGCACCACCCCGGUGAACAGCUCCUCGC |
| | CCUUGCUCACUGGCAGAGCCCUCC |
| 1284 | CACAGAUUCUUCCGGCGUGUAUACCUUCUGCUGCCUCCUCCGCCGCU |
| | GCCUCCUCCGCCGCUGCCUCCUCC |
| 1285 | AUCUCCACAGAUUCUUCCGGCGUGUAUACCUUCUGCUGCCUCCUCCG |
| | CCGCUGCCUCCUCCGCCGCUGCCU |
| 1286 | CGGGGAUCUCCACAGAUUCUUCCGGCGUGUAUACCUUCUGCUGCCUC |
| | CUCCGCCGCUGCCUCCUCCGCCGC |
| 1287 | GCGACCGGGGAUCUCCACAGAUUCUUCCGGCGUGUAUACCUUCUGC |
| | UGCCUCCUCCGCCGCUGCCUCCUCC |
| 1288 | CGGUGGCGACCGGGGAUCUCCACAGAUUCUUCCGGCGUGUAUACCU |
| | UCUGCUGCCUCCUCCGCCGCUGCCU |
| 1289 | GCUCACGGUGGCGACCGGGGAUCUCCACAGAUUCUUCCGGCGUGUA |
| | UACCUUCUGCUGCCUCCUCCGCCGC |
| 1290 | CCCUUGCUCACGGUGGCGACCGGGGAUCUCCACAGAUUCUUCCGGCG |
| | UGUAUACCUUCUGCUGCCUCCUCC |
| 1291 | CAGCUCCUCGCCCUUGCUCACGGUGGCGACCGGGGAUCUCCACAGAU |
| | UCUUCCGGCGUGUAUACCUUCUGC |
| 1292 | GUGAACAGCUCCUCGCCCUUGCUCACGGUGGCGACCGGGGAUCUCCA |
| | CAGAUUCUUCCGGCGUGUAUACCU |
| 1293 | CCCCGGUGAACAGCUCCUCGCCCUUGCUCACGGUGGCGACCGGGGAU |
| | CUCCACAGAUUCUUCCGGCGUGUA |
| 1294 | CACCACCCCGGUGAACAGCUCCUCGCCCUUGCUCACGGUGGCGACCG |
| | GGGAUCUCCACAGAUUCUUCCGGC |
| 1295 | AUGGGCACCACCCCGGUGAACAGCUCCUCGCCCUUGCUCACGGUGGC |
| | GACCGGGGAUCUCCACAGAUUCUU |
| 1296 | CCAGGAUGGGCACCACCCCGGUGAACAGCUCCUCGCCCUUGCUCACG |
| | GUGGCGACCGGGGAUCUCCACAGA |
| 1297 | CUCGACCAGGAUGGGCACCACCCCGGUGAACAGCUCCUCGCCCUUGC |
| | UCACGGUGGCGACCGGGGAUCUCC |
| 1298 | GAUGGGCACCACCCCGGUGAACAGCUCCUCGCCCUUGCUCACUGGCA |
| | GAGCCCUCCAGCAUCGCGAGCAGGCGCUGCCUCCUCCGCCCUGCAGC |
| | UUGUACAGCUCGUCCAU |
| 1299 | GAUGGGCACCACCCCGGUGAACAGCUCCUCGCCCUUGCUCACUGGCA |
| | GAGCCCUGCAGCAUCGCGAGCAGGCGCUGCCUCCUCCGCCCUGCAGC |
| | UUGUACAGCUCGUCCAU |
| 1300 | GAUGGGCACCACCCCGGUGAACAGCUCCUCGCCCUUGCUCACUGGCA |
| | GAGCCCUUCAGCAUCGCGAGCAGGCGCUGCCUCCUCCGCCCUGCAGC |
| | UUGUACAGCUCGUCCAU |
| 1301 | GAUGGGCACCACCCCGGUGAACAGCUCCUCGCCCUUGCUCACUGGCA |
| | GAGCCCUACAGCAUCGCGAGCAGGCGCUGCCUCCUCCGCCCUGCAGC |
| | UUGUACAGCUCGUCCAU |
| 1302 | GAUGGGCACCACCCCGGUGAACAGCUCCUCGCCCUUGCUCACUGGCA |
| | GAGCCCUCCGGCAUCGCGAGCAGGCGCUGCCUCCUCCGCCCUGCAGC |
| | UUGUACAGCUCGUCCAU |
| 1303 | GAUGGGCACCACCCCGGUGAACAGCUCCUCGCCCUUGCUCACUGGCA |
| | GAGCCCUGCGGCAUCGCGAGCAGGCGCUGCCUCCUCCGCCCUGCAGC |
| | UUGUACAGCUCGUCCAU |
| 1304 | GAUGGGCACCACCCCGGUGAACAGCUCCUCGCCCUUGCUCACUGGCA |
| | GAGCCCUUCGGCAUCGCGAGCAGGCGCUGCCUCCUCCGCCCUGCAGC |
| | UUGUACAGCUCGUCCAU |
| 1305 | GAUGGGCACCACCCCGGUGAACAGCUCCUCGCCCUUGCUCACUGGCA |
| | GAGCCCUACGGCAUCGCGAGCAGGCGCUGCCUCCUCCGCCCUGCAGC |
| | UUGUACAGCUCGUCCAU |
| 1306 | GAUGGGCACCACCCCGGUGAACAGCUCCUCGCCCUUGCUCACUGGCA |
| | GAGCCCUCCUGCAUCGCGAGCAGGCGCUGCCUCCUCCGCCCUGCAGC |
| | UUGUACAGCUCGUCCAU |
| 1307 | GAUGGGCACCACCCCGGUGAACAGCUCCUCGCCCUUGCUCACUGGCA |
| | GAGCCCUGCUGCAUCGCGAGCAGGCGCUGCCUCCUCCGCCCUGCAGC |
| | UUGUACAGCUCGUCCAU |
| 1308 | GAUGGGCACCACCCCGGUGAACAGCUCCUCGCCCUUGCUCACUGGCA |
| | GAGCCCUACUGCAUCGCGAGCAGGCGCUGCCUCCUCCGCCCUGCAGC |
| | UUGUACAGCUCGUCCAU |
| 1309 | GAUGGGCACCACCCCGGUGAACAGCUCCUCGCCCUUGCUCACUGGCA |
| | GAGCCCUUCUGCAUCGCGAGCAGGCGCUGCCUCCUCCGCCCUGCAGC |
| | UUGUACAGCUCGUCCAU |
| 1310 | GAUGGGCACCACCCCGGUGAACAGCUCCUCGCCCUUGCUCACUGGCA |
| | GAGCCCUCCCGCAUCGCGAGCAGGCGCUGCCUCCUCCGCCCUGCAGC |
| | UUGUACAGCUCGUCCAU |
| 1311 | GAUGGGCACCACCCCGGUGAACAGCUCCUCGCCCUUGCUCACUGGCA |
| | GAGCCCUGCCGCAUCGCGAGCAGGCGCUGCCUCCUCCGCCCUGCAGC |
| | UUGUACAGCUCGUCCAU |
| 1312 | GAUGGGCACCACCCCGGUGAACAGCUCCUCGCCCUUGCUCACUGGCA |
| | GAGCCCUUCCGCAUCGCGAGCAGGCGCUGCCUCCUCCGCCCUGCAGC |
| | UUGUACAGCUCGUCCAU |
| 1313 | GAUGGGCACCACCCCGGUGAACAGCUCCUCGCCCUUGCUCACUGGCA |
| | GAGCCCUACCGCAUCGCGAGCAGGCGCUGCCUCCUCCGCCCUGCAGC |
| | UUGUACAGCUCGUCCAU |
| 1314 | UACCGCUACAGCCACGCUGAUUUCAGCUAUACCUGCCCGGUAUAAA |
| | GGGACGUUCACACCGCGAUGUUCUCUGCUGGGGAAUUGCGCGAUAU |
| | UCAGGAUUAAAAGAAGUGC |
| 1315 | ACUACAGUUGCUCCGAUAUUUAGGCUACGUCAAUAGGCACUAACUU |
| | AUUGGCGCUGGUGAACGGACUUCCUCUCGAGUACCAGAAGAUGACU |
| | ACAAAACUCCUUUCCAUUGCGAGUAUCGGAGUCUGGCUCAGUUUGG |
| | CCAGGGAGGCACU |
| 1316 | CGGAAGAGGGUGGGGCCGCGGUGGCCAGGGAGCCGGCGCCGCCACG |
| | CGCGG |
| 1317 | CAGCUGAGGCCGGAAGAGGGUGGGGCCGCGGUGGCCAGGGAGCCGG |
| | CGCCGCCACGCGCGGGUGGGGGGGA |
| 1318 | GGAGGCGAAAGCAGCCCGGACAGCUGAGGCCGGAAGAGGGUGGGGC |
| | CGCGGUGGCCAGGGAGCCGGCGCCGCCACGCGCGGGUGGGGGGGAC |
| | UGGGGUUGCUCGCGGGCUC |
| 1319 | GAGGCGCAGCAUCCACAGGCGGAGGCGAAAGCAGCCCGGACAGCUG |
| | AGGCCGGAAGAGGGUGGGGCCGCGGUGGCCAGGGAGCCGGCGCCGC |
| | CACGCGCGGGUGGGGGGGACUGGGGUUGCUCGCGGGCUCCGGGCGG |
| | GCGGCGGGCGCCG |
| 1320 | UCUUGCCUACGCCACCAGCUCCAACCACCACAAGUUUAUAUUCAGUC |
| | AUUU |
| 1321 | GAUUCUGAAUUAGCUGUAUCGUCAAGGCACUCUUGCCUACGCCACC |
| | AGCUCCAACCACCACAAGUUUAUAUUCAGUCAUUUUCAGCAGGCCU |
| | CUCUCCCGCACCUGGGAGC |
| 1322 | AUCAUAUUCGUCCACAAAAUGAUUCUGAAUUAGCUGUAUCGUCAAG |
| | GCACUCUUGCCUACGCCACCAGCUCCAACCACCACAAGUUUAUAUUC |
| | AGUCAUUUUCAGCAGGCCUCUCUCCCGCACCUGGGAGCCGCUGAGCC |
| | UCUGGCCCCGC |
| 1323 | UCGGCAUGGUAUGAAGUACUUCGUCCAGGAGCUGGAGGGCCCGGUG |
| | UAAGU |
| 1324 | GGGUCUGCAAUCGGCAUGGUAUGAAGUACUUCGUCCAGGAGCUGGA |
| | GGGCCCGGUGUAAGUGAAUUUCAAU |
| 1325 | GACCUCAGUCUAAAGGUUGUGGGUCUGCAAUCGGCAUGGUAUGAAG |
| | UACUUCGUCCAGGAGCUGGAGGGCCCGGUGUAAGUGAAUUUCAAUC |
| | CAGCAAGGUGUUUCUUUGA |
| 1326 | UAAGGGCCCCAACGGUAAAAGACCUCAGUCUAAAGGUUGUGGGUCU |
| | GCAAUCGGCAUGGUAUGAAGUACUUCGUCCAGGAGCUGGAGGGCCC |
| | GGUGUAAGUGAAUUUCAAUCCAGCAAGGUGUUUCUUUGAUGCUCUG |
| | UCUUGGGUAAUCC |
| 1327 | UGGGGGGUUCGGCUGCCGACAUCAGCAAUUGCUCUGCCACCAUCUC |
| | AGCCC |
| 1328 | AGCAGGGCCGUGGGGGGUUCGGCUGCCGACAUCAGCAAUUGCUCUG |
| | CCACCAUCUCAGCCCAUCCUCCGAA |
| 1329 | AGUAGAAGGCCAAGAGCCACAGCAGGGCCGUGGGGGGUUCGGCUGC |
| | CGACAUCAGCAAUUGCUCUGCCACCAUCUCAGCCCAUCCUCCGAAGU |
| | GAAUGAACAGGAACCAGC |
| 1330 | CCUCCCAUCACGGGGGCCGUAGUAGAAGGCCAAGAGCCACAGCAGG |
| | GCCGUGGGGGGUUCGGCUGCCGACAUCAGCAAUUGCUCUGCCACCA |
| | UCUCAGCCCAUCCUCCGAAGUGAAUGAACAGGAACCAGCUCUCAAA |
| | GGGACCUCCGCAG |
| 1331 | GCCAAACACCACATGCTTGCCATCTAGCCAGGCTGTCTTGACTGTCGT |
| | GATGAAGAACTGGGAGCCGTTGGTGTCCTTGCCTGCGTTGGCCATGCT |
| | CACCCAGCCAGGCCCGTAGTGCTTCAGTTTGAAGTTCTCATCGGGGAA |
| | GCGCTCA |
| 1332 | GGGAGTGGGTCCGCTCCACCAGATGCCAGCACCGGGGCCAGTGCAGC |
| | TCAGAGCCCTGTGGCGGACTACAGGGCCCGCACAGACGGTCACTCAA |
| | AGAAAGATGTCCCTGTGCCCTACTCCTTGGCGATGGCAAAGGGCTTCT |
| | CCACCTCGA |
| 1333 | TGCATTTTGTAAAATAGATACTAGCAGATTGTCCCAAGATGTGTACAG |
| | CTCATTCTCACAGCCCAGCGAGGGCACCTACTCCACAAATGCGTGGCC |
| | ACAGGTCATCACCTGTCCTGTGGCCCTGGCGAGCCTGATCCCTCACGC |
| | CGGGCAC |
| 1334 | GCTCATTCTCACAGCCCAGCGAGGGCACTTACTCCACAAATGCGTGGC |
| | CACAGGTCATCACCTGTCCTGTGGCCCCGGCGAGCCTGATCCCTCACG |
| | CCGGGCACCCACACGGCCTGCGTGCCTTCTAGACTTGAGTTCGCAGCT |
| | CTTTAAG |
| 1335 | TCGGCCGGGCCCTGGGGGCGGTGGGCGCTGGCCAGGACGCCCACCGT |
| | GTGGTTGCTGTCCAGGACGGTCCCGGCCCGCGACACTTCGGCCCAGAG |
| | CTGCTCCTCATCCAGCAGCGCCAGCAGCCCCATGGCCGTGAGCACCGG |
| | CTTGCGCA |
| 1336 | UGACCAGUCUUAAGAUCUUUCUUGACCUGCACCAUAAGAACUUCUC |
| | CAAAGGUACCAAAAUACUCUUUCAGGUCCUGUUCGGUUGUUUUCCA |
| | UGGGAGACCCAACACUAUU |
| 1337 | GAUGGGCACCACCCCGGUGAACAGCUCCUCGCCCUUGCUCACUGGCA |
| | GAGCCCUCGAGCAUCGCGAGCAGGCGCUGCCUCCUCCGCCCUGCAGC |
| | UUGUACAGCUCGUCCAU |
| 1338 | GAUGGGCACCACCCCGGUGAACAGCUCCUCGCCCUUGCUCACUGGCA |
| | GAGCCCUGGAGCAUCGCGAGCAGGCGCUGCCUCCUCCGCCCUGCAGC |
| | UUGUACAGCUCGUCCAU |
| 1339 | GAUGGGCACCACCCCGGUGAACAGCUCCUCGCCCUUGCUCACUGGCA |
| | GAGCCCUUGAGCAUCGCGAGCAGGCGCUGCCUCCUCCGCCCUGCAGC |
| | UUGUACAGCUCGUCCAU |
| 1340 | GAUGGGCACCACCCCGGUGAACAGCUCCUCGCCCUUGCUCACUGGCA |
| | GAGCCCUAGAGCAUCGCGAGCAGGCGCUGCCUCCUCCGCCCUGCAGC |
| | UUGUACAGCUCGUCCAU |
| 1341 | GAUGGGCACCACCCCGGUGAACAGCUCCUCGCCCUUGCUCACUGGCA |
| | GAGCCCUCGGGCAUCGCGAGCAGGCGCUGCCUCCUCCGCCCUGCAGC |
| | UUGUACAGCUCGUCCAU |
| 1342 | GAUGGGCACCACCCCGGUGAACAGCUCCUCGCCCUUGCUCACUGGCA |
| | GAGCCCUGGGGCAUCGCGAGCAGGCGCUGCCUCCUCCGCCCUGCAGC |
| | UUGUACAGCUCGUCCAU |
| 1343 | GAUGGGCACCACCCCGGUGAACAGCUCCUCGCCCUUGCUCACUGGCA |
| | GAGCCCUUGGGCAUCGCGAGCAGGCGCUGCCUCCUCCGCCCUGCAGC |
| | UUGUACAGCUCGUCCAU |
| 1344 | GAUGGGCACCACCCCGGUGAACAGCUCCUCGCCCUUGCUCACUGGCA |
| | GAGCCCUAGGGCAUCGCGAGCAGGCGCUGCCUCCUCCGCCCUGCAGC |
| | UUGUACAGCUCGUCCAU |
| 1345 | GAUGGGCACCACCCCGGUGAACAGCUCCUCGCCCUUGCUCACUGGCA |
| | GAGCCCUCGUGCAUCGCGAGCAGGCGCUGCCUCCUCCGCCCUGCAGC |
| | UUGUACAGCUCGUCCAU |
| 1346 | GAUGGGCACCACCCCGGUGAACAGCUCCUCGCCCUUGCUCACUGGCA |
| | GAGCCCUGGUGCAUCGCGAGCAGGCGCUGCCUCCUCCGCCCUGCAGC |
| | UUGUACAGCUCGUCCAU |
| 1347 | GAUGGGCACCACCCCGGUGAACAGCUCCUCGCCCUUGCUCACUGGCA |
| | GAGCCCUAGUGCAUCGCGAGCAGGCGCUGCCUCCUCCGCCCUGCAGC |
| | UUGUACAGCUCGUCCAU |
| 1348 | GAUGGGCACCACCCCGGUGAACAGCUCCUCGCCCUUGCUCACUGGCA |
| | GAGCCCUUGUGCAUCGCGAGCAGGCGCUGCCUCCUCCGCCCUGCAGC |
| | UUGUACAGCUCGUCCAU |
| 1349 | GAUGGGCACCACCCCGGUGAACAGCUCCUCGCCCUUGCUCACUGGCA |
| | GAGCCCUCGCGCAUCGCGAGCAGGCGCUGCCUCCUCCGCCCUGCAGC |
| | UUGUACAGCUCGUCCAU |
| 1350 | GAUGGGCACCACCCCGGUGAACAGCUCCUCGCCCUUGCUCACUGGCA |
| | GAGCCCUGGCGCAUCGCGAGCAGGCGCUGCCUCCUCCGCCCUGCAGC |
| | UUGUACAGCUCGUCCAU |
| 1351 | GAUGGGCACCACCCCGGUGAACAGCUCCUCGCCCUUGCUCACUGGCA |
| | GAGCCCUUGCGCAUCGCGAGCAGGCGCUGCCUCCUCCGCCCUGCAGC |
| | UUGUACAGCUCGUCCAU |
| 1352 | GAUGGGCACCACCCCGGUGAACAGCUCCUCGCCCUUGCUCACUGGCA |
| | GAGCCCUAGCGCAUCGCGAGCAGGCGCUGCCUCCUCCGCCCUGCAGC |
| | UUGUACAGCUCGUCCAU |
| 1353 | GAUGGGCACCACCCCGGUGAACAGCUCCUCGCCCUUGCUCACUGGCA |
| | GAGCCCUCGAGCAUCGCGAGCAGGCGCUGCCUCCUCCGCCCUGCAGC |
| | UUGUACAGCUCGUCCAU |
| 1354 | GAUGGGCACCACCCCGGUGAACAGCUCCUCGCCCUUGCUCACUGGCA |
| | GAGCCCUGGAGCAUCGCGAGCAGGCGCUGCCUCCUCCGCCCUGCAGC |
| | UUGUACAGCUCGUCCAU |
| 1355 | GAUGGGCACCACCCCGGUGAACAGCUCCUCGCCCUUGCUCACUGGCA |
| | GAGCCCUUGAGCAUCGCGAGCAGGCGCUGCCUCCUCCGCCCUGCAGC |
| | UUGUACAGCUCGUCCAU |
| 1356 | GAUGGGCACCACCCCGGUGAACAGCUCCUCGCCCUUGCUCACUGGCA |
| | GAGCCCUAGAGCAUCGCGAGCAGGCGCUGCCUCCUCCGCCCUGCAGC |
| | UUGUACAGCUCGUCCAU |
| 1357 | GAUGGGCACCACCCCGGUGAACAGCUCCUCGCCCUUGCUCACUGGCA |
| | GAGCCCUCGUGCAUCGCGAGCAGGCGCUGCCUCCUCCGCCCUGCAGC |
| | UUGUACAGCUCGUCCAU |
| 1358 | GAUGGGCACCACCCCGGUGAACAGCUCCUCGCCCUUGCUCACUGGCA |
| | GAGCCCUCGGGCAUCGCGAGCAGGCGCUGCCUCCUCCGCCCUGCAGC |
| | UUGUACAGCUCGUCCAU |
| 1359 | GAUGGGCACCACCCCGGUGAACAGCUCCUCGCCCUUGCUCACUGGCA |
| | GAGCCCUCGCGCAUCGCGAGCAGGCGCUGCCUCCUCCGCCCUGCAGC |
| | UUGUACAGCUCGUCCAU |
| 1360 | GAUGGGCACCACCCCGGUGAACAGCUCCUCGCCCUUGCUCACUGGCA |
| | GAGCCCUCGUGCAUCGCGAGCAGGCGCUGCCUCCUCCGCCCUGCAGC |
| | UUGUACAGCUCGUCCAU |
| 1361 | GAUGGGCACCACCCCGGUGAACAGCUCCUCGCCCUUGCUCACUGGCA |
| | GAGCCCUGGUGCAUCGCGAGCAGGCGCUGCCUCCUCCGCCCUGCAGC |
| | UUGUACAGCUCGUCCAU |
| 1362 | GAUGGGCACCACCCCGGUGAACAGCUCCUCGCCCUUGCUCACUGGCA |
| | GAGCCCUUGUGCAUCGCGAGCAGGCGCUGCCUCCUCCGCCCUGCAGC |
| | UUGUACAGCUCGUCCAU |
| 1363 | GAUGGGCACCACCCCGGUGAACAGCUCCUCGCCCUUGCUCACUGGCA |
| | GAGCCCUAGUGCAUCGCGAGCAGGCGCUGCCUCCUCCGCCCUGCAGC |
| | UUGUACAGCUCGUCCAU |
| 1364 | GAUGGGCACCACCCCGGUGAACAGCUCCUCGCCCUUGCUCACUGGCA |
| | GAGCCCUCGAGCAUCGCGAGCAGGCGCUGCCUCCUCCGCCCUGCAGC |
| | UUGUACAGCUCGUCCAU |
| 1365 | GAUGGGCACCACCCCGGUGAACAGCUCCUCGCCCUUGCUCACUGGCA |
| | GAGCCCUCGCGCAUCGCGAGCAGGCGCUGCCUCCUCCGCCCUGCAGC |
| | UUGUACAGCUCGUCCAU |
| 1366 | GAUGGGCACCACCCCGGUGAACAGCUCCUCGCCCUUGCUCACUGGCA |
| | GAGCCCUCGGGCAUCGCGAGCAGGCGCUGCCUCCUCCGCCCUGCAGC |
| | UUGUACAGCUCGUCCAU |
| 1367 | GAUUCUGAAUUAGCUGUAUCGUCAAGGCACUCGUGCCGACGCCACC |
| | AGCUCCAACCACCACAAGUGGAGAGUCAGUCAUUUUCAGCAGGCCU |
| | CUCUCCCGCACCUGGGAGC |
| 1368 | GAUUCUGAAUUAGCUGGAUCGUCAAGGCACUCGGGCCGACGCCACC |
| | AGCUCCAACCACCACAAGUGGAGAGUCAGUCAUUUUCAGCAGGCCU |
| | CUCUCCCGCACCGGGGAGC |
| 1369 | GGGAGCAGCCUCUGGCAUUCUGGGAGCUUCAUCUGGACCUGGGUCU |
| | UCAGUGAACCAUUGUUCAAUAUCGUCCGGGGACAGCAUCAAAUCAU |
| | CCAUUGCUUGGGACGGCAA |
| 1370 | GGGAGCAGCCUCUGGCAUUCUGGGAGCUUCAUCUGGACCUGGGUCU |
| | UCAGUGAACCAUUGUUCAAGAUCGUCCGGGGACAGCAUCAAAUCAU |
| | CCAUUGCUUGGGACGGCAA |
| 1371 | GGGAGCAGCCUCUGGCAGUCGGGGAGCUUCAUCUGGACCUGGGUCU |
| | UCAGUGAACCAUUGUUCAAGAUCGUCCGGGGACAGCAUCAAAUCAU |
| | CCAGUGCUUGGGACGGCAA |
| 1372 | CAUAUUACAGAAUACCUUGAUAGCAUCCAAUUUGCAUCCUUGGUUA |
| | GGGUCAACCCAGUAUUCUCCACUCUUGAGUUCAGGAUGGCAGAAUU |
| | UCAGGUCUCUGCAGUUUCU |
| 1373 | GUGAAGAUAAGCCAGUCCUCUAGUAACAGAAUGAGCAAGACGGCAA |
| | GAGCUUACCCAGUCACUUGUGUGGAGACUUAAAUACUUGCAUAAAG |
| | AUCCAUUGGGAUAGUACUC |
| 1374 | GUGAACGUCAAACUGUCGGACCAAUAUGGCAGAAUCUUCUCUCAUC |
| | UCAACUUUCCAUAUCCGUAUCAUGGAAUCAUAGCAUCCUGUAACUA |
| | CUAGCUCUCUUACAGCUGG |
| 1375 | GCCAAUGAUCUCGUGAGUUAUCUCAGCAGUGUGAGCCAUCAGGGUG |
| | AUGACAUCCCAGGCGAUCGUGUGGCCUCCAGGAGCCCAGAGCAGGA |
| | AGUUGAGGAGAAGGUGCCU |
| 1376 | CAAGACGGUGAACCACUCCAUGGUCUUCUUGUCGGCUUUCUGCACU |
| | GUGUACCCCCAGAGCUCCGUGUUGCCGACAUCCUGGGGUGGCUUCCA |
| | CUCCAGAGCCACAUUAAG |
| 1377 | AGGAUUCUCUUUUGAAGUAUUGCUCCCCCAGUGGAUUGGGUGGCUC |
| | CAUUCACUCCAAUGCUGAGCACUUCCACAGAGUGGGUUAAAGCGGC |
| | UCCGAACACGAAACGUGUA |
| 1378 | GACGCCCACCGUGUGGUUGCUGUCCAGGACGGUCCCGGCCUGCGACA |
| | CUUCGGCCCAGAGCUGCUCCUCAUCCAGCAGCGCCAGCAGCCCCAUG |
| | GCCGUGAGCACCGGCUU |
| 1379 | GACGCCCACCGUGUGGUUGCUGUCCAGGACGGUCCCGGCCUGCGACA |
| | CUUCGGCCCAGAGCUGCUCCUCAUCUGCGGGGCGGGGGGGGGCCGUC |
| | GCCGCGUGGGGUCGUUG |
| 1380 | GGGTGATGGGTGCTGGCCAGGACACCCACTGTATGATTGCTGTCCAAC |
| | ACAGCCCCAGCCTTTGAGACCTCTGCCCAGAGTTGTTCTCCATCCAAC |
| | AGGGCCATGAGCCCCATGACTGTGAGTACTGGCTTTCGCAGCAACTGC |
| | ACATGGG |
| 1381 | ACTACAGTTGCTCCGATATTTAGGCTACGTCAATAGGCACTAACTTATT |
| | GGCGCTGGTGAACGGACTTCCTCTCGAGTACCAGAAGATGACTACAAA |
| | ACTCCTTTCCATTGCGAGTATCGGAGTCTGGCTCAGTTTGGCCAGGGA |
| | GGCACT |
| 1382 | CTGCAGGGCGGAGGAGGCAGCGGCGGAGGAGGCAGCGGCGGAGGAG |
| | GCAGCAGAAGGTATACACGCCGGAAGAATCTGTAGAGATCCCCGGTC |
| | GCCACC |
| 1383 | CTGCAGGGCGGAGGAGGCAGCGGCGGAGGAGGCAGCGGCGGAGGAG |
| | GCAGCGCCTGCTCGCGATGCTAGAGGGCTCTGCCA |
| 1384 | CTGCAGGGCGGAGGAGGCAGCGCCTGCTCGCGATGCTAGAGGGCTCT |
| | GCCA |
| 1385 | AAACCGAGGGAUCAUAGGGGACUGAAUCCACCAUUCUUCUCCCAAU |
| | CCCUGCAACUCCUUCUUCCCCUGC |
| 1386 | ATGGACGAGCTGTACAAGCTGCAGGGCGGAGGAGGCAGCGCCTGCTC |
| | GCGATGCTATAGGGCTCTGCCAGTGAGCAAGGGCGAGGAGCTGTTCAC |
| | CGGGGTGGTGCCCATC |
| 1387 | UAGCUGUAUCGUCAAGGCACUCUUGCCUACGCCACCAGCUCCAACCA |
| | CCACAAGUUUAUAUUCAGUCAUUUUCAGCAGGCCUCUCUCCCGC |
| 1388 | GAUUCUGAAUUAGCUGUAUCGUCAAGGCACUCUUGCCUACGCCACC |
| | AGCUCCAACUACCACAAGUUUAUAUUCAGUCAUUUUCAGCAGGCCU |
| | CUCUCCCGCACCUGGGAGC |
| 1389 | UCCACAAAAUGAUUCUGAAUUAGCUGUAUCGUCAAGGCACUCUUGC |
| | CUACGCCACCAGCUCCAACUACCACAAGUUUAUAUUCAGUCAUUUUC |
| | AGCAGGCCUCUCUCCCGCACCUGGGAGCCGCUGAGCCU |
| 1390 | AUCAUAUUCGUCCACAAAAUGAUUCUGAAUUAGCUGUAUCGUCAAG |
| | GCACUCUUGCCUACGCCACCAGCUCCAACCACCACAAGUUUAUAUUC |
| | AGUCAUUUUCAGCAGGCCUCUCUCCCGCACCUGGGAGCCGCUGAGCC |
| | UCUGGCCCCGC |
| 1391 | CUAUUGUUGGAUCAUAUUCGUCCACAAAAUGAUUCUGAAUUAGCUG |
| | UAUCGUCAAGGCACUCUUGCCUACGCCACCAGCUCCAACCACCACAA |
| | GUUUAUAUUCAGUCAUUUUCAGCAGGCCUCUCUCCCGCACCUGGGA |
| | GCCGCUGAGCCUCUGGCCCCGCCGCCGCCUUC |
| 1392 | UAGGAAUCCUCUAUUGUUGGAUCAUAUUCGUCCACAAAAUGAUUCU |
| | GAAUUAGCUGUAUCGUCAAGGCACUCUUGCCUACGCCACCAGCUCCA |
| | ACCACCACAAGUUUAUAUUCAGUCAUUUUCAGCAGGCCUCUCUCCCG |
| | CACCUGGGAGCCGCUGAGCCUCUGGCCCCGCCGCCGCCUUCAGUGCC |
| | UGCG |
| 1393 | GAGGCGCAGCAUCCACAGGCGGAGGCGAAAGCAGCCCGGACAGCUG |
| | AGGCCGGAAGAGGGUGGGGCCGCGGUGGCCAGGGAGCCGGCGCCGC |
| | CACGCGCGGGUGGGGGGGACUGGGGUUGCUCGCGGGCUCCGGGCGG |
| | GCGGCGGGCGCCG |
| 1394 | UCCUGUAGCUAAGGCCACAAAAUUAUCCACUGUUUUUGGAACAGUC |
| | UUUCCGAAGAGACCAAAGAUCACCCGGCCCACAUCUUCAUCUCCAAU |
| | UCGUAGGUCAAAAUACACCUUGACGGUGACUUUGGGCCCCUUCUUC |
| | UUCUCAUCGGCC |
| 1395 | GCCCUGGAUCAUGAAGUCCUUGAUUACACGAUGGAAUUUGCUGUUU |
| | UUGUAGCCAAAUCCUUUCUCUCCUGUAGCCAAGGCCACAAAAUUAU |
| | CCACUGUUUUUGGAACAGUCUUUCCGAAGAGACCAAAGAUCACCCG |
| | GCCUACAUCUUCA |
| 1396 | GCGCAAGUUAGGUUUUGUCAAGAAAGGGUGUAACGCAACCAAGUCA |
| | UAGUCCGCCUAGAAGCAUUUGCGGUG |
| 1397 | GCCAUGCCAAUCUCAUCUUGUUUUCUGCGCAAGUUAGGUUUUGUCA |
| | AGAAAGGGUGUAACGCAACCAAGUCAUAGUCCGCCUAGAAGCAUUU |
| | GCGGUGGACGAUGGAGGGGCCGGACUCGUCAUACUCCUG |
| 1398 | GGACUUCCUGUAACAACGCAUCUCAUAUUUGGAAUGACCAUUAAAA |
| | AAACAACAAUGUGCAAUCAAAGUC |
| 1399 | CAAGGUGCGGCUCCGGCCCCUCCCCUCUUCAAGGGGUCCACAUGGCA |
| | ACUGUGAGGAGGGGAGAUUCAGUG |
| 1400 | UAGCUGUAUCGUCAAGGCACUCGUGCCGACGCCACCAGCUCCAACCA |
| | CCACAAGGGGAGAGUCAGUCAGGGUCAGCAGGCCUCUCUCCCGC |
| 1401 | UAGCUGUAUCGUCAAGGCACUCUUGCCGACGCCACCAGCUCCAACCA |
| | CCACAAGUGUAUAGUCAGUCAUUUUCAGCAGGCCUCUCUCCCGC |
| 1402 | UAGCUGGAUCGUCAAGGCACUCGUGCCGACGCCACCAGCUCCAACCA |
| | CCACAAGGGGAGAGGCAGUCAGGGUCAGCAGGCCUCUCUCCCGC |
| 1403 | GAUUCUGAAUUAGCUGUAUCGUCAAGGCACUCUUGCCGACGCCACC |
| | AGCUCCAACCACCACAAGUGUAUAGUCAGUCAUUUUCAGCAGGCCU |
| | CUCUCCCGCACCUGGGAGC |
| 1404 | GAUUCUGAAUUAGCUGUAUCGUCAAGGCACUCGUGCCGACGCCACC |
| | AGCUCCAACCACCACAAGUGGAGAGUCAGUCAUUUUCAGCAGGCCU |
| | CUCUCCCGCACCUGGGAGC |
| 1405 | GAUUCUGAAUUAGCUGUAUCGUCAAGGCACUCGUGCCGACGCCACC |
| | AGCUCCAACCACCACAAGGGGAGAGUCAGUCAGGGUCAGCAGGCCU |
| | CUCUCCCGCACCUGGGAGC |
| 1406 | GCUCCCCGGUGCGGGAGAGAGGCCUGCUGACCCUGACUGCCUCUCCC |
| | CUUGUGGUGGUUGGAGCUGGUGGCGUCGGCACGAGUGCCUUGACGA |
| | UCCAGCUAAUUCAGAAUC |
| 1407 | GCAGAGCCUCCAGC |
| 1408 | CUCACUGGCAGAGCCUCCAGC |
| 1409 | CCCUUGCUCACUGGCAGAGCCUCCAGC |
| 1410 | CUCUCGCCCUUGCUCACUGGCAGAGCCUCCAGC |
| 1411 | CUCUCGCCCUUGCUCACUGGCAGAGCCUCCAGCAUCGC |
| 1412 | UGAACAGCUCUCGCCCUUGCUCACUGGCAGAGCCUCCAGCAUCGC |
| 1413 | UGAACAGCUCCUCGCCCUUGCUCACUGGCAGAGCCCUCCAGCAUCGC |
| | GAGCAGGCGCUGCCUCCUCCGCC |

TABLE 12

| SEQ ID | |
|---|---|
| NO. | Sequence 5′→3′ |
| 1414 | ACAAAUGGGGACGAGGGGGGGGGGGGGCC |
| 1415 | CGGAGAGCAGAGAGGGAGCG |
| 1416 | AAAAAAAAAAGATCTTGAAACTGTTTTAAGGTTGGCCGA |
| | TCTTAAAAAA |
| 1417 | UCAUAAUCAAUUUAUUAUUUUCUUUUAUUUUAUUCACAU |
| | AAUUUUGUUUUU |

The chemical transformation on a base may result in at least a partial knockdown of the edited RNA sequence. The chemical transformation may result in a substantially complete knockdown of the edited RNA sequence. The chemical transformation may result in a partial knockdown of the edited RNA sequence that is sufficient to impart a therapeutic effect to a subject receiving an engineered polynucleotide (e.g., a circular engineered guide RNA). An at least partial knockdown of an edited RNA sequence may result in a reduced level of an expressed protein or protein fragment thereof. A reduced level may be from about 5% to 100%. A reduced level may be from about 10% to 100%. A reduced level may be from about 15% to 100%. A reduced level may be from about 20% to 100%. A reduced level may be from about 25% to 100%. A reduced level may be from about 30% to 100%. A reduced level may be from about 40% to 100%. A reduced level may be from about 50% to 100%. A reduced level may be from about 60% to 100%. A reduced level may be from about 70% to 100%. A reduced level may be from about 80% to 100%.

An engineered polynucleotide (e.g., a circular engineered guide RNA) may comprise a targeting domain (an antisense region) for targeting a specific sequence region or base in a nucleic acid sequence for an RNA editing entity to perform a chemical transformation. The engineered polynucleotide may also comprise a recruiting domain. A targeting domain may comprise a sequence length that may be longer than an antisense RNA, a short hairpin RNA, an siRNA, miRNA, or snoRNA. A targeting domain may comprise a sequence length sufficient for the engineered guide RNA to form a secondary structure. In some cases, a base can refer to a nucleotide. In some cases, a nucleotide can refer to a base. A targeting domain may comprise a sequence length from about 20 nucleotides to about 1,000 nucleotides in length. A targeting domain may comprise a sequence length from about 50 nucleotides to about 1,000 nucleotides in length. A targeting domain may comprise a sequence length from about 100 nucleotides to about 1,000 nucleotides in length. A targeting domain may comprise a sequence length from about 200 nucleotides to about 1,000 nucleotides in length. A targeting domain may comprise a sequence length from about 500 nucleotides to about 1,000 nucleotides in length. A targeting domain may comprise a sequence length of at least about: 50, 60, 70, 80, 90, 100, 110, 120, 130, 140, 150, 160, 170, 180, 190, 200 nucleotides in length.

At least a portion of an engineered guide RNA (such as a targeting domain, a recruiting domain, or both) may comprise a secondary structure. A secondary structure may comprise a stem-loop, a cruciform, a toe hold, a mismatch bulge, more than one of any of these, or any combination thereof. A circular engineered guide RNA, although circular, may retain a substantially similar secondary structure as compared to a substantially similar engineered guide RNA that is not circular.

In some embodiments, the engineered polynucleotide can comprise or produce an antisense RNA sequence complementary to a target mRNA sequence to be modified except for a mismatch at the site of desired chemical modification of the target sequence. In some embodiments, the antisense RNA sequence can be circular. In another embodiment, the antisense RNA sequence can optionally comprise additional mismatches with respect to the target RNA sequence at position with hyper-editable adenosine nucleotides. In still another embodiment, the optional mismatches can comprise a “G” instead opposite an “A” in the target RNA sequence, while the targeted “A” in the target RNA is opposed by a mismatch “C”. In still another embodiment, a circular antisense guide RNA can comprise a mismatch at an adenosine to be chemically modified and a plurality of loops of 6-12 base pairs interspersed (e.g., −5 and +30 from the site to be modified and then every 15 bp 5′ and/or 3′ from the −5 and +30 loops). In still another embodiment, the circular antisense guide RNA comprises a plurality of interspersed loops that are created by positioning guanosine mismatches opposite hyperedited adenosines in the target RNA strand.

A guide RNA of the disclosure may not comprise (lacks) an end susceptible to hydrolytic degradation. In some cases, a guide RNA of the disclosure may comprise a secondary structure that is less susceptible to hydrolytic degradation than a mRNA naturally present in a cell. A guide RNA of the disclosure may not comprise (lacks) a reducing hydroxyl capable of being exposed to a solvent, such as a 5′ reducing hydroxyl or a 3′ hydroxyl. In some cases, a 5′ hydroxyl, a 3′ hydroxyl, or both, can be joined through a phosphorus-oxygen bond. In some embodiments, a 5′ hydroxyl, a 3′ hydroxyl, or both, can be modified into a phosphoester with a phosphorus-containing moiety. A guide RNA of the disclosure may not comprise (lacks) an exposed end. A guide RNA of the disclosure may not comprise (lacks) a 5′ end and a 3′ end. A guide RNA of the disclosure may retain a secondary structure—irrespective of whether the guide may be circular or not. For example, a circular guide RNA may comprise a secondary structure that is a stem loop, a cruciform, a toe hold, a mismatch bulge, more than one of any of these, or any combination thereof. A circular guide RNA may comprise a secondary structure that is substantially linear. A circular guide RNA may comprise a secondary structure that is modified to improve recruitment of an RNA editing entity or a secondary structure that partially mimics a native structure capable of recruiting an RNA editing entity.

In some cases, a circular guide RNA may comprise a half-life at least about: 2×, 3×, 4×, 5×, 6×, 7×, 8×, 9×, or 10× greater than a comparable guide RNA that is not circular. A half-life of a circular guide RNA may be from about 2× to about 5× greater than a comparable guide RNA that is not circular. A half-life of a circular guide RNA delivered to a cell or to a subject may be from about 3× to about 6× greater than a comparable guide RNA that is not circular.

A circular guide RNA delivered to a cell or to a subject may comprise a half-life in the cell or the subject of at least about: 40 minutes, 50 minutes, 60 minutes, 1.5 hours (hr), 2 hr, 4 hr, 6 hr, 8 hr, 10 hr, 12 hr, 16 hr, 18 hr, 20 hr, 24 hr, 1.25 days, 1.5 days, 2 days, 3 days, 4 days, 5 days, 6 days, 7 days, 10 days, 14 days, or more. A half-life of a circular guide RNA delivered to a cell or to a subject may be from about 1 hr to about 6 hrs. A half-life of a circular guide RNA delivered to a cell or to a subject may be from about 1 hr to about 24 hrs. A half-life of a circular guide RNA delivered to a cell or to a subject may be from about 1 hr to about 2 days. A half-life of a circular guide RNA delivered to a cell or to a subject may be from about 6 hr to about 24 hrs. A half-life of a circular guide RNA delivered to a cell or to a subject may be from about 6 hr to about 5 days.

In some embodiments, an engineered polynucleotide may comprise chirality. In some embodiments, any center atom, which can be chiral can be independently in the R or S configuration. In some cases, chiral may comprise an atom in a molecule that may be bonded to four different types of atoms or chains of atoms. In some instances, an engineered polynucleotide, such as a guide RNA may be a single diastereomer or may be predominantly one diastereomer. In some instances, an engineered polynucleotide may have a diastereomeric excess of from about: 510% to about 100%, 51% to about 60%, 60% to about 75%, 70% to about 90% or about 80% to about 99%. Diastereomeric excess can be a measurement of purity used for chiral substances. In some cases, it may reflect the degree to which a sample contains one diastereomer in greater amounts than another diastereomer. In some cases, a single pure diastereomer may have a diastereomeric excess of 100%. A sample with 70% of one diastereomer and 30% of the other may have a diastereomeric excess of 40% (70% ?30%).

An engineered guide RNA may comprise one or more modifications. In some cases, an engineered guide herein does not comprise a chemical modification. A modification may include a modified base. A modification may include a sugar modification, such as adding a glucose or other sugar-based moiety to one or more bases of the engineered guide RNA. A modification may include a protein coating over at least a portion of the engineered guide RNA. One or more nucleotides of an engineered guide RNA may comprise a methyl group, a fluoro group, a methoxyethyl group, an ethyl group, a phosphate group, an amide group, an ester group, or any combination thereof. A modification may increase stability or half-life of the engineered guide RNA as compared to a substantially similar engineered guide RNA without the modification.

Disclosed herein are methods for the circularization of guide RNAs. In some embodiments, an engineered guide RNA can be configured to undergo circularization in a cell. A construct for forming a circular RNA sequence may comprise a nucleotide sequence encoding for: (a) a guide RNA sequence for circularization comprising (i) an RNA editing entity recruiting domain, (ii) a ligation sequence, and (b) a ribozyme or catalytically active fragment thereof. In some cases, the nucleotide sequence may encode for two or more ligation sequences. In some cases, the nucleotide sequence may encode for two or more ribozymes. The two of more ligation sequences may be different. The two or more ribozymes may be different. A 5′ end, a 3′ end, or both of a guide RNA sequence may be flanked by a ligation sequence. A 5′ end or a 3′ end of a ligation sequence may be flanked by a ribozyme or catalytically active fragment thereof.

A construct for forming a circular RNA sequence may comprise a nucleotide sequence encoding for (a) an RNA sequence for circularization, (b) a ligation sequence, and (c) a tRNA, aptamer, or catalytically active fragment thereof. In some cases, the nucleotide sequence may encode for two ligation sequences. In some cases, the nucleotide sequence may encode for two self-cleaving entities (such as two tRNAs, two aptamers, or a combination). The nucleotide sequence may encode for two different ligation sequences. The nucleotide sequence may encode for two different self-cleaving entities, such as two different tRNAs, two different aptamers, or a combination. A 5′ end, a 3′ end, or both of a guide RNA sequence may be flanked by a ligation sequence. A 5′ end or a 3′ end of a ligation sequence may be flanked by a tRNA, aptamer, or other self-cleaving entity.

A circular RNA may be formed directly or indirectly by forming a linkage (such as a covalent linkage) between more than one end of the RNA sequence, such as a 5′ end and a 3′ end. The RNA sequence may comprise an engineered guide RNA (such as a recruiting domain, targeting domain, or both). The linkage may be formed by employing an enzyme, such as a ligase. In some cases, an enzyme can be a biologically active fragment of an enzyme. The enzyme may be recruited to the RNA sequence to form the linkage. A circular RNA may be formed by ligating more than one end of an RNA sequence using a linkage element. A linkage element may employ click chemistry to form the circular RNA. The linkage element may be an azide-based linkage. A circular RNA may be formed by genetically encoding or chemically synthesizing the circular RNA. A circular RNA may be formed by employing a self-cleaving entity, such as a ribozyme, tRNA, aptamer, catalytically active fragment of any of these, or any combination thereof. A self-cleaving ribozyme may comprise an RNase P.

In some embodiments, guides may be circular guides. For example, sequences having circular constructs can comprise elements of a P3 ribozyme, Alu element, antisense guide, target C mismatch, and/or a P1 ribozyme.

One or more methods may be employed to achieve forming a circular sequence (such as a circular RNA or circular DNA). A construct may encode for a sequence to make circular, such as a guide RNA sequence. The guide RNA may include a targeting domain and an RNA editing entity recruiting domain. The RNA editing entity recruiting domain may include an Alu domain, an APOBEC recruiting domain, a GluR2 domain, a Cas13 recruiting domain, or any combination thereof. The construct may encode for at least one ligation sequence, in some cases two ligation sequences. The construct may encode for at least one self-cleaving molecule, in some cases two self-cleaving molecules. The self-cleaving molecule may include a ribozyme, a tRNA, or any other self-cleaving molecule. In some cases, the self-cleaving molecule may be the tRNA. In some cases, at least one of: a 5′ end or a 3′ end of the sequence to make circular may be flanked by a ligation sequence, such as a sequence recognized by a ligase, such as an endogenous ligase. In some cases, at least one of: a 5′ end or a 3′ end of the ligation sequence may be flanked by the sequence encoding the self-cleaving molecule.

A suitable self-cleaving molecule may include a ribozyme. A ribozyme may include a RNase P, a rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, a HDV ribozyme, a Twister ribozyme, a Twister sister ribozyme, a VS ribozyme, a Pistol ribozyme, a Hatchet ribozyme, a viroid, or any combination thereof.

A suitable ligase (or synthetase) may include a ligase that forms a covalent bond. A covalent bond may include a carbon-oxygen bond, a carbon-sulfur bond, a carbon-nitrogen bond, a carbon-carbon bond, a phosphoric ester bond, or any combination thereof.

A pathway to construct a circular RNA sequence (such as a guide) may start with a tRNA splicing endonuclease binding to a specific recognition sequence and creating a 5′ hydroxyl group and 2′-3′ cyclic phosphate on cleaved ends. These cleaved ends may be ligated together by a ligase, such as an endogenous ligase (for example, a ubiquitously expressed RNA ligase RtcB). The advantage of employing this strategy may be the lack of additional enzymes required. The RNA transcripts may be expressed containing an RNA of interest flanked by a self-cleaving molecule, such as ribozymes. Addition of a sequence encoding a ribozyme may create an autocatalytic RNA. In some cases, the ribozymes may be P3 Twister and P1 Twister that may undergo spontaneous autocatalytic cleavage. The resulting RNA may contain the 5′ and 3′ ends that may then be ligated a ligase (such as ubiquitously expressed endogenous RNA ligase RtcB). Increasing the stability of the adRNA may have an impact on in vivo studies since editing depends on long term expression. The method may include forming a pre-strained circular adRNA (e.g., wherein the antisense region is part of a stable duplex and is unavailable to bind to a target).

In some cases, at least a portion of a recruiting domain can comprise at least about 80% sequence identify to an encoding sequence that recruits an ADAR, that recruits an APOBEC, or a combination thereof.

Compositions herein can be used to treat a disease or condition in a subject. For example, a viral vector comprising a precursor circular engineered guide RNA can be administered to treat a disease described herein. In some cases, once transcribed, the circular engineered guide RNA can be used to facilitate an edit of a target RNA sequence. In some instances, an edit can produce a full-length polypeptide or correct a missense mutation. In some cases, a composition described herein can be a pharmaceutical composition.

A pharmaceutical composition can comprise a first active ingredient. The first active ingredient can comprise a vector as described herein, or an engineered guide RNA. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient.

A composition described herein can compromise an excipient. In some cases, an excipient can comprise a pharmaceutically acceptable excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some cases, a carrier or a diluent can comprise an excipient. In some cases, a carrier or diluent can comprise a water, a salt solution (e.g., a saline), an alcohol or any combination thereof.

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some cases, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some cases, a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some cases, a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some cases, a pharmaceutical formulation can comprise a disintegrant as an excipient. In some cases, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some cases, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some cases, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some cases, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

A composition may comprise a combination of the active agent, e.g., a circular engineered guide RNA of this disclosure, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

In some embodiments, a pharmaceutical composition can be formulated in milligrams (mg), milligram per kilogram (mg/kg), copy number, or number of molecules. In some cases, a composition can comprise about 0.01 mg to about 2000 mg of the active agent. In some cases, a composition can comprise about: 0.01 mg, 0.1 mg, 1 mg, 10 mg, 100 mg, 500 mg, 1000 mg, 1500 mg, or about 2000 mg of the active agent.

In some cases, an engineered guide RNA delivered to a cell or to a subject may recruit an RNA editing entity, such as an endogenous RNA editing entity. In some cases, an engineered guide RNA may be co-delivered with an RNA editing entity. In some cases, circular guide RNAs may recruit a greater amount of an RNA editing entity as compared to a guide RNA that is not circular. In some cases, an engineered guide RNA may be configured to recruit a sufficient amount of an endogenous RNA editing entity to perform the editing, such as delivery of the engineered guide RNA to a tissue location that may be comprise a low amount of endogenous RNA editing enzymes. In some cases, an engineered guide RNA may be co-delivered with an RNA editing entity. In some cases, an RNA editing entity may be separately delivered to a cell or to a subject. In some cases, an engineered guide RNA may be associated with or directly linked to an RNA editing entity and the associated or directly linked composition may be delivered to a cell or to a subject.

A subject, host, individual, and patient may be used interchangeably herein to refer to any organism eukaryotic or prokaryotic. In some cases, subject may refer to an animal, such as a mammal. A mammal can be administered a vector, engineered guide RNA, cell or composition as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder. In other embodiments, a subject has or is suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

In some embodiments, method of treating a human in need thereof can comprise administering to the human a vector encoding a circular engineered guide RNA or a linear precursor thereof that comprises an antisense region with complementarity to a region of a target RNA sequence. In some instances, a target RNA sequence can comprise a transcript of ALDOA, DAXX, FANCC, CTNNB1, SMAD4, TARDBP, or IDUA. In some cases, the method can further comprise administering an RNA editing entity or a polynucleotide encoding an RNA editing entity to the human in need thereof. In some cases, human has or is suspected of having a disease or condition that comprises a Mucopolysaccharidosis type I (MPS I). In some cases, the disease or condition MPS I can comprise Hurler syndrome, Hurler-Scheie syndrome, Scheie syndrome, or any combination thereof. In some cases, the disease or condition can comprise Fanconi anemia, a colorectal cancer (CRC), a pilomatrixoma (PTR), a medulloblastoma (MDB), an ovarian cancer, a pilomatrixoma, a neurodevelopmental disorder, a hemorrhagic telangiectasia, a juvenile polyposis syndrome, Myhre syndrome, or an amyotrophic lateral sclerosis (ALS). In some cases, a neurodevelopmental disorder can comprise a neurodevelopmental disorder with spastic diplegia and visual defect.

In some embodiments, compositions herein can be used to treat disease and conditions. A disease or condition can comprise a neurodegenerative disease, a muscular disorder, a metabolic disorder, an ocular disorder, or any combination thereof. The disease or condition can comprise cystic fibrosis, albinism, alpha-1-antitrypsin deficiency, Alzheimer disease, Amyotrophic lateral sclerosis (ALS), Asthma, 0-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCA1 and 2 linked breast cancer and ovarian cancer). In some cases, a disease or condition can comprise Mucopoysaccharidosis type I (MPSI). In some cases, the MPSI can comprise Hurler syndrome, Hurler-Scheie syndrome, Scheie syndrome, or any combination thereof. The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof. Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition may comprise a muscular dystrophy. A muscular dystrophy may include myotonic, Duchenne, Becker, Limb-girdle, facioscapulohumeral, congenital, oculopharyngeal, distal, Emery-Dreifuss, or any combination thereof. A disease or condition may comprise pain, such as a chronic pain. Pain may include neuropathic pain, nociceptive pain, or a combination thereof. Nociceptive pain may include visceral pain, somatic pain, or a combination thereof.

A vector can be employed to deliver an engineered polynucleotide. A vector can comprise DNA, such as double stranded DNA or single stranded DNA. A vector can comprise RNA. In some cases, the RNA can comprise one or more base modifications. The vector can comprise a recombinant vector. In some cases, the vector can be a vector that is modified from a naturally occurring vector. The vector can comprise at least a portion of a non-naturally occurring vector. Any vector can be utilized. In some cases, the vector can comprise a viral vector, a liposome, a nanoparticle, an exosome, an extracellular vesicle, or any combination thereof. In some embodiments, plasmid vectors can be prepared from commercially available vectors. In other embodiments, viral vectors can be produced from baculoviruses, retroviruses, adenoviruses, AAVs, or a combination thereof. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves. Alphavirus vectors, such as Semliki Forest virus-based vectors and Sindbis virus-based vectors, have also been developed for use in gene therapy and immunotherapy. In aspects where gene transfer is mediated by a retroviral vector, a vector construct can refer to the polynucleotide comprising the retroviral genome or part thereof, and a gene of interest. In some cases, a vector can contain both a promoter and a cloning site into which a polynucleotide can be operatively linked. Such vectors are capable of transcribing RNA in vitro or in vivo and are commercially available. In some cases, a viral vector can comprise an adenoviral vector, an adeno-associated viral vector (AAV), a lentiviral vector, a retroviral vector, a portion of any of these, or any combination thereof. In some cases, a nanoparticle vector can comprise a polymeric-based nanoparticle, an aminolipid based nanoparticle, a metallic nanoparticle (such as gold-based nanoparticle), a portion of any of these, or any combination thereof. In some cases, a vector can comprise an AAV vector. A vector can be modified to include a modified VP1 protein (such as an AAV vector modified to include a VP1 protein). An AAV can comprise a serotype—such as an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, a derivative of any of these, or any combination thereof.

In some embodiments, a vector can comprise a nucleic acid that encodes a linear precursor of a circular engineered guide RNA. In some embodiments, a nucleic acid can comprise a linear precursor of a circular engineered guide RNA. In some cases, the nucleic acid can be double stranded. In some instances, the nucleic acid can be DNA or RNA. In some cases, a nucleic acid can comprise more than one copy of the precursor circular engineered guide RNA. For example, a nucleic acid can comprise 2, 3, 4, 5, or more copies of the precursor circular engineered guide RNA. In some instances, the nucleic acid can comprise a U6 promoter, a CMV promotor or any combination thereof.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant coding regions in the desired host cell are numerous and familiar to those skilled in the art. Virtually any promoter capable of driving these genetic elements is suitable for use in the disclosure. For example, a pol III promoter, a U6 promoter, a CMV promoter, a T7 promoter, an H1 promoter, can be used to drive expression. Termination control regions may also be derived from various genes native to the preferred hosts.

Administration of an engineered polynucleotide comprising a guide RNA can be effected in one dose, continuously or intermittently throughout the course of treatment. Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can vary and depend on the disease or condition. Routes of administration can vary with the composition used for treatment, the purpose of the treatment, the health condition or disease stage of the subject being treated, and target cell or tissue. Non-limiting examples of routes of administration include oral administration, nasal administration, injection, and topical application.

Administration can refer to methods that can be used to enable delivery of compounds or compositions to the desired site of biological action (such as DNA constructs, viral vectors, or others). These methods can include topical administration (such as a lotion, a cream, an ointment) to an external surface of a surface, such as a skin. These methods can include parenteral administration (including intravenous, subcutaneous, intrathecal, intraperitoneal, intramuscular, intravascular or infusion), oral administration, inhalation administration, intraduodenal administration, and rectal administration. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition.

Administration or application of a composition disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered or applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

An in vitro half-life of a circular RNA sequence may be at least about: 1×, 1.5×, 2×, 2.5×, 3×, 3.5×, 4×, 5×, 10×, 20× longer or more as compared to a substantially comparable linear RNA sequence. An in vivo half-life of a circular RNA sequence may be at least about: 1×, 1.5×, 2×, 2.5×, 3×, 3.5×, 4×, 5×, 10×, 20× longer or more as compared to a substantially comparable linear RNA sequence. A dosage of a composition comprising a circular RNA sequence administered to a subject in need thereof may be at least about: 1×, 1.5×, 2×, 2.5×, 3×, 3.5×, 4×, 5×, 10×, or 20× less as compared to a composition comprising a substantially comparable linear RNA sequence administered to the subject in need thereof. A composition comprising a circular RNA sequence administered to a subject in need thereof may be given as a single time treatment as compared to a composition comprising a substantially comparable linear RNA sequence given as a two-time treatment or more.

In some embodiments, a kit can comprise a guide RNA. In some instances, a kit can comprise an engineered circular polynucleotide, a precursor engineered circular guide RNA, a construct for forming a circular guide RNA sequence, a vector comprising an engineered polynucleotide, a nucleic acid of the engineered polynucleotide, a pharmaceutical composition and a container. In some cases, a container can be sterile. In some instances, a container can be plastic, glass, metal, or any combination thereof. In some cases, a kit can comprise instructions for use, such as instructions for administration to a subject in need thereof. In some embodiments, a method of making a kit can comprise adding a polynucleotide described herein into a container.

### EXAMPLES

Production of AAV vectors: AAV8 particles were produced using HEK293FT cells via the triple-transfection method and purified via an iodixanol gradient. Confluency at transfection was about 50%. Two hours before transfection, cell medium was exchanged with Dulbecco's modified Eagle's medium supplemented with 10% fetal bovine serum and 100× Antibiotic-Antimycotic (Gibco). All viruses were produced in 5×15 cm plates, where each plate was transfected with 10 μg of pXR-8, 10 μg of recombinant transfer vector and 10 μg of pHelper vector using polyethylenimine (PEI) (1 μg/μl linear PEI in ultrapure water, pH 7, using hydrochloric acid) at a PEI:DNA mass ratio of 4:1. The mixture was incubated for 10 minutes at room temperature and subsequently applied dropwise onto the cell media. The virus was harvested after 72 hours and purified using an iodixanol density gradient ultracentrifugation method. The virus was then dialyzed with 1× phosphate buffered saline (pH 7.2) supplemented with 50 mM sodium chloride and 0.0001% Pluronic F68 (Thermo Fisher) using 50 kDA filters (Millipore), to a final volume of ˜1 ml, and quantified by quantitative PCR using primers specific to the ITR region, against a standard (ATCC VR-1616): AAV-ITR-F, 5′-CGGCCTCAGTGAGCGA-3′ (SEQ ID NO:1542); AAV-ITR-R, 5′-GGAACCCCTAGTGATGGAGTT-3′ (SEQ ID NO:1543).

Animal experiments: All animal procedures were performed in accordance with protocols approved by the Institutional Animal Care and Use Committee of the University of California, San Diego. All mice were acquired from Jackson Labs. AAVs were injected retro-orbitally into both C57BL/6J mice and IDUA-W392X mice (B6.129S-Iduatml. 1Kmke/J), 6-8 weeks of age, at a dose of 1.0E13 vector genomes per mouse. Mice were monitored three times a week for the duration of the experiment (2 weeks).

Luciferase assay: HEK293FT cells were grown in DMEM supplemented with 10% FBS and 1% Antibiotic-Antimycotic (Thermo Fisher) in an incubator at 37° C. and 5% CO₂ atmosphere. All in vitro luciferase experiments were carried out in HEK293FT cells seeded in 96 well plates, at 25-30% confluency, using 200 ng total plasmid and 0.4 μl of commercial transfection reagent Lipofectamine 2000 (Thermo Fisher). Specifically, every well received 100 ng each of the Cluc-W85X (TAG) reporter and the adRNA plasmids. 48 hours post transfections, 20 μl of supernatant from cells was added to a Costar black 96 well plate (Corning). For the readout, 50 μl of Cypridina Glow Assay buffer was mixed with 0.5 μl Vargulin substrate (Thermo Fisher) and added to the 96 well plate in the dark. The luminescence was read within 10 minutes on Spectramax i3× or iD3 plate readers (Molecular Devices) with the following settings: 5 s mix before read, 5 s integration time, 1 mm read height.

Transfections: Unless otherwise stated, experiments were carried out in HEK293FT cells which were grown in DMEM supplemented with 10% FBS and 1% Antibiotic-Antimycotic (Thermo Fisher) in an incubator at 37° C. and 5% CO₂ atmosphere. HEK293FT cells were seeded in 24 well plates and transfected using 1000 ng adRNA plasmid or 48 pmol of IVT RNA and 2 ul of commercial transfection reagent Lipofectamine 2000 (Thermo Fisher). Cells were transfected at 25-30% confluence. Plasmid transfection experiments were harvested 48 hours post transfections while IVT RNA experiments were harvested 24 hours post transfections. For 96 hour long experiments, cells were passaged at a 1:4 ratio, 48 hours post transfections. Cells after plasmid electroporation were harvested at 48 hours, while IVT RNA experiments were harvested 24 hours post electroporation.

Electroporation: K562 cells were grown in RPMI supplemented with 10% FBS and 1% Antibiotic-Antimycotic (Thermo Fisher) in an incubator at 37° C. and 5% CO₂ atmosphere. 200,000 cells were electroporated with 1000 ng adRNA plasmid or 48 pmol of IVT RNA using the Amaxa SF cell Line 4D-Nucleofector X kit (Lonza) as per the manufacturer's instructions.

In vitro transcription: Sense RNA fragments and circular adRNA were made by in vitro transcription using the HiScribe T7 Quick High Yield RNA Synthesis Kit (NEB) as per the manufacturer's protocol. DNA templates for the IVT reaction carried the T7 promoter sequence at the 5′ end and were created by PCR amplification of the desired sequence from plasmids or cDNA. PCR products were purified using a PCR Purification Kit (Qiagen) and then used for IVT.

GAG assay: The GAG assay was performed briefly as follows: harvested mouse tissues were homogenized in 1 ml PBS with a syringe and 16 gauge (1.6 mm) needle. Tissue homogenates were then incubated on ice for 20 min with Triton X-100 added to a final concentration of 1%. Protein concentration in the supernatant clarified via centrifugation was estimated using the Bradford assay. Supernatants were digested in 1 mg/ml Proteinase K (Qiagen) for 12 h at 55° C. then boiled for 10 min to inactivate the enzyme. Nucleic acids were digested using Benzonase nuclease (Sigma) at 37° C. for 1 h followed by 10 min boiling to inactivate the enzyme. Total amount of GAG in each sample was measured using the Blyscan GAG assay kit (Biocolor).

RNA extraction and quantification of editing: RNA from cells was extracted using the RNeasy Mini Kit (Qiagen) while extraction from tissues was carried out using QIAzol Lysis Reagent and purified using RNeasy Plus Universal Mini Kit (Qiagen), according to the manufacturer's protocol. 500-1000 ng RNA was incubated with 1 μl of 5 μM of a target specific sense RNA (synthesized via IVT) at 95° C. for 3 minutes followed by 4° C. for 5 minutes. This step was carried out to capture the circular adRNA which if tightly bound to the target mRNA would block reverse transcription. cDNA was then synthesized using the Protoscript II First Strand cDNA synthesis Kit (NEB). 1 μl of cDNA was amplified by PCR with primers that amplify about 300-600 bp surrounding the sites of interest (outside the length of the antisense domain) using OneTaq PCR Mix (NEB). The numbers of cycles were tested to ensure that they fell within the linear phase of amplification. PCR products were purified using a PCR Purification Kit (Qiagen) and sent out for Sanger sequencing. The RNA editing efficiency was quantified using the ratio of peak heights G/(A+G). RNA-seq libraries were prepared from 250 ng of RNA, using the NEBNext Poly(A) mRNA magnetic isolation module and NEBNext Ultra II Directional RNA Library Prep Kit for Illumina. Samples were pooled and loaded on an Illumina Novaseq 6000 (100 bp paired-end run) to obtain 40-45 million reads per sample.

qPCRs: 1 μl of 1:4 diluted cDNA was used to set up a 10 μl qPCR reaction using iTaq Universal SYBR Supermix (Biorad). Primers were designed to keep the amplicon length within 300 bp. 2 technical replicates were carried out for each sample.

Extraction of nuclear and cytoplasmic RNA: 48 hours post transfections, cells were harvested and nuclear and cytoplasmic RNA fractions were extracted using the PARIS kit (Thermo Fisher) as per the manufacturer's protocol. The extracted RNA was treated with DNase and 100 ng was converted to cDNA using the Protoscript II First Strand cDNA synthesis Kit (NEB).

Mapping of RNA-seq reads: Sequence read pairs from stranded RNA-seq libraries were mapped to the reference human genome hg38 by running STAR aligner version 2.7.3a with the following command line options: --clip3pAdapterSeq AGATCGGAAGAGCACACGTCTGAACTCCAGTCA (SEQ ID NO:1544) AGATCGGAAGAGCGTCGTGTAGGGAAAGAGTGT (SEQ ID NO:1545) (to trim Illumina adapter sequences from the 3′ ends of the reads in each pair), --quantMode GeneCounts (to collect read counts for each gene), --alignSJDBoverhangMin 1 (following ENCODE standard practice), --peOverlapNbasesMin=10 --peOverlapMMp=0.05 (to correctly align pairs of overlapping reads), --outSAMmultNmax 1 (to limit output of multimapping reads), --alignEndsType EndToEnd (to avoid soft-clipping of reads), --outFilterMismatchNmax -1 --outFilterMismatchNoverReadLmax 0.2 --outFilterMultimapNmax 1 (to increase the likelihood of successful alignment for reads containing A-to-I editing events). The genome index for STAR aligner was built using transcript annotations from Gencode release 32 for the human genome assembly GRCh38. Each aligned read was retained for downstream analysis even when the corresponding mate in the pair could not be successfully aligned. Samtools version 1.10 was used to sort the aligned reads by genomic coordinate and to mark duplicated single or paired reads. The file ReadsPerGene.out.tab generated by STAR aligner contains three types of read counts for each gene: counts collected without considering read strands, counts based on the first strand of each read pair, and counts based on the second strand. The counts based on the first strand were found to be zero for most genes, while the counts based on the second strand were comparable to the unstranded counts, thus confirming that the sequence of first (second) read in each pair of the stranded RNA-seq libraries had the same orientation as the first (second) cDNA strand, as expected from the NEBNext Ultra II Directional RNA Library Prep Kit. The RNA-seq reads obtained from mice were processed as above, except for the following differences: the version of STAR aligner was 2.7.7a; the transcript annotations were from Gencode release M27 for the mouse genome assembly GRCm39; the version of samtools was 1.11.

Analysis of differential gene expression: RNA-seq libraries from mice were analyzed for differential gene expression using the Bioconductor package DESeq2 version 1.28.1. The per-gene counts of aligned read for each of four samples were collected by STAR aligner version 2.7.7a into a corresponding ReadsPerGene.out.tab file. The read counts corresponding to “the 2nd read strand aligned with RNA” were loaded for all samples into a DESeq2::DESeqDataSet object. Genes with less than 10 read counts in all samples were discarded. The counts for the remaining genes were processed using R function DESeq2::DESeq with default parameters. This function estimates size factors that account for differences in RNA-seq library size between the samples, estimates the dispersion parameters of the negative binomial distributions assumed for the read counts, fits generalized linear models (GLMs) to such counts, and calculates Wald statistics. The comparison between untreated and treated mice was carried out using R function DESeq2::results with default parameters, except that the significance cutoff for independent filtering optimization was set to 0.01. Shrinkage of effect sizes was carried out using R function DESeq2::lfcShrink with default parameters, thus employing the method of Approximate Posterior Estimator for GLM. GO analysis was performed using Enrichr.

Quantification of changes in RNA editing: To quantify significant changes in RNA editing, the BAM files containing reads aligned to the reference genome were processed as follows. Reads marked as duplicates were ignored. To minimize the bias of library size on statistical comparisons between different samples, the remaining reads from each sample were down-sampled, using samtools view with option -s, to the smallest number of such reads available for any sample. The down-sampling fraction used for each sample was calculated by dividing the smallest number of uniquely aligned reads among all samples by the number of uniquely aligned reads available for the sample being down-sampled. However, reads for the control sample, which was used for all comparisons, were not down-sampled.

The first step to quantify A-to-I editing events is to count the actual bases occurring on RNA transcripts at positions that, according to the reference genome, are expected to harbor an adenine base. Thus, for transcripts oriented as the forward (reverse) reference strand, base counts must be collected at reference A-sites (T-sites). As noted above, the first (second) read in each pair of the stranded RNA-seq libraries has the same orientation as the first (second) cDNA strand, the opposite (same) orientation as the transcript from which each cDNA molecule is synthesized. Also, the Illumina sequencing technology yields a pair of reads from opposite strands of the sequenced DNA molecule. Therefore, to handle transcripts oriented as the forward reference strand, base counts were collected at reference A-sites using the second (first) read in a pair, if that read was mapped to the forward (reverse) reference strand. Conversely, to handle transcripts oriented as the reverse reference strand, base counts were collected at reference T-sites using the first (second) read in a pair, if that read was mapped to the forward (reverse) reference strand.

The C library htslib (github.com/samtools/htslib), version 1.12 was used to enumerate the aligned reads that overlapped each base position in the reference genome. Reference sites covered by less than ten reads were ignored. The value of the SAM tag MD, “String for mismatching positions”, was recorded by samtools calmd, version 1.11, in each alignment record, and was used to determine the reference base at each position of an aligned sequence read. Base deletions and insertions relative to the reference genome were ignored. Sequenced bases with a Phred quality score less than 13 were ignored. For each sample, an initial list of base counts from reads overlapping each selected reference A- and T-site was generated.

The initial lists of base counts from all samples were then used to generate a final list of reference A- and T-sites where such base counts were available for all samples, and where at least one sample had a non-zero count of G (C) at reference A-sites (T-sites). The total number of reference sites in the final list was 1600217 and 1455241 for human and mice samples respectively.

At each selected reference site in the final list, a pairwise comparison between the base counts for each treatment sample and those for the control sample was carried out using Fisher's exact test, as implemented in R function fisher.test, with a 2-by-2 contingency table containing the counts of G (C) at reference A-sites (T-sites) in the first row, the counts of all other bases at those sites in the second row, the base counts for the control sample in the first column, and the base counts for the compared treatment sample in the second column. The resulting p-values were adjusted for multiple comparisons using the method of Benjamini and Hochberg, as implemented in R function p.adjust. The proportion of the number of G (C) bases relative to the number of all bases was also calculated at each A-site (T-site). Reference A-sites (T-sites) with a significant change in such base proportion for at least one comparison between a treatment sample and the control sample were selected by requiring an adjusted p-value less than 0.01 and a fold change greater than 1.1 in either direction. To visually compare each treatment sample with the control sample, 2D histograms of the observed base proportions at all reference A- and T-sites in the final list were generated using ggplot2. Note: The on-target editing efficiency values obtained in the RNA seq are highly inflated due to a large number of reads coming from the cadRNAs mapping onto the target and thus were omitted from the 2D histograms. Long-read deep sequencing or Sanger sequencing was instead utilized to measure on-target editing.

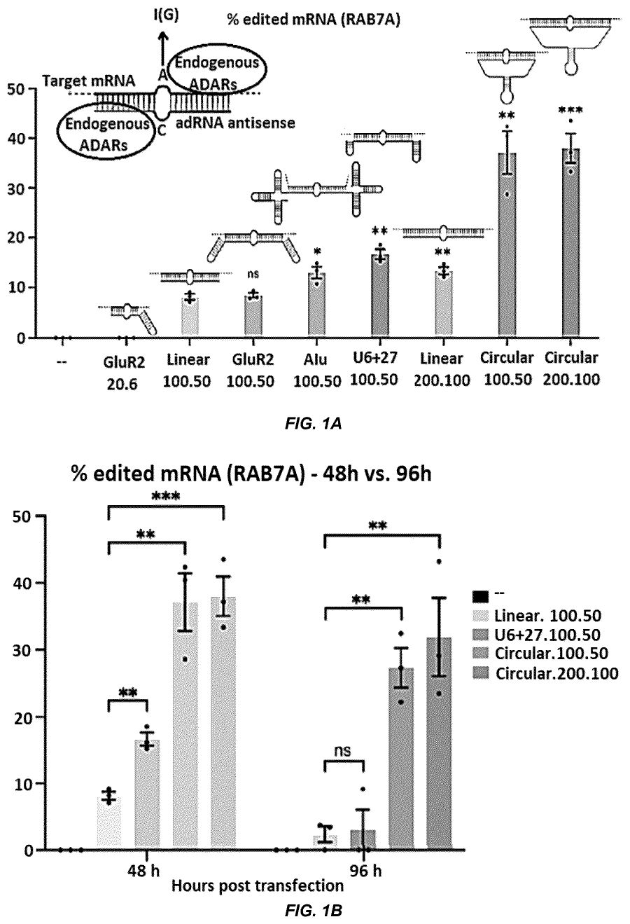

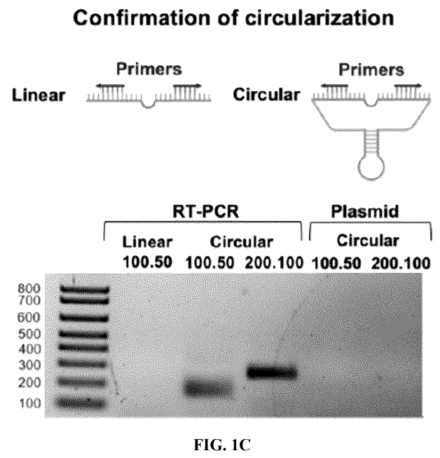

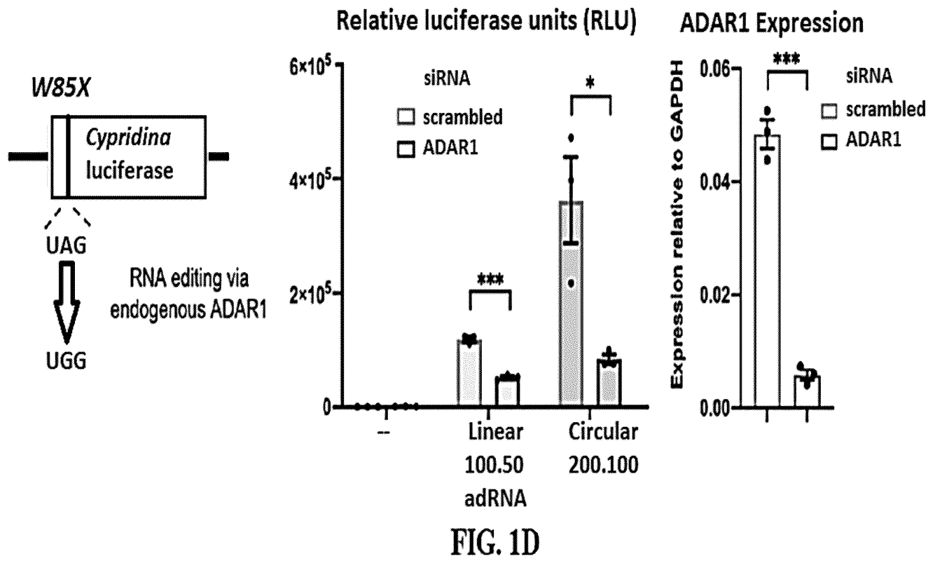

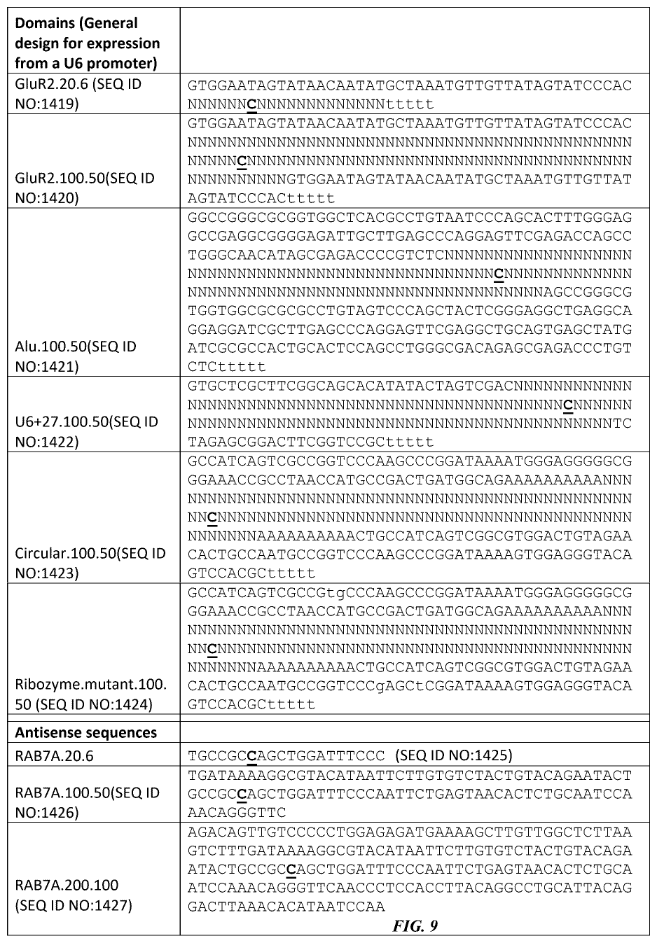

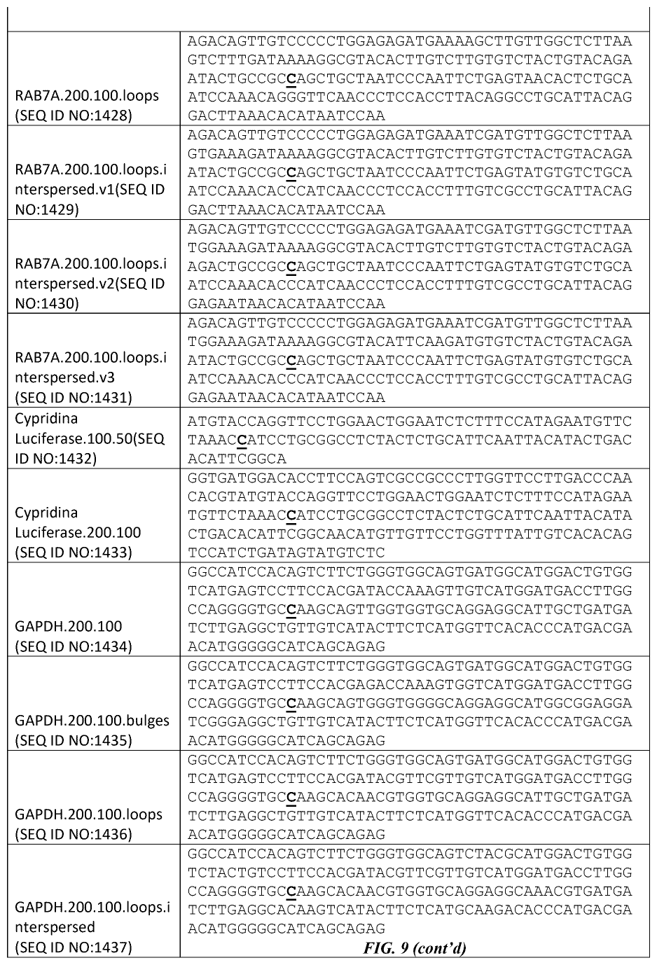

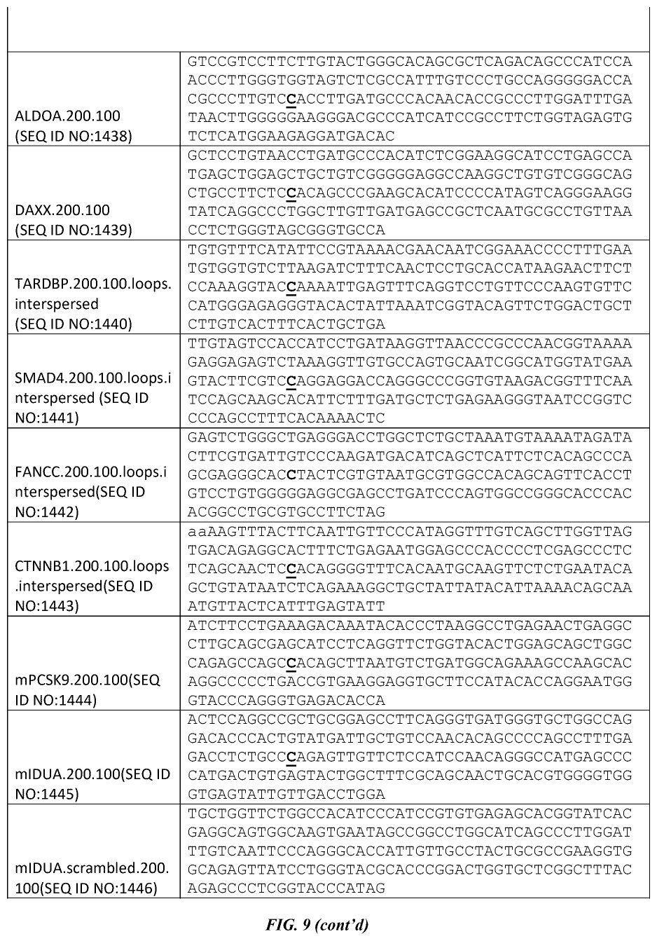

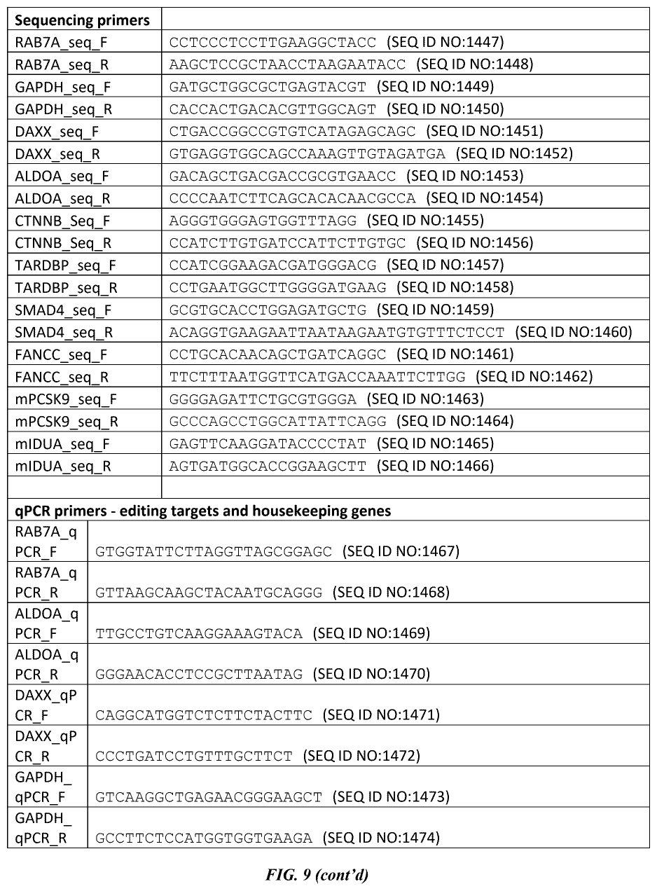

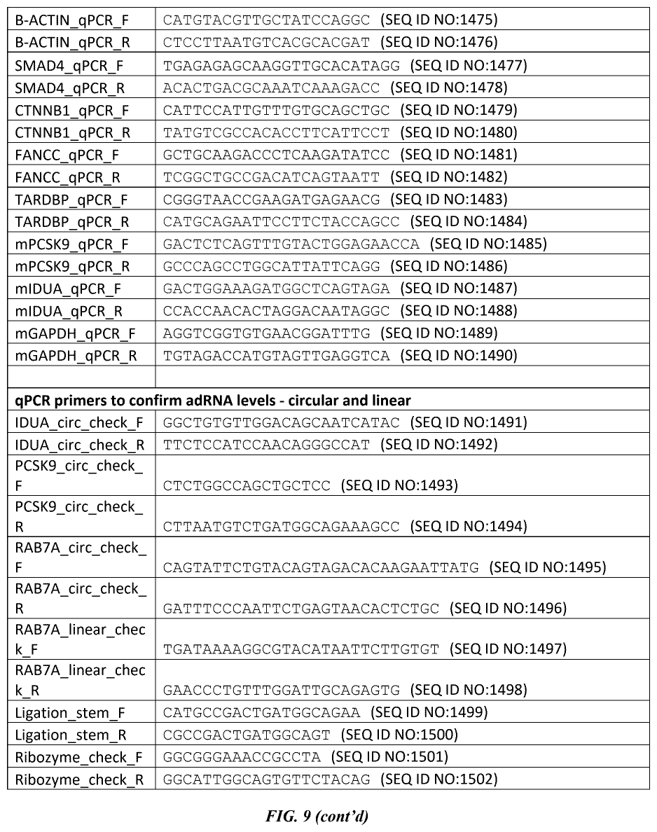

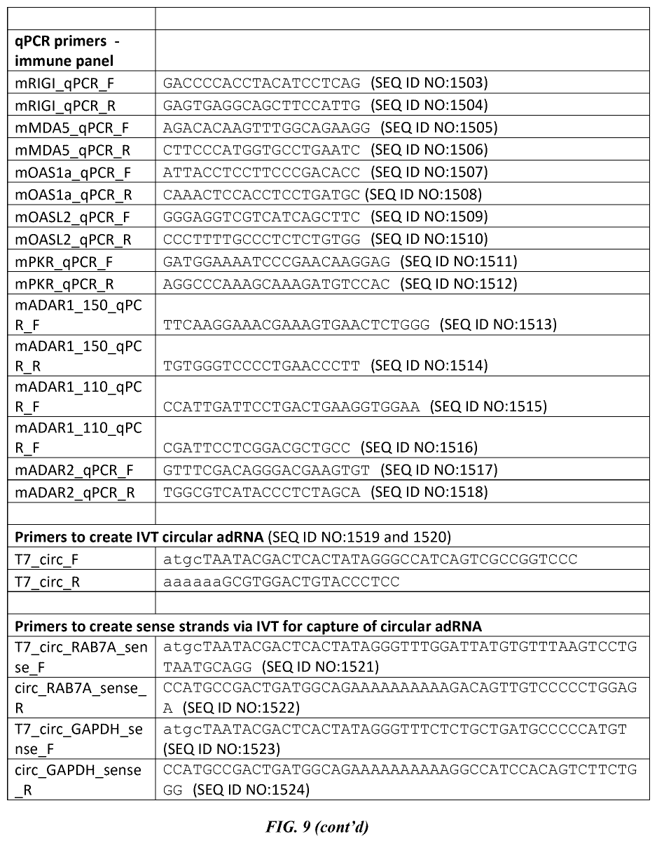

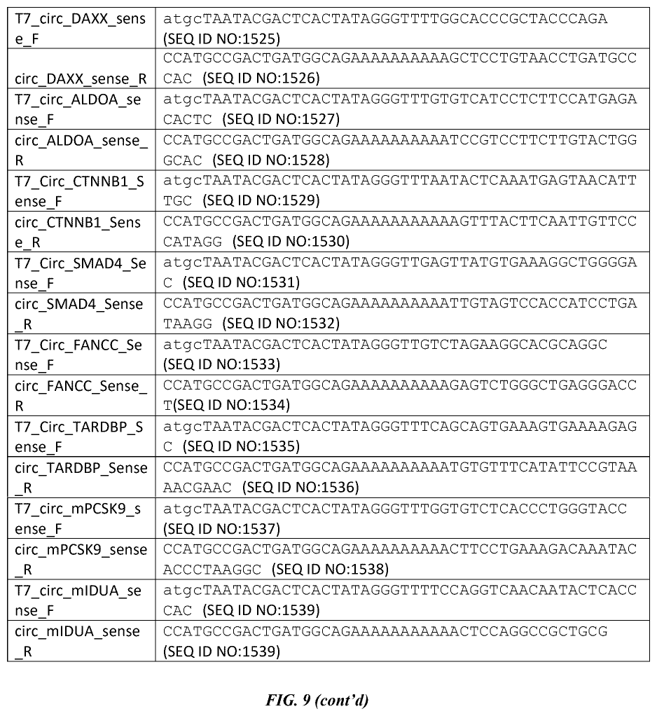

Using a long antisense guide RNA design that can recruit endogenous ADARs as a base format, two guide RNA engineering strategies were explored to enhance RNA editing efficiencies (FIG. 1A): one, recruiting domains were coupled that are derived from native RNAs sites that can be edited by ADARs; and two, domains were coupled that stabilize and confer increased half-life of the guide RNAs (FIG. 9).

Towards the former recruiting domains were evaluated from the naturally occurring ADAR2 substrate GluR2 pre-messenger RNA, and Alu elements, which can be substrates for ADAR1. The Alu adRNAs were created by positioning the antisense domain within the Alu consensus sequence and eliminating any poly U stretches. These modified guide RNAs were screened by assaying editing at an adenosine in the 3′UTR of the RAB7A transcript in HEK293FT cells. The GluR2 domain coupled to a short antisense of length 20 bp with the A-C mismatch located 6 bp from the 5′ end of the antisense domain (GluR2.20.6) was unable to recruit endogenous ADARs resulting in no detectable RNA editing, while long antisense RNAs with a centrally located A-C mismatch (linear.100.50) resulted in modest ˜10% RNA editing. Coupling the GluR2 domains to the long antisense version (GluR2.100.50) did not further enhance RNA editing yields, however the addition of Alu domains (Alu.100.50) marginally enhanced the efficiency of RNA editing (1.5-fold). While significant, these designs had only a modest improvement over the base format of simple long antisense guide RNAs.

Next an evaluation of the impact of persistence of guide RNAs was performed, as this in turn could also impact target RNA search as well as their net target residence times. In particular, genetically encoded adRNAs are typically expressed via the polymerase III promoter, and thus transcribed guides lack a 5′ cap and a 3′ poly-A tail and correspondingly have very short half-lives. To improve guide RNA persistence the following were evaluated: 1) increasing the length of the guide RNAs (linear.200.100); 2) coupling a U6+27 cassette (U6+27.100.50) which has been shown to improve stability of siRNA; and 3) engineering circular versions (circular.100.50 and circular.200.100) as these would be intrinsically resistant to cellular exonucleases. Specifically, circular ADAR recruiting guide RNAs (cadRNAs) were engineered by flanking the linear adRNAs by twister ribozymes, which upon autocatalytic cleavage leave termini that are ligated by the ubiquitous endogenous RNA ligase RtcB to yield circular guide RNAs. Comparing the three different guide designs both the increase of adRNA length and the addition of U6+27 to the long antisense adRNA led to a 1.5-fold and 2-fold respective improvement in editing of the RAB7A transcript over the linear.100.50 designs (FIG. 1A). Notably, using circular adRNA with antisense lengths 100 bp and 200 bp (e.g., circular.100.50 and circular.200.100), resulted in an even more robust 3.5-fold improvement in efficiency over the linear.100.50 designs and a 2-fold improvement over the Alu.100.50 and U6+27.100.50 designs (FIG. 1A). Persistence of significant levels in RNA editing was observed at both 48 hours and 96 hours post transfection via these constructs, while editing via linear guide RNAs was almost undetectable by the 96 hour time point (FIG. 1B). It was confirmed that U6 transcribed ribozyme flanked adRNAs were covalently circular in cells, forming cadRNAs, which were detected via RT-PCR by designing outward facing primers that selectively amplified only the circular structure (FIG. 1C).

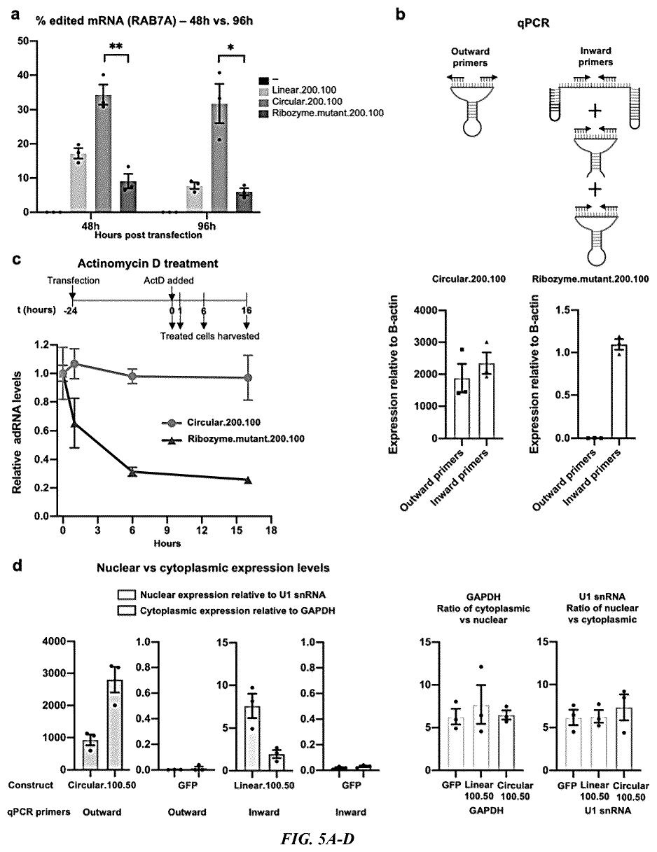

To confirm that circularization improved RNA editing (FIGS. 1A-B), the antisense sequence were flanked with catalytically inactive mutants of the twister ribozymes (ribozyme.mutant.200.100). This led to a significant decrease in RNA editing at both 48 and 96 hours post transfections with observed RNA editing levels similar to the linear versions (FIG. 5A). qPCR analysis confirmed the absence of circular adRNAs in cells transfected with ribozyme.mutant.200.100 (FIG. 5B). Additionally, in cells transfected with circular.200.100 plasmid, a significant fraction of the U6 transcribed adRNA was present in the circular form (FIG. 5B). To further ascertain that the long half-lives of the cadRNAs were responsible for persistent RNA editing observed, cells transfected with circular.200.100 and ribozyme.mutant.200.100 plasmids were treated with actinomycin D, a transcription inhibitor. Within 6 hours post-treatment a significant reduction in the amounts of the ribozyme.mutant.200.100 adRNA was observed while the levels of circular.200.100 adRNA remained constant (FIG. 5C). The intracellular localization of cadRNAs was evaluated and detected at high levels both in the nucleus and the cytoplasm (FIG. 5D).

Thus, it was confirmed that RNA editing via the circular guide RNAs, similar to the linear guide RNAs, was mediated by endogenous ADAR1 recruitment. Towards this, a luciferase based reporter assay was performed, where the guide RNAs were assayed for their ability to repair a premature stop codon (UAG) in the cypridina luciferase (cluc) transcript in the presence of scrambled and ADAR1 specific siRNAs. A significant drop-in luciferase activity was observed in the presence of ADAR1 siRNA, confirming that RNA editing via long antisense adRNAs and circular adRNAs was dependent on endogenous ADAR1 levels (FIG. 1D).

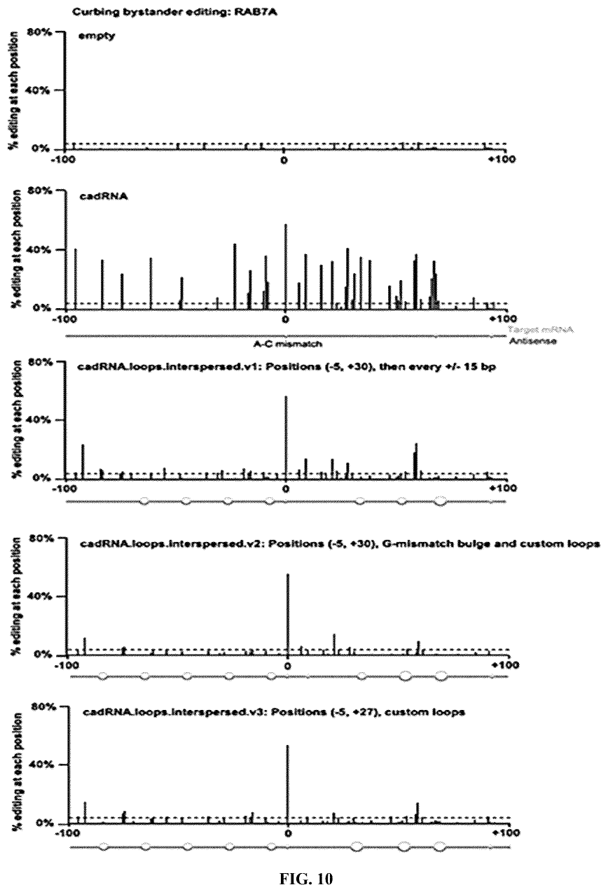

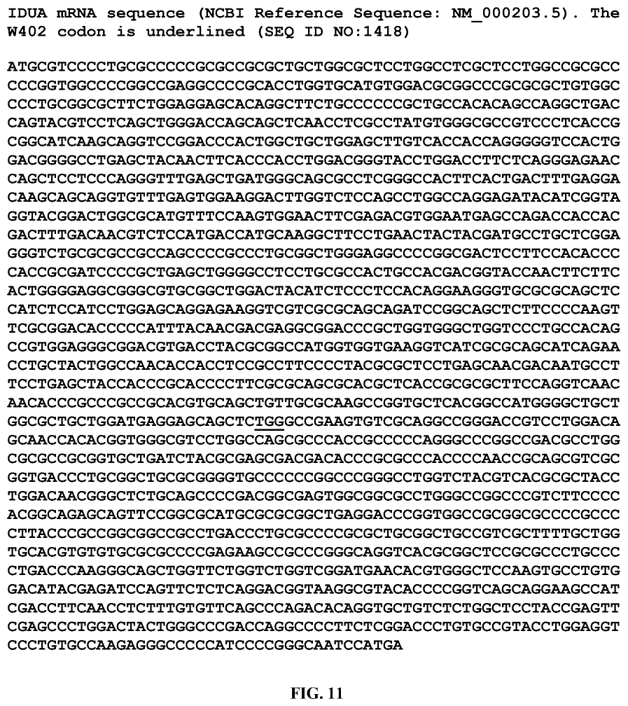

Experiments were then performed to evaluate the specificity profile of cadRNAs at both the transcriptome-wide and target transcript levels. Towards the former, a circular.100.50 and a circular.200.100 sample along with an untransfected HEK293FT sample were analyzed by deep RNA-seq. Notably, in contrast with enzyme overexpression where 103-104 transcriptome-wide off-targets are observed, a 2-3 orders of magnitude lower off-target editing via the cadRNAs was observed and at levels similar to the linear long antisense guide RNAs (FIG. 2A). Notably, over 80% of the adenosines detected as off-targets in these analyses were located in the RAB7A transcript itself which is indicative of bystander editing via cadRNA that was also confirmed via Sanger sequencing (FIG. 10). This is attributable to the long and perfectly paired dsRNA stretch created upon adRNA-target binding. By creating a G-mismatch opposite all non-target adenosines (cadRNA.bulges) this bystander editing was eliminated, however this also led to a significant drop in the on-target editing efficiency to about 50% of the unmodified circular.200.100 version (FIG. 2B-C). To address this, the antisense region was engineered to more closely mimic dsRNA structures of natural ADAR substrates. 8 bp loops were engineered and positioned both 5 bp upstream and 30 bp downstream of the target adenosine (cadRNA.loops). This design led to a significant reduction in bystander editing within the 36 bp region between the bulges, with the on-target editing being double that achieved by simply placing opposing G mismatches (FIG. 2B-C). However, significant bystander editing was still observed in the adenosines flanking the 36 bp region. It was hypothesized that it might be possible to eliminate these via positioning of 8 bp loops all along the antisense domain at intervals of 15 bp flanking the 36 bp central region that carries the target adenosine (cadRNA.loops.interspersed). Indeed, this new design significantly reduced bystander editing in the 200 bp dsRNA stretch formed between the target mRNA and the antisense domain, while maintaining on-target editing levels similar to the unmodified circular.200.100 construct (FIGS. 2B-C, and 12). Taken together, a combination of appropriately positioned 8-12 bp loops to create breaks within the long stretch of dsRNA, along with certain A-specific bulges can thus be utilized to eliminate bystander editing in a target specific manner (FIGS. 2B-C, and 12).

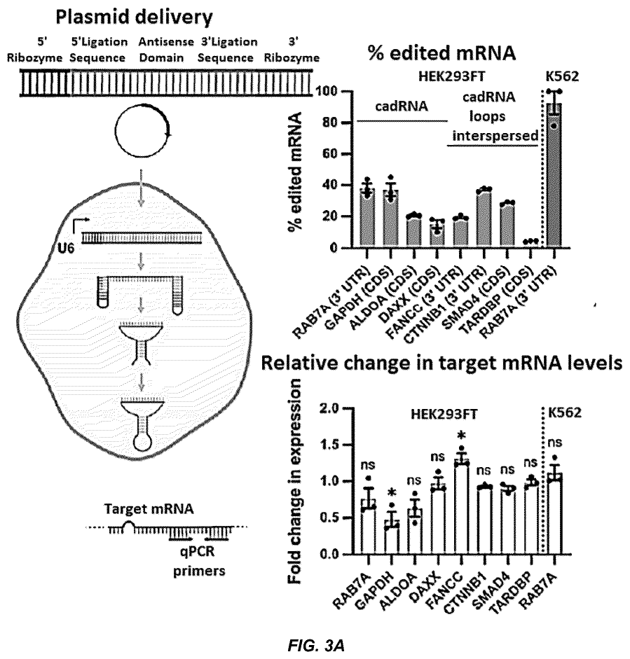

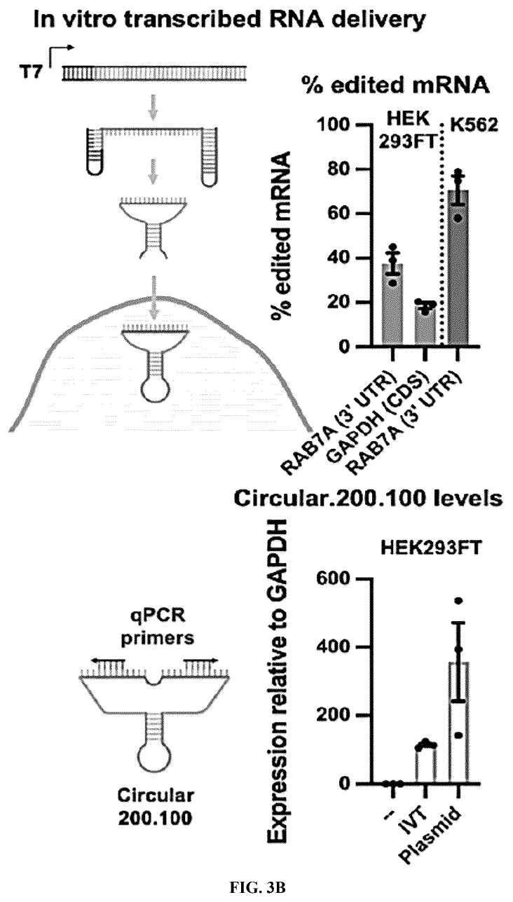

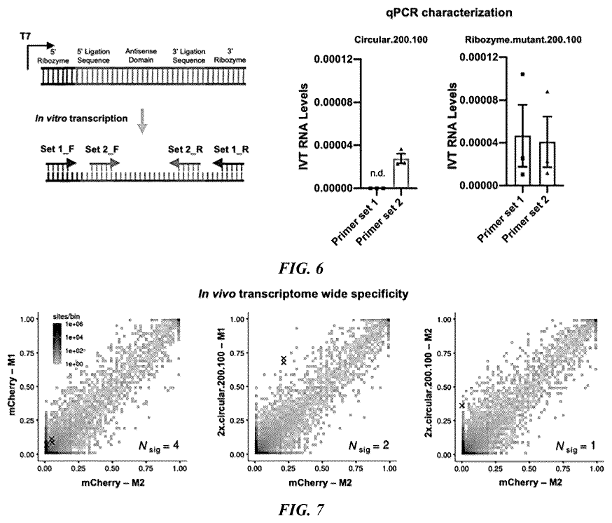

Next, the robustness and generalizability of the cadRNA format was confirmed via their ability to successfully edit adenosines in the 3′ UTR and coding sequence (CDS) of seven additional transcripts—GAPDH, ALDOA, DAXX, FANCC, CTNNB1, SMAD4 and TARDBP in HEK293FT cells (FIG. 3A). Furthermore, in addition to delivery via a genetically encoded format in plasmids, analysis of in vitro transcribed (IVT) circular adRNA was performed to determine if it also would be similarly functional. The ribozymes flanking the antisense domain were rapidly cleaved upon transcription and these cleaved products were then delivered to cells where they underwent in situ circularization in the cells (FIGS. 3B, and 6). 24 hours post transfection, editing of the RAB7A and GAPDH transcripts was observed using IVT adRNAs in HEK293FTs (FIG. 3A) and also confirmed circularization of the IVT adRNAs via qPCR. Additionally, the plasmid and IVT adRNAs based editing of RAB7A in K562 cells using electroporation was similarly robust at 90% and 70% RNA editing yields respectively (FIG. 3A, 3B). A majority of the tested loci did not show significant knockdown of the targeted transcripts via the cadRNAs (FIG. 3A).

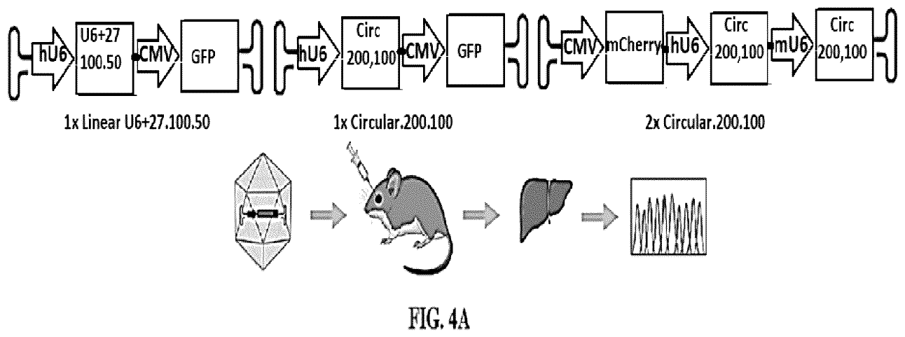

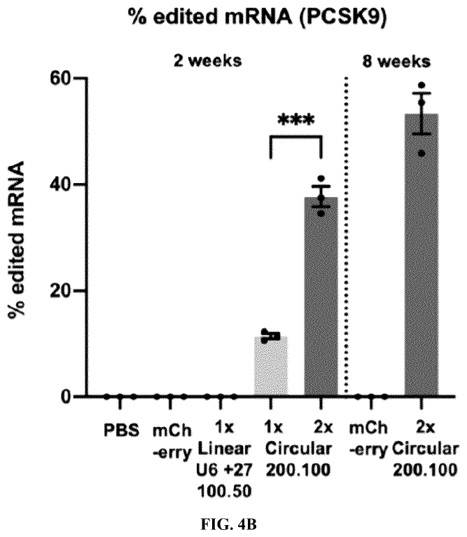

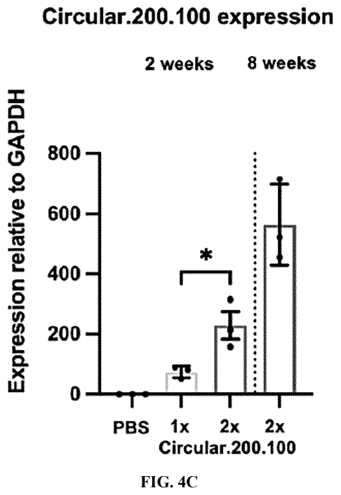

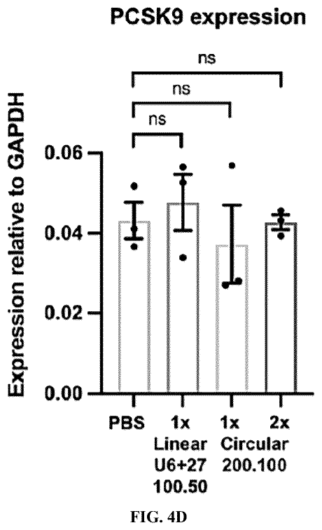

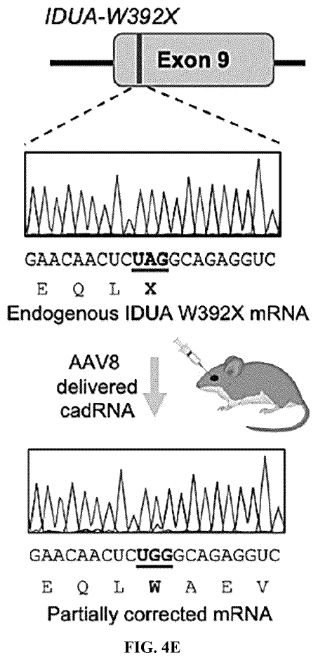

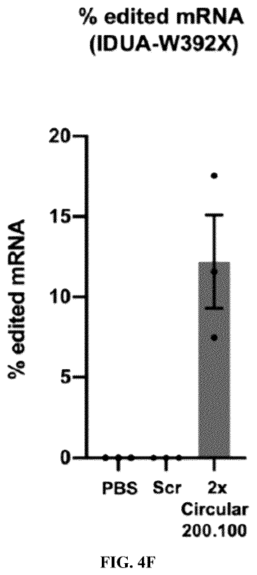

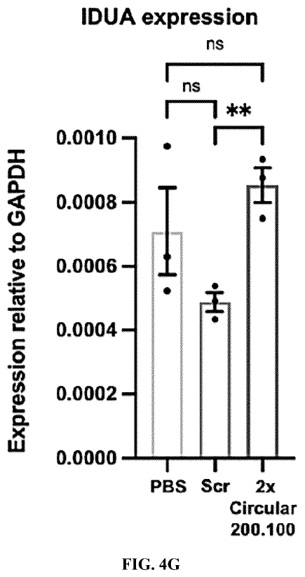

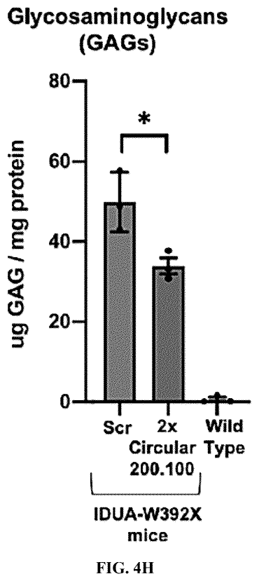

Given the vastly improved efficiency and durability of RNA editing via cadRNAs, experiments were performed to determine if these constructs could enable in vivo RNA editing. Since no co-delivery of proteins is required, successful demonstration here could enable a powerful gene therapy approach. Additionally, for the cadRNAs, one could leverage the already established delivery modalities and accruing knowledge from the field of shRNAs and ASOs that similarly only require delivery of nucleic acids to target tissues. To explore this, an adenosine in the 3′ UTR of the mPCSK9 transcript was targeted via AAV8 mediated delivery of adRNAs to the mouse liver. RNA editing yields were then systematically compared via linear.U6+27.100.50, one copy of circular.200.100, and two copies of circular.200.100 guide RNAs (FIG. 4A). 2 weeks post injections, mice livers were harvested, no editing was detected in the PBS injected mice, in mice injected with AAV8-mCherry, and notably in the mice injected with AAV8-linear.U6+27.100.50 guide RNAs no measureable RNA editing was detected (FIG. 4b). Highly efficient 11% and 38% on-target editing was observed via the AAV8 delivered single copy (1×) and two copy (2×) circular.200.100 guide RNAs, respectively. Additionally, editing via AAV8-2x.circular.200.100 was persistent, with mPCSK9 editing levels of 53% observed 8 weeks post injections. Robust expression of the cadRNAs was observed via qPCR, and the addition of a second copy of the circular.200.100 led to a 3-fold increase in expression levels, suggesting that persistent and robust guide RNA expression can enable efficient in vivo RNA editing (FIG. 3C). cadRNAs delivered via AAVs did not alter the expression levels of the mPCSK9 transcript in mice livers (FIG. 3D).

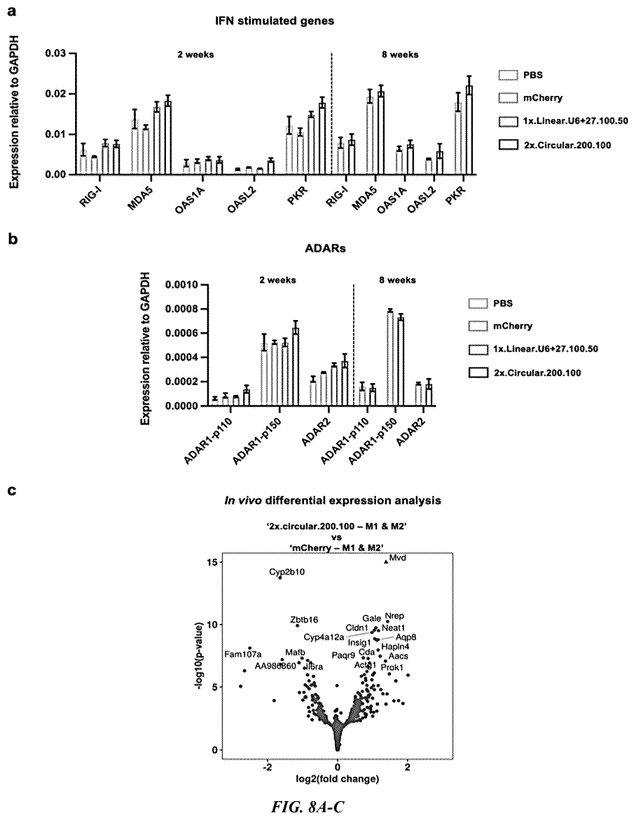

To evaluate the specificity profiles of the cadRNAs in vivo and also systematically study their effects on gene expression, RNA seq was performed on 4 C57BL6/J litter-mates, 2 injected with AAV8-mCherry and 2 with AAV8-2x.circular.200.100, 2 weeks post injections. Precise transcript-specific editing of the PCSK9 mRNA was observed in these mice (FIG. 7). Furthermore, qPCRs was carried out on several IFN-stimulated genes, especially those involved in sensing dsRNA such as RIG-I, MDA5, OAS1A, OSL, OASL2, PKR. In the short-term experiments, no significant changes were observed in the levels of many of these genes, but that there is an increase in the levels of MDA5 and PKR observed in the mice injected with AAV8-2x.circular.200.100 as compared to the AAV8-mCherry control group. However, in the long term experiments no significant changes were observed in the levels of any of these genes when compared to the AAV control group (FIG. 8A). Additionally, that presence of the cadRNAs did not significantly alter the expression of ADAR1-p110, ADAR1-p150 and ADAR2 as compared to the AAV control group (FIG. 8B). Differential expression analyses also confirmed no alterations in gene groups involved in sensing foreign RNA (FIG. 8C).

Building on these results, a mouse model of Hurler syndrome was used. Hurler syndrome is a form of mucopolysaccharidosis type 1 (MPS1), a rare genetic disorder that results in the buildup of large sugar molecules called glycosaminoglycans (GAGs) in lysosomes. This occurs due to a lack of the enzyme alpha-L-iduronidase which is encoded by the IDUA gene. W402X is a commonly occurring mutation in the IDUA gene in Hurler syndrome patients and there exists a corresponding mouse model bearing the IDUA-W392X mutation (FIG. 3E). With a goal to repair the IDUA-W392X premature stop codon, 2 copies of IDUA.circular.200.100 guide RNA was packaged into AAV8 and injected into IDUA-W392X mice systemically. As a control a AAV8-2x.scrambled.circular.200.100 was used. Two weeks post injection, mice livers were harvested and a robust 7-17% correction of the premature stop codon was observed in the mice injected with the AAV8-2x.IDUA.circular.200.100 adRNA (FIG. 3E-F). Expression of the circular.200.100 adRNA did not alter the expression levels of the IDUA transcript (FIG. 3G). GAG levels were also measured in these mice, and about 33% less GAG accumulation was measured in the treated animals over the 2-week period as compared to the scrambled control mice, indicating successful partial restoration of alpha-L-iduronidase activity (FIG. 3H).

While embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A circular engineered guide RNA comprising an antisense region with partial complementarity to a region of an IDUA target RNA sequence.

2-137. (canceled)

138. The circular engineered guide RNA of claim 1, wherein the IDUA target RNA sequence comprises the RNA sequence of SEQ ID NO: 1418 or SEQ ID NO: 1445.

139. The circular engineered guide RNA of claim 1, wherein the circular engineered guide RNA comprises at least 80% sequence identity to the reverse complement of SEQ ID NO: 1418, at least about 80% sequence identity to 50-200 nucleotides of SEQ ID NO: 1418 containing nucleotides 1204-1206, at least 80% sequence identity to the reverse complement of SEQ ID NO: 1445, or at least about 80% sequence identity to 50-200 nucleotides of SEQ ID NO: 1445.

140. The circular engineered guide RNA of claim 1, wherein the circular engineered guide RNA is configured to facilitate editing of a base of a target nucleotide in the IDUA target RNA sequence by an RNA editing entity, thereby generating an edited IDUA target RNA sequence.

141. The circular engineered guide RNA of claim 140, wherein a protein translated from the edited IDUA target RNA sequence is longer than a protein translated from an unedited IDUA target RNA sequence as demonstrated in an in vitro assay.

142. The circular engineered guide RNA of claim 1, wherein the antisense region comprises a sequence length from about 20 nucleotides to about 1,000 nucleotides in length.

143. The circular engineered guide RNA of claim 1, wherein the circular engineered guide RNA comprising an antisense region of about 100 bp or more has at least about: a 2-fold increase, a 3-fold increase, or a 3.5-fold increase in RNA editing as compared to a comparable linear engineered guide RNA as measured by an in vitro assay.

144. The circular engineered guide RNA of claim 1, wherein the circular engineered guide RNA comprises at least one 8-bp loop.

145. A nucleic acid encoding a linear precursor of the circular engineered guide RNA of claim 1.

146. The nucleic acid of claim 145, wherein the nucleic acid comprises two copies of the circular engineered guide RNA.

147. A vector comprising the nucleic acid of claim 145.

148. The vector of claim 147, wherein the vector is a viral vector.

149. The vector of claim 148, wherein the viral vector is an adeno-associated virus (AAV) vector comprising an AAV8 serotype, or a derivative thereof.

150. The vector of claim 148, wherein the viral vector is an AAV vector comprising an AAV1 serotype, an AAV2 serotype, AAV3 serotype, AAV4 serotype, AAV5 serotype, an AAV6 serotype, AAV7 serotype, an AAV9 serotype, a derivative of any of these, or any combination thereof.

151. A method of treating a human in need thereof comprising:
administering to the human a vector encoding a circular engineered guide RNA or linear precursor thereof that comprises an antisense region with partial complementarity to a region of an IDUA target RNA sequence.

152. The method of claim 151, wherein the human has or is suspected of having a disease or condition that comprises a Mucopolysaccharidosis type I (MPS I), preferably wherein the MPS I comprises Hurler syndrome, Hurler-Scheie syndrome, Scheie syndrome, or any combination thereof.

153. A recombinant RNA polynucleotide construct for editing a target RNA comprising
a) a 5′ ribozyme region;
b) a 5′ ligation sequence adjacent to the 5′ ribozyme region;
c) an antisense/targeting domain comprising an adenosine deaminase acting on RNA (ADAR) guide sequence that is used to edit an IDUA target RNA sequence, wherein the antisense/targeting domain is adjacent to the 5′ ligation sequence;
d) a 3′ ligation sequence that is adjacent to the antisense/targeting domain; and
e) a 3′ ribozyme region adjacent to the 3′ ligation sequence,
wherein the RNA construct recruits ADARs,
wherein the 5′ ribozyme and 3′ ribozyme regions upon autocatalytic cleavage leave termini that can be ligated together by an RNA ligase to yield a circular RNA construct,
wherein the antisense/targeting domain comprises a modification mismatch and a plurality of off-target-inhibitory mismatches, and
wherein the circular RNA construct is configured to facilitate editing of a base of a target nucleotide in the IDUA target RNA sequence by the ADAR, thereby generating an edited IDUA target RNA sequence.

154. The RNA construct of claim 153, wherein the IDUA target RNA sequence comprises an RNA sequence of SEQ ID NO: 1418 or SEQ ID NO: 1445.

155. The RNA construct of claim 153, wherein the antisense/targeting domain comprises at least 80% sequence identity to the reverse complement of SEQ ID NO: 1418 or at least about 80% sequence identity to 50-200 nucleotides of SEQ ID NO: 1418 containing nucleotides 1204-1206.

156. The RNA construct of claim 153, wherein the antisense/targeting domain comprises at least 80% sequence identity to the reverse complement of SEQ ID NO: 1445 or at least about 80% sequence identity to 50-200 nucleotides of SEQ ID NO: 1445.
